(19) United States
(12) Patent Application Publication
(10) Pub. No.: US 2005/0209274 A1
(43) Pub. Date: Sep. 22, 2005

(54) Antagonists of melanin concentrating hormone effects on the melanin concentrating hormone receptor

(76) Inventors: John K. Lynch, Kenosha, WI (US); Christine A. Collins, Skokie, IL (US); Jennifer C. Freeman, Grayslake, IL (US); Ju Gao, Gurnee, IL (US); Rajesh R. Iyengar, Lindenhurst, IL (US); Andrew S. Judd, Grayslake, IL (US); Philip R. Kym, Grayslake, IL (US); Mathew M. Mulhern, Hainesville, IL (US); Hing L. Sham, Vernon Hills, IL (US); Andrew J. Souers, Evanston, IL (US); Gang Zhao, Northbrook, IL (US); Dariusz Wodka, Plainsboro, NJ (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/065,918

(22) Filed: Feb. 25, 2005

Related U.S. Application Data
(60) Provisional application No. 60/547,968, filed on Feb. 26, 2004.

(51) Int. Cl. A61K 31/4709; A61K 31/452; C07D 45/02

(52) U.S. Cl. 514/312; 514/320; 546/196; 546/156; 546/153

(57) ABSTRACT

The present invention is directed to compounds of formula (I), 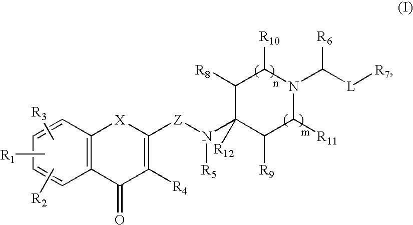 which antagonize of the effects of melanin-concentrating hormone (MCH) through the melanin concentrating hormone receptor which is useful for the prevention or treatment of eating disorders, weight gain, obesity, abnormalities in reproduction and sexual behavior, thyroid hormone secretion, diuresis and water/electrolyte homeostasis, sensory processing, memory, sleeping, arousal, anxiety, depression, seizures, neurodegeneration and psychiatric disorders.

This application claims priority from U.S. Provisional Patent Application Ser. No. 60/547,968, filed Feb. 26, 2004, incorporated herein by reference.

### TECHNICAL FIELD

The present invention relates to the antagonism of the effects of melanin-concentrating hormone (MCH) through the melanin concentrating hormone receptor which is useful for the prevention or treatment of eating disorders, weight gain, obesity, abnormalities in reproduction and sexual behavior, thyroid hormone secretion, diuresis and water/electrolyte homeostasis, sensory processing, memory, sleeping, arousal, anxiety, depression, seizures, neurodegeneration and psychiatric disorders.

### BACKGROUND OF THE INVENTION

Obesity is a major cause and contributor to health problems such as type II diabetes, coronary heart disease, increased incidence of certain forms of cancer, and respiratory complications. It is a disease that is increasing at an alarming rate due to increased availability of high-fat diets, genetic susceptibility, and a more sedentary way of life in modern society. Obesity can be defined as weight gain resulting from a mismatch of energy intake and energy expenditure. Food intake and energy metabolism are regulated, in part, by the interaction of neuropeptides and their receptors. Recently, the role that the hormone leptin plays in controlling appetite has been elucidated.

Leptin is a peptide hormone produced by fat cells, regulating both food intake and and metabolism by acting on leptin receptors in the hypothalamus. Increased fat stores leads to increased secretion of leptin, resulting in a signal to the hypothalamus to decrease food intake, whereas decreases in adiposity result in lower leptin levels and a stimulation of food intake. Melanin-concentrating hormone (MCH) has been identified as an orexigenic peptide that counterbalances the activity of leptin.

MCH is a cyclic 19 amino acid neuropeptide expressed in the zona incerta and lateral hypothalamus in response to both energy restriction and leptin deficiency. MCH is known to stimulate feeding when injected into the lateral ventricle of rats and the mRNA for MCH is upregulated in the hypothalamus of genetically obese mice (ob/ob) and in fasted control animals. Mice lacking MCH are hypophagic and lean with increased metabolic rate, whereas animals over-expressing MCH gain excess weight on both standard and high fat diets. MCH is thought to have effects on other nervous system functions as well (Nahon J L., The melanin-concentrating hormone: from the peptide to the gene. Crit Rev Neurobiol 8:221-262, 1994). An orphan G-protein coupled receptor (GPCR) was recently identified as a receptor for MCH.

Although there exists pharmacologic therapies used to treat obesity, none of the current therapies achieve the U.S. Food and Drug Administration criteria for benefit measured by a 5% difference in mean weight loss, as weight loss efficacy is diminished by reduction of patient adherence to pharmacological therapy due to side effects of the drugs. Some of the side effects associated with current therapies include increased heart rate and blood pressure, and uncontrolled excretion of fat in stools. Thus, there exists a medical need for agents capable of preventing or treating eating disorders, weight gain and obesity, that at the same time, have improved efficacy and safety.

Therefore, current investigation of novel MCH antagonists which may lead to an orally active new and better treatment of eating disorders, weight gain and obesity would be of great importance to current society.

### SUMMARY OF THE INVENTION

The present invention is directed to compounds of formula (I), 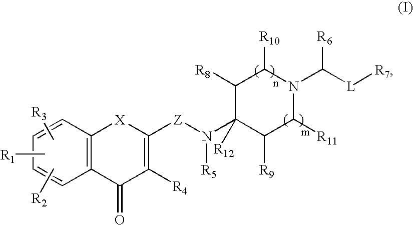
or a therapeutically acceptable salt or prodrug thereof, wherein
L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—;
X is selected from the group consisting of —O— and —N(R₁₃)—;
Z is selected from the group consisting of —CH₂—, —C(N—R_{c})—, —C(O)— and —C(S)—;
m is 1 or 2;
n is 0, 1, or 2
R₁, R₂ and R₃ are each independently selected from the group consisting of hydrogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, halogen, haloalkyl, haloalkoxy, haloalkylthio, nitro, RₐR_{b}N—, RₐR_{b}NC(O)— or R₁ and R₂ taken together with any intervening atoms form a ring selected from the group consisting of heteroaryl and heterocycle;
R₄ is selected from the group consisting of hydrogen, alkyl, alkylcarbonyl-NH—, alkylsulfonyl-NH—, aryl and halogen;
R₅ is selected from the group consisting of hydrogen and alkyl;
R₆ is selected from the group consisting of hydrogen, alkyl, aryl, cycloalkyl, heteroaryl and heterocycle;
R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle;
R₈ is selected from the group consisting of hydrogen, alkyl and alkoxy;
R₉ is selected from the group consisting of hydrogen and alkyl;
R₁₀ and R₁₁ are independently selected from the group consisting of hydrogen, alkyl, alkoxylalkyl, or R₁₀ and R₁₁ taken together with any intervening atoms form a 5, 6, or 7-membered ring;
R₁₂ is selected from the group consisting of hydrogen and alkyl;
R₁₃ is selected from the group consisting of hydrogen, alkyl, aryl, cycloalkyl, heteroaryl and heterocycle;
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; and
R_{c} is selected from the group consisting of hydrogen and alkyl;
provided that at least one of R₁, R₂ or R₃ are not hydrogen.

Another embodiment of the present invention encompasses the use of the compounds of formula (I) for the treatment of obesity comprising administration of said compounds to a patient in need of such treatment.

A further embodiment of the present invention encompasses the use of the compounds of formula (I) for the treatment of disorders that are mediated by MCH through the MCH receptor such as abnormalities in reproduction and sexual behavior, thyroid hormone secretion, diuresis and water/electrolyte homeostasis, sensory processing, memory, sleeping and arousal, anxiety and depression, seizure and in treatment of neurodegeneration or psychiatric disorders comprising administering a therapeutically effective amount of a compound of formula (I) to a patient in need thereof.

According to another embodiment, the present invention is directed to a pharmaceutical composition comprising a therapeutically effective amount of a compound of formula (I) in combination with a pharmaceutically suitable carrier.

### DETAILED DESCRIPTION OF THE INVENTION

The principal embodiment of the present invention is directed toward compounds of formula (I) and their use in the treatment of disorders mediated by MCH comprising administration of a therapeutically effective amount of a compound of formula (I) to one in need of such treatment.

Therefore, the principal embodiment of the present invention is directed to compounds of formula (I), 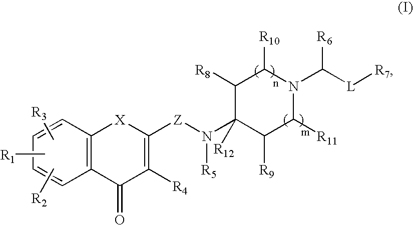
or a therapeutically acceptable salt or prodrug thereof, wherein L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—; X is selected from the group consisting of —O— and —N(R₁₃)—; Z is selected from the group consisting of —CH₂—, —C(N—R_{c})—, —C(O)— and —C(S)—; m is 1 or 2; n is 0, 1, or 2; R₁, R₂ and R₃ are each independently selected from the group consisting of hydrogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, halogen, haloalkyl, haloalkoxy, haloalkylthio, nitro, RₐR_{b}N—, RₐR_{b}NC(O)— or R₁ and R₂ taken together with any intervening atoms form a ring selected from the group consisting of heteroaryl and heterocycle; R₄ is selected from the group consisting of hydrogen, alkyl, alkylcarbonyl-NH—, alkylsulfonyl-NH—, aryl and halogen; R₅ is selected from the group consisting of hydrogen and alkyl; R₆ is selected from the group consisting of hydrogen, alkyl, aryl, cycloalkyl, heteroaryl and heterocycle; R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle; R₈ is selected from the group consisting of hydrogen, alkyl and alkoxy; R₉ is selected from the group consisting of hydrogen and alky; R₁₀ and R₁₁ are independently selected from the group consisting of hydrogen, alkyl, alkoxylalkyl, or R₁₀ and R₁₁ taken together with any intervening atoms form a 5, 6, or 7-membered ring; R₁₂ is selected from the group consisting of hydrogen and alkyl; R₁₃ is selected from the group consisting of hydrogen, alkyl, aryl, cycloalkyl, heteroaryl and heterocycle; Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; and R_{c} is selected from the group consisting of hydrogen and alkyl; provided that at least one of R₁, R₂ or R₃ are not hydrogen.

Another embodiment of the present invention is directed toward a compound of formula (II), wherein 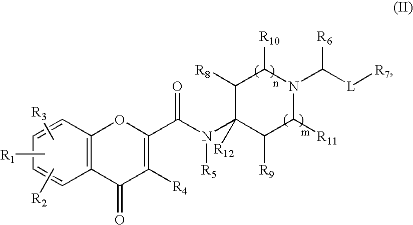
or a therapeutically acceptable salt or prodrug thereof, wherein L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—; m is 1 or 2; n is 0, 1, or 2; R₁, R₂ and R₃ are each independently selected from the group consisting of hydrogen, halogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N—, RₐR_{b}NC(O)— or R₁ and R₂ taken together with any intervening atoms form a heterocycle; R₄ is selected from the group consisting of hydrogen, alkyl, alkylcarbonyl-NH—, alkylsulfonyl-NH—, aryl and halogen; R₅ is selected from the group consisting of hydrogen and alkyl; R₆ is selected from the group consisting of hydrogen, alkyl, aryl, cycloalkyl, heteroaryl and heterocycle; R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle; R₈ is selected from the group consisting of hydrogen, alkyl and alkoxy; R₉ is selected from the group consisting of hydrogen and alky; R₁₀ and R₁₁ are each independently selected from the group consisting of hydrogen, alkyl, alkoxylalkyl, or R₁₀ and R₁₁ taken together with any intervening atoms form a 5, 6, or 7-membered ring; R₁₂ is selected from the group consisting of hydrogen and alky; and Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attacked form a ring selected from the group consisting of heteroaryl and heterocycle; provided that at least one of R₁, R₂ or R₃ are not hydrogen.

Another embodiment of the present invention is directed toward a compound of formula (II), or a therapeutically acceptable salt or prodrug thereof, wherein L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—; m is 1;
n is 1; R₁, R₂ and R₃ are each independently selected from the group consisting of hydrogen, halogen, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N—, RₐR_{b}NC(O)— or R₁ and R₂ taken together with any intervening atoms form a heterocycle; R₄ is selected from the group consisting of hydrogen, alkyl, alkylcarbonyl-NH—, alkylsulfonyl-NH—, aryl and halogen; R₅ is selected from the group consisting of hydrogen and alkyl; R₆ is selected from the group consisting of hydrogen and alkyl; R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle; R₈ is selected from the group consisting of hydrogen, alkyl and alkoxy; R₉ is selected from the group consisting of hydrogen and alkyl; R₁₀ and R₁₁ are each independently selected from the group consisting of hydrogen, alkyl and alkoxylalkyl, or R₁₀ and R₁₁ taken together with any intervening atoms form a 5, 6, or 7-membered ring; R₁₂ is hydrogen; and Rₐ and R_{b} are each individually selected from the group consisting of hydrogen, alkoxycarbonyl, alkyl, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

A further embodiment of the present invention is directed toward a compound of formula (II), or a therapeutically acceptable salt or prodrug thereof, wherein L is a bond; m is 1; n is 1; R₁, R₂ and R₃ are each independently selected from the group consisting of hydrogen, halogen, alkoxy, alkyl, cyano, haloalkyl, haloalkoxy nitro, RₐR_{b}N— and RₐR_{b}NC(O)—; R₄ is selected from the group consisting of hydrogen, alkyl, alkylcarbonyl-NH—, alkylsulfonyl-NH—, aryl and halogen; R₅ is hydrogen; R₆ is selected from the group consisting of hydrogen and alkyl; R₇ is selected from the group consisting of aryl, heteroaryl and heterocycle, or R₆ and R₇ together with the atoms to which they are attached form cycloalkyl; R₈ is hydrogen; R₉ is selected from the group consisting of hydrogen and alkyl; R₁₀ and R₁₁, are hydrogen; R₁₂ is hydrogen; and Rₐ and R_{b} are each individually selected from the group consisting of hydrogen, alkoxycarbonyl, alkyl, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

A further embodiment of the present invention is directed toward a compound of formula (II), or a therapeutically acceptable salt or prodrug thereof, wherein L is a bond; m is 1; n is 1; R₁, R₂ and R₃ are each independently selected from the group consisting of hydrogen, halogen, alkoxy, alkyl, cyano, haloalkyl, haloalkoxy nitro, RₐR_{b}N— and RₐR_{b}NC(O)—; R₄ is selected from the group consisting of hydrogen, alkyl, alkylcarbonyl-NH—, alkylsulfonyl-NH—, aryl and halogen; R₅ is hydrogen; R₆ is selected from the group consisting of hydrogen and alkyl; R₇ is selected from the group consisting of aryl, heteroaryl and heterocycle; R₈ is hydrogen; R₉ is selected from the group consisting of hydrogen and alkyl; R₁₀ and R₁₁ are hydrogen; R₁₂ is hydrogen; and Rₐ and R_{b} are each individually selected from the group consisting of hydrogen, alkoxycarbonyl, alkyl, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

A further embodiment of the present invention is directed toward a compound of formula (II), or a therapeutically acceptable salt or prodrug thereof, wherein L is a bond; m is 1; n is 1; R₁, R₂ and R₃ are each independently selected from the group consisting of hydrogen, halogen, alkoxy, alkyl, cyano, haloalkyl, haloalkoxy nitro, RₐR_{b}N— and RₐR_{b}NC(O)—; R₄ is selected from the group consisting of hydrogen, alkyl, alkylcarbonyl-NH—, alkylsulfonyl-NH—, aryl and halogen; R₅ is hydrogen; R₆ and R₇ together with the atoms to which they are attached form cycloalkyl; R₈ is hydrogen; R₉ is selected from the group consisting of hydrogen and alkyl; R₁₀ and R₁₁ are hydrogen; R₁₂ is hydrogen; and Rₐ and R_{b} are each individually selected from the group consisting of hydrogen, alkoxycarbonyl, alkyl, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

Accordingly, another embodiment of the present invention is directed toward a compound of formula (III), 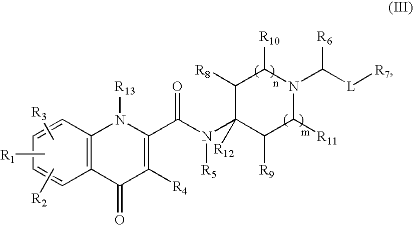
or a therapeutically acceptable salt or prodrug thereof, wherein L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—; m is 1 or 2; n is 0, 1, or 2; R₁, R₂ and R₃ are each independently selected from the group consisting of hydrogen, halogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N— and RₐR_{b}NC(O)— or R₁ and R₂ taken together with any intervening atoms form a ring selected from the group consisting of heteroaryl and heterocycle; R₄ is selected from the group consisting of hydrogen, alkyl, alkylcarbonyl-NH—, alkylsulfonyl-NH—, aryl and halogen; R₅ is selected from the group consisting of hydrogen and alkyl; R₆ is selected from the group consisting of hydrogen, alkyl, aryl, cycloalkyl, heteroaryl and heterocycle; R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle; R₈ is selected from the group consisting of hydrogen, alkyl and alkoxy; R₉ is selected from the group consisting of hydrogen and alkyl; R₁₀ and R₁₁ are each independently selected from the group consisting of hydrogen, alkyl and alkoxylalkyl, or R₁₀ and R₁₁ taken together with any intervening atoms form a 5, 6, or 7-membered ring; R₁₂ is selected from the group consisting of hydrogen and alkyl; R₁₃ is selected from the group consisting of hydrogen, alkyl, aryl, cycloalkyl, heteroaryl and heterocycle; and Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

Another embodiment of the present invention is directed toward a compound of formula (III), or a therapeutically acceptable salt or prodrug thereof, wherein L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—; m is 1; n is 1; R₁, R₂ and R₃ are each independently selected from the group consisting of hydrogen, halogen, alkoxy, alkyl, alkylcarbonyl, alkylcarbonyl-NH—, alkyl-NH-carbonyl, alkyl-S(O)₂—NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N— and RₐR_{b}NC(O)— or R₁ and R₂ taken together with any intervening atoms form a ring selected from the group consisting of heteroaryl and heterocycle; R₄ is selected from the group consisting of hydrogen, alkyl, alkylcarbonyl-NH—, alkylsulfonyl-NH—, aryl and halogen; R₅ is selected from the group consisting of hydrogen and alkyl; R₆ is selected from the group consisting of hydrogen and alkyl; R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle; R₈ is selected from the group consisting of hydrogen, alkyl and alkoxy; R₉ is selected from the group consisting of hydrogen and alkyl; R₁₀ and R₁₁ are each independently selected from the group consisting of hydrogen, alkyl and alkoxylalkyl, or R₁₀ and R₁₁ taken together with any intervening atoms form a 5, 6, or 7-membered ring; R₁₂ is hydrogen; and Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

A further embodiment of the present invention is directed toward a compound of formula (III), or a therapeutically acceptable salt or prodrug thereof, wherein L is a bond; m is 1; n is 1; R₁, R₂ and R₃ are each independently selected from the group consisting of hydrogen, halogen, alkoxy, alkyl, cyano, haloalkyl, haloalkoxy, nitro, RₐR_{b}N— and RₐR_{b}NC(O)—; R₄ is selected from the group consisting of hydrogen, alkyl, alkylcarbonyl-NH—, alkylsulfonyl-NH—, aryl and halogen; R₅ is hydrogen; R₆ is selected from the group consisting of hydrogen and alkyl; R₇ is selected from the group consisting of aryl, heteroaryl and heterocycle, or R₆ and R₇ together with the atoms to which they are attached form cycloalkyl; R₈ is hydrogen; R₉ is selected from the group consisting of hydrogen and alkyl; R₁₀ and R₁₁ are hydrogen; R₁₂ is hydrogen; and Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

Another embodiment of the present invention is directed toward a compound of formula (III), or a therapeutically acceptable salt or prodrug thereof, wherein L is a bond; m is 1; n is 1; R₁, R₂ and R₃ are each independently selected from the group consisting of hydrogen, halogen, alkoxy, alkyl, cyano, haloalkyl, haloalkoxy, nitro, RₐR_{b}N— and RₐR_{b}NC(O)—; R₄ is selected from the group consisting of hydrogen, alkyl, alkylcarbonyl-NH—, alkylsulfonyl-NH—, aryl and halogen; R₅ is hydrogen; R₆ is selected from the group consisting of hydrogen and alkyl; R₇ is selected from the group consisting of aryl, heteroaryl and heterocycle; R₈ is hydrogen; R₉ is selected from the group consisting of hydrogen and alkyl; R₁₀ and R₁₁ are hydrogen; R₁₂ is hydrogen; and Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

Another embodiment of the present invention is directed toward a compound of formula (III), or a therapeutically acceptable salt or prodrug thereof, wherein L is a bond; m is 1; n is 1; R₁, R₂ and R₃ are each independently selected from the group consisting of hydrogen, halogen, alkoxy, alkyl, cyano, haloalkyl, haloalkoxy, nitro, RₐR_{b}N— and RₐR_{b}NC(O)—; R₄ is selected from the group consisting of hydrogen, alkyl, alkylcarbonyl-NH—, alkylsulfonyl-NH—, aryl and halogen; R₅ is hydrogen; R₆ and R₇ together with the atoms to which they are attached form cycloalkyl; R₈ is hydrogen; R₉ is selected from the group consisting of hydrogen and alkyl; R₁₀ and R₁₁ are hydrogen; R₁₂ is hydrogen; and Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; provided that at least one of R₁, R₂ or R₃ are not hydrogen.

In another embodiment of the present invention is directed to the compounds of formula (I), wherein R₇ is aryl or arylalkyl wherein the aryl and the aryl of arylalkyl is further substituted with a group consisting of alkenyl, alkenyloxy, alkoxy, alkoxycarbonyl, alkyl, alkylcarbonyl, alkylcarbonyloxy, alkynyl, aryloxy, arylalkenyl, carboxy, carboxyalkyl, cyano, cyanoalkyl, formyl, halogen, haloalkyl, haloalkoxy, heterocycle, heterocyclealkyl, heterocyclealkyl-N(R_{g})—, heterocycle-N(R_{g})-alkyl-, heterocycle-N(R_{g})-alkoxy-, heterocyclealkoxy, hydroxy, hydroxyalkylene, nitro, RₑR_{f}N—, RₑR_{f}N-alkyl-, RₑR_{f}N-alkoxy-, RₑR_{f}N-alkyl-N(R_{g})—, RₑR_{f}N-alkyl-N(R_{g}carbonyl, RₑR_{f}NC(O)—, RₑR_{f}C(O)N—, RₑR_{f}NS(O)₂—, RₑS(O)₂N—, RₑS(O)₂N-alkyl-, RₑS(O)₂N-alkoxy-, RₑS(O)₂-alkyl-, RₑS(O)₂-alkoxy-, phenyl, and a heterocyclic ring, wherein R_{g}, Rₑ, and R_{f} are defined herein.

According to one embodiment of the present invention, there is provided a method of treating disorders by inhibiting the effects of melanin concentrating hormone (MCH) through the melanin concentrating hormone receptor, comprising administrering a therapeutically effective amount of a compound of formula (I).

According to one embodiment of the present invention, there is provided a method of treating obesity by inhibiting the effects of melanin concentrating hormone (MCH) through the melanin concentrating hormone receptor, comprising administrering a therapeutically effective amount of a compound of formula (I).

According to one embodiment of the present invention, there is provided a method of treating abnormalities in reproduction and sexual behavior, thyroid hormone secretion, diuresis and water/electrolyte homeostasis, sensory processing, memory, sleeping and arousal, anxiety and depression, seizure and in treatment of neurodegeneration or psychiatric disorders by inhibiting the effects of melanin concentrating hormone (MCH) through the melanin concentrating hormone receptor, comprising administrering a therapeutically effective amount of a compound of formula (I).

According to one embodiment of the present invention, there is provided a pharmaceutical composition comprising a therapeutically effective amount of a compound of formula (I) in combination with a pharmaceutically suitable carrier.

### Definitions

As used throughout this specification and the appended claims, the following terms have the following meanings:

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenyloxy,” as used herein, refers to an alkenyl group, as defined herein, appended to the parent molecular moiety through an oxy group, as defined herein.

The term “alkenylene,” denotes a divalent group derived from a straight or branched chain hydrocarbon of from 2 to 10 carbon atoms containing at least one double bond. Representative examples of alkenylene include, but are not limited to, —CH═CH—, —CH═CH₂CH₂—, and —CH═C(CH₃)CH₂—.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxylalkyl,” as used herein, refers to an alkoxy group, as defined herein, appended to the parent molecular moiety through an alkyl group, as defined herein.

The term “alkoxyalkylcarbonyl,” as used herein, refers to an alkoxyalkyl group, as defined herein, appended to the parent molecular moiety through a carbonyl group, as defined herein. Representative examples of alkoxyalkylcarbonyl include, but are not limited to, methoxyacetyl, ethoxyacetyl, 1-oxo-3-propoxypropyl, 1-oxo-3-tert-butoxypropyl.

The term “alkoxyalkylcarbonylalkyl,” as used herein, refers to an alkoxyalkylcarbonyl group, as defined herein, appended to the parent molecular moiety through an alkyl group, as defined herein.

The term “alkoxyalkylsulfonyl,” as used herein, refers to an alkoxyalkyl group, as defined herein, appended to the parent molecular moiety through a sulfonyl group, as defined herein.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl,” as used herein, refers to an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxycarbonylalkoxy,” as used herein, refers to an alkoxycarbonyl group, as defined herein, appended to the parent molecular moiety through an alkoxy group, as defined herein.

The term “alkoxycarbonyloxy,” as used herein, refers to an alkoxycarbonyl group, as defined herein, appended to the parent molecular moiety through an oxy group, as defined herein.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” denotes a divalent group derived from a straight or branched chain hydrocarbon of from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl,” as used herein, refers to an alkylcarbonyl group, as defined herein, appended to the parent molecular moiety through an alkyl group, as defined herein.

The term “alkylcarbonyloxy,” as used herein, refers to an alkylcarbonyl group, as defined herein, appended to the parent molecular moiety through an oxy group, as defined herein.

The term “alkylcarbonyl-NH—,” as used herein, refers to an alkylcarbonyl group, as defined herein, appended to the parent molecular moiety through a —NH— group.

The term “alkyl-NH—,” as used herein, refers to an alkyl group, as defined herein, appended to the parent molecular moiety through an —NH— group.

The term “alkyl-NH-carbonyl,” as used herein, refers to an alkyl-NH— group, as defined herein, appended to the parent molecular moiety through a carbonyl group, as defined herein.

The term “alkyl-NH-sulfonyl,” as used herein, refers to an alkyl-NH-group, as defined herein, appended to the parent molecular moiety through a sulfonyl group, as defined herein.

The term “alkylthio,” as used herein, refers to an alkyl group, as defined herein, appended to the parent molecular moiety through a sulfur atom.

The term “alkylsulfonyl,” as used herein, refers to an alkyl group, as defined herein, appended to the parent molecular moiety through a sulfonyl group, as defined herein.

The term “alkylsulfinyl,” as used herein, refers to an alkyl group, as defined herein, appended to the parent molecular moiety through a sulfinyl group, as defined herein.

The term “alkylsulfonyl-NH—,” as used herein, refers to an alkylsulfonyl group, as defined herein, appended to the parent molecular moiety through a —NH— group.

The term “alkylene,” denotes a divalent group derived from a straight or branched chain hydrocarbon of from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkynylene,” denotes a divalent group derived from a straight or branched chain hydrocarbon of from 2 to 10 carbon atoms containing at least one triple bond. Representative examples of alkynylene include, but are not limited to, —C≡C—, —CH₂C≡C—, —CH(CH₃)CH₂C≡C—, —C≡CCH₂—, and —C≡CCH(CH₃)CH₂—.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “amidino,” or “imino,” as used herein, refers to H₂N—C(═NH)—, appended to the parent molecular moiety.

The term “aryl” as used herein, means a phenyl group, or a bicyclic or a tricyclic fused ring system wherein one or more of the fused rings is a phenyl group. Bicyclic fused ring systems are exemplified by a phenyl group fused to a cycloalkyl group, as defined herein, a heteroaryl group, as defined herein, a heterocycle group, as defined herein or another phenyl group. Tricyclic fused ring systems are exemplified by a bicyclic fused ring system fused to a cycloalkyl group, as defined herein, a heteroaryl group, as defined herein, a heterocycle group, as defined herein or another phenyl group. Representative examples of aryl include, but are not limited to, anthracenyl, azulenyl, fluorenyl, indanyl, indenyl, naphthyl, phenyl and tetrahydronaphthyl.

The aryl groups of this invention can be optionally substituted with 1, 2, or 3 substituents which are each independently members selected from the group consisting of alkenyl, alkenyloxy, alkoxy, alkoxyalkyl, alkoxyalkylcarbonyl, alkoxyalkylsulfonyl, alkoxycarbonyl, alkoxycarbonylalkyl, alkoxycarbonylalkoxy, alkoxycarbonyloxy, alkoxy-N═alkoxy, alkyl, alkylcarbonyl, alkylcarbonylalkyl, alkylcarbonyloxy, alkynyl, aryl, arylalkoxy, arylalkenyl, arylalkyl, arylcarbonyl, aryloxy, carboxy, carboxyalkyl, cyano, cyanoalkyl, formyl, haloalkoxy, haloalkyl, halogen, heteroaryl, heteroarylalkyl, heteroarylcarbonyl, heteroarylalkyl-N(R_{g})—, heteroaryl-N(R_{g})-alkyl-, heteroaryl-N(R_{g})-alkoxy-, heteroarylalkoxy, heterocycle, heterocyclealkyl, heterocyclecarbonyl, heterocyclealkyl-N(R_{g})—, heterocycle-N(R_{g})-alkyl-, heterocycle-N(R_{g})-alkoxy-, heterocyclealkoxy, hydroxy, hydroxyalkyl, hydroxyhaloalkyl, amidino, nitro, RₑR_{f}N—, RₑR_{f}N-alkyl-, RₑR_{f}N-alkoxy-, RₑR_{f}N-alkyl-N(R_{g})—, RₑR_{f}N-alkyl-N(R_{g})-carbonyl, RₑR_{f}N-carbonyl, RₑR_{f}N-carbonylalkyl, RₑR_{f}N-sulfonyl, RⱼC(O)NH—, RⱼS(O)₂NH—, RⱼS(O)₂NH-alkyl, RⱼS(O)₂NH-alkoxy, RⱼS(O)₂-alkyl, RⱼS(O)₂-alkoxy, wherein the substituent aryl, the aryl of arylalkoxy, the aryl of arylalkenyl, the aryl of arylalkyl, the aryl of arylcarbonyl, the aryl of aryloxy, the heteroaryl, the heteroaryl of heteroarylalkyl, the heteroaryl of heteroarylcarbonyl, the heteroaryl of heteroarylalkyl-N(R_{g})—, the heteroaryl of heteroaryl-N(R_{g})-alkyl-, the heteroaryl of heteroaryl-N(R_{g})-alkoxy-, the heteroaryl of heteroarylalkoxy, the heterocycle, the heterocycle of heterocyclealkyl, the heterocycle of heterocyclecarbonyl, the heterocycle of heterocyclealkyl-N(R_{g})—, the heterocycle of heterocycle-N(R_{g})-alkyl-, the heterocycle of heterocycle-N(R_{g})-alkoxy- and the heterocycle of heterocyclealkoxy, can be optionally substituted with 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl and nitro. Rₑ and R_{f} are each individually a member selected from the group consisting of hydrogen, alkyl, alkylcarbonyl, alkylcarbonyloxyalkylcarbonyl, alkoxycarbonyl, alkoxyalkylcarbonyl, aryl, arylalkyl, cycloalkylalkyl-NH-alkylcarbonyl, haloalkyl, haloalkylcarbonyl, heteroaryl, heteroarylalkyl, heterocycle, heterocyclealkyl, heterocyclealkylcarbonyl. R_{g} is selected from the group consisting of hydrogen, alkyl and alkylcarbonyl. Rⱼ is selected from the group consisting of hydrogen, alkyl, aryl-NH-alkyl, heteroaryl-NH-alkyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkoxy,” as used herein, refers to an aryl group, as defined herein, appended to the parent molecular moiety through an alkoxy group, as defined herein.

The term “arylalkenyl” as used herein, refers to an aryl group, as defined herein, appended to the parent molecular moiety through an alkenyl group, as defined herein. Representative examples of arylalkenyl include, but are not limited to, prop-1-enylbenzene, 1-(prop-1-enyl)naphthalene and the like.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl and naphthoyl.

The term “arylcarbonylalkyl” as used herein, refers to an arylcarbonyl group, as defined herein, appended to the parent molecular moiety through an alkyl group, as defined herein. Representative examples of arylcarbonylalkyl include, but are not limited to, propiophenone, 1-(1-naphthyl)propan-1-one and the like.

The term “aryloxy,” as used herein, refers to an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxyalkyl,” as used herein, refers to an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl and 3-bromophenoxymethyl.

The term “arylsulfonyl” as used herein, refers to an aryl group, as defined herein, appended to the parent molecular moiety through a sulfonyl group, as defined herein. Representative examples of arylsulfonyl include but are not limited to (ethylsulfonyl)benzene, 1-(ethylsulfonyl)naphthalene and the like.

The term “arylcarbonyl,” as used herein, refers to an aryl group, as defined herein, appended to the parent molecular moiety through a carbonyl group, as defined herein.

The term “arylcarbonyl-NH—,” as used herein, refers to an arylcarbonyl group, as defined herein, appended to the parent molecular moiety through a —NH— group.

The term “aryl-C═N—O—,” as used herein, refers to an aryl group, as defined herein, appended to the parent molecular moiety through a —C═N—O— group, as defined herein.

The term “aryl-NH—,” as used herein, refers to an aryl group, as defined herein, appended to the parent molecular moiety through a —NH— group.

The term “aryl-NH-carbonyl,” as used herein, refers to an aryl-NH— group, as defined herein, appended to the parent molecular moiety through a carbonyl group, as defined herein.

The term “aryl-NH-sulfonyl,” as used herein, refers to an aryl-NH— group, as defined herein, appended to the parent molecular moiety through a sulfonyl group, as defined herein.

The term “aryloxy,” as used herein, refers to an aryl group, as defined herein, appended to the parent molecular moiety through an oxygen atom.

The term “arylthio,” as used herein, refers to an aryl group, as defined herein, appended to the parent molecular moiety through a sulfur atom.

The term “arylthioalkylcarbonyl,” as used herein, refers to an arylthioalkyl group, as defined herein, appended to the parent molecular moiety through a carbonyl group, as defined herein.

The term “arylsulfonyl,” as used herein, refers to an aryl group, as defined herein, appended to the parent molecular moiety through a sulfonyl group, as defined herein.

The term “arylsulfonyl-NH—,” as used herein, refers to an arylsulfonyl group, as defined herein, appended to the parent molecular moiety through a —NH-group.

The term “arylalkylcarbonyl,” as used herein, refers to an arylalkyl group, as defined herein, appended to the parent molecular moiety through a carbonyl group, as defined herein.

The term “arylalkylcarbonyl-NH—,” as used herein, refers to an arylalkylcarbonyl group, as defined herein, appended to the parent molecular moiety through a —NH— group.

The term “arylalkyl-NH—,” as used herein, refers to an arylalkyl group, as defined herein, appended to the parent molecular moiety through a —NH— group.

The term “arylalkyl-NH-carbonyl,” as used herein, refers to an arylalkyl-NH— group, as defined herein, appended to the parent molecular moiety through a carbonyl group, as defined herein.

The term “arylalkyl-NH-sulfonyl,” as used herein, refers to an arylalkyl-NH— group, as defined herein, appended to the parent molecular moiety through a sulfonyl group, as defined herein.

The term “arylalkoxy,” as used herein, refers to an aryl group, as defined herein, appended to the parent molecular moiety through an alkoxy group, as defined herein.

The term “arylalkylthio,” as used herein, refers to an arylalkyl group, as defined herein, appended to the parent molecular moiety through a sulfur atom.

The term “arylalkylsulfonyl,” as used herein, refers to an arylalkyl group, as defined herein, appended to the parent molecular moiety through a sulfonyl group, as defined herein.

The term “arylalkylsulfonyl-NH—,” as used herein, refers to an arylalkylsulfonyl group, as defined herein, appended to the parent molecular moiety through a —NH— group.

The term “biarylalkyl” as used herein, refers to two aryl groups, as defined herein, appended to the parent molecular moiety through an alkyl group, as defined herein. Representative examples of biarylalkyl include but are not limited to (1-phenylbutyl)benzene and the like.

The term “carbonyl,” or “carboxy,” as used herein, refers to a —C(O)— group.

The term “carbonylalkyl,” or “carboxyalkyl,” as used herein, refers to a carbonyl group, as defined herein, appended to the parent molecular moiety through an alkyl group, as defined herein.

The term “cyano,” as used herein, refers to a —CN group.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic fused ring systems are exemplified by a cycloalkyl group appended to the parent molecular moiety, which is fused to an additional cycloalkyl group, as defined herein, an aryl group, as defined herein, a heteroaryl, as defined herein, or a heterocycle as defined herein. Tricyclic fused ring systems are exemplified by a cycloalkyl bicyclic fused ring system fused to an additional cycloalkyl group, as defined herein, an aryl group, as defined herein, a heteroaryl, as defined herein, or a heterocycle as defined herein. The additional fused cycloalkyl group may be substituted but may not be fused to another ring. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of this invention can be optionally substituted with 1, 2, or 3 substituents independently a member selected from alkenyl, alkenyloxy, alkoxy, alkoxyalkyl, alkoxyalkylcarbonyl, alkoxyalkylsulfonyl, alkoxycarbonyl, alkoxycarbonylalkyl, alkoxycarbonylalkoxy, alkoxy-N═alkoxy, alkyl, alkylcarbonyl, alkylcarbonylalkyl, alkylcarbonyloxy, alkynyl, aryl, arylalkoxy, arylalkenyl, arylalkyl, arylcarbonyl, aryloxy, carboxy, carboxyalkyl, cyano, cyanoalkyl, formyl, haloalkoxy, haloalkyl, halogen, heteroaryl, heteroarylalkyl, heteroarylcarbonyl, heteroarylalkyl-N(R_{g})—, heteroarylalkoxy, heterocycle, heterocyclealkyl, heterocyclecarbonyl, heterocyclealkoxy, hydroxy, hydroxyalkyl, amidino, RₑR_{f}N—, RₑR_{f}N-alkyl-, RₑR_{f}N-alkoxy-, RₑR_{f}NC(O)—, wherein the substituent aryl, the aryl of arylalkoxy, the aryl of arylalkenyl, the aryl of arylalkyl, the aryl of arylcarbonyl, the aryl of aryloxy, the heteroaryl, the heteroaryl of heteroarylalkyl, the heteroaryl of heteroarylcarbonyl, the heteroaryl of heteroarylalkyl-N(R_{g})—, the heteroaryl of heteroarylalkoxy, the heterocycle, the heterocycle of heterocyclealkyl, the heterocycle of heterocyclecarbonyl and the heterocycle of heterocyclealkoxy, can be optionally substituted with 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl and nitro. Rₑ and R_{f} are each individually a member selected from the group consisting of hydrogen, alkyl, alkylcarbonyl, alkylcarbonyloxyalkylcarbonyl, alkoxycarbonyl, alkoxyalkylcarbonyl, aryl, arylalkyl, cycloalkylalkyl-NHalkylcarbonyl, haloalkyl, haloalkylcarbonyl, heteroaryl, heteroarylalkyl, heterocycle, heterocyclealkyl, heterocyclealkylcarbonyl. R_{g} is selected from the group consisting of hydrogen, alkyl and alkylcarbonyl.

The term “cycloalkylcarbonyl,” as used herein, refers to a cycloalkyl group, as defined herein, appended to the parent molecular moiety through a carbonyl group, as defined herein. The term “cycloalkylcarbonyl-NH—,” as used herein, refers to a cycloalkylcarbonyl group, as defined herein, appended to the parent molecular moiety through a —NH— group.

The term “cycloalkyl-NH—,” as used herein, refers to a cycloalkyl group, as defined herein, appended to the parent molecular moiety through a —NH— group.

The term “cycloalkyl-NH-carbonyl,” as used herein, refers to a cycloalkyl-NH— group, as defined herein, appended to the parent molecular moiety through a carbonyl group, as defined herein.

The term “cycloalkyl-NH-sulfonyl,” as used herein, refers to a cycloalkyl-NH— group, as defined herein, appended to the parent molecular moiety through a sulfonyl group, as defined herein.

The term “cycloalkoxy,” as used herein, refers to a cycloalkyl group, as defined herein, appended to the parent molecular moiety through an oxy group, as defined herein.

The term “cycloalkylthio,” as used herein, refers to a cycloalkyl group, as defined herein, appended to the parent molecular moiety through a sulfur atom.

The term “cycloalkylsulfonyl,” as used herein, refers to a cycloalkyl group, as defined herein, appended to the parent molecular moiety through a sulfonyl group, as defined herein.

The term “cycloalkylsulfonyl-NH—,” as used herein, refers to a cycloalkylsulfonyl group, as defined herein, appended to the parent molecular moiety through a —NH— group.

The term “cycloalkenylcarbonyl,” as used herein, refers to a cycloalkenyl group, as defined herein, appended to the parent molecular moiety through a carbonyl group, as defined herein.

The term “cycloalkenylcarbonyl-NH—,” as used herein, refers to a cycloalkenylcarbonyl group, as defined herein, appended to the parent molecular moiety through a —NH— group.

The term “cycloalkenyl-NH—,” as used herein, refers to a cycloalkenyl group, as defined herein, appended to the parent molecular moiety through a —NH— group.

The term “cycloalkenyl-NH-carbonyl,” as used herein, refers to a cycloalkenyl-NH— group, as defined herein, appended to the parent molecular moiety through a carbonyl group, as defined herein.

The term “cycloalkenyl-NH-sulfonyl,” as used herein, refers to a cycloalkenyl-NH— group, as defined herein, appended to the parent molecular moiety through a sulfonyl group, as defined herein.

The term “cycloalkenyloxy,” as used herein, refers to a cycloalkenyl group, as defined herein, appended to the parent molecular moiety through an oxygen atom.

The term “cycloalkenylthio,” as used herein, refers to a cycloalkenyl group, as defined herein, appended to the parent molecular moiety through a sulfur atom.

The term “cycloalkenylsulfonyl,” as used herein, refers to a cycloalkenyl group, as defined herein, appended to the parent molecular moiety through a sulfonyl group, as defined herein.

The term “cycloalkenylsulfonyl-NH—,” as used herein, refers to a cycloalkenylsulfonyl group, as defined herein, appended to the parent molecular moiety through a —NH— group.

The term “formyl,” as used herein, refers to a —C(O)H group.

The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkylcarbonyl,” as used herein, refers to a haloalkyl, as defined herein, appended to the parent molecular moiety through a carbonyl group, as defined herein.

The term “haloalkylsulfonyl,” as used herein, refers to a haloalkyl, as defined herein, appended to the parent molecular moiety through a sulfonyl group, as defined herein.

The term “heteroaryl,” as used herein, means an aromatic monocyclic ring or an aromatic bicyclic ring. The aromatic monocyclic rings are five or six membered rings wherein 1, 2, 3, or 4 atoms are independently selected from the group consisting of N, O and S. The five membered aromatic monocyclic rings have two double bonds and the six membered aromatic monocyclic rings have three double bonds. Bicyclic fused heteroaryl ring systems are exemplified by monocyclic heteroaryl ring fused to an aryl group, as defined herein or to another heteroaryl monocyclic ring, as defined herein. The aromatic monocyclic rings and the aromatic bicyclic rings may be connected to the parent molecular moiety through either a carbon or nitrogen atom. Representative examples of heteroaryl include, but are not limited to, benzimidazole, benzoxazole, benzoxazole-(2)-one, benzothiazolyl, benzo(1,2,5)-thiadiazole, benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, tetrazolyl, thiadiazolyl, thiazolyl, thienopyridinyl, thienyl, triazolyl and triazinyl.

The heteroaryl groups of the present invention are optionally substituted with 1, 2, 3, or 4 substituents independently selected from the group consisting of alkenyl, alkenyloxy, alkoxy, alkoxyalkyl, alkoxyalkylcarbonyl, alkoxyalkylsulfonyl, alkoxycarbonyl, alkoxycarbonylalkyl, alkoxycarbonylalkoxy, alkoxycarbonyloxy, alkoxy-N═alkoxy, alkyl, alkylcarbonyl, alkylcarbonylalkyl, alkylcarbonyloxy, alkynyl, aryl, arylalkoxy, arylalkenyl, arylalkyl, arylcarbonyl, aryloxy, carboxy, carboxyalkyl, cyano, cyanoalkyl, formyl, haloalkoxy, haloalkyl, halogen, heteroaryl, heteroarylalkyl, heteroarylcarbonyl, heteroarylalkyl-N(R_{g})—, heteroaryl-N(R_{g})-alkyl-, heteroaryl-N(R_{g})-alkoxy-, heteroarylalkoxy, heterocycle, heterocyclealkyl, heterocyclecarbonyl, heterocyclealkyl-N(R_{g})—, heterocycle-N(R_{g})-alkyl-, heterocycle-N(R_{g})-alkoxy-, heterocyclealkoxy, hydroxy, hydroxyalkyl, hydroxyhaloalkyl, amidino, nitro, RₑR_{f}N—, RₑR_{f}N-alkyl-, RₑR_{f}N-alkoxy-, RₑR_{f}N-alkyl-N(R_{g})—, RₑR_{f}N-alkyl-N(R_{g})—C(O)—, RₑR_{f}NC(O)—, RₑR_{f}NC(O)-alkyl, RₑR_{f}NS(O)₂—, RⱼC(O)NH—, RⱼS(O)₂NH—, RⱼS(O)₂NH-alkyl-, RⱼS(O)₂NH-alkoxy-, RⱼS(O)₂-alkyl-, RⱼS(O)₂-alkoxy-, wherein the substituent aryl, the aryl of arylalkoxy, the aryl of arylalkenyl, the aryl of arylalkyl, the aryl of arylcarbonyl, the aryl of aryloxy, the heteroaryl, the heteroaryl of heteroarylalkyl, the heteroaryl of heteroarylcarbonyl, the heteroaryl of heteroarylalkyl-N(R_{g})—, the heteroaryl of heteroaryl-N(R_{g})-alkyl-, the heteroaryl of heteroaryl-N(R_{g})-alkoxy-, the heteroaryl of heteroarylalkoxy, the heterocycle, the heterocycle of heterocyclealkyl, the heterocycle of heterocyclecarbonyl, the heterocycle of heterocyclealkyl-N(R_{g})—, the heterocycle of heterocycle-N(R_{g})-alkyl-, the heterocycle of heterocycle-N(R_{g})-alkoxy- and the heterocycle of heterocyclealkoxy, can be optionally substituted with 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl and nitro. Rₑ and R_{f} are each individually a member selected from the group consisting of hydrogen, alkyl, alkylcarbonyl, alkylcarbonyloxyalkylcarbonyl, alkoxycarbonyl, alkoxyalkylcarbonyl, aryl, arylalkyl, cycloalkylalkyl-NHalkylcarbonyl, haloalkyl, haloalkylcarbonyl, heteroaryl, heteroarylalkyl, heterocycle, heterocyclealkyl, heterocyclealkylcarbonyl. R_{g} is selected from the group consisting of hydrogen, alkyl and alkylcarbonyl. Rⱼ is selected from the group consisting of hydrogen, alkyl, arylNHalkyl and heteroarylNHalkyl.

The term “heteroarylalkyl,” as used herein, refers to a heteroaryl group, as defined herein, appended to the parent molecular moiety through an alkyl group, as defined herein.

The term “heteroarylalkoxy,” as used herein, refers to a heteroaryl group, as defined herein, appended to the parent molecular moiety through an alkoxy group, as defined herein.

The term “heteroarylcarbonyl,” as used herein, refers to a heteroaryl group, as defined herein, appended to the parent molecular moiety through a carbonyl group, as defined herein.

The term “heterocycle,” as used herein, refers to a non-aromatic monocyclic ring or a nonaromatic bicyclic ring. The monocyclic ring is a three, four, five, six, seven, or eight membered ring containing 1 or 2 heteroatoms independently selected from the group consisting of N, O and S. The bicyclic heterocycle rings are composed of a monocyclic heterocycle ring fused to another monocyclic heterocycle, as defined herein, a heteroaryl ring, as defined herein, or an aryl group, as defined herein. Representative examples of heterocycle include, but are not limited to, azetidinyl, 1,3-benzodioxolyl, benzooxazolidin-2-one, 3H-benzothiazolyl-2-one, 2,3-dihydrobenzo(1,4)dioxinyl, dihydroquinolin-2-one, 1H-quinolinyl-2-one, 4H-benzo(1,4)oxazin-3-one, 3H-benzooxazol-2-one, dihydrobenzofuran, chromen-2-one, 1,3-dioxolanyl, 1,4-dioxanyl, hexahydro-1H-azepinyl, hexahydroazocin-(2H)-yl, imidazoldinyl, morpholinyl, morpholine-3-one, oxazolidin-2-one, piperazinyl, piperidinyl, pyrrolidinyl, tetrahydrofuranyl, tetrahydrofuran-2-yl, tetrahydrofuran-3-yl, tetrahydro-2H-pyranyl, tetrahydro-2H-pyran-2-yl, tetrahydro-2H-pyran-4-yl, tetrahydrothienyl, tetrahydrothien-2-yl, tetrahydrothien-3-yl, thiazol-2-one, and thiomorpholinyl.

The heterocycles of this invention are optionally substituted with 1, 2, 3, or 4 substituents independently selected from the group consisting of alkenyl, alkenyloxy, alkoxy, alkoxyalkyl, alkoxyalkylcarbonyl, alkoxyalkylsulfonyl, alkoxycarbonyl, alkoxycarbonylalkyl, alkoxycarbonylalkoxy, alkoxycarbonyloxy, alkoxy-N═alkoxy, alkyl, alkylcarbonyl, alkylcarbonylalkyl, alkylcarbonyloxy, alkynyl, aryl, arylalkoxy, arylalkenyl, arylalkyl, arylcarbonyl, aryloxy, carboxy, carboxyalkyl, cyano, cyanoalkyl, formyl, haloalkoxy, haloalkyl, halogen, heteroaryl, heteroarylalkyl, heteroarylcarbonyl, heteroarylalkyl-N(R_{g})—, heteroaryl-N(R_{g})-alkyl-, heteroaryl-N(R_{g})-alkoxy-, heteroarylalkoxy, heterocycle, heterocyclealkyl, heterocyclecarbonyl, heterocyclealkyl-N(R_{g})—, heterocycle-N(R_{g})-alkyl-, heterocycle-N(R_{g})-alkoxy-, heterocyclealkoxy, hydroxy, hydroxyalkyl, hydroxyhaloalkyl, amidino, nitro, RₑR_{f}N—, RₑR_{f}N-alkyl-, RₑR_{f}N-alkoxy-, RₑR_{f}N-alkyl-N(R_{g})—, RₑR_{f}N-alkyl-N(R_{g})—C(O)—, RₑR_{f}NC(O)—, RₑR_{f}NC(O)-alkyl, RₑR_{f}NS(O)₂—, RⱼC(O)NH—, RⱼS(O)₂NH—, RⱼS(O)₂NH-alkyl-, RⱼS(O)₂NH-alkoxy-, RⱼS(O)₂-alkyl-, RⱼS(O)₂-alkoxy-, wherein the substituent aryl, the aryl of arylalkoxy, the aryl of arylalkenyl, the aryl of arylalkyl, the aryl of arylcarbonyl, the aryl of aryloxy, the heteroaryl, the heteroaryl of heteroarylalkyl, the heteroaryl of heteroarylcarbonyl, the heteroaryl of heteroarylalkyl-N(R_{g})—, the heteroaryl of heteroaryl-N(R_{g})-alkyl-, the heteroaryl of heteroaryl-N(R_{g})-alkoxy-, the heteroaryl of heteroarylalkoxy, the heterocycle, the heterocycle of heterocyclealkyl, the heterocycle of heterocyclecarbonyl, the heterocycle of heterocyclealkyl-N(R_{g})—, the heterocycle of heterocycle-N(R_{g})-alkyl-, the heterocycle of heterocycle-N(R_{g})-alkoxy- and the heterocycle of heterocyclealkoxy, can be optionally substituted with 1, or 2 substitutents independently selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl and nitro. Rₑ and R_{f} are each individually a member selected from the group consisting of hydrogen, alkyl, alkylcarbonyl, alkylcarbonyloxyalkylcarbonyl, alkoxycarbonyl, alkoxyalkylcarbonyl, aryl, arylalkyl, cycloalkylalkyl-NH-alkylcarbonyl, haloalkyl, haloalkylcarbonyl, heteroaryl, heteroarylalkyl, heterocycle, heterocyclealkyl, heterocyclealkylcarbonyl. R_{g} is selected from the group consisting of hydrogen, alkyl and alkylcarbonyl. Rⱼ is selected from the group consisting of hydrogen, alkyl, aryl-NH-alkyl and heteroaryl-NH-alkyl.

The term “heterocyclealkyl,” as used herein, refers to a heterocycle, as defined herein, appended to the parent molecular moiety through an alkyl group, as defined herein. Representative examples of heterocycle-alkyl include, but are not limited to, pyridin-3-ylmethyl and 2-pyrimidin-2-ylpropyl.

The term “heterocyclealkoxy,” as used herein, refers to a heterocycle group, as defined herein, appended to the parent molecular moiety through an alkoxy group, as defined herein.

The term “heterocyclecarbonyl,” as used herein, refers to a heterocycle group, as defined herein, appended to the parent molecular moiety through a carbonyl group, as defined herein.

The term “heterocyclecarbonyl-NH—,” as used herein, refers to a heterocyclecarbonyl group, as defined herein, appended to the parent molecular moiety through a —NH— group. The term “heterocycle-NH—,” as used herein, refers to a heterocycle group, as defined herein, appended to the parent molecular moiety through a —NH— group.

The term “heterocyclealkyl-NH-aryl-,” as used herein, refers to a heterocyclealkyl-NH—, as defined herein, appended to the parent molecular moiety through an aryl group, as defined herein.

The term “heterocycle-NH-carbonyl,” as used herein, refers to a heterocycle-NH— group, as defined herein, appended to the parent molecular moiety through a carbonyl group, as defined herein.

The term “heterocyclealkyl-NH-carbonylaryl,” as used herein, refers to a heterocycle-NH-carbonyl group, as defined herein, appended to the parent molecular moiety through an aryl group, as defined herein.

The term “heterocycle-NH-sulfonyl,” as used herein, refers to a heterocycle-NH— group, as defined herein, appended to the parent molecular moiety through a sulfonyl group, as defined herein.

The term “heterocycleoxy,” as used herein, refers to a heterocycle group, as defined herein, appended to the parent molecular moiety through an oxygen atom.

The term “heterocyclethio,” as used herein, refers to a heterocycle group, as defined herein, appended to the parent molecular moiety through a sulfur atom.

The term “heterocyclesulfonyl,” as used herein, refers to a heterocycle group, as defined herein, appended to the parent molecular moiety through a sulfonyl group, as defined herein.

The term “heterocyclesulfonyl-NH—,” as used herein, refers to a heterocyclesulfonyl group, as defined herein, appended to the parent molecular moiety through an —NH— group, as defined herein.

The term “heterocyclealkylcarbonyl,” as used herein, refers to a heterocycle-alkyl group, as defined herein, appended to the parent molecular moiety through a carbonyl group, as defined herein.

The term “heterocyclealkylcarbonyl-NH—,” as used herein, refers to a heterocyclealkylcarbonyl group, as defined herein, appended to the parent molecular moiety through a —NH— group, as defined herein.

The term “heterocyclealkyl-NH—,” as used herein, refers to a heterocyclealkyl group, as defined herein, appended to the parent molecular moiety through a —NH— group, as defined herein.

The term “heterocyclealkyl-NH-carbonyl,” as used herein, refers to a heterocyclealkyl-NH— group, as defined herein, appended to the parent molecular moiety through a carbonyl group, as defined herein.

The term “heterocyclealkyl-NH-sulfonyl,” as used herein, refers to a heterocyclealkyl-NH— group, as defined herein, appended to the parent molecular moiety through a sulfonyl group, as defined herein.

The term “heterocyclealkyloxy,” as used herein, refers to a heterocyclealkyl group, as defined herein, appended to the parent molecular moiety through an oxygen atom.

The term “heterocyclealkylthio,” as used herein, refers to a heterocyclealkyl group, as defined herein, appended to the parent molecular moiety through a sulfur atom.

The term “heterocyclealkylsulfonyl,” as used herein, refers to a heterocyclealkyl group, as defined herein, appended to the parent molecular moiety through a sulfonyl group, as defined herein.

The term “heterocyclealkylsulfonyl-NH—,” as used herein, refers to a heterocyclealkylsulfonyl group, as defined herein, appended to the parent molecular moiety through a —NH— group.

The term “hydroxy,” as used herein, refers to an —OH group. The term “hydroxyalkyl,” as used herein, refers to a hydroxy group, as defined herein, appended to the parent molecular moiety through an alkyl group, as defined herein.

Representative examples of hydroxyalkyl include, but are not limited to, hydroxybutyl, hydroxypentyl and hydroxyhexyl.

The term “hydroxyhaloalkyl,” as used herein, refers to an alkyl group, as defined herein, appended to the parent molecular moiety through an alkyl group, as defined herein.

The term “nitro,” as used herein, refers to a —NO₂ group.

The term “RₐR_{b}N—,” as used herein, refers to Rₐ and R_{b}, as defined herein, appended to the parent molecular moiety through a nitrogen atom.

The term “oxo,” as used herein, refers to a ═O moiety. The term “sulfinyl,” as used herein, refers to a —S(O)— group.

The term “sulfonyl,” as used herein, refers to a —SO₂— group.

The term “alkylthio,” as used herein, refers to an alkyl group, as defined herein, appended to the parent molecular moiety through a sulfur atom.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isothionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds can also exist as therapeutically suitable esters and prodrugs. The term “therapeutically suitable esters and prodrug,” refers to those esters and prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to the parent compounds of formula (I-II) for example, by hydrolysis in blood. The term “therapeutically suitable ester,” refers to compounds which are rapidly transformed in vivo to the parent compounds of formula (I-II) for example, by hydrolysis in blood. The term “therapeutically suitable ester,” refers to alkoxycarbonyl groups appended to the parent molecule on an available carbon atom. More specifically, a “therapeutically suitable ester,” may exist on one or more available aryl, cycloalkyl and heterocycle group as defined herein.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described herein below and resolved by techniques well-known in the art.

Geometric isomers can exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracistemally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents which dissolve or disperse in the injectable media.

Antagonism of the effects of MCH through the MCH receptor by the compounds of the present invention can be delayed by using a liquid suspension of crystalline or amorphous material with poor water solubility. The rate of absorption of the compounds depends upon their rate of dissolution which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions which are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable nonirritating excipient which is solid at ordinary temperature but fluid in the rectum.

The present compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally comprise tableting lubricants and aids. Capsules can also optionally contain opacifying agents which delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Disorders caused or exacerbated by MCH are treated or prevented in a patient by administering to the patient, a therapeutically effective amount of compound of the present invention in such an amount and for such time as is necessary to achieve the desired result. The term “therapeutically effective amount,” refers to a sufficient amount of a compound to effectively emeliorate disorders mediated by MCH, by antagonizing the effect of MCH through the MCH receptor at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the present compounds in single or divided doses can be in amounts, for example, from 0.01 to 50 mg/kg body weight or more usually from 0.1 to 25 mg/kg body weight. In general, treatment regimens comprise administration to a patient in need of such treatment from about 10 mg to about 1000 mg of the compounds per day in single or multiple doses.

### Determination of Biological Activity

Assay for Release of Intracellular Calcium:

Activation of the melanin concentrating hormone receptor (MCHR) by MCH induces the release of Ca⁺⁺ from intracellular stores. This intracellular calcium release is measured using a fluorometric imaging plate reader (FLIPR™, Molecular Devices Corp.) in conjunction with the Ca⁺⁺-sensitive dye Fluo-4. Release of Ca⁺⁺ from intracellular stores causes an increase in fluorescence of the dye that is proportional to Ca⁺⁺ concentration. In particular, the assay is carried out as follows: The cells are cultured in MEM/10% fetal bovine serum/50 μg/mL gentamicin/200 μg/ml Zeocin. The cells are plated at 100,000 cells/well in poly-D-lysine coated, 96 FLIPR™ assay plates (BD Biosciences, Bedford, Mass.). After two days, cells are loaded with the Calcium Assay Reagent (100 μL+100 μL culture medium; Molecular Devices Corporation, Sunnyvale, Calif.) for at least one hour at room temperature. Test compounds are prepared at 6 μM in 6% dimethyl sulfoxide. The cell plate is placed in the FLIPR and 50 μl/well of test compound is delivered. The calcium signal is followed for 3 minutes to assay for potential agonist activity by the test compounds. Then, 50 μl/well of 600 nM human MCH (in Dulbecco's phosphate-buffered saline (PBS) containing 0.1% bovine serum albumin (BSA)) is added and the ligand-induced calcium signal is followed for an additional 3 minutes. Antagonist activity, as determined by the test compound's ability to inhibit MCH-induced Ca⁺⁺ flux, is calculated as percent inhibition as described by the following formula:
% inhibition=[1−((fTC−−fB)/(fMCH−fB))]×100
fTC=MCH-induced fluorescence in the presence of test compound;
fMCH=MCH-induced fluorescence in the absence of test compound; and
fB=baseline fluorescence.

MCH (100 nM) usually elicits a response of 9,000-11,000 relative fluorescence units (RFU) with a baseline of approximately 200 RFU. Calcium Assay Reagent fluorescence is measured at 488 nm, with an exposure of 0.40 sec. and F-stop=2.0 and the laser set at 0.20-0.40 W constant light output. It should be noted that both antagonists and inverse agonists would be expected to produce similar results in this assay. Both types of agent have been found to be useful therapeutically for inhibition of signaling by various GPCR's.

TABLE 1
% Inhibition of 100 nM MCH at 1 uM

| | | | | |
|---|---|---|---|---|
| −0.211 | 6.219 | 10.455 | 13.162 | 14.011 |
| 20.583 | 25.535 | 32.886 | 35.913 | 38.351 |
| 39.681 | 40.118 | 40.266 | 41.306 | 41.368 |
| 42.660 | 46.032 | 48.080 | 50.047 | 50.538 |
| 53.110 | 53.178 | 54.617 | 55.357 | 55.647 |
| 58.781 | 58.939 | 61.292 | 61.340 | 62.156 |
| 62.445 | 62.684 | 63.045 | 63.357 | 64.959 |
| 67.136 | 67.804 | 68.295 | 69.205 | 69.234 |
| 69.467 | 69.692 | 69.831 | 70.186 | 70.257 |
| 70.480 | 70.532 | 70.639 | 70.779 | 71.848 |
| 71.895 | 72.512 | 72.865 | 75.237 | 75.734 |
| 76.619 | 77.021 | 77.193 | 79.162 | 79.188 |
| 79.458 | 80.345 | 80.412 | 80.610 | 80.653 |
| 80.914 | 82.757 | 82.985 | 83.586 | 83.722 |
| 84.257 | 84.740 | 84.785 | 84.800 | 85.044 |
| 86.023 | 86.052 | 86.250 | 86.490 | 86.957 |
| 87.540 | 88.408 | 88.468 | 88.889 | 88.950 |
| 89.534 | 89.550 | 89.700 | 89.752 | 89.932 |
| 90.616 | 90.876 | 91.809 | 92.202 | 93.351 |
| 93.733 | 93.840 | 94.016 | 94.096 | 95.273 |
| 95.870 | 95.967 | 96.155 | 96.321 | 96.405 |
| 96.843 | 97.268 | 97.505 | 97.755 | 97.830 |
| 97.896 | 97.937 | 98.012 | 98.297 | 98.348 |
| 98.407 | 98.421 | 98.480 | 98.509 | 98.511 |
| 98.542 | 98.561 | 98.666 | 98.745 | 98.861 |
| 98.868 | 98.870 | 98.871 | 98.879 | 99.431 |
| 99.514 | 99.539 | 99.927 | 99.981 | 100.047 |
| 100.080 | 100.193 | 100.391 | 100.394 | 100.425 |
| 100.542 | 100.544 | 100.603 | 100.618 | 100.951 |
| 100.978 | 101.312 | 101.358 | 101.780 | 102.915 |
| 104.259 | | | | |

According to Table 1, the compounds of the present invention inhibit MCH induced fluorescence at a dose of 1 μM. In a preferred range, compounds of the present invention inhibit MCH induced fluorescence in a range of 75-100% inhibition of MCH at a dose of 1 μM. In a more preferred range, compounds of the present invention inhibit MCH induced fluorescence in a range of 90-100% inhibition of MCH at a dose of 1 μM.

As antagonists of MCH action upon the MCH receptor, therefore, the compounds of the present invention are useful in treating disorders that are mediated by MCH through the MCH receptor. Disorders that are mediated by MCH through the MCH receptor are obesity, abnormalities in reproduction and sexual behavior, thyroid hormone secretion, diuresis and water/electrolyte homeostasis, sensory processing, memory, sleeping and arousal, anxiety and depression, seizure and in treatment of neurodegeneration or psychiatric disorders. Therefore the compounds of the present invention are useful in treating obesity, abnormalities in reproduction and sexual behavior, thyroid hormone secretion, diuresis and water/electrolyte homeostasis, sensory processing, memory, sleeping and arousal, anxiety and depression, seizure and in treatment of neurodegeneration or psychiatric disorders.

Therapeutic agents acting through MCH receptor may also be useful in treatment of abnormalities in reproduction and sexual behavior (Murray, J. F.; Mercer J. G., Adan R. A., Datta J. J., Aldairy C, Moar K M, Baker B I, Stock M J, Wilson, C. A.; The effect of leptin on luteinizing hormone release is exerted in the zona incerta and mediated by melanin-concentrating hormone. J Neuroendocrinol 12:1133-1139, 2000.; Gonzalez, M. I., Baker, B. I., Wilson, C. A.; Stimulatory effect of melanin-concentrating hormone on luteinising hormone release. Neuroendocrinology 66:254-262, 1997.; Murray, J. F., Adan, R. A., Walker, R., Baker, B. I., Thody, A. J., Nijenhuis, W. A., Yukitake, J., Wilson, C. A.; Melanin-concentrating hormone, melanocortin receptors and regulation of luteinizing hormone release. J Neuroendocrinol 12:217-223, 2000.; Nahon, J. L.; The melanin-concentrating hormone: from the peptide to the gene. Crit Rev Neurobiol 8:221-262, 1994.)

Therapeutic agents acting through MCH receptor may also be useful in treatment of thyroid hormone secretion (Kennedy, A. R., Todd, J. F., Stanley, S. A., Abbott, C. R., Small, C. J., Ghatei, M. A., Bloom, S. R.; Melanin-concentrating hormone (MCH) suppresses thyroid stimulating hormone (TSH) release, in vivo and in vitro, via the hypothalamus and the pituitary. Endocrinology 142:3265-3268. 2001).

Therapeutic agents acting through MCH receptor may also be useful in treatment of diuresis and water/electrolyte homeostasis (Hervieu, G., Volant, K., Grishina, O., Descroix-Vagne, M., Nahon, J. L.; Similarities in cellular expression and functions of melanin-concentrating hormone and atrial natriuretic factor in the rat digestive tract. Endocrinology 137:561-571, 1996.; and Parkes, D. G.; Diuretic and natriuretic actions of melanin concentrating hormone in conscious sheep. J Neuroendocrinol 8:57-63, 1996).

Therapeutic agents acting through MCH receptor may also be useful in treatment of sensory processing (Miller, C. L., Hruby, V. J., Matsunaga, T. O., Bickford, P. C.; Alpha-MSH and MCH are functional antagonists in a CNS auditory gating paradigm. Peptides 14:431-440, 1993.; Kokkotou, E. G., Tritos, N. A., Mastaitis, J. W., Slieker, L., Maratos-Flier, E.; Melanin-concentrating hormone receptor is a target of leptin action in the mouse brain. Endocrinology 142:680-686, 2001).

Therapeutic agents acting through MCH receptor may also be useful in treatment of memory (Monzon, M. E., De Barioglio, S. R.; Response to novelty after i.c.v. injection of melanin-concentrating hormone (MCH) in rats. Physiol Behav 67:813-817, 1999).

Therapeutic agents acting through MCH receptor may also be useful in treatment of sleeping and arousal (Bittencourt, J. C., Presse, F., Arias, C., Peto, C., Vaughan, J., Nahon, J. L., Vale, W., Sawchenko, P. E.; The melanin-concentrating hormone system of the rat brain: an immuno- and hybridization histochemical characterization. J Comp Neurol 319:218-245, 1992.; Nahon, J. L.; The melanin-concentrating hormone: from the peptide to the gene. Crit Rev Neurobiol 8:221-262, 1994).

Therapeutic agents acting through MCH receptor may also be useful in treatment of anxiety and depression (Monzon, M. E., Varas, M. M., De Barioglio, S. R.; Anxiogenesis induced by nitric oxide synthase inhibition and anxiolytic effect of melanin-concentrating hormone (MCH) in rat brain. Peptides 22:1043-1047, 2001.; Monzon, M. E., De Barioglio, S. R.; Response to novelty after i.c.v. injection of melanin-concentrating hormone (MCH) in rats. Physiol Behav 67:813-817, 1999.; Borowsky, B., Durkin, M. M., Ogozalek, K., Marzabadi, M. R., DeLeon, J., Lagu, B., Heurich, R., Lichtblau, H., Shaposhnik, Z., Daniewska, I., Blackburn, T. P., Branchek, T. A., Gerald, C., Vaysse, P. J., Forray, C.; Antidepressant, anxiolytic and anorectic effects of a melanin-concentrating hormone-1 receptor antagonist. Nat. Med. 8:825-830, 2002).

Therapeutic agents acting through MCH receptor may also be useful in treatment of seizure (Knigge, K. M., Wagner, J. E; Melanin-concentrating hormone (MCH) involvement in pentylenetetrazole (PTZ)-induced seizure in rat and guinea pig. Peptides 18:1095-1097, 1997) and in treatment of neurodegeneration or psychiatric disorders (Nahon, J. L.; The melanin-concentrating hormone: from the peptide to the gene. Crit Rev Neurobiol 8:221-262, 1994).

Synthetic Methods

Abbreviations which have been used in the descriptions of the scheme and the examples that follow are: dba for dibenzylideneacetone; DMSO for dimethylsulfoxide; NMP for N-methylpyrrolidinone; DMF for N,N-dimethylformamide; DCC for 1,3-dicyclohexylcarbodiimide, DIC for 2-dimethylaminoisopropyl chloride hydrochloride, HATU for O-(7-azabenzotriazol-1-yl)-N,N,N′,N′-tetramethyluronium hexafluorophosphate, HBTU for O-benzotriazole-1-yl)-N,N,N′,N′-tetramethyluronium hexafluorophosphate, TFA for trifluoroacetic acid; THF for tetrahydrofuran; EDCI for 1-(3-dimethylaminopropyl)-3-ethylcarbodiimide hydrochloride; HOAt for 1-hydroxy-7-azabenzotriazole and h OBt for 1-hydroxybenzotriazole hydrate.

The compounds and processes of the present invention will be better understood in connection with the following synthetic schemes and experimentals which together illustrate the methods by which the compounds of the invention may be prepared. The groups R₁, R₂, R₃, R₄, R₅, R₆, R₇, R₈, R₉, R₁₀, R₁₁, R₁₂, X and L are as defined above unless otherwise noted below.

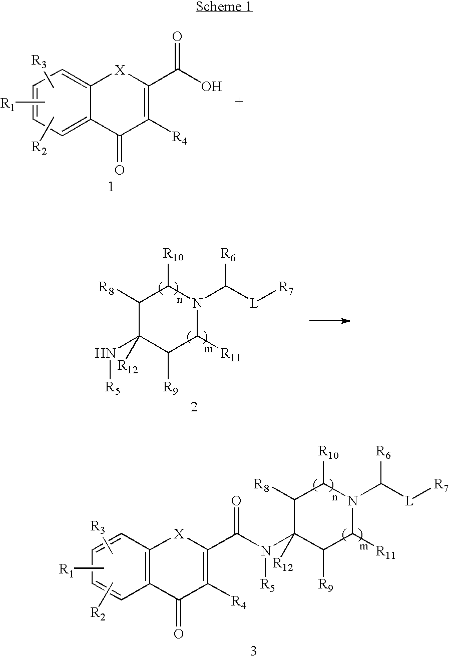

As shown in Scheme 1, compounds of formula 1 when treated with compounds of formula 2 and reagents that will couple amines to carboxylic acids such as EDCI, DCC, DIC, HATU, HBTU, an auxiliary nucleophile such as but not limited to HOBt and HOAt and a base such as but not limited to diisopropylethylamine, triethylamine, N-methylmorpholine in solvents such as but not limited to N,N-dimethylformamide and methylene chloride will provide the compound of formula 3 which are representative of the compounds of the present invention. Alternativly, compounds of formula 1 may be treated with oxalyl chloride in dichloromethane in the presence of a catalytic amount DMF followed by treatment with compounds of formula 2 in the presence of a base such as N-methylmorpholine in dichlormethane to provide compounds of formula 3.

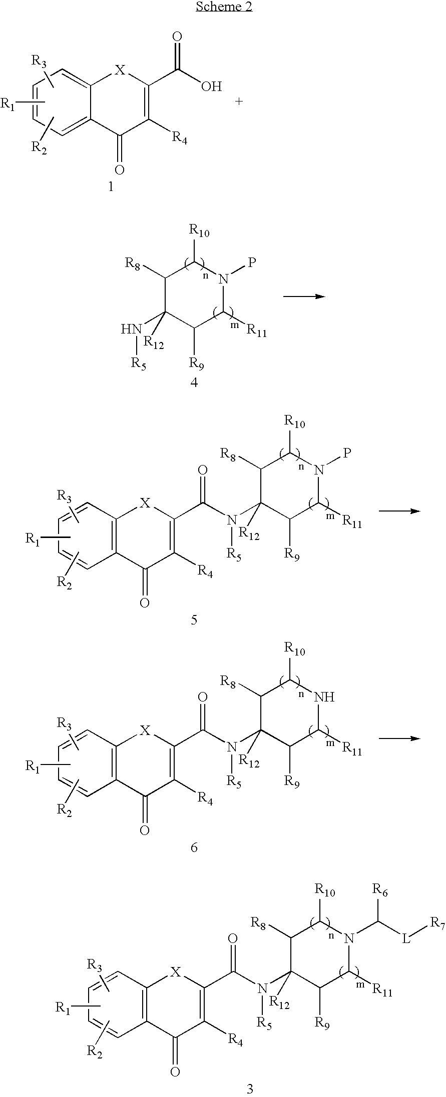

As shown in Scheme 2, compounds of formula 1 can be treated with compounds of formula 4 using the conditions outlined in Scheme 1 to provide compounds of formula 5. The symbol P of compound of formula 4 and 5 is a nitrogen protecting group such as but not limited to acetyl, tert-butyloxy carbonyl or benzyloxycarbonyl, or as described in Greene, T. W. and Wuts, G. M. “Protective groups in Organic Synthesis”, third ed. John Wiley & Sons, 1999. The method of use of such protecting group and conditions decribing the removal of the group are also described in the abovementioned reference or may be known to one skilled in the art. Compounds of formula 5 can be deprotected using conditions described in the reference or as known to one skilled in the art to provide compounds of formula 6. Compounds of formula 6 may treated according to conditions as described in Scheme 3 or 4 to provide compounds of formula 3.

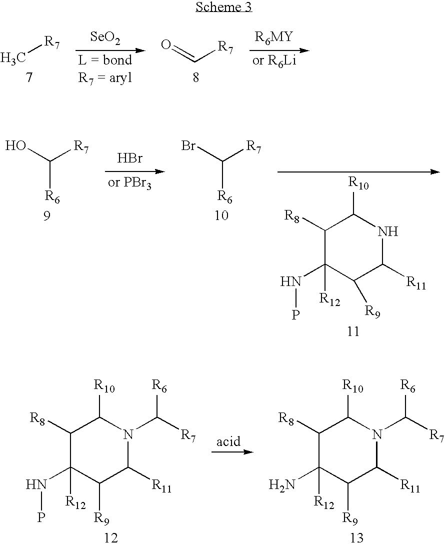

As shown in Scheme 3, compounds of formula 7 wherein R₇ is aryl, when treated with an oxidizing reagent such as but not limited to selenium dioxide will provide compounds of formula 8. Compounds of formula 8 when treated with organometallic reagents of formula R₆MgY or R₆Li, wherein Y is chloro or bromo and R₆ is defined within the scope of this invention, in solvents such as but not limited to THF will provide compounds of formula 9. Compounds of formula 9 when treated with hydrobromic acid or phosphorous tribromide will provide compounds of formula 10. Compounds of formula 10 when treated with an amine of formula 11 and a base such as but not limited to potassium carbonate in DMF or triethylamine under heated conditions in solvents such as THF will provide compounds of formula 12. The nitrogen protecting group (P), of the compound of formula 12 which may consist of a tert-butyloxycarbonyl, acetyl or benzyloxycarbonyl may be removed using conditions known to those skilled in the art or are described in the “Greene” reference listed above will provide compounds of formula 13. For example the deprotection of compounds of formula 12 which contain a tert-butyloxycarbonyl may be converted into a compound of formula 13 when treated with an acid such as trifluoroacetic acid in dichloromethane or hydrochloric acid in acetic acid. The compound of formula 13 may be coupled to the compound of formula 1 as described in Scheme 1 to provide compounds of formula 3 which are representative of the compounds of the present invention.

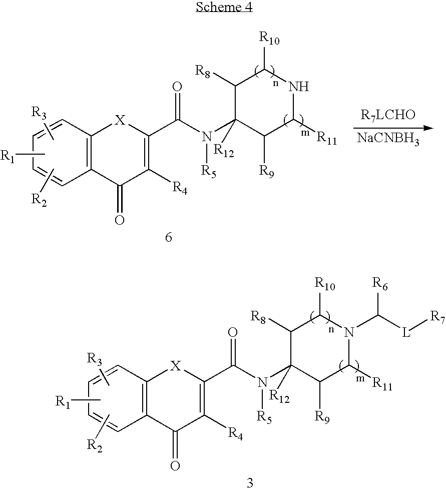

As shown in Scheme 4, compounds of formula 6 may also be treated with an aldehyde of formula R₇LCHO and a reducing agent such as but not limited to sodium cyanoborohydride in solvents such as but not limited to THE to provide compounds of formula 3. This reaction sequence enables one skilled in the art to generate compounds of the present invention without alkylation conditions as described in the previous Schemes.

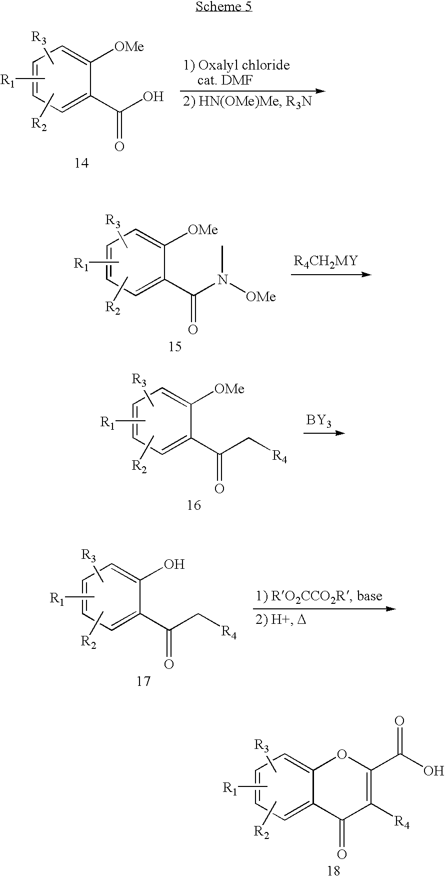

As shown in Scheme 5, compounds of formula 14 when treated with oxalyl chloride dichloromethane containing a catalytic amount of DMF followed by the addition of N-methyl O-methyl hydroxylamine in the presence of a base such as but not limited to triethylamine will provide compounds of formula 15. Compounds of formula 15 when treated with reagents of formula R₄CH₂MgY wherein R₄ is defined above and Y is halogen will provide compounds of formula 16. Compounds of formula 16 when treated with reagents such as boron tribromide or aluminum trichloride in solvents such as but not limited to dichlromethane will provide compounds of formula 17. Compounds of formula 17 when treated with compounds of formula R′O₂CCO₂R′ (wherein R′ is methyl or ethyl) and a base such as sodium methoxide in methanol followed by treatment with an acid such as hydrochloric acid or acetic acid and heat followed by hydrolysis will provide compounds of formula 18. Compounds of formula 18 may be subjected to the conditions described in Scheme 1 or Scheme 2 to provide compounds of the present invention.

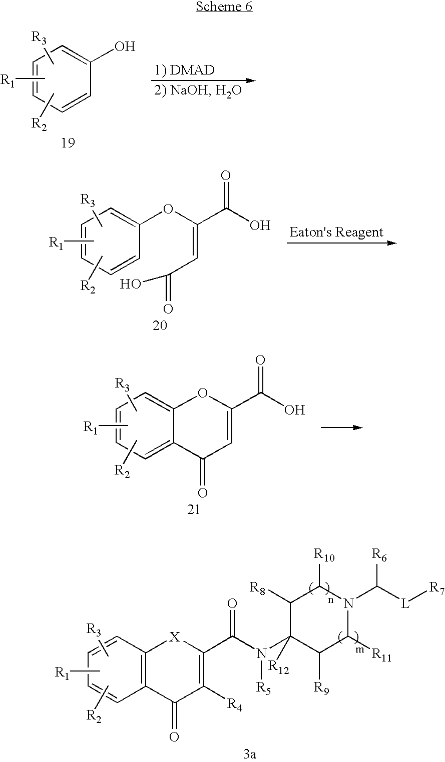

As shown in Scheme 6, compounds of formula 19 when treated dialkyl acetylenedicarboxylate under heated conditions followed by treatment with sodium hydroxide will provide compounds of formula 20. Compounds of formula 20 when treated with phosphorous pentoxide in methane sulfonic acid (Eaton's reagent) will provide compounds of formula 21. Compounds of formula 21 may be treated according to the conditions described in Scheme 1 or Scheme 2 to provide compounds of the formula 3a which are representative of compounds of the present invention which contain an oxygen in place of X.

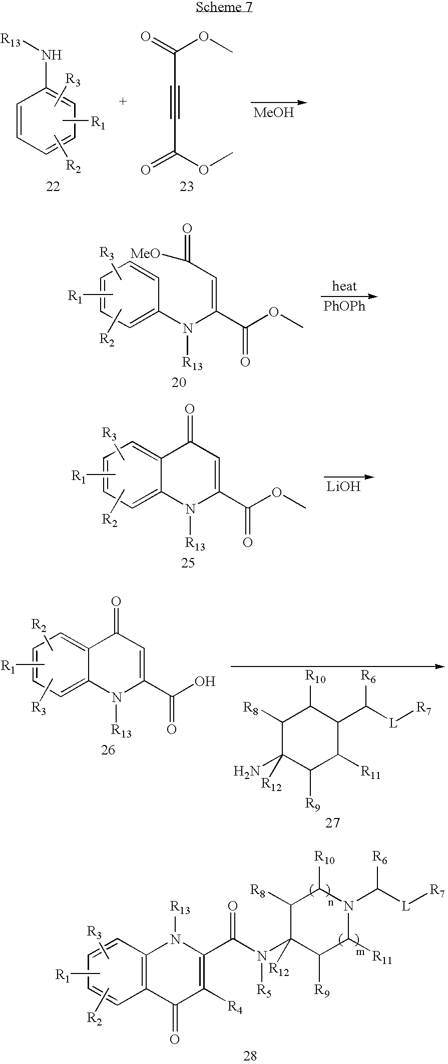

As shown in Scheme 7, compounds of formula 22 when treated with dimethyl acetylenedicarboxylate (compound of formula 23) in methanol will provide compounds of formula 24. Compounds of formula 24 when heated in solvents such as but not limited to phenyl ether will provide compounds of formula 25. Compounds of formula 25 when treated lithium hydroxide or sodium hydroxide in solvents such as but not limited to aqueous methanol or aqueous isopropanol will provide compounds of formula 26. Compounds of formula 26 when treated to conditions outlined in Scheme 1 or Scheme 2 will provide compounds of formula 28 which are representative of the compounds of the present invention.

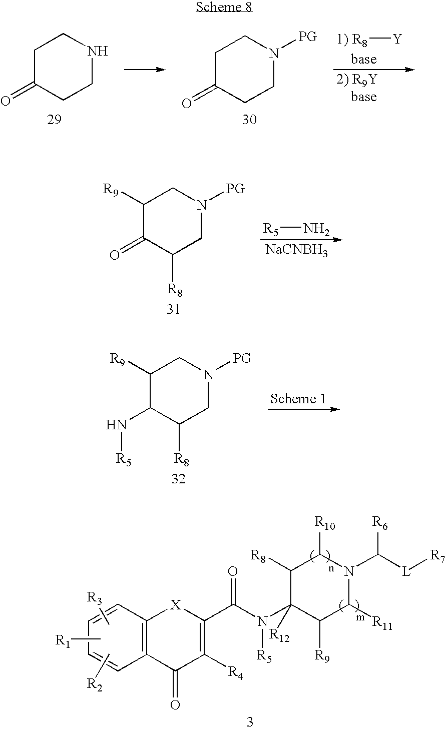

As shown in Scheme 8, compounds of formula 29 when first protected using a nitrogen protecting group as known to one skilled in the art or as descirbed in Greene, T. W. and Wuts, G. M. “Protective groups in Organic Synthesis”, third ed. John Wiley & Sons, 1999 followed by sequential treatment with R₈Y and base such as but not limited to lithium diisopropylamine followed by R₉Y and base such as but not limited to lithium diisopropylamine, wherein R₈ and R₉ are defined within the scope of this invention will provide compounds of formula 31. Compounds of formula 31 when treated with an amine of formula R₅—NH₂ and a reduceing agent such as but not limited to sodium cyanoborohydride, sodium triacetoxyborohydride or Palladium on carbon and a hydrogen atmosphere in solvents such as but not limited to THF will provide compounds of formula 32. Compounds of formula 32 when subjected to the conditions outlined in Scheme 2 will provide compounds of formula 3 which are representative of the compounds of the present invention.

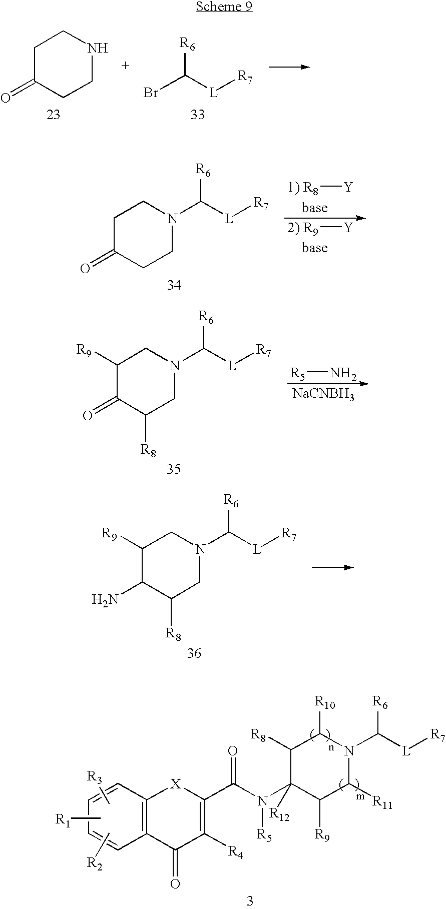

As shown in Scheme 9, compounds of formula 23 when treated with compounds of formula 33 and a base such as but not limited to triethylamine in solvents including THF will provide compounds of formula 34. Compounds of formula 34 when treated sequentially with R₈-Y and a base such as but not limited to lithium diisopropylamine followed by R₉—Y and a base such as but not limited to lithium diisopropylamine in solvents such as but not limited to THE, wherein R₈ and R₉ are defined with the scope of this invention and Y is a halogen will provide compounds of formula 35. Compounds of formula 35 when treated with reagents of formula R₅—NH2 and a reducing agent such as but not limited to sodium cyanoborohydride will provide compounds of formula 36. Compounds of formula 36 when treated to conditions described in Scheme 1 will provide compounds of formula 3 which are representative of compounds of the present invention.

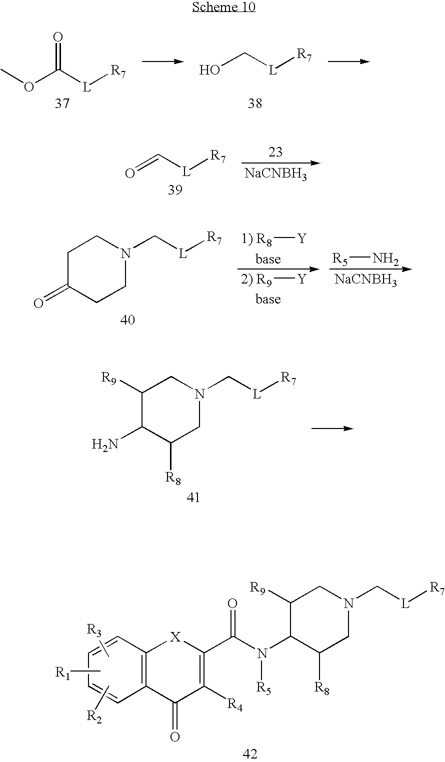

As shown in Scheme 10, compounds of formula 37 when subjected to reducing conditions such as but not limited to diisobutyl aluminum hydride will provide compounds of formula 38. Compounds of formula 38 when subjected to oxidative conditions as known to one skilled in the art or as described by the Swern reaction (oxalyl chloride in DMSO followed by the addition of triethylamine) will provide compounds of formula 39. Compounds of formula 39 when treated with compounds of formula 23 and a reducing agent such as but not limited to sodium cyanoborohydride will provide compounds of formula 40. Compounds of formula 40 when treated according to conditions outlined in Scheme 9 to introduce R₈ and R₉ followed by treatment with compounds of formula R₅—NH₂ and a reducing agent such as sodium cyanoborohydride or as described above will provide compounds of formula 41. Compounds of formula 41 when treated according to Scheme 1 will provide compounds of formula 42 which are representative of compounds of the present invention.

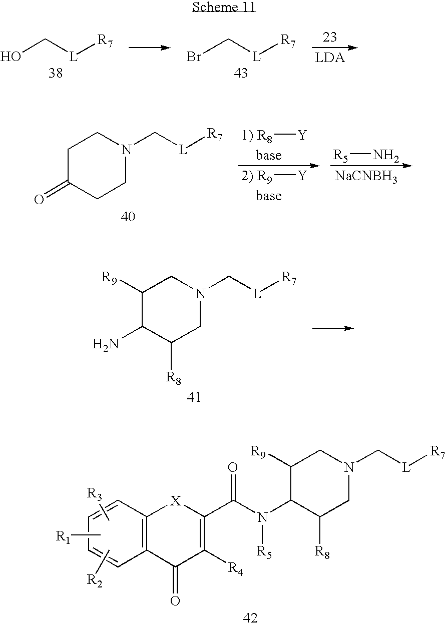

Alternatively, compounds of formula 38 can be treated with reagents such as hydrogen bromide or phosphorous tribromide to provide compounds of formula 43. Compounds of formula 43 when treated with compounds of formula 23 and a base such as but not limited to LDA will provide compounds of formula 40. Compounds of formula 40 can be subjected to the conditions outlined in Scheme 10 to introduce R₈ and R₉ followed by reductive amination to provide compounds of formula 41 which when treated according to conditions described in Scheme 1 will provide compounds of formula 42 which are representative of compounds of the present invention.

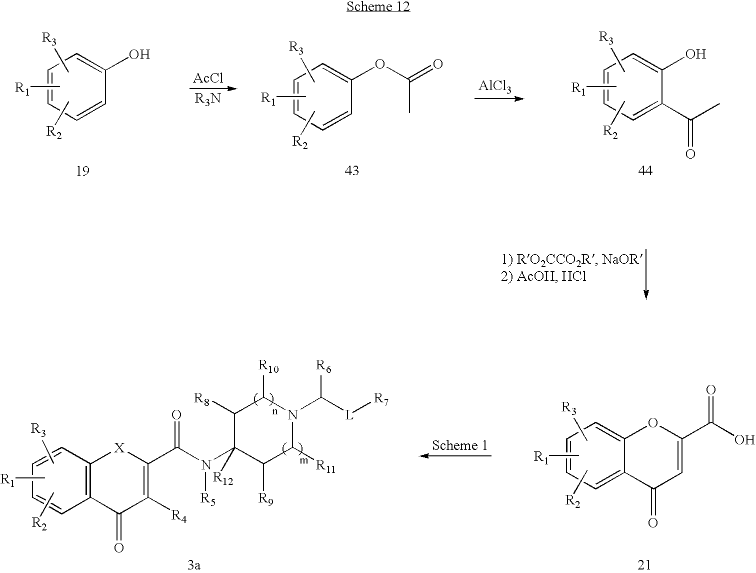

As shown in Scheme 12, compounds of formula 19 when treated with acetyl chloride and a tertiary amine such as but not limited to triethylamine or diisopropylethylamine in an appropriate solvent will provide compounds of formula 43. Compounds of formula 43 are treated with aluminum chloride in solvents such as but not limited to benzene, toluene or dichloroethane will provide compounds of formula 44. Compounds of formula 44 when treated with a compound of formula R′O₂CCO₂R′ (wherein R′ is alkyl) and a base such as sodium alkoxide followed by treatment with aqueous HCl and acetic acid will provide compounds of formula 21. Compounds of formula 21 when treated with compounds of formula 2 according to conditions outlined in Scheme 1, will provide compounds of formula 3 which are representative of compounds of the present invention.

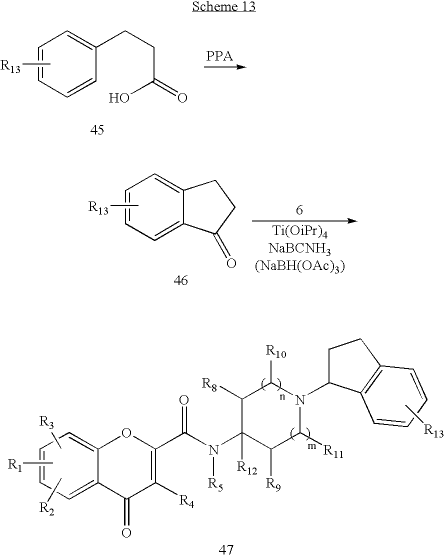

As outlined in Scheme 13, compounds of formula 45 when treated with polyphosphorous acid, or undergo any Freidel-Crafts acylation type of conditions, will provide compounds of formula 46. Compounds of formula 46 when treated with compounds of formula 6 in the presence of titanium (IV) isopropoxide and a reducing agent such as but not limited to sodium cyanoborohydride or sodium triacetoxyborohydride will provide compounds of formula 47 which are representative of compounds of the present invention.

### EXPERIMENTALS

### Example 1

### N-[1-(1,3-benzodioxol-5-ylmethyl)piperidin-4-yl]-7-chloro-4-oxo-4H-chromene-2-carboxamide

### Example 1A

### 4-chloro-N,2-dimethoxy-N-methylbenzamide

To an oven-dried 250 mL round bottom flask containing 4-chloro-2-methoxybenzoic acid (5 g, 26.8 mmol), dichloromethane (95 mL), and DMF (0.25 mL) which was purged with nitrogen and cooled to 0° C. was added oxalyl chloride (13.4 mL, 26.8 mmol of a 2 M solution in dichloromethane). The mixture was stirred at 0° C. for 30 minutes and then allowed to warm to room temperature. After 1 hour at room temperature, the mixture was cooled to 0° C., and N,O-dimethylhydroxylamine hydrochloride (2.9 g, 29.5 mmol) was added followed by the addition of diisopropylethylamine (10.3 mL, 59.0 mmol). The mixture was allowed to warm to room temperature and stirred for 72 hours. The mixture was diluted with dichloromethane, washed with 1M aqueous HCl, 1M aqueous K₂CO₃, dried (Na₂SO₄), and concentrated to provide the title compound as a solid. ¹H NMR (300 MHz, DMSO-d₆) δ ppm 3.17 (s, 3H), 3.47 (s, 3H), 3.82 (m, 3H), 7.04 (dd, J=8.14, 1.70 Hz, 1H), 7.17 (d, J=1.70 Hz, 1H), 7.25 (d, J=8.14 Hz, 1H); MS (ESI) m/z 230 [M+H]⁺.

### Example 1B

### 1-(4-chloro-2-methoxyphenyl)ethanone

An oven-dried 100 mL round bottom flask containing Example 1A (1.25 g, 5.44 mmol) and dry THF (22 mL) was cooled to −78° C., and air was evacuated and replaced with nitrogen (3×). Methyl magnesium bromide (2.0 mL of a 3M solution in ethyl ether, 5.99 mmol) was added dropwise and after 30 minutes the cold bath was removed, and the reaction was allowed to warm to room temperature and stirred for 16 hours. To the mixture was diluted with 2M aqueous HCl followed by the addition of Ethyl acetate (50 mL). The separated organic layer was washed with distilled water (1×50 mL), washed with saturated aqueous NaHCO₃ (1×50 mL), washed with brine (1×50 mL), dried (MgSO₄), filtered, and concentrated to a residue that was purified by Flashmaster (20 gram cartridge, 0 to 100% Ethyl acetate in hexane over 35 minutes). ¹H NMR (300 MHz, DMSO-d₆) δ ppm 3.31 (s, 3H), 3.92 (s, 3H), 7.09 (dd, J=8.14, 1.70 Hz, 1H), 7.27 (d, J=1.70 Hz, 1H), 7.60 (d, J=8.14 Hz, 1H); MS (APCI) m/z 185 [M+H]⁺.

### Example 1C

### 1-(4-chloro-2-hydroxyphenyl)ethanone

Boron tribromide (7.6 mL, 7.6 mmol of a 1M solution in dichloromethane) was added to Example 1B (0.7 g, 3.8 mmol) in dichloromethane (8 mL) at 0° C. The mixture was stirred cold for 1 hour, and was allowed to warm to room temperature for 0.5 hour. The solution was added carefully to 2N aqueous HCl (50 mL) and ice (50 g) and extracted with dichloromethane (2×50 mL). The combined organic layers were washed with saturated aqueous NaHCO₃ (2×50 mL), washed with brine (1×50 mL), dried (MgSO₄), filtered, and concentrated to a dark residue (485 mg) that was purified by Flashmaster (20 gram cartridge, 0 to 20% Ethyl acetate in hexane over 25 minutes.) The title compound was collected as a dark oil. ¹H NMR (300 MHz, DMSO-d₆) δ ppm 2.62 (s, 3H), 7.03 (d, J=8.48 Hz, 1H), 7.07 (d, J=2.37 Hz, 1H), 7.89 (d, J=8.48 Hz, 1H), 12.04 (s, 1H); MS (APCI) m/z 169 [M−H]⁺.

### Example 1D

### ethyl 7-chloro-4-oxo-4H-chromene-2-carboxylate

A mixture of Example 1C (469 mg, 2.75 mmol) and diethyl oxalate (1.90 mL, 13.8 mmol) was added to a stirred solution of sodium ethoxide (3.6 mL, 11.0 mmol of a 21% solution by weight in EtOH), and the mixture was warmed to 40° C. After 1 hour, the solution was cooled, filtered and the yellow solid was washed with ethyl ether. The organic layer was diluted with dichloromethane (50 mL), washed with 10% aqueous acetic acid and concentrated to a solid that was combined with glacial acetic acid (3 mL), hydrochloric acid (0.1 mL) and heated at 90° C. After 4 hours the dark solution was cooled, combined with distilled water (5 mL). The resulting white precipitate was collected by filtration and air-dried overnight. ¹H NMR (300 MHz, DMSO-d₆) δ ppm 1.35 (t, J=7.12 Hz, 3H), 4.40 (q, J=7.12 Hz, 1H), 6.98 (s, 1H), 7.60 (dd, J=8.65, 1.86 Hz, 1H), 8.00 (d, J=2.03 Hz, 1H), 8.05 (d, J=8.48 Hz, 1H); MS (APCI) m/z 253 [M+H]⁺.

### Example 1E

### 7-chloro-4-oxo-4H-chromene-2-carboxylic acid

6M Aqueous HCl (2 mL) was added to Example 1D in acetic acid (4 mL) and the mixture heated to 80° C. for 4.5 hours. The mixture was cooled to room temperature, diluted with distilled water (6 mL) and the resulting beige precipitate (204 mg, 89%) collected by filtration and dried under reduced pressure at 50° C. ¹H NMR (300 MHz, DMSO-d₆) δ ppm 6.92 (s, 1H), 7.58 (dd, J=8.65, 1.86 Hz, 1H), 7.95 (d, J=1.70 Hz, 1H), 8.04 (d, J=8.48 Hz, 1H); MS (APCI) m/z 225 [M+H]⁺, 224 [M−H]⁺, 178.9 [M-CO₂H]⁺.

### Example 1F

### tert-butyl 4-{[(7-chloro-4-oxo-4H-chromen-2-yl)carbonyl]amino}piperidine-1-carboxylate

Diisopropylethylamine (330 μL, 1.80 mmol) was added to a mixture of Example 1E (200 mg, 0.899 mmol), 4-amino-1-N-Boc-piperidine (190 mg, 0.944 mmol), 1-hydrobenzotriazole (152 mg, 1.12 mmol), 1-[3-(dimethylamino)propyl]-3-ethylcarbodiimide hydrochloride (215 mg, 1.12 mmol) in DMF (2.5 mL). The mixture was stirred for 16 hours, diluted with dichloromethane (40 mL), washed with 1N aqueous NaOH (2×40 mL), washed with 1N aqueous HCl (1×40 mL), washed with brine (1×40 mL), dried (MgSO₄), filtered, and concentrated to a dark solid. ¹H NMR (300 MHz, DMSO-d₆) δ ppm 1.40 (m, 9H), 1.46 (m, 2H), 1.78 (m, 2H), 2.89 (m, 2H), 3.99 (m, 3H), 6.84 (s, 1H), 7.59 (dd, J=8.82, 2.03 Hz, 1H), 7.89 (d, J=1.70 Hz, 1H), 8.05 (d, J=8.48 Hz, 1H), 8.83 (d, J=8.14 Hz, 1H); MS (APCI) m/z 351 [M-tBu]⁺, 307 [M-Boc]⁺, 405 [M−H]⁺.

### Example 1G

### 7-chloro-4-oxo-N-piperidin-4-yl-4H-chromene-2-carboxamide

Trifluoroacetic acid (2 mL) was added to a stirred solution of Example 1F (238 mg, 0.585 mmol) and dichloromethane (4 mL) at 0° C. After 1 hour, volatiles were removed and the resulting solid was diluted with 1N aqueous HCl (40 mL) and washed with ethyl ether (1×20 mL). The aqueous layer was combined with saturated aqueous K₂CO₃ until basic (pH=10), and extracted with Ethyl acetate (3×20 mL) and dichloromethane (2×20 mL). The combined organic layers were washed with brine (1×20 mL), dried (MgSO₄), filtered, and concentrated to provide the title compound. ¹H NMR (300 MHz, DMSO-d₆) δ ppm 1.83 (m, 2H), 1.96 (m, 2H), 3.00 (m, 2H), 3.39 (m, 2H), 4.09 (m, 1H), 6.86 (s, 1H), 7.59 (dd, J=8.81, 2.03 Hz, 1H), 7.91 (d, J=2.03 Hz, 1H), 8.04 (d, J=8.48 Hz, 1H), 8.78 (s, 1H), 9.06 (d, J=7.80 Hz, 1H); MS (APCI) m/z 306.9 [M+H]⁺.

### Example 1

### N-[1-(1,3-benzodioxol-5-ylmethyl piperidin-4-yl]-7-chloro-4-oxo-4H-chromene-2-carboxamide

Sodium triacetoxyborohydride (1.23 g, 5.82 mmol) was added to a slurry of Example 1G (1.19 g, 3.88 mmol), piperonal (583 mg, 3.88 mmol), sodium sulfate (1.10 g, 7.76 mmol), acetic acid (0.8 mL), and THF (40 mL) and the mixture stirred for 24 hours. Methanol (4 mL) was added to the mixture followed by the addition of dichloromethane (120 mL). The organic mixture was washed with 1N aqueous NaOH (3×100 mL), brine (1×100 mL), dried (MgSO₄), filtered, and concentrated to an residue that was purified by the Flashmaster (20 gram column, eluting 0 to 100% Ethyl acetate in hexane for 10 minutes, then 100% Ethyl acetate for 10 minutes). The title compound (345 mg, 20%) was collected as a white solid. ¹H NMR (300 MHz, DMSO-d₆) δ ppm 1.64 (m, 2H), 1.78 (m, 2H), 2.00 (m, 2H), 2.83 (d, J=11.53 Hz, 2H), 3.39 (s, 2H), 3.78 (m, 1H), 5.99 (s, 2H), 6.75 (dd, J=7.97, 1.53 Hz, 1H), 6.82 (s, 1H), 6.85 (d, J=8.14 Hz, 2H), 7.59 (dd, J=8.48, 2.03 Hz, 1H), 7.91 (d, J=1.70 Hz, 1H), 8.04 (d, J=8.82 Hz, 1H), 8.81 (d, J=7.80 Hz, 1H); MS (APCI) m/z 441 [M+H]⁺, 439 [M−H]⁺.

### Example 2

### N-[1-(1,3-benzodioxol-5-ylmethyl)piperidin-4-yl]-7-methoxy-4-oxo-4H-chromene-2-carboxamide

### Example 2A

### 7-methoxy-4-oxo-4H-chromene-2-carboxylic acid ethyl ester

To a solution of 7-hydroxy-4-oxo-4H-chromene-2-carboxylic acid ethyl ester (10 g, 42.7 mmol) in 90 mL of acetone under nitrogen was added K₂CO₃ (13 g, 93.8 mmol) followed by iodomethane (3.3 mL, 53.4 mmol) and the mixture heated to reflux for 19 hours. The mixture was allowed to cool to room temperature, 50 mL of ethyl acetate was added, the solid filtered off, rinsed with 1/1 ethyl acetate/acetone and air dried to provide the title compound. ¹H NMR (300 MHz, DMSO-D6) δ ppm 1.35 (t, J=7.12 Hz, 3H) 3.93 (s, 3H) 4.40 (q, J=7.12 Hz, 2H) 6.90 (s, 1H) 7.12 (dd, J=8.82, 2.37 Hz, 1H) 7.24 (d, J=2.37 Hz, 1H) 7.96 (d, J=8.82 Hz, 1H) MS (ESI, MeOH/NH4OH) m/z 248 [M+].

### Example 2B

### 7-methoxy-4-oxo-4H-chromene-2-carboxylic acid

To a solution of Example 2A (7-methoxy-4-oxo-4H-chromene-2-carboxylic acid ethyl ester, 10 g, 40 mmol) in 120 mL of THF and 40 ml of distilled water was added 3.4 g of lithium hydroxide hydrate and the mixture was stirred for 2 hours. The mixture was diluted by slow addition of 15 mL of 3M H₂SO₄ portionwise via pipet and the resulting solid was filtered off, washed with distilled water and air dried to provide the title compound. ¹H NMR (300 MHz, DMSO-D6) δ ppm 3.91 (s, 3H) 6.84 (s, 1H) 7.10 (dd, 1H) 7.22 (d, 1H) 7.95 (d, 1H); MS (ESI, MeOH/NH4OH) m/z 220 [M+], 219 [M−].

### Example 2C

### 4-[(7-Methoxy-4-oxo-4H-chromene-2-carbonyl)-amino]-piperidine

To a solution of Example 2B (7-methoxy-4-oxo-4H-chromene-2-carboxylic acid, 7.5 g, 34 mmol) in 136 mL of dimethylformamide under an atmosphere of nitrogen was added 4-amino-1-BOC-piperidine (6.8 g, 34 mmol), hydroxybenzotriazole (4.6 g, 34 mmol), diisopropylethylamine (6.5 mL, 37.4 mmol), and then ethyldimthylaminocarbodiimide hydrochloride (6.8 g, 35.7 mmol). The mixture was stirred for 17 hours, diluted with ethyl acetate, washed sequentially with 25 ml portions of each 1M H₂SO4, 1M K2CO3, brine. The organic layer was dried (Na₂SO₄, filtered and concentrated under reduced pressure to provide 13 g of a brown residue. This residue dissolved in 90 mL dichloromethane, under an atmosphere of nitrogen was cooled to 0° C. followed by the addition of 45 mL of trifluoroacetic acid in three portions. The mixture was stirred for 15 minutes at 0° C. then allowed to warm to room temperature and stirred for 1.5 hours. The mixture was concentrated under reduced pressure to a brown oil which was dissolved in dichloromethane (300 mL) and washed with 1M K₂CO₃, aqueous extracted with dichloromethane/Methanol (95/5, 3×300 mL). The combined organic extracts were dried (Na₂SO₄), filtered and concentrated under reduced pressure to provide the title compound. ¹H NMR (300 MHz, DMSO-D6) δ ppm 1.51 (m, 2H) 1.77 (m, 2H) 2.55 (m, 2H) 2.99 (m, 2H) 3.83 (m, 1H) 3.93 (s, 3H) 6.76 (s, 1H) 7.10 (dd, 3H) 7.24 (d, 1H) 7.95 (d, 1H) 8.83 (d, 1H); MS (ESI, MeOH/NH4OH) m/z 303 [M+H], 301 [M−H], 603 [2M−H].

### Example 2D

### N-[1-(1,3-benzodioxol-5-ylmethyl)piperidin-4-yl]-7-methoxy-4-oxo-4H-chromene-2-carboxamide

To a solution of Example 2C (7.5 d, 24.8 mmol) in 70 mL dichloromethane, 2 mL glacial acetic acid and 30 mL tetrahydrofuran was added piperonal (3.7 g, 24.8 mmol) followed by NaBH(OAc)₃ (10.6 g, 49.6 mmol). The mixture was stirred for 64 hours, diluted by slow addition of 1M K₂CO₃, diluted with 400 mL of dichloromethane, washed with additional 1M K₂CO₃. The combined aqueous extracts were back extracted with additional dichloromethane, and the combined organic extracts dried (Na₂SO₄), filtered and concentrated under reduced pressure to provide 11.1 g of an orange foam. This foam was dissolved in a minimal amount of dichloromethane, placed on a 70 g Isolute silica gel column and eluted with 0 to 100% ethyl acetate in hexane (0 to 20 minutes), 100% ethyl acetate (20-35 minutes), 100% dichloromethane (35-40 minutes), 0 ramp to 5% methanol/dichloromethane (40-60 mintes), 5/95 methanol/dichloromethane (60-70 minutes) at 30 mL/minutes and 254 nM detection wavelength to provide the title compound. ¹H NMR (300 MHz, DMSO-D6) δ ppm 1.63 (m, 2H) 1.80 (m, 2H) 2.00 (m, 2H) 2.85 (m, 2H) 3.39 (bs, 2H) 3.76 (m, 1H) 3.93 (s, 3H) 5.98 (s, 2H) 6.73 (m, 2H) 6.85 (m, 2H) 7.10 (dd, 1H) 7.24 (d, 1H) 7.95 (d, 1H) 8.83 (d, 1H); MS (ESI, MeOH/NH4OH) m/z 437 [M+H], 459 [M+Na], 435 [M−H].

### Example 3

### 7-methoxy-4-oxo-N-[1-(1-quinolin-6-ylethyl) piperidin-4-yl]-4H-chromene-2-carboxamide

### Example 3A

### Quinoline-6-carbaldehyde

A mixture of 6-methyl-quinoline (70.0 g, 0.489 mol) and pulverized selenium dioxide (56.4 g, 0.513 mol) was stirred and heated to 150-155° C. for 24 hours after which the mixture was cooled to 55° C. followed by the addition of Ethyl acetate (125 mL). The mixture was stirred for 5-10 minutes after which the ethyl acetate solution was decanted and this procedure repeat 5 times. The combined ethyl acetate extracts were washed with 5% sodium bicarbonate solution (2×125 mL) and 15% sodium chloride solution (170 mL). Decolorizing carbon (25 g) was added and the mixture was filtered through celite which was washed with ethyl acetate (200 mL). The combined ethyl acetate solution was concentrated under reduced pressure to provide a residue which was recrystallized from ethyl acetate and heptane to provide quinoline-6-carbaldehyde. ¹H NMR (400 MHz, CHLOROFORM-D) δ ppm 7.50 (dd, J=4.25, 8.37, 1H), 8.18 (m, 2H), 8.29 (dd, J=1.78, 8.37 Hz, 1H), 8.33 (d, J=1.24, 1H), 9.02 (dd, J=1.78, 4.25, 1H), 10.17 (s, 1H); MS (DCI) 158 (M+H)⁺.

### Example 3B

### 1-Quinolin-6-yl-ethanol

To a stirred solution of quinoline-6-carbaldehyde (2.42 g, 15.4 mmol) in tetrahydrofuran (20 mL) at 2° C. was added a 3 M solution of methyl magnesium bromide in tetrahydrofuran (7.7 mL, 23.1 mmol) while maintaining an internal temperature of less than 12° C. The solution was stirred for 20 minutes after which saturated ammonium chloride (50 mL) was added followed by the addition of 15% ammonium chloride. The mixture was extracted with ethyl acetate (2×75 mL) and the combined ethyl acetate extracts were washed with 15% potassium carbonate, 7% sodium chloride, dried over sodium sulfate, filtered and concentrated to provide 1-quinolin-6-yl-ethanol. ¹H NMR (400 MHz, CHLOROFORM-D) δ ppm 1.58 (d, J=6.45 Hz, 3H), 2.73 (s, 1H) 5.09 (q, J=6.45 Hz, 1H) 7.36 (dd, J=8.23, 4.25 Hz, 1H) 7.70 (dd, J=8.78, 2.06 Hz, 1H) 7.78 (d, J=1.92 Hz, 1H) 8.04 (d, J=8.78 Hz, 1H) 8.10 (dm, J=8.23 Hz, 1H) 8.82 (dd, J=4.25, 1.78 Hz, 1H); MS (ESI) 173 (M+H)⁺.

### Example 3C

### 6-(1-Bromo-ethyl)-quinoline hydrobromide salt

To 1-quinolin-6-yl-ethanol (2.00 g, 11.5 mmol) was added hydrogen bromide (25.3 mL of 30 wt. % solution in acetic acid, 127 mm) and the solution was heated to 50-60° C. for 5 hours. The mixture was cooled to ambient and purged with nitrogen. Toluene (30 mL) was added and the solution was concentrated under reduced pressure (repeat 2×). The solid obtained was dried under reduced pressure at 25° C. for 16 hours to provide 6-(1-bromo-ethyl)-quinoline hydrobromide salt. ¹H NMR (400 MHz, DMSO-D6) δ ppm 2.12 (d, J=6.86 Hz, 3H), 5.78 (q, J=6.86 Hz, 1H), 8.04 (dd, J=8.37, 5.08 Hz, 1H), 8.24 (m, 2H), 8.43 (s, 1H), 9.06 (dd, J=8.30, 1.03 Hz, 1H), 9.28 (dd, J=5.08, 1.51 Hz, 1H); MS (ESI) 236 (M+H)⁺.

### Example 3

### 7-methoxy-4-oxo-N-[1-(1-quinolin-6-ylethyl) piperidin-4-yl]-4H-chromene-2-carboxamide

A solution of Example 2C (500 mg, 1.66 mmol) in 7 mL DMF was charged with quinoline bromide hydrobomide 3C (317 mg, 1.66 mmol) and K₂CO₃ (505 mg, 3.65 mmol). The mixture was stirred under nitrogen for 42 hours, diluted with dichloromethane, washed with distilled water, dried (Na₂SO₄), filtered and concentrated under reduced pressure to provide 1.17 g of a brown oil. Flash silica gel chromatography with dichloromethane and methanol as eluent provided the title compound. ¹H NMR (300 MHz, DMSO-D6) δ ppm 1.41 (d, 3H) 1.67 (m, 2H) 1.83 (m, 2H) 2.07 (m, 2H) 2.84 (m, 1H) 3.02 (m, 1H) 3.72 (m, 2H) 3.91 (s, 3H) 6.74 (s, 1H) 7.10 (dd, 1H) 7.23 (d, 1H) 7.51 (dd, 1H) 7.79 (dd, 1H) 7.86 (d, 1H) 7.93 (d, 1H) 7.99 (d, 1H) 8.34 (m, 1H) 8.80 (d, 1H) 8.87 (dd, 1H); MS (ESI, MeOH/NH4OH) m/z 458 [M+H], 480 [M+Na], 456 [M−H].

### Example 4

### 7-chloro-N-[1-(2,3-dihydro-1,4-benzodioxin-6-ylmethyl) piperidin-4-yl]-4-oxo-4H-chromene-2-carboxamide

Sodium triacetoxyborohydride (19 mg, 91.3 μmol) was added to a mixture of Example 1G (14 mg, 45.6 μmol), 1,4-benzodioxan-6-carbaldehyde (7.5 mg, 45.6 μmol), sodium sulfate (13 mg, 91 μmol), acetic acid (20 μL), and THF (1 mL). After 24 hours, methanol (0.2 mL) was added, and the mixture was diluted with Ethyl acetate (20 mL). The mixture was washed with 1N aqueous NaOH (1×20 mL), washed with brine (1×20 mL), dried (MgSO₄), filtered, and concentrated under reduced pressure. The residue was loaded on a 0.5 gram MP-TsOH cartridge, and the cartridge was washed with dichloromethane. Next, the cartridge was treated with 2M ammonia in methanol, and the filtrate was concentrated to a residue. Finally, the residue was loaded on a 1 gram sep pack (SiO₂), and elution with Ethyl acetate to provided the title compound as a solid. ¹H NMR (300 MHz, DMSO-d₆) δ ppm 1.65 (m, 2H), 1.78 (m, 2H), 2.00 (m, 2H), 2.83 (m, 2H), 3.36 (s, 2H), 3.77 (m, 1H), 4.22 (s, 4H), 6.77 (m, 4H), 7.59 (dd, J=8.65, 1.86 Hz, 1H), 7.91 (d, J=2.03 Hz, 1H), 8.04 (d, J=8.48 Hz, 1H), 8.81 (d, J=7.80 Hz, 1H); MS (APCI) m/z 457 [M+H]⁺, 453 [M−H]⁺.

### Example 6

### 7-methoxy-N-[1-(2-naphthylmethyl)piperidin-4-yl]-4-oxo-4H-chromene-2-carboxamide

A solution of Example 2C (30 mg, 0.1 mmol) in 1 mL of dichloromethane containing 2% acetic acid was charged with 2-napthaldehyde (15 mg, 0.1 mmol) followed by NaBH(OAc)₃ (42 mg, 0.2 mmol). The mixture was stirred for 18 hours followed by the addition of 250 uL 1M K₂CO₃, diluted with 2 mL dichloromethane, dried (Na₂SO₄), filtered through a plug of silica gel (1 g, SepPak), rinsed through with 95:5 dichloromethane:methanol and concentrated to provide the title compound. ¹H NMR (300 MHz, DMSO-D6) δ ppm 1.70 (m, 2H) 1.81 (m, 2H) 2.11 (m, 2H) 2.89 (m, 2H) 3.65 (s, 2H) 3.79 (m, 1H) 3.93 (s, 3H) 6.76 (s, 1H) 7.11 (dd, 1H) 7.24 (d, 1H) 7.50 (m, 3H) 7.79 (m, 1H) 7.91 (m, 4H) 8.82 (d, 1H); MS (ESI, MeOH/NH4OH) m/z 443 [M+H], 465 [M+Na], 441 [M−H], 477 [M+Cl].

### Example 7

### N-[1-(1,3-benzodioxol-5-ylmethyl)piperidin-4-yl]-6-fluoro-4-oxo-4H-chromene-2-carboxamide

### Example 7A

### 4-[(6-Fluoro-4-oxo-4H-chromene-2-carbonyl)-amino]piperidine-1-carboxylic acid tert-butyl ester

To a solution of 6-fluoro-4-oxo-4H-chromene-2-carboxylic acid (2.8 g, 12.5 mmol) and 4-amino-1-BOC-piperidine (2.5 g, 12.5 mmol) in 50 mL DMF was added hydroxybenzotriazole (1.7 g, 12.5 mmol) and ethyl(dimethylaminopropyl)carbodiimide hydrochloride (2.5 g, 13.1 mmol). The mixture was stirred under nitrogen for 22 hours, diluted with ethyl acetate, washed with 1M HCl, 1M K₂CO₃, brine, dried (Na₂SO₄), filtered and concentrated under reduced pressure to provide the title compound. ¹H NMR (300 MHz, DMSO-D6) δ ppm 1.42 (s, 9H) 1.52 (m, 2H) 1.81 (m, 2H) 2.86 (m, 2H) 3.99 (m, 3H) 6.85 (s, 1H) 7.83 (m, 3H) 8.91 (d, 1H); MS (ESI, MeOH/NH4OH) m/z 413 [M+Na], 389 [M−H], 425 [M+Cl].

### Example 7B

### 4-[(6-fluoro-4-oxo-4H-chromene-2-carbonyl)-amino]-piperidine

A solution of Example 7A (2.5 g, 6.4 mmol) in 18 mL of dichloromethane at 0° C. was treated with 9 mL of trifluoroacetic acid and the mixture stirred for 2 hours, concentrated to an orange oil which was diluted with 95/5 dichloromethane/Methanol, washed with 1M K₂CO₃. The combined aqueous extracted were back extracted with additional dichloromethane, and the combined organic extracts dried (Na₂SO₄), filtered, concentrated under reduced pressure to provide the title compound (1.4 g, 75%). ¹H NMR (300 MHz, DMSO-D6) δ ppm 1.56 (m, 2H) 1.80 (m, 2H) 2.63 (m, 2H) 3.06 (m, 2H) 3.87 (m, 1H) 6.85 (s, 1H) 7.87 (m, 3H) 8.96 (m, 1H); MS (ESI, MeOH/NH4OH) m/z 291 [M+H], 581 [2M+H], 603 [2M+H], 289 [M−H], 579 [2M−H].

### Example 7

### N-[1-(1,3-benzodioxol-5-ylmethyl)piperidin-4-yl]-6-fluoro-4-oxo-4H-chromene-2-carboxamide

To a solution of Example 7B (29 mg, 0.1 mmol) in 0.5 mL THF containing 2% acetic acid was added with piperonal (15 mg, 0.1 mmol), NaBH(OAc)₃ (42 mg, 0.2 mmol) and Na₂SO₄ (28 mg, 0.2 mmol). The mixture was stirred for 40 hours followed by the addition of 30 uL of 3M H₂SO₄, shaken for 5 minutes, then 750 uL of 1M K₂CO₃ was added, shaken 10 minutes, 2 mL 95/5 dichloromethane/Methanol was added followed by drying agent (Na₂SO₄), filtered through a plug of silica gel (2 g, SepPak), rinsed through with 95/5 dichloromethane/Methanol and concentrated. Flash silica gel eluting with dichloromethane/Methanol to provide the title compound. ¹H NMR (300 MHz, DMSO-D6) δ ppm 1.64 (m, 2H) 1.79 (m, 2H) 1.99 (m, 2H) 2.85 (m, 2H) 3.39 (s, 2H) 3.77 (m, 1H) 5.97 (s, 1H) 5.99 (s, 2H) 6.75 (m, 2H) 6.85 (m, 3H) 7.74 (m, 1H) 7.85 (m, 1H) 8.88 (d, 1H); MS (ESI, MeOH/NH4OH) m/z 425 [M+H], 423 [M−H], 459 [M+Cl].

### Example 8

### 7-chloro-N-{1-[(1-methyl-1H-indol-5-yl)methyl]piperidin-4-yl}-4-oxo-4H-chromene-2-carboxamide

Example 8 (1.2 mg, 6%) was made according to the procedure described in Example 4, substituting Example 17A, N-methylindole-5-carboxaldehyde (7.3 mg, 45.6 μmol), for 1,4-benzodioxan-6-carbaldehyde. ¹H NMR (300 MHz, DMSO-d₆) δ ppm 1.63 (m, 2H), 1.77 (m, 2H), 1.98 (m, 2H), 2.86 (m, 2H), 3.53 (s, 2H), 3.76 (m, 1H), 3.76 (s, 3H), 6.36 (d, J=3.05 Hz, 1H), 6.81 (m, 1H), 7.11 (dd, J=8.48, 1.36 Hz, 1H), 7.28 (d, J=3.05 Hz, 1H), 7.36 (d, J=8.48 Hz, 1H), 7.42 (s, 1H), 7.58 (dd, J=8.82, 2.03 Hz, 1H), 7.89 (d, J=1.70 Hz, 1H), 8.03 (d, J=8.48 Hz, 1H), 8.79 (d, J=7.80 Hz, 1H); MS (APCI) m/z 450 [M−H]⁺.

### Example 9

### N-[1-(1,3-benzodioxol-5-ylmethyl)piperidin-4-yl]-6-chloro-7-methyl-4-oxo-4H-chromene-2-carboxamide

### Example 9A

### 1-(1,3-benzodioxol-5-ylmethyl)piperidin-4-amine

Acetic acid (2 mL) was added to a suspension of 4-Boc-aminopiperidine (5.0 g, 25.0 mmol), piperonal (3.75 g, 25.0 mmol), sodium sulfate (7.1 g, 50 mmol), and THF (100 mL). After 20 minutes sodium triacetoxyborohydride (10.6 g, 50.0 mmol) was added. After 16 hours, methanol (4 mL) was added and the mixture was stirred for 24 hours. The solution was diluted with dichloromethane (150 mL), washed with 1N aqueous NaOH (2×150 mL), washed with brine (1×100 mL), dried (MgSO₄), filtered, and concentrated under reduced pressure to provide a white solid (9.0 g) that was dissolved in dichloromethane (50 mL), cooled to 0° C., and combined with trifluoroacetic acid (25 mL). The mixture was stirred for 1 hour, concentrated under reduced preassure and the residue combined with 2M HCl in ethyl ether (30 mL) and ethyl ether (30 mL). The resulting white precipitate was collected by filtration and air-dried overnight to provide the title compound as the dihydrochloride salt. ¹H NMR (300 MHz, DMSO-d₆) δ ppm 1.94 (m, 2H), 2.06 (m, 2H), 2.95 (m, 2H), 3.21 (m, 2H), 3.40 (m, 1H), 4.14 (d, J=5.42 Hz, 2H), 6.07 (s, 2H), 6.99 (m, 2H), 7.24 (s, 1H), 8.28 (s, 2H); MS (APCI) m/z 235 [M+H]⁺.

### Example 9

### N-[1-(1,3-benzodioxol-5-ylmethyl)piperidin-4-yl]-6-chloro-7-methyl-4-oxo-4H-chromene-2-carboxamide

6-Chloro-7-methylchromone-2-carboxylic acid (63 mg, 0.263 mmol) and Example 9A (162 mg, 0.526 mmol) were subjected to the procedure outlined in Example 1F to provide a yellow residue (69 mg) that was recrystallized from hot acetonitrile to provide the title compound. ¹H NMR (300 MHz, DMSO-d₆) δ ppm 1.63 (m, 2H), 1.76 (m, 2H), 1.99 (m, 2H), 2.82 (d, J=11.87 Hz, 2H), 3.30 (s, 3H), 3.38 (s, 2H), 3.76 (m, J=7.80 Hz, 1H), 5.98 (s, 2H), 6.74 (m, 1H), 6.81 (m, 2H), 6.85 (s, 1H), 7.79 (s, 1H), 7.95 (s, 1H), 8.85 (d, J=7.80 Hz, 1H); MS (APCI) m/z 455 [M+H]⁺, 453 [M−H]⁺.

### Example 10

### N-[1-(1,3-benzodioxol-5-ylmethyl)piperidin-4-yl]-7-(difluoromethoxy)-4-oxo-4H-chromene-2-carboxamide

### Example 10A

### Ethyl 7-(difluoromethoxy)-4-oxo-4H-chromene-2-carboxylate

A solution of ethyl 7-hydroxy-4-oxo-4H-chromene-2-carboxylate (2.00 g, 4.27 mmol) in DMF (10 mL) at −78° C. was treated with liquid chlorodifluoromethane (10 mL) and K₂CO₃ (1.77 g, 12.82 mmol). The cold bath was removed, and the suspension was allowed to warm to ambient temperature and stir for 2 hours. The mixture was diluted with Ethyl acetate (100 mL) and water (100 mL), the layers were separated and the organic layer was washed with sat. NaHCO₃ (50 mL), brine (50 mL), dried (Na₂SO₄), filtered and concentrated under reduced pressure. Purification by silica gel chromatography (20% Ethyl acetate/Hexanes) provided the title compound. MS (DCI/NH₃) m/z 285 [M+H]⁺.

### Example 10B

### 7-(difluoromethoxy)-4-oxo-4H-chromene-2-carboxylic acid

To a solution of Example 10A (ethyl 7-(difluoromethoxy)-4-oxo-4H-chromene-2-carboxylate 0.5 g, 1.75 mmol) in 10 mL of THF and 3 mL of distilled water was added 0.155 g of lithium hydroxide hydrate and the resulting mixture was stirred for 0.5 hour. The mixture was diluted by slow addition of 1 mL of 1M H₂SO₄ and the resulting solid was filtered off, washed with distilled water and air dried to provide the title compound. MS (ESI, MeOH/NH4OH) m/z 256 [M⁺].

### Example 10

### N-[1-(1,3-benzodioxol-5-ylmethyl)piperidin-4-yl]-7-(difluoromethoxy)-4-oxo-4H-chromene-2-carboxamide

1-Benzo[1,3]dioxol-5-ylmethyl-piperidin-4-ylamine (Example 9A) and 7-(difluoromethoxy)-4-oxo-4H-chromene-2-carboxylic acid (Example 10) were processed according to the procedure described in Example 1F to provide the title compound. ¹H NMR (300 MHz, DMSO-D6) δ ppm 1.80 (m, 2H), 2.07 (m, 2H), 3.09 (m, 2H), 3.43 (m, 2H), 3.99 (m, 1H), 4.22 (m, 2H) 6.09 (s, 2H) 6.85 (s, 1H) 6.96 (m, 1H) 7.02 (dd, 1H) 7.08 (d, 1H) 7.35 (dd, 1H) 7.50 (m, 1H), 7.51 (t, 1H), 8.10 (d, 1H), 9.08 (d, 1H); MS (ESI) m/z 473 [M+H]⁺.

### Example 11

### N-[1-(1,3-benzodioxol-5-ylmethyl)piperidin-4-yl]-7-chloro-3-methyl-4-oxo-4H-chromene-2-carboxamide

### Example 11a

### 1-(4-Chloro-2-hydroxy-phenyl)-propan-1-one

Ethyl magnesium bromide (1M solution in THF, 2.6 mL, 2.61 mmol) was added to a stirred solution of 4-chloro-2,N-dimethoxy-N-methyl-benzamide Example 1A (0.4 g, 1.74 mmol) in THF (4 mL) at 0° C. The mixture was allowed to warm up to 10° C. over 3 hours diluted by sequential drop wise addition of saturated aqueous NH₄Cl, 1N HCL and water. The mixture was extracted with ethyl acetate (3×25 mL) and the combined organic extracts washed with water, brine, dried (MgSO₄), filtered and concentrated under reduced pressure to a clear oil.

Boron tribromide (1M solution in dichloromethane, 2.6 mmol, 2.6 mL) was added in portions to a 0° C. solution of the above oil (0.345 g, 1.74 mmol) in dichlormethane (5 mL) allowed to warm to ambient temperature over 15 hours and diluted with saturated aqueous. The mixture was extracted with dichloromethane (4×25 mL) and the organic extracts washed with saturated NaHCO₃, water, brine, dried (MgSO₄), filtered and concentrated under reduced pressure to provide an oil which crystallized on standing. ¹H NMR (300 MHz, DMSO-d₆) δ ppm 1.08 (t, J=7.12 Hz, 1H), 3.08 (q, J=7.35 Hz, 1H), 7.01 (dd, J=8.65, 2.20 Hz, 1H), 7.06 (d, J=2.03 Hz, 1H), 7.89 (d, J=8.48 Hz, 1H), 12.03 (s, 1H).

### Example 11b

### 7-Chloro-3-methyl-4-oxo-4H-chromene-2-carboxylic acid

Sodium ethoxide (21% solution in ethanol, 3 mL, 8.4 mmol) was added in portions to a solution of Example 11a (0.39 g, 2.1 mmol) in ethanol (2 mL) at ambient temperature followed by the addition of diethyl oxalate (1.4 mL, 10.5 mmol). The mixture was heated to reflux for 2 hours, at 50° C. for 12 hours and cooled to ambient temperature followed by the addition of 3N HCl. The mixture was extracted with ethyl acetate (3×40 mL), and the organic extracts dried (MgSO₄), filtered and concentrated to a thick paste. The paste was taken up in acetic acid (5 mL) and concentrated HCl (0.5 mL) and heated to reflux for 2 hours after which it was cooled to ambient temperature, diluted with water and extracted with ethyl acetate (3×30 mL). The combined organic extracts were washed with water, brine, dried (MgSO₄) and concentrated under reduced pressure to a black paste. The black paste and LiOH.H₂O (0.18 g, 4.2 mmol) were taken up in THF/H₂O (3:1, 16 mL) and stirred at ambient temperature for 2 hours. The solvents were removed under reduced pressure, the residue diluted with water, acidified with 3N HCl (pH=2) and filtered. The filtrate was extracted with ethyl acetate (3×30 mL), the combined organic extracts were washed with water, brine, dried (MgSO₄), filtered and concentrated to provide the title compound. ¹H NMR (300 MHz, DMSO-d₆) δ ppm 2.22 (s, 3H), 7.55 (dd, J=8.48, 2.03 Hz, 1H), 7.88 (d, J=1.70 Hz, 1H), 8.05 (d, J=8.48 Hz, 1H).

### Example 11

### N-[1-(1,3-benzodioxol-5-ylmethyl)piperidin-4-yl]-7-chloro-3-methyl-4-oxo-4H-chromene-2-carboxamide

Oxalyl chloride (2M solution in dichloromethane, 0.1 mL, 0.19 mmol) was added drop wise to a solution of Example 11b (0.03 g, 0.12 mmol) in dichloromethane (2 mL) and DMF (0.1 mL) and the mixture was stirred at ambient temperature for 1 hour after which 1-benzo[1,3]dioxol-5-ylmethyl-piperidin-4-ylamine bis hydrochloride salt (0.038 g, 0.12 mmol) was added in a single portion to the mixture followed by the addition of Hunig's base (diisopropylethylamine) (0.065 mL, 0.37 mmol) and the resulting mixture stirred for 1 hour. The mixture was then diluted with water, extracted with ethyl acetate (4×25 mL), the combined organic extracts were washed with water, brine, dried (MgSO₄), filtered and concentrated under reduced pressure and purified on RP-HPLC to provide the title compound. ¹H NMR (500 MHz, DMSO-d₆) δ ppm 1.77 (m, 2H), 2.07 (m, 5H), 3.07 (s, 2H), 3.99 (d, J=20.28 Hz, 1H), 4.21 (s, 3H), 6.08 (s, 2H), 7.00 (m, 2H), 7.08 (m, 1H), 7.56 (dd, J=8.42, 1.87 Hz, 1H), 7.88 (d, J=1.87 Hz, 1H), 8.06 (d, J=8.42 Hz, 1H), 9.04 (d, J=7.18 Hz, 1H), 9.39 (s, 1H); MS (ESI) m/e 567.3 (M−1+TFA)⁺.

### Example 12

### 7-chloro-4-oxo-N-[1-(1-quinolin-6-ylethyl)piperidin-4-yl]-4H-chromene-2-carboxamide

### Example 12A

### [1-(1-Quinolin-6-yl-ethyl)-piperidin-4-yl]-carbamic acid tert-butyl ester

To a mixture of 6-(1-bromo-ethyl)-quinoline hydrobromide salt (10.78 g, 34 mmol), 4-(N-Boc-amino)-piperidine (7.00 g, 35 mmol) and potassium carbonate (325 mesh, 14.08 g, 102 mmol) was added DMF (240 mL) and the mixture was stirred at ambient temperature for 17 hours. The mixture was diluted with water (750 mL) and extracted with ethyl acetate (2×250 mL). The combined ethyl acetate layers were washed with water (3×270 mL, 1×210 mL) and concentrated under reduced pressure. Ethyl acetate (60 mL) was added and the mixture concentrated to a solid (repeat 1×). The solid obtained was recrystallized from ethyl acetate (70 mL) to provide 8.71 g of [1-(1-quinolin-6-yl-ethyl)-piperidin-4-yl]-carbamic acid tert-butyl ester as a white solid. ¹H NMR (400 MHz, Chloroform-D²) δ ppm 1.43 (s, 9H; d, J=6.4 Hz, 3H, m, 2H), 1.83 (d, J=12.49 Hz, 1H), 1.95 (d, J=12.35 Hz, 1H), 2.10 (q, J=11.11 Hz, 2H), 2.71 (d, J=12.76 Hz, 1H), 2.99 (d, J=12.76 Hz, 1H), 3.42 (bs, 1H), 3.56 (q, J=6.40 Hz, 1H), 4.41 (s, 1H), 7.37 (dd, J=8.30, 4.19 Hz, 1H), 7.66 (d, J=1.65 Hz, 1H), 7.74 (dd, J=8.64, 1.92 Hz, 1H), 8.04 (d, J=8.64 Hz, 1H), 8.11 (dd, J=8.37, 1.37 Hz, 1H), 8.86 (dd, J=4.19, 1.72 Hz, 1H), MS (ESI) 356 (M+H)⁺.

### Example 12B

### 1-(1-quinolin-6-ylethyl)piperidin-4-amine

Trifluoroacetic acid (1 mL) was added to a mixture of Example 12A (180 mg, 0.506 mmol) and dichloromethane at 0° C. After 1 hour volatiles were removed under reduced pressure, and the resulting residue taken up in 2M ethereal HCl (1 mL). The white precipitate formed and was collected by filtration. ¹H NMR (300 MHz, DMSO-d₆) δ ppm 1.82 (d, J=6.44 Hz, 3H) 2.00 (m, 2H) 2.12 (s, 2H) 2.89 (m, J=15.60 Hz, 2H) 3.22 (m, 2H) 3.53 (m, 1H) 3.77 (m, 1H) 4.71 (m, 1H) 7.80 (dd, J=8.31, 4.58 Hz, 1H) 8.26 (m, 3H) 8.66 (d, J=8.14 Hz, 1H) 9.12 (d, J=4.41 Hz, 1H) 11.37 (s, 1H); MS (APCI) m/z 256 [M+H]⁺.

### Example 12

### 7-chloro-4-oxo-N-[1-(1-quinolin-6-ylethyl)piperidin-4-yl]-4H-chromene-2-carboxamide

Example 1E (112 mg, 0.50 mmol) was combined with Example 12B (0.50 mmol) and processed according to the procedure of Example 9 to provide the title compound. ¹H NMR (300 MHz, DMSO-d₆) δ ppm 1.40 (d, J=6.78 Hz, 3H), 1.62 (m, 2H), 1.80 (m, 2H), 2.06 (m, 2H), 2.81 (m, 1H), 2.99 (m, 1H), 3.71 (m, J=6.44 Hz, 2H), 6.80 (s, 1H), 7.51 (dd, J=8.31, 4.24 Hz, 1H), 7.58 (dd, J=8.48, 2.03 Hz, 1H), 7.79 (dd, J=8.82, 2.03 Hz, 1H), 7.85 (d, J=1.70 Hz, 1H), 7.89 (d, J=1.70 Hz, 1H), 7.98 (d, J=8.48 Hz, 1H), 8.03 (d, J=8.48 Hz, 1H), 8.33 (d, J=7.46 Hz, 1H), 8.79 (d, J=8.14 Hz, 1H), 8.85 (dd, J=4.07, 1.70 Hz, 1H); MS (APCI) m/z 462 [M+H]⁺, 460 [M−H]⁺.

### Example 14

### N-[1-(1H-indol-5-ylmethyl)piperidin-4-yl]-7-methoxy-4-oxo-4H-chromene-2-carboxamide

A solution of Example 2C (30 mg, 0.1 mmol) in 1 mL of dichloromethane containing 2% acetic acid was charged with indole-5-carboxaldehyde (14 mg, 0.1 mmol) followed by NaBH(OAc)₃ (42 mg, 0.2 mmol). The mixture was stirred for 18 hours then diluted by the addition of 250 uL 1M K₂CO₃, diluted with 2 mL dichloromethane, dried (Na₂SO₄), filtered through a plug of silica gel (1 g, SepPak), rinsed through with 95:5 dichlrormethane:methanol and concentrated to provide the title compound. ¹H NMR (300 MHz, DMSO-D6) δ ppm 1.65 (m, 2H) 1.79 (m, 2H) 2.03 (m, 2H) 2.88 (m, 2H) 3.53 (s, 2H) 3.77 (m, 1H) 3.93 (s, 3H) 6.74 (s, 1H) 7.04 (d, 1H) 7.11 (dd, 1H) 7.23 (d, 1H) 7.32 (m, 2H) 7.45 (d, 1H) 7.93 (m, 2H) 8.81 (d, 1H) 11.1 (s, 1H); MS (ESI, MeOH/NH4OH) m/z 432 [M+H], 454 [M+Na], 430 [M−H].

### Example 15

### N-[1-(2,3-dihydro-1,4-benzodioxin-6-ylmethyl)piperidin-yl]-7-methoxy-4-oxo-4H-chromene-2-carboxamide

A solution of Example 2C (60 mg, 0.2 mmol) in 1 mL of 95/4 dichloromethane/methanol containing 2% acetic acid was charged with 1,4-benzodioxan-6-carboxaldehyde (33 mg, 0.2 mmol) followed by NaBH(OAc)₃ (84 mg, 0.4 mmol) and Na₂SO₄ (56 mg. 0.4 mmol). The reaction was stirred for 22 hours then additional 1,4-benzodioxan-6-carboxaldehyde (33 mg, 0.2 mmol) and NaBH(OAc)3 (84 mg, 0.4 mmol) added. Stirred another 24 hours and then quenched by addition of 1M K₂CO₃, shaken for 30 minutes, diluted with 95/5 dichloromethane/methanol, the bottom organic layer pipeted onto a silica gel plug (2 g, SepPak), rinsed with 90/10 dichloromethane/methanol and concentrated. The resultant yellow residue was purified by flash silica gel chromatography using dichloromethane/methanol as eluent to provide the title compound. ¹H NMR (300 MHz, DMSO-D6) δ ppm 1.65 (m, 2H) 1.77 (m, 2H) 1.99 (m, 2H) 2.81 (m, 2H) 3.37 (m, 2H) 3.76 (m, 1H) 3.93 (s, 3H) 4.22 (s, 4H) 6.76 (m, 4H) 7.11 (dd, J=8.81, 2.37 Hz, 1H) 7.23 (d, J=2.37 Hz, 1H) 7.94 (d, J=8.81 Hz, 1H) 8.81 (d, J=8.14 Hz, 1H); MS (ESI, MeOH/NH4OH) m/z 451 [M+H], 473 [M+Na], 449 [M−H].

### Example 16

### N-{1-[(2,2-difluoro-1,3-benzodioxol-5-yl)methyl]piperidin-4-yl}-7-methoxy-4-oxo-4H-chromene-2-carboxamide

A solution of Example 2C (30 mg, 0.1 mmol) in 1 mL of dichloromethane containing 2% acetic acid was charged with 2,2-difluoro-1,3-benzodioxole-5-carboxaldehyde (18 mg, 0.1 mmol) followed by NaBH(OAc)₃ (42 mg, 0.2 mmol). The reaction was stirred for 18 hours then quenched by addition of 250 uL 1M K₂CO₃, diluted with 2 mL dichloromethane, dried (Na₂SO₄), filtered through a plug of silica gel (1 g, SepPak), rinsed through with 95:5 dichloromethane:methanol and concentrated to provide the title compound. ¹H NMR (300 MHz, DMSO-D6) δ ppm 1.68 (m, 2H) 1.78 (m, 2H) 2.06 (m, 2H) 2.84 (m, 2H) 3.51 (s, 2H) 3.78 (m, 1H) 3.93 (s, 3H) 6.75 (s, 1H) 7.13 (m, 2H) 7.23 (d, J=2.37 Hz, 1H) 7.34 (m, 2H) 7.95 (d, 1H) 8.82 (d, J=8.14 Hz, 1H); MS (ESI, MeOH/NH4OH) m/z 473 [M+H], 495 [M+Na], 471 [M−H].

### Example 17

### 7-methoxy-N-{1-[(1-methyl-1H-indol-5-yl)methyl]piperidin-4-yl}-4-oxo-4H-chromene-2-carboxamide

### Example 17A

### 1-methyl-5-carboxaldehyde-indole

A solution of indole-5-carboaldehyde (2.2 g, 15.2 mmol) in 60 mL DMF at 0° C. was charged with NaH (60% dispersion on mineral oil, 760 mg, 19 mmol) portionwise. Initial gas evolution. Stirred 30 minutes after which iodomethane (1 mL, 2.27 g, 16 mmol) was added via syringe. After 1 hour 15 minutes, quenched by slow addition of distilled water (gas evolution). After gas evolution ceased, 200 mL distilled water added, beige precipitate filtered off and dried in a vacuum oven to provide the title compound. ¹H NMR (300 MHz, BENZENE-D₆) δ ppm 3.86 (s, 3H) 6.67 (d, J=3.39 Hz, 1H) 7.50 (d, J=3.05 Hz, 1H) 7.61 (d, J=8.48 Hz, 1H) 7.69 (m, 1H) 8.18 (s, 1H) 9.98 (s, 1H); MS (ESI, MeOH/NH4OH) m/z 160 [M+H], 177 [M+NH4].

### Example 17B

### 7-methoxy-N-{1-[(1-methyl-1H-indol-5-yl)methyl]piperidin-4-yl}-4-oxo-4H-chromene-2-carboxamide

A solution of Example 2C (60 mg, 0.2 mmol) in 1 mL of 95/4 dichloromethane/methanol containing 2% acetic acid was charged with Example 17A (32 mg, 0.2 mmol) followed by NaBH(OAc)₃ (84 mg, 0.4 mmol) and Na2SO4 (56 mg. 0.4 mmol). The reaction was stirred for 22 hours then additional 1,4-benzodioxan-6-carboxaldehyde (33 mg, 0.2 mmol) and NaBH(OAc)₃ (84 mg, 0.4 mmol) added. Stirred another 24 hours and then quenched by addition of 1M K₂CO₃, shaken for 30 minutes, diluted with 95/5 dichloromethane/methanol, bottom organic layer pipeted onto a silica gel plug (2 g, SepPak), rinsed with 90/10 dichloromethane/methanol and concentrated. The resultant yellow residue was purified by flash silica gel chromatography using dichloromethane/methanol as eluent to provide the title compound. ¹H NMR (300 MHz, DMSO-D6) δ ppm 1.66 (m, 2H) 1.77 (m, 2H) 2.02 (m, 2H) 2.87 (m, 2H) 3.54 (s, 2H) 3.77 (s, 3H) 3.8 (m, 1H) 3.93 (s, 3H) 6.37 (d, J=2.71 Hz, 1H) 6.75 (s, 1H) 7.11 (m, 2H) 7.22 (d, 1H) 7.29 (d, J=3.05 Hz, 1H) 7.37 (d, J=8.14 Hz, 1H) 7.44 (s, 1H) 7.94 (d, J=9.15 Hz, 1H) 8.80 (d, J=7.80 Hz, 1H); MS (ESI, MeOH/NH4OH) m/z 446 [M+H], 468 [M+Na], 444 [M−H].

### Example 18

### N-[1-(1,3-benzodioxol-5-ylmethyl)piperidin-4-yl]-8-chloro-7-methoxy-4-oxo-4H-chromene-2-carboxamide

### Example 18A

### 8-chloro-7-methoxy-4-oxo-4H-chromene-2-carboxylic acid

To a stirred suspension of 7-methoxy-4-oxo-4H-chromene-2-carboxylic acid (0.10 g, 0.455 mmol) in acetic acid (1.5 ml) at room temperature was added N-chlorosuccinimide (63 mg. 0.477 mmol), and the reaction was heated to 85 C for 6 hours. The heating bath was removed, and upon reaching room temperature the reaction was diluted with H₂O (2 mL). The solid was filtered and air-dried to yield 8-chloro-7-methoxy-4-oxo-4H-chromene-2-carboxylic acid as a white solid. ¹H NMR (300 MHz, DMSO-d₆) δ ppm 4.04 (s, 3H), 6.89 (s, 1H), 7.42 (d, J=9.16 Hz, 1H), and 8.01 (d, J=8.82 Hz, 1H).

### Example 18

### N-[1-(1,3-benzodioxol-5-ylmethyl)piperidin-4-yl]-8-chloro-7-methoxy-4-oxo-4H-chromene-2-carboxamide

To a stirred solution of 8-chloro-7-methoxy-4-oxo-4H-chromene-2-carboxylic acid (20 mg, 0.078 mmol), 1-benzo[1,3]dioxol-5-ylmethyl-piperidin-4-ylamine bis-hydrochloride salt (23 mg, 0.075 mmol), NMM (0.041 mL, 0.38 mmol), and HOBt (13 mg, 0.094 mmol) in DMF (1 mL) was added EDCI (18 mg, 0.094 mmol). The reaction was heated to 55 C for 12 hours, and concentrated under reduced pressure. Purification of the residue by RP-HPLC provided 8-chloro-7-methoxy-4-oxo-4H-chromene-2-carboxylic acid 1-(benzo[1,3]dioxol-5-ylmethyl-piperidin-4-yl)-amide as a solid. ¹H NMR (300 MHz, DMSO-d₆) δ ppm 1.81 (s, 2H), 2.08 (m, J=8.82 Hz, 2H), 2.73 (m, 2H), 3.09 (m, 2H), 3.95 (m, 1H), 4.04 (m, 3H), 4.21 (d, J=4.75 Hz, 2H), 6.09 (s, 2H), 6.87 (m, 1H), 7.02 (m, 3H), 7.43 (d, J=9.00 Hz, 1H), 8.02 (d, J=9.00 Hz, 1H), and 8.80 (d, J=7.46 Hz, 1H). MS (ESI) m/z 471 [M+H]⁺.

### Example 19

### N-[1-(3-fluoro-4-methoxybenzyl)piperidin-4-yl]-7-methoxy-4-oxo-4H-chromene-2-carboxamide

A solution of Example 2C (60 mg, 0.2 mmol) in 1 mL of 95/4 dichloromethane/methanol containing 2% acetic acid was charged with 3-fluoro-4-methoxybenzaldehyde (31 mg, 0.2 mmol) followed by NaBH(OAc)₃ (84 mg, 0.4 mmol) and Na₂SO₄ (56 mg. 0.4 mmol). The reaction was stirred for 22 hours then additional 1,4-benzodioxan-6-carboxaldehyde (33 mg, 0.2 mmol) and NaBH(OAc)3 (84 mg, 0.4 mmol) added. The mixture was stirred 24 hours and then diluted by addition of 1M K₂CO₃, shaken for 30 minutes, diluted with 95/5 dichloromethane/methanol, bottom organic layer pipeted onto a silica gel plug (2 g, SepPak), rinsed with 90/10 dichloromethane/methanol and concentrated. The resultant yellow residue was purified by flash silica gel chromatography using dichloromethane/methanol as eluent to provide the title compound. ¹H NMR (300 MHz, DMSO-D6) δ ppm 1.65 (m, 2H) 1.78 (m, 2H) 2.02 (m, 2H) 2.83 (m, 2H) 3.42 (s, 2H) 3.76 (m, 1H) 3.82 (s, 3H) 3.93 (s, 3H) 6.75 (s, 1H) 7.11 (m, 4H) 7.23 (d, J=2.37 Hz, 1H) 7.94 (d, J=8.81 Hz, 1H) 8.81 (d, J=8.14 Hz, 1H); MS (ESI, MeOH/NH4OH) m/z 441 [M+H], 463 [M+Na], 439 [M−H].

### Example 20

### N-[1-(4-chlorobenzyl)piperidin-4-yl]-7-methoxy-4-oxo-4H-chromene-2-carboxamide

A solution of Example 2C (60 mg, 0.2 mmol) in 1 mL of 95/5 dichloromethane/methanol containing 2% acetic acid was charged with 4-chlorobenzaldehyde (28 mg, 0.2 mmol) followed by NaBH(OAc)₃ (84 mg, 0.4 mmol) and Na₂SO₄ (56 mg. 0.4 mmol). The mixture was stirred for 22 hours then additional 1,4-benzodioxan-6-carboxaldehyde (33 mg, 0.2 mmol) and NaBH(OAc)₃ (84 mg, 0.4 mmol) added. The mixture was stirred for 24 hours and then diluted by addition of 1M K₂CO₃, shaken for 30 minutes, diluted with 95/5 dichloromethane/methanol, bottom organic layer pipeted onto a silica gel plug (2 g, SepPak), rinsed with 90/10 dichloromethane/methanol and concentrated. The resultant yellow residue was purified by flash silica gel chromatography using dichloromethane/methanol as eluent to provide the title compound. ¹H NMR (300 MHz, DMSO-D6) δ ppm 1.67 (m, 2H) 1.80 (m, 2H) 2.05 (m, 2H) 2.83 (m, 2H) 3.48 (s, 2H) 3.79 (m, 1H) 3.93 (s, 3H) 6.75 (s, 1H) 7.11 (dd, J=8.81, 2.37 Hz, 1H) 7.23 (d, J=2.37 Hz, 1H) 7.36 (m, 4H) 7.94 (d, J=8.81 Hz, 1H) 8.81 (d, J=7.80 Hz, 1H); MS (ESI, MeOH/NH4OH) m/z 427 [M+H], 449 [M+Na], 425 [M−H].

### Example 21

### N-[1-(3-bromobenzyl)piperidin-4-yl]-7-methoxy-4-oxo-4H-chromene-2-carboxamide

To a solution of Example 2C (30 mg, 0.1 mmol) in 1 mL of dichloromethane containing 2% acetic acid was added with 3-bromobenzaldehyde (18 mg, 0.1 mmol) followed by NaBH(OAc)₃ (42 mg, 0.2 mmol) and the mixture was stirred for 18 hours. 250 uL of 1M K₂CO₃ was added and the mixture was diluted with 2 mL dichloromethane which was dried (Na₂SO₄), filtered through a plug of silica gel (1 g, SepPak), rinsed through with 95:5 dichloromethane:methanol and concentrated to provide the title compound. ¹H NMR (300 MHz, DMSO-D6) δ ppm 1.65 (m, 2H) 1.79 (m, 2H) 2.07 (m, 2H) 2.81 (m, 2H) 3.50 (s, 2H), 3.8 (m, 1H), 3.93 (s, 3H) 6.75 (s, 1H) 7.11 (dd, J=8.81, 2.37 Hz, 1H) 7.23 (d, J=2.37 Hz, 1H) 7.30 (m, 2H) 7.44 (m, 1H) 7.52 (m, 1H) 7.95 (d, J=8.82 Hz, 1H) 8.82 (d, 1H); MS (ESI, MeOH/NH4OH) m/z 473 [M+H], 495 [M+Na].

### Example 22

### 6-fluoro-N-{1-[(1-methyl-1H-indol-5-yl)methyl]piperidin-4-yl}-4-oxo-4H-chromene-2-carboxamide

To a solution of Example 7B (29 mg, 0.1 mmol) in 0.5 mL THF containing 2% acetic acid was added Example 17A (16 mg, 0.1 mmol), NaBH(OAc)₃ (42 mg, 0.2 mmol) and Na₂SO₄ (28 mg, 0.2 mmol). After 40 hours, the mixture was quenched by addition of 30 uL of 3M H₂SO₄, shaken for 5 minutes, then 750 uL of 1M K₂CO₃ was added, shaken 10 minutes, 2 mL 95/5 dichloromethane/methanol was added followed by drying agent (Na₂SO₄), filtered through a plug of silica gel (2 g, SepPak), rinsed through with 95/5 dichloromethane/methanol and concentrated under reduced pressure. Flash silica gel eluting with dichloromethane/methanol provided the title compound. ¹H NMR (300 MHz, DMSO-D6) δ ppm 1.66 (m, 2H) 1.77 (m, 2H) 2.00 (m, 2H) 2.89 (m, 2H) 3.54 (s, 2H) 3.77 (m, 4H) 6.37 (d, J=3.05 Hz, 1H) 6.83 (s, 1H) 7.11 (dd, J=8.48, 1.70 Hz, 1H) 7.29 (d, J=3.05 Hz, 1H) 7.41 (m, 2H) 7.72 (m, 1H) 7.84 (m, 2H) 8.87 (d, J=7.80 Hz, 1H); MS (ESI, MeOH/NH4OH) m/z 434 [M+H], 432 [M−H].

### Example 23

### 7-chloro-N-[1-(3-fluoro-4-methoxybenzyl)piperidin-4-yl]-4-oxo-4H-chromene-2-carboxamide

Example 23 (5.4 mg, 27%) was made according to the procedure described in Example 4, substituting 3-fluoro-4-methoxybenzaldehyde (7.0 mg, 45.6 μmol) for 1,4-benzodioxan-6-carbaldehyde. ¹H NMR (300 MHz, DMSO-d₆) δ ppm 1.65 (m, 2H), 1.79 (m, 2H), 2.03 (m, 2H), 2.82 (m, 2H), 3.42 (s, 2H), 3.78 (m, 1H), 3.82 (s, 3H), 6.82 (s, 1H), 7.11 (m, 3H), 7.59 (dd, J=8.82, 2.03 Hz, 1H), 7.90 (d, J=2.03 Hz, 1H), 8.04 (d, J=8.48 Hz, 1H), 8.81 (d, J=7.80 Hz, 1H); MS (APCI) m/z 445 [M+H]⁺, 443 [M−H]⁺.

### Example 24

### N-{1-[(2,2-difluoro-1,3-benzodioxol-5-yl)methyl]piperidin-4-yl}-7-(difluoromethoxy)-4-oxo-4H-chromene-2-carboxamide

Example 24 was prepared according to the procedure described in Example 10, substituting 1-[(2,2-difluoro-1,3-benzodioxol-5-yl)methyl]piperidin-4-ylamine for 1-benzo[1,3]dioxol-5-ylmethyl-piperidin-4-ylamine. ¹H NMR (300 MHz, DMSO-D6) δ ppm 1.84 (m, 2H), 2.08 (m, 2H), 3.09 (m, 2H), 3.48 (m, 2H), 3.99 (m, 1H), 4.33 (m, 2H), 6.85 (s, 1H) 7.35 (m, 2H), 7.51 (m, 2H) 7.54 (t, 1H), 7.57 (m, 1H), 8.10 (d, 1H), 9.07 (d, 1H); MS (ESI) m/z 509 [M+H]⁺.

### Example 25

### N-[1-(2-fluoro-4-methoxybenzyl)piperidin-4-yl]-7-methoxy-4-oxo-4H-chromene-2-carboxamide

To a solution of Example 2C (30 mg, 0.1 mmol) in 1 mL of dichloromethane containing 2% acetic acid was added with 2-fluoro-4-methoxybenzaldehyde (15 mg, 0.1 mmol) followed by NaBH(OAc)₃ (42 mg, 0.2 mmol). The mixture was stirred for 18 hours then quenched by addition of 250 uL 1M K₂CO₃, diluted with 2 mL dichloromethane, dried (Na₂SO₄), filtered through a plug of silica gel (1 g, SepPak), rinsed through with 95:5 dichloromethane:methanol and concentrated to provide the title compound. ¹H NMR (300 MHz, DMSO-D6) δ ppm 1.64 (m, 2H) 1.77 (m, 2H) 2.01 (m, 2H) 2.84 (m, 2H) 3.46 (s, 2H) 3.75 (m, 4H) 3.93 (s, 3H) 6.78 (m, 3H) 7.10 (dd, J=8.98, 2.54 Hz, 1H) 7.26 (m, 2H) 7.94 (d, J=8.81 Hz, 1H) 8.81 (d, J=7.80 Hz, 1H); MS (ESI, MeOH/NH4OH) m/z 441 [M+H], 463 [M+Na], 439 [M−H].

### Example 26

### 7-chloro-4-oxo-N-[1-(quinolin-6-ylmethyl)piperidin-4-yl]-4H-chromene-2-carboxamide

Example 26 (6.1 mg, 30%) was made analogous to Example 4, substituting 6-quinoline carboxaldehyde (7.0 mg, 45.6 μmol) for 1,4-benzodioxan-6-carbaldehyde. ¹H NMR (300 MHz, DMSO-d₆) δ ppm 1.71 (m, 2H), 1.82 (m, 2H), 2.12 (m, 2H), 2.88 (m, 2H), 3.68 (s, 2H), 3.82 (m, 1H), 6.82 (s, 1H), 7.51 (dd, J=8.31, 4.24 Hz, 1H), 7.58 (dd, J=8.48, 2.03 Hz, 1H), 7.74 (dd, J=8.65, 1.86 Hz, 1H), 7.85 (s, 1H), 7.89 (d, J=1.70 Hz, 1H), 7.98 (d, J=8.81 Hz, 1H), 8.03 (d, J=8.48 Hz, 1H), 8.33 (d, J=8.14 Hz, 1H), 8.82 (d, J=7.80 Hz, 1H), 8.86 (dd, J=4.07, 1.70 Hz, 1H); MS (APCI) m/z 448 [M+H]⁺.

### Example 27

### N-[1-(4-acetylbenzyl)-piperidin-4-yl]-7-methoxy-4-oxo-4H-chromene-2-carboxamide

To a solution of Example 2C (60 mg, 0.2 mmol) in 1 mL of 95/4 dichloromethane/methanol containing 2% acetic acid was added 4-acetylbenzaldehyde (30 mg, 0.2 mmol) followed by NaBH(OAc)₃ (84 mg, 0.4 mmol) and Na₂SO₄ (56 mg. 0.4 mmol). The mixture was stirred for 22 hours followed by the addition of 1,4-benzodioxan-6-carboxaldehyde (33 mg, 0.2 mmol) and NaBH(OAc)₃ (84 mg, 0.4 mmol). The mixture was stirred another 24 hours and then quenched by addition of 1M K₂CO₃, shaken for 30 minutes, diluted with 95/5 dichloromethane/methanol. The bottom organic layer was pipeted onto a silica gel plug (2 g, SepPak), rinsed with 90/10 dichloromethane/methanol and concentrated under reduced pressure. The resultant yellow residue was purified by flash silica gel chromatography using dichloromethane/methanol as eluent to provide the title compound. ¹H NMR (300 MHz, DMSO-D6) δ ppm 1.67 (m, 2H) 1.79 (m, 2H) 2.08 (m, 2H) 2.54 (s, 3H) 2.84 (m, 2H) 3.57 (s, 2H) 3.78 (m, 1H) 3.92 (s, 3H) 6.76 (s, 1H) 7.11 (dd, J=8.81, 2.37 Hz, 1H) 7.23 (d, J=2.37 Hz, 1H) 7.46 (m, 2H) 7.93 (m, 3H) 8.82 (d, J=7.80 Hz, 1H); MS (ESI, MeOH/NH4OH) m/z 435 [M+H], 457 [M+Na], 439 [M−H].

### Example 28

### 7-methoxy-4-oxo-N-[1-(quinolin-6-ylmethyl)piperidin-4-yl]-4H-chromene-2-carboxamide

To a solution of Example 2C (161 mg, 0.53 mmol) in 1 mL of dichloromethane and 1 mL THF containing 2% acetic acid was added quinoline-6-carboxaldehyde (Example 3A) (84 mg, 0.53 mmol) followed by NaBH(OAc)₃ (223 mg, 1.06 mmol). The mixture was stirred for 65 hours then quenched by slow addition of 1M K₂CO₃, diluted with dichloromethane, washed with additional 1M K₂CO₃, dried (Na₂SO₄), and concentrated to provide 235 mg of an orange foam. Flash silica gel chromatography with hexane/ethyl acetate followed by dichloromethane/methanol to provide the title compound. ¹H NMR (300 MHz, DMSO-D6) δ ppm 1.68 (m, 2H) 1.81 (m, 2H) 2.11 (m, 2H) 2.89 (m, 2H) 3.69 (s, 2H) 3.82 (m, 1H) 3.93 (s, 3H) 6.76 (s, 1H) 7.10 (dd, J=8.98, 2.54 Hz, 1H) 7.23 (d, J=2.37 Hz, 1H) 7.52 (dd, J=8.31, 4.24 Hz, 1H) 7.75 (dd, J=8.48, 1.70 Hz, 1H) 7.86 (s, 1H) 7.95 (d, J=8.82 Hz, 1H) 7.99 (d, J=8.81 Hz, 1H) 8.33 (d, J=7.46 Hz, 1H) 8.81 (d, J=7.80 Hz, 1H) 8.87 (dd, J=4.24, 1.53 Hz, 1H); MS (ESI, MeOH/NH4OH) m/z 444 [M+H], 466 [M+Na], 442 [M−H].

### Example 29

### N-[1-(1,3-benzodioxol-5-ylmethyl)piperidin-4-yl]-6-chloro-4-oxo-4H-chromene-2-carboxamide

### Example 29A

### tert-butyl 4-{[(6-chloro-4-oxo-4H-chromen-2-yl)carbonyl]amino}piperidine-1-carboxylate

To a solution of 6-chlor-4-oxo-4H-chromene-2-carboxylic acid (2.8 g, 12.5 mmol) and 4-amino-1-BOC-piperidine (2.5 g, 12.5 mmol) in 50 mL DMF was added hydroxybenzotriazole (1.7 g, 12.5 mmol) and ethyl(dimethylaminopropyl)carbodiimide hydrochloride (2.5 g, 13.1 mmol). The rmixture was stirred under nitrogen for 22 hours, diluted with ethyl acetate, washed with 1M HCl, 1M K₂CO₃, brine, dried (Na₂SO₄), filtered and concentrated to provide the title compound. ¹H NMR (300 MHz, DMSO-D6) δ ppm 1.42 (s, 9H) 1.50 (m, 2H) 1.80 (m, 2H) 2.81 (m, 2H) 3.97 (m, 3H) 6.87 (s, 1H) 7.80 (d, J=8.82 Hz, 1H) 7.95 (m, 2H) 8.91 (d, J=8.14 Hz, 1H); MS (ESI, MeOH/NH4OH) m/z 429 [M+Na], 405 [M−H].

### Example 29B

### 6-chloro-4-oxo-N-piperidin-4-yl-4H-chromene-2-carboxamide

To a solution of Example 29A (2.5 g, 6.1 mmol) in 16 mL of dichloromethane at 0° C. was added trifluoroacetic acid (8 mL). The mixture was allowed to slowly warm to room temperature, stirred for 2 hours, then concentrated to a yellow oil. The oil was diluted in 95/5 dichloromethane/methanol, washed with 1M K₂CO₃, aqueous extracted with additional dichloromethane, organic extracts dried (Na₂SO₄) and concentrated to provide the title compound. ¹H NMR (300 MHz, DMSO-D6) δ ppm 1.50 (m, 2H) 1.76 (m, 2H) 3.00 (m, 2H) 3.85 (m, 3H) 6.86 (s, 1H) 7.83 (d, J=8.82 Hz, 1H) 7.93 (d, J=2.71 Hz, 1H) 7.97 (m, 1H) 8.91 (d, J=8.14 Hz, 1H); MS (ESI, MeOH/NH4OH) m/z 307 [M+H], 305 [M−H].

### Example 29C

### N-[1-(1,3-benzodioxol-5-ylmethyl)piperidin-4-yl]-6-chloro-4-oxo-4H-chromene-2-carboxamide

To a solution of Example 29B (31 mg, 0.1 mmol) in 0.5 mL THF containing 2% acetic acid was added piperonal (15 mg, 0.1 mmol), NaBH(OAc)₃ (42 mg, 0.2 mmol) and Na₂SO₄ (28 mg, 0.2 mmol). After 40 hours, the mixture was quenched by addition of 30 uL of 3M H₂SO₄, shaken for 5 minutes, then 750 uL of 1M K₂CO₃ was added, shaken 10 minutes, 2 mL 95/5 dichloromethane/methanol was added followed by drying agent (Na₂SO₄), filtered through a plug of silica gel (2 g, SepPak), rinsed through with 95/5 dichloromethane/methanol and concentration to provide the title compound. ¹H NMR (300 MHz, DMSO-D6) δ ppm 1.65 (m, 2H) 1.77 (m, 2H) 1.99 (m, 2H) 2.83 (m, 2H) 3.39 (s, 2H) 3.78 (m, 1H) 5.97 (s, 1H) 5.99 (s, 2H), 6.80 (m, 1H), 6.90 (m, 2H), 7.82 (d, J=8.81 Hz, 1H) 7.97 (m, 2H) 8.89 (d, J=8.14 Hz, 1H); MS (ESI, MeOH/NH4OH) m/z 441 [M+H], 439 [M−H].

### Example 30

### 7-(difluoromethoxy)-4-oxo-N-[1-(1-quinolin-6-ylethyl piperidin-4-yl]-4H-chromene-2-carboxamide

Example 30 was prepared according to the procedure described in Example 10, substituting Example 12B for 1-benzo[1,3]dioxol-5-ylmethyl-piperidin-4-ylamine. ¹H NMR (300 MHz, DMSO-d₆) δ ppm 1.79 (d, 3H), 1.91 (m, 2H), 2.07 (m, 4H), 3.04 (m, 1H) 3.34 (m, 2H), 4.76 (m, 1H), 6.83 (s, 1H) 7.35 (dd, 1H), 7.51 (t, 1H), 7.65 (dd, 1H), 7.93 (dd, 1H), 8.10 (d, 1H), 8.15 (m, 3H), 8.45 (m, 1H), 9.00 (dd, 1H), 9.04 (d, 1H);

### Example 31

### N-[1-(4-bromobenzyl)piperidin-4-yl]-7-methoxy-4-oxo-4H-chromene-2-carboxamide

To a solution of Example 2C (30 mg, 0.1 mmol) in 1 mL of dichloromethane containing 2% acetic acid was added 4-bromobenzaldehyde (18 mg, 0.1 mmol) followed by NaBH(OAc)₃ (42 mg, 0.2 mmol). The mixture was stirred for 18 hours then quenched by addition of 250 uL 1M K₂CO₃, diluted with 2 mL dichloromethane, dried (Na₂SO₄), filtered through a plug of silica gel (1 g, SepPak), rinsed through with 95:5 dichloromethane:methanol and concentrated to provide the title compound. ¹H NMR (300 MHz, DMSO-D6) δ ppm 1.65 (m, 2H) 1.78 (m, 2H) 2.02 (m, 2H) 2.81 (m, 2H) 3.45 (s, 2H) 3.76 (m, 1H) 3.92 (s, 3H) 6.74 (s, 1H) 7.09 (dd, 1H) 7.24 (m, 3H) 7.50 (m, 2H) 7.93 (d, 1H) 8.78 (d, J=8.06 Hz, 1H); MS (ESI, MeOH/NH4OH) m/z 471 [M+H], 469 [M−H].

### Example 32

### N-[1-(1,3-benzodioxol-5-ylmethyl)piperidin-4-yl]-7-chloro-6-fluoro-4-oxo-1,4-dihydroquinoline-2-carboxamide

A 10 mL culture tube with screw cap was charged with 7-chloro-6-fluoro-4-oxo-1,4-dihydro-quinoline-2-carboxylic acid (40.0 mg, 0.166 mmol), 1-Benzo[1,3]dioxol-5-ylmethyl-piperidin-4-ylamine (42.9 mg, 0.183 mmol), EDCI (35.0 mg, 0.183 mmol), HOBT (24.7 mg, 0.183 mmol), NMM (42.6 mg, 0.398 mmol) and 2 mL of DMF, and the reaction vessel placed on a shaker for 14 hours. After this time, the DMF was removed in vacuo and the residue was dissolved in 1.5 mL of a 1:1 mixture of DMSO/MeOH and purified by preparative reverse-phase HPLC and converted to the free base product. ¹H NMR (400 MHz, DMSO-d₆) δ ppm 1.59-1.66 (m, 2H), 1.80 (d, J=9.99, 2H), 2.04 (t, 2H), 2.82 (d, J=11.85, 2H), 3.40 (s, 2H), 3.75-3.83 (m, 1H), 5.99 (s, 2H), 6.76 (d, J=9.35, 1H), 6.84-6.86 (m, 2H), 6.93 (br s, 1H), 7.89 (d, J=9.38, 1H), 8.18 (d, J=6.55, 1H), 8.76 (s, 1H), 11.94 (br s, 1H); MS (ESI) m/e 458.0 (M+H)⁺.

### Example 33

### 7-chloro-6-fluoro-N-[1-(2-fluoro-4-methoxybenzyl)piperidin-4-yl]-4-oxo-1,4-dihydroquinoline-2-carboxamide

The title compound was prepared according to the procedure for Example 32 substituting 1-(2-Fluoro-4-methoxy-benzyl)-piperidin-4-ylamine for 1-Benzo[1,3]dioxol-5-ylmethyl-piperidin-4-ylamine. ¹H NMR (500 MHz, DMSO-d₆) δ ppm 1.59-1.66 (m, 2H), 1.80 (d, J=12.17, 2H), 2.07 (t, 2H), 2.82 (d, J=11.86, 2H), 3.46 (s, 2H), 3.76-3.80 (m, 4H), 6.76-6.80 (m, 2H), 6.90 (br s, 1H), 7.28 (t, J=6.55, 1H), 7.89 (d, J=9.67, 1H), 8.18 (d, J=6.55, 1H), 8.75 (s, 1H), 11.88 (br s, 1H); MS (ESI) m/e 462.0 (M+H)⁺.

### Example 34

### N-[1-(1,3-benzodioxol-5-ylmethyl)piperidin-4-yl]-7-methoxy-4-oxo-1,4-dihydroquinoline-2-carboxamide

### Example 34A

### methyl 7-methoxy-4-oxo-1,4-dihydroquinoline-2-carboxylate

A 250 mL round bottom flask with a stir bar was charged with 2.00 g of m-anisidine (16.2 mmol) and 58.0 mL of methanol. To the mixture was added 2.31 g But-2-ynedioic acid dimethyl ester (16.3 mmol), and the mixture allowed to stir at room temperature for 3 hours until the starting material was consumed by TLC (85/15 hexanes/Ethyl acetate). The solvent was then evaporated under reduced pressure to provide 4.30 g of an orange oil that consisted of a mixture of E and Z isomers. From this material, 3.00 g (11.3 mmol) was added to a 250 mL round bottom flask followed by 25 mL of diphenyl ether. The mixture was slowly heated to 220° C. in an oil bath. After 2 hours, the mixture was allowed to cool to room temperature, and was then submerged in an ice bath for 20 minutes. The precipitate (solid diphenyl ether) was separated via suction filtration, and the filtrate was loaded on a biotage column and eluted with 10-60% ethyl acetate/hexanes to provide 0.520 g of the title compound. NMR (300 MHz, DMSO-D6) δ ppm 3.85 (s, 3H), 3.96 (s, 3H), 6.56 (s, 1H), 6.97 (m, 1H), 7.41 (d, J=2.37 Hz, 1H), 7.98 (d, J=9.15 Hz, 1H), 11.84 (br. s, 1H); MS (DCI/NH₃) m/z 234 [M+H]⁺.

### Example 34B

### 7-methoxy-4-oxo-1,4-dihydroquinoline-2-carboxylic acid

To a 4-dram vial charged with 1.46 mL of THF was added 73.0 mg of Example 34A (0.313 mmol) in portions. Full dissolution was achieved with gentle heating (heat gun), and then 0.500 mL of deionized H₂O was added followed by 26.0 mg LiOH*H₂O (0.620 mmol). The reaction mixture was allowed to shake at room temperature for 2 hours. After this time, 3 equiv of HCl (aqueous, 1N) was added and the product acid precipitated as a white solid. The precipitate was collected by suction filtration and washed with water. Drying in a vacuum oven provided the title product as white solid. ¹H NMR (300 MHz, DMSO-D6) δ ppm 3.84 (s, 3H), 6.55 (s, 1H), 6.96 (dd, J=8.81, 2.37 Hz, 1H), 7.43 (d, J=2.71 Hz, 1H), 7.97 (d, J=8.81 Hz, 1H), 11.73 (br s, 1H); MS (DCI/NH₃) m/z 220 [+H]⁺.

### Example 34

### N-[1-(1,3-benzodioxol-5-ylmethyl)piperidin-4-yl]-7-methoxy-4-oxo-1,4-dihydroquinoline-2-carboxamide

A 10 mL culture tube with a screw cap was charged with Example 34B (36.0 mg, 0.140 mmol), 1-Benzo[1,3]dioxol-5-ylmethyl-piperidin-4-ylamine (38.0 mg, 0.160 mmol), EDCI (30.0 mg, 0.160 mmol), HOBT (22.0 mg, 0.106 mmol), NMM (34.0 mg, 0.320 mmol) and 2 mL of DMF, and the reaction vessel was placed on a shaker for 6 hours. After this time, the DMF was removed under reduced pressure and the residue was dissolved in 1.5 mL of a 1:1 mixture of DMSO/methanol and purified by preparative reverse-phase HPLC. ¹H NMR (300 MHz, DMSO-d₆) δ ppm 1.75-2.10 (m, 4H), 3.05-3.43 (m, 4H), 3.85 (s, 3H), 4.03 (m, 1H), 4.20 (d, J=4.68, 2H), 6.08 (s, 2H), 6.77 (s, 1H), 6.97-7.02 (m, 3H), 7.08 (d, J=1.25, 1H), 7.43 (d, J=2.49, 1H), 7.98 (m, 1H), 8.74 (d, J=5.93, 0.2H), 8.92 (d, J=7.49, 0.8H), 9.48 (s, 1H); MS (DCI/NH₃) m/z 436 [M+H]⁺.

### Example 35

### N-[1-(1,3-benzodioxol-5-ylmethyl)piperidin-4-yl]-7-chloro-4-oxo-1,4-dihydroquinoline-2-carboxamide

### Example 35A

### 2-(3-Chloro-phenylamino)-but-2-enedioic acid dimethyl ester

The title compound was prepared according to the procedure for Example 32A substituting 3-chloro-phenylamine for 3-chloro-4-fluoro-phenylamine. ¹H NMR (300 MHz, DMSO-d₆) δppm major isomer: 3.66 (s, 3H), 3.69 (s, 2H), 5.42 (s, 1H), 6.83 (m, 1H), 7.09 (m, 2H), 7.29 (t, J=7.97 Hz, 1H), 9.55 (s, 1H); minor isomer (partial list): 3.54 (s, 0.44H), 3.79 (s, 0.41H), 5.24 (s, 0.14H), 7.15 (m, 0.35H), 7.39 (t, J=7.97 Hz, 0.14H), 9.50 (s, 0.17H).

### Example 35B

### 7-Chloro-4-oxo-1,4-dihydro-quinoline-2-carboxylic acid methyl ester

The title compound was prepared according to the procedure for Example 32B substituting 3-chloro-phenylamine for 3-chloro-4-fluoro-phenylamine. ¹H NMR (300 MHz, DMSO-d₆) δppm 3.96 (s, 3H), 6.65 (s, 1H), 7.40 (d, J=8.48 Hz, 1H), 8.00 (d, J=1.70 Hz, 1H), 8.07 (d, J=8.48 Hz, 1H), 12.13 (s, 1H).

### Example 35C

### 7-Chloro-4-oxo-1,4-dihydro-quinoline-2-carboxylic acid

The title compound was prepared according to the procedure for Example 32C substituting 3-chloro-phenylamine for 3-chloro-4-fluoro-phenylamine. ¹H NMR (300 MHz, DMSO-d₆) δ ppm 6.63 (s, 1H), 7.38 (dd, J=8.82, 2.03 Hz, 1H), 8.01 (d, J=2.03 Hz, 1H), 8.07 (d, J=8.82 Hz, 1H), 12.02 (s, 1H).

### Example 35

### N-[1-(1,3-benzodioxol-5-ylmethyl)piperidin-4-yl]-7-chloro-4-oxo-1,4-dihydroquinoline-2-carboxamide

The title compound was prepared according to the procedure for Example 32 substituting 7-chloro-4-oxo-1,4-dihydro-quinoline-2-carboxylic acid for 7-chloro-6-fluoro-4-oxo-1,4-dihydro-quinoline-2-carboxylic acid. ¹H NMR (300 MHz, DMSO-d₆) δppm 1.80 (m 2H), 2.07 (m, 2H), 3.11 (m, 2H), 3.40 (m, 2H), 4.03 (m, 1H), 4.20 (m, 2H), 6.09 (s, 2H), 6.86 (br s, 1H), 6.99 (m, 2H), 7.07 (d, J=1.36 Hz, 1H), 7.40 (m, 1H), 8.03 (d, J=1.70 Hz, 1H), 8.08 (m, 1H), 9.00 (d, J=7.46 Hz, 1H), 9.38 (br s, 1H); MS (ESI) m/e 439.9 (M+H)⁺.

### Example 36

### N-[1-(2-fluoro-4-methoxybenzyl)piperidin-4-yl]-4-oxo-7-(trifluoromethoxy)-1,4-dihydroquinoline-2-carboxamide

### Example 36A

### methyl 4-oxo-7-(trifluoromethoxy)-1,4-dihydroquinoline-2-carboxylate

A 250 mL round bottom flask was charged with 3-Trifluoromethoxy-phenylamine (6.20 g, 35.0 mmol), But-2-ynedioic acid dimethyl ester (5.00 g, 35.0 mmol) and 125 mL of methanol. The mixture was stirred at room temperature overnight after which the methanol was removed under reduced pressure, and the residue was purified by flash chromatography (ethyl acetate/Hexane, 1/1) to provide 9.00 g (28.2 mmol) of a pale yellow oil. This material was dissolved in 60 mL of diphenyl ether and then heated to 220° C. for 4 hours and cooled to room temperature. A 1/1 mixture of ethyl acetate and hexane (100 mL) was added to the mixture, and the resulting solid, a mixture of regioisomers, was collected by filtration. The solid was then separated by recrystallization in methanol to provide the title compound. ¹H NMR (300 MHz, DMSO-d₆) δ ppm 3.96 (s, 3H), 6.69 (s, 1H), 7.33 (dd, 1H, J₁=9.16, J₂=1.83), 7.93 (m, 1H), 8.18 (d, 1H, J=9.16); MS (DCI/NH₃) m/z 288 [M+H]⁺.

### Example 36B

### 4-oxo-7-(trifluoromethoxy)-1,4-dihydroquinoline-2-carboxylic acid

Example 36A (1.20 g, 4.20 mmol) and LiOH.H₂O (0.530 g, 12.6 mmol) was suspended in a 3:3:1 THF/ethanol/H₂O solution (35 mL). The mixture was stirred at room temperature for 0.5 hours, acidified with 10% HCl to pH=2, and one third of the solvent was removed under reduced pressure. The solid was collected by filtration, rinsed with water, and dried in vacuum oven to provide the title product. ¹H NMR (300 MHz, DMSO-d₆) δ ppm 6.65 (s, 1H), 7.31 (m, 1H), 7.95 (m, 1H), 8.18 (d, 1H, J=8.81), 12.08 (m, 1H); MS (DCI/NH₃) m/z 274 [M+H]⁺.

### Example 36

### N-[1-(2-fluoro-4-methoxybenzyl)piperidin-4-yl]-4-oxo-7-(trifluoromethoxy)-1,4-dihydroquinoline-2-carboxamide

A 10 mL culture tube with screw cap was charged with Example 36C (38.0 mg, 0.140 mmol), 1-(2-Fluoro-4-methoxy-benzyl)-piperidin-4-ylamine (40.0 mg, 0.160 mmol), EDCI (30.0 mg, 0.160 mmol), HOBT (22.0 mg, 0.160 mmol), NMM (34.0 mg, 0.320 mmol) and 2 mL of DMF, and the reaction vessel was placed on a shaker for 6 hours. After this time, the DMF was removed under reduced pressure and the residue was dissolved in 1.5 mL of a 1:1 mixture of DMSO/MeOH and purified by preparative reverse-phase HPLC. ¹H NMR (300 MHz, DMSO-d₆) δ ppm 1.70-2.10 (m, 4H), 2.60-3.50 (m, 4H), 4.05 (m, 1H), 4.20 (d, 2H, J=4.75), 6.09 (s, 2H), 6.95-7.10 (m, 4H), 7.85 (m, 1H), 7.96 (m, 1H), 8.19 (d, 1H, J=8.82), 9.01 (d, 1H, J=7.12), 9.30 (s, 1H); MS (DCI/NH₃) m/z 490 [M+H]⁺.

### Example 37

### N-[1-(1,3-benzodioxol-5-ylmethyl)piperidin-4-yl]-4-oxo-7-(trifluoromethoxy-1,4-dihydroquinoline-2-carboxamide

The title compound was prepared according to the procedure for Example 36 substituting 1-benzo[1,3]dioxol-5-ylmethyl-piperidin-4-ylamine for 1-(2-Fluoro-4-methoxy-benzyl)-piperidin-4-ylamine. ¹H NMR (300 MHz, DMSO-d₆) δ ppm 1.74-2.10 (m, 4H), 3.10-3.60 (m, 5H), 3.82 (s, 3H), 4.28 (d, 2H, J=3.73), 6.90-7.02 (m, 3H), 7.35 (m, 1H), 7.50 (t, 1H, J=8.48), 7.96 (m, 1H), 8.19 (d, 1H, J=8.48), 8.99 (m, 1H), 9.44 (m, 1H); MS (DCI/NH₃) m/z 494 [M+H]⁺.

### Example 38

### N-[1-(2-fluoro-4-methoxybenzyl)piperidin-4-yl]-7-methoxy-4-oxo-1,4-dihydroquinoline-2-carboxamide

The title compound was prepared according to the procedure for Example 34 substituting 1-(2-Fluoro-4-methoxy-benzyl)-piperidin-4-ylamine for 1-Benzo[1,3]dioxol-5-ylmethyl-piperidin-4-ylamine. ¹H NMR (300 MHz, DMSO-d₆) δ ppm 1.74-2.10 (m, 4H), 3.15-3.47 (m, 4H), 3.82 (s, 3H), 3.85 (s, 3H), 4.07 (m, 1H), 4.27 (m, 2H), 6.77 (m, 1H), 6.91-6.99 (m, 3H), 7.42 (d, 1H, J=2.18), 7.50 (t, 1H, J=8.74), 7.97 (d, 1H, J=9.04), 8.76 (d, 0.2H, J=5.93), 8.91 (d, 0.8H, J=7.49), 9.57 (s, 1H); MS (DCI/NH₃) m/z 440 [M+H]⁺.

### Example 39

### 7-methoxy-N-[1-(4-methoxybenzyl)piperidin-4-yl]-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 4-methoxybenzaldehyde (0.0132 mmol) dissolved in dichloromethane/methanol (1:1/v:v). The core (Example 2C, 20 mg, 0.066 mmol) dissolved in dichloromethane/methanol (1:1/v:v) was then added to the reaction mixture, followed by an addition of acetic acid (3 eq.) in dichloromethane/methanol (1:1/v:v). The microwave reaction vessel was capped; and was then heated with stirring at 100 degrees Celcius for 600 seconds on an Emrys Optimizer Microwave Reactor (Personal Chemistry). The resin-suspension was then filtered and transferred with methanol to 20 mL vials and concentrated down using a Savant Speed Vac. Purification was done by Agilent Mass triggered HPLC/MS using acetonitrile/H2O with 0.1% TFA as eluent on a C18 column. ¹H NMR (500 MHz, PYRIDINE-d₅) δ ppm 2.07 (m, 4H), 2.26 (m, 2H), 3.05 (m, 2H), 3.58 (s, 3H), 3.62 (s, 2H), 3.66 (s, 3H), 4.29 (m, 1H), 6.64 (d, J=2.50 Hz, 1H), 6.92 (d, J=8.73 Hz, 2H), 6.95 (dd, J=9.05, 2.18 Hz, 1H), 7.34 (d, J=8.74 Hz, 2H), 7.37 (s, 1H), 8.19 (d, J=8.74 Hz, 1H), 9.76 (d, J=7.80 Hz, 1H); MS (ESI) m/z 423 [M+H]⁺, 421 [M−H]⁺.

### Example 40

### 7-methoxy-N-{1-[(5-methylthien-2-yl)methyl]piperidin-4-yl}-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was adde 5-methyl-thiophene-2-carboxaldehyde (0.0132 mmol) dissolved in dichloromethane/methanol (1:1/v:v). The core (Example 2C, 20 mg, 0.066 mmol) dissolved in dichloromethane/methanol (1:1/v:v) was added to the mixture, followed by an addition of acetic acid (3 eq.) in dichloromethane/methanol (1:1/v:v). The microwave reaction vessel was capped; and was then heated with stirring at 100 degrees Celcius for 600 seconds on an Emrys Optimizer Microwave Reactor (Personal Chemistry). The resin-suspension was then filtered and transferred with MeOH to 20 mL vials and concentrated down using a Savant Speed Vac. Purification was done by Agilent Mass triggered HPLC/MS using acetonitrile/H2O with 0.1% TFA as eluent on a C18 column. ¹H NMR (500 MHz, PYRIDINE-d₅) δ ppm 1.94 (m, J=11.85, 3.43 Hz, 2H), 2.10 (m, 2H), 2.18 (t, J=11.23 Hz, 2H), 2.33 (s, 3H), 3.04 (m, J=11.85 Hz, 2H), 3.51 (s, 3H), 3.72 (s, 2H), 4.28 (m, 1H), 6.23 (m, 1H), 6.65 (d, J=3.12 Hz, 1H), 6.81 (d, J=3.12 Hz, 1H), 6.98 (dd, J=8.89, 2.34 Hz, 1H), 7.45 (s, 1H), 8.25 (d, J=8.73 Hz, 1H), 9.64 (d, J=7.49 Hz, 1H); MS (ESI) m/z 413 [M+H]⁺, 411 [M−H]⁺.

### Example 41

### N-[1-(2-chlorobenzyl)piperidin-4-yl]-7-methoxy-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 2-chlorobenzaldehyde (0.0132 mmol) dissolved in dichloromethane/methanol (1:1/v:v). The core (Example 2C, 20 mg, 0.066 mmol) dissolved in dichloromethane/methanol (1:1/v:v) was then added to the mixture, followed by an addition of acetic acid (3 eq.) in dichloromethane/methanol (1:1/v:v). The microwave reaction vessel was capped; and was then heated with stirring at 100 degrees Celcius for 600 seconds on an Emrys Optimizer Microwave Reactor (Personal Chemistry). The resin-suspension was then filtered and transferred with methanol to 20 mL vials and concentrated down using a Savant Speed Vac. Purification was done by Agilent Mass triggered HPLC/MS using acetonitrile/H2O with 0.1% TFA as eluent on a C18 column. ¹H NMR (500 MHz, PYRIDINE-d₅) δ ppm 1.99 (m, 2H), 2.05 (m, 2H), 2.19 (m, 2H), 2.92 (d, J=11.85 Hz, 2H), 3.59 (s, 3H), 3.61 (s, 2H), 4.25 (m, 1H), 6.64 (d, J=2.18 Hz, 1H), 6.97 (dd, J=8.89, 2.34 Hz, 1H), 7.18 (m, J=2.18 Hz, 2H), 7.37 (m, 1H), 7.39 (s, 1H), 7.49 (m, 1H), 8.20 (d, J=8.73 Hz, 1H), 9.70 (d, J=7.49 Hz, 1H); MS (ESI) m/z 427 [M+H]⁺, 425 [M−H]⁺.

### Example 42

### N-[1-(2-bromobenzyl)piperidin-4-yl]-7-methoxy-4-oxo-4H-chromene-2-carboxamide

To a 5 mL microwave reaction vessel which contained 85.61 mg (3.0 eq.) of MP-CNBH₃ resin (subst. 2.55 mmoles/g) and a micro-Teflon-coated stir bar, was added 2-bromobenzaldehyde (0.0132 mmol) dissolved in dichloromethane/methanol (1:1/v:v). The core (Example 2C, 20 mg, 0.066 mmol) dissolved in dichloromethane/methanol (1:1/v:v) was then added to the reaction mixture, followed by an addition of acetic acid (3 eq.) in dichloromethane/methanol (1:1/v:v). The microwave reaction vessel was capped; and was then heated with stirring at 100 degrees Celcius for 600 seconds on an Emrys Optimizer Microwave Reactor (Personal Chemistry). The resin-suspension was then filtered and transferred with methanol to 20 mL vials and concentrated down using a Savant Speed Vac. Purification was done by Agilent Mass triggered HPLC/MS using acetonitrile/H2O with 0.1% TFA as eluent on a C18 column. ¹H NMR (500 MHz, PYRIDINE-d₅) δ ppm 1.95 (m, 2H), 2.08 (m, 2H), 2.19 (m, 2H), 2.92 (m, 2H), 3.59 (s, 2H), 3.60 (s, 3H), 4.29 (m, 1H), 6.56 (d, J=2.50 Hz, 1H), 7.01 (dd, J=8.74, 2.50 Hz, 1H), 7.14 (m, 1H), 7.27 (m, 1H), 7.46 (s, 1H), 7.50 (d, J=7.49 Hz, 1H), 7.62 (m, 1H), 8.26 (d, J=8.73 Hz, 1H), 9.67 (d, J=7.80 Hz, 1H); MS (ESI) m/z 473 [M+H]⁺, 471 [M−H]⁺.

### Example 43

### N-{1-[(4-bromothien-2-yl)methyl]piperidin-4-yl}-7-methoxy-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 4-bromothiophene-2-carboxaldehyde (0.0132 mmol) dissolved in dichloromethane/methanol (1:1/v:v). The core (Example 2C, 20 mg, 0.066 mmol) dissolved in dichloromethane/methanol (1:1/v:v) was then added to the reaction mixture, followed by an addition of acetic acid (3 eq.) in dichloromethane/methanol (1:1/v:v). The microwave reaction vessel was capped; and was then heated with stirring at 100 degrees Celcius for 600 seconds on an Emrys Optimizer Microwave Reactor (Personal Chemistry). The resin-suspension was then filtered and transferred with methanol to 20 mL vials and concentrated down using a Savant Speed Vac. Purification was done by Agilent Mass triggered HPLC/MS using acetonitrile/H₂O with 0.1% TFA as eluent on a C18 column. ¹H NMR (500 MHz, PYRIDINE-d₅) δ ppm 1.82 (m, J=11.70, 3.59 Hz, 2H), 2.05 (m, J=10.76, 10.76 Hz, 4H), 2.87 (m, 2H), 3.51 (s, 3H), 3.56 (s, 2H), 4.25 (m, 1H), 6.27 (d, J=2.50 Hz, 1H), 6.93 (s, 1H), 6.99 (dd, J=8.73, 2.50 Hz, 1H), 7.43 (d, J=1.25 Hz, 1H), 7.45 (s, 1H), 8.25 (d, J=9.05 Hz, 1H), 9.54 (s, 1H); MS (ESI) m/z 477 [M+H]⁺, 475 [M−H]⁺.

### Example 44

### N-(1-benzylpiperidin-4-yl)-7-methoxy-4-oxo-4H-chromene-2-carboxamide

To a 5 mL microwave reaction vessel which contained 85.61 mg (3.0 eq.) of MP-CNBH₃ resin (2.55 mMoles/g) and a micro-Teflon-coated stir bar, was added benzaldehyde (0.0132 mmol) in dichloromethane/methanol (1:1/v:v), Example 2C (20 mg, 0.066 mmol) in dichloromethane/methanol (1:1/v:v) followed by an addition of acetic acid (3 eq.) in dichloromethane/methanol (1:1/v:v). The vessel was capped and was heated with stirring at 100 degrees Celcius for 600 seconds on an Emrys Optimizer Microwave Reactor (Personal Chemistry). The resin-suspension was filtered and concentrated using a Savant Speed Vac. Purification was done by Agilent Mass triggered HPLC/MS using acetonitrile/H₂O with 0.1% TFA as eluent on a C18 column. ¹H NMR (500 MHz, PYRIDINE-d₅) δ ppm 2.16 (m, 4H), 2.39 (m, 2H), 3.13 (m, J=12.48 Hz, 2H), 3.65 (s, 3H), 3.77 (s, 2H), 4.36 (m, 1H), 6.72 (d, J=2.50 Hz, 1H), 7.02 (dd, J=8.89, 2.34 Hz, 1H), 7.35 (m, 3H), 7.43 (s, 1H), 7.48 (d, J=6.55 Hz, 2H), 8.25 (d, J=8.73 Hz, 1H), 9.84 (d, J=7.49 Hz, 1H); MS (ESI) m/z 393 [M+H]⁺, 415 [M+Na]⁺, 391 [M−H]⁺.

### Example 45

### 7-methoxy-N-[1-(3-methoxybenzyl)piperidin-4-yl]-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 3-methoxybenzaldehyde (0.0132 mmol) in dichloromethane/methanol (1:1/v:v), Example 2C (20 mg, 0.066 mmol) in dichloromethane/methanol (1:1/v:v) followed by acetic acid (3 eq.) in dichloromethane/methanol (1:1/v:v). The vessel was capped and was heated with stirring at 100 degrees Celcius for 600 seconds on an Emrys Optimizer Microwave Reactor (Personal Chemistry). The resin-suspension was filtered and concentrated down using a Savant Speed Vac. Purification was done by Agilent Mass triggered HPLC/MS using acetonitrile/H₂O with 0.1% TFA as eluent on a C18 column. ¹H NMR (500 MHz, PYRIDINE-d₅) δ ppm 2.17 (m, 4H), 2.43 (m, 2H), 3.17 (m, J=12.17 Hz, 2H), 3.67 (m, 3H), 3.74 (s, 3H), 3.79 (s, 2H), 4.37 (m, 1H), 6.72 (d, J=2.50 Hz, 1H), 6.98 (dd, J=8.27, 2.03 Hz, 1H), 7.02 (dd, J=8.73, 2.50 Hz, 1H), 7.09 (d, J=7.80 Hz, 1H), 7.21 (s, 1H), 7.32 (t, J=7.96 Hz, 1H), 7.43 (s, 1H), 8.26 (d, J=9.05 Hz, 1H), 9.84 (d, J=7.80 Hz, 1H); MS (ESI) m/z 423 [M+H]⁺, 421 [M−H]⁺.

### Example 46

### N-{1-[(4,5-dimethyl-2-furyl)methyl]piperidin-4-yl}-7-methoxy-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 4,5-dimethylfuran-2-carboxaldehyde (0.0132 mmol) in dichloromethane/methanol (1:1/v:v), Example 2C (20 mg, 0.066 mmol) in dichloromethane/methanol (1:1/v:v) followed by an addition of acetic acid (3 eq.) in dichloromethane/methanol (1:1/v:v). The vessel was capped and was then heated with stirring at 100 degrees Celcius for 600 seconds on an Emrys Optimizer Microwave Reactor (Personal Chemistry). The resin-suspension was then filtered and concentrated down using a Savant Speed Vac. Purification was done by Agilent Mass triggered HPLC/MS using acetonitrile/H₂O with 0.1% TFA as eluent on a C18 column. ¹H NMR (500 MHz, PYRIDINE-d₅) δ ppm 1.84 (d, J=4.99 Hz, 3H), 2.06 (s, 3H), 2.10 (m, 2H), 2.15 (m, J=2.81 Hz, 2H), 2.48 (m, 2H), 3.23 (m, J=12.17 Hz, 2H), 3.50 (s, 3H), 3.82 (s, 2H), 4.35 (m, 1H), 6.18 (d, J=2.50 Hz, 1H), 6.20 (s, 1H), 6.97 (d, J=8.89, 2.34 Hz, 1H), 7.43 (s, 1H), 8.24 (d, J=8.73 Hz, 1H), 9.83 (d, J=7.49 Hz, 1H); MS (ESI) m/z 411 [M+H]⁺, 409 [M−H]⁺.

### Example 47

### N-[1-(3-chlorobenzyl)piperidin-4-yl]-7-methoxy-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 3-chlorobenzaldehyde (0.0132 mmol) in dichloromethane/methanol (1:1/v:v), Example 2C (20 mg, 0.066 mmol) in dichloromethane/methanol (1:1/v:v) followed by an addition of acetic acid (3 eq.) in dichloromethane/methanol (1:1/v:v). The vessel was capped and was then heated with stirring at 100 degrees Celcius for 600 seconds on an Emrys Optimizer Microwave Reactor (Personal Chemistry). The resin-suspension was then filtered and concentrated down using a Savant Speed Vac. Purification was done by Agilent Mass triggered HPLC/MS using acetonitrile/H₂O with 0.1% TFA as eluent on a C18 column. ¹H NMR (500 MHz, PYRIDINE-d₅) δ ppm 1.99 (m, 4H), 2.11 (m, 2H), 2.88 (m, J=11.85 Hz, 2H), 3.46 (s, 2H), 3.58 (s, 3H), 4.23 (m, J=7.49 Hz, 1H), 6.64 (d, J=2.50 Hz, 1H), 6.95 (dd, J=8.89, 2.34 Hz, 1H), 7.22 (m, J=4.99 Hz, 2H), 7.26 (m, J=4.52, 2.03 Hz, 1H), 7.37 (s, 1H), 7.42 (s, 1H), 8.19 (d, J=9.05 Hz, 1H), 9.69 (d, J=7.80 Hz, 1H); MS (ESI) m/z 428 [+H]⁺, 425 [M−H]⁺.

### Example 48

### 7-methoxy-N-[1-(1-naphthylmethyl)piperidin-4-yl]-4-oxo-4H-chromene-2-carboxamide

To a 5 mL microwave reaction vessel which contained 85.61 mg (3.0 eq.) of MP-CNBH₃ resin (subst. 2.55 mMoles/g) and a micro-Teflon-coated stir bar, was added 1-napthaldehyde (0.0132 mmol) in dichloromethane/methanol (1:1/v:v), Example 2C (20 mg, 0.066 mmol) in dichloromethane/methanol (1:1/v:v) followed by an addition of acetic acid (3 eq.) in dichloromethane/methanol (1:1/v:v). The vessel was capped; and was then heated with stirring at 100 degrees Celcius for 600 seconds on an Emrys Optimizer Microwave Reactor (Personal Chemistry). The resin-suspension was then filtered and transferred with MeOH to 20 mL vials and concentrated down using a Savant Speed Vac. Purification was done by Agilent Mass triggered HPLC/MS using acetonitrile/H₂O with 0.1% TFA as eluent on a C18 column. ¹H NMR (500 MHz, PYRDINE-d₅) δ ppm 1.87 (m, 2H), 2.07 (d, J=1.54 Hz, 2H), 2.27 (t, J=11.54 Hz, 2H), 3.05 (d, J=11.54 Hz, 2H), 3.52 (s, 3H), 4.00 (s, 2H), 4.32 (m, J=7.49 Hz, 1H), 6.29 (d, J=2.50 Hz, 1H), 6.98 (dd, J=8.74, 2.50 Hz, 1H), 7.46 (m, 2H), 7.52 (m, 3H), 7.86 (d, J=8.11 Hz, 1H), 7.91 (m, 1H), 8.25 (d, J=8.73 Hz, 1H), 8.42 (d, J=7.49 Hz, 1H), 9.53 (d, J=7.49 Hz, 1H); MS (ESI) m/z 443 [M−H]⁺.

### Example 49

### N-[1-(1,3-benzodioxol-5-ylmethyl)piperidin-4-yl]-7-chloro-6-cyano-4-oxo-4H-chromene-2-carboxamide

### Example 49A

### 5-acetyl-2-chloro-4-hydroxy-benzonitrile

To a solution of 2-chloro-4-hydroxy-benzonitrile (5.0 g, 32.5 mmol) and triethylamine (4.52 mL, 32.5 mmol) in dichloromethane (32 mL) at room temperature was added acetyl chloride (2.54 mL, 35.8 mmol). The mixture was stirred at room temperature overnight and then concentrated to a yellow residue. The residue was diluted with diethyl ether (75 mL), and the solid NH₄Cl was filtered. The eluent was concentrated to a yellow oil. The oil was dissolved in dichloromethane (23 mL) and added to a room temperature suspension of AlCl₃ (6.5 g, 48.9 mmol) in dichloromethane (10 mL). The mixture was stirred vigorously for 1 hour and then a short-path distillation head was attached to the round-bottomed flask. The dichloromethane was distilled (bath temp=70° C.) from the mixture, and the resulting slurry was heated to 170° C. for 3 hours. The reaction was cooled to room temperature and then diluted with 100 mL of 50% aqueous HCl. The solid was filtered, and air-dried. Purification of the residue by MPLC (1:1 Hexane:Ethyl acetate) provided 5-acetyl-2-chloro-4-hydroxy-benzonitrile as a solid. ¹H NMR (300 MHz, DMSO-d₆) δ ppm 2.64 (s, 3H), 7.34 (s, 1H), 8.39 (s, 1H), and 12.57 (s, 1H).

### Example 49B

### 7-chloro-6-cyano-4-oxo-4H-chromene-2-carboxylic acid ethyl ester

To a suspension of 5-acetyl-2-chloro-4-hydroxy-benzonitrile (1.54 g, 8.06 mmol) in diethylglyoxalate (6 mL) at room temperature was added sodium ethoxide (12.6 mL, 32.24 mmol, 20 weight % in EtOH). The reaction was stirred at 50° C. for 0.5 hour, and was then cooled to room temperature. The mixture was diluted with diethyl ether (15 mL) and the yellow solid was filtered, washed with additional diethyl ether (10 mL) and then dried under reduced pressure. The solid was suspended in a mixture of concentrated HCl (1 mL) in acetic acid (8 mL) and heated to reflux (bath temperature=115 C) for 2 hours. The mixture was cooled to ambient temperature and diluted with H₂O (10 mL). The resulting solid was filtered and air-dried to provide 7-chloro-6-cyano-4-oxo-4H-chromene-2-carboxylic acid ethyl ester as a solid. ¹H NMR (300 MHz, DMSO-d₆) δ ppm 1.35 (t, J=7.12 Hz, 3H), 4.40 (q, J=7.12 Hz, 2H), 7.06 (s, 1H), 8.38 (s, 1H), 8.60 (s, 1H).

### Example 49C

### 7-Chloro-6-cyano-4-oxo-4H-chromene-2-carboxylic acid

A 100 mL round bottom flask was charged with 7-chloro-6-cyano-4-oxo-4H-chromene-2-carboxylic acid ethyl ester (800 mg, 2.88 mmol), 40 mL glacial acetic acid, and 10 mL 6N HCl. The solution was refluxed overnight, and then cooled to ambient temperature. The solid was filtered, washed with H₂O (5 mL), and air-dried to provide 7-chloro-6-cyano-4-oxo-4H-chromene-2-carboxylic acid as a solid. ¹H NMR (300 MHz, DMSO-d₆) δ ppm 1.90 (s, 1H), 7.00 (s, 1H), 8.30 (s, 1H), 8.60 (s, 1H); MS (ESI) m/z 248 [M−H]⁻.

### Example 49

### N-[1-(1,3-benzodioxol-5-ylmethyl)piperidin-4-yl]-7-chloro-6-cyano-4-oxo-4H-chromene-2-carboxamide

To a 4 mL vial with screw cap was added 7-chloro-6-cyano-4-oxo-4H-chromene-2-carboxylic acid (25.0 mg, 0.100 mmol), 1-benzo[1,3]dioxol-5-ylmethyl-piperidin-4-ylamine dihydrochloric acid salt (46.0 mg, 0.150 mmol), EDCI (19.2 mg, 0.100 mmol), HOBT (13.6 mg, 0.100 mmol), NMM (48.0 mg, 0.475 mmol) and 0.5 mL of DMF. The vial was placed on a shaker at 40° C. for 16 hours. The solution was diluted with acetonitrile (1 mL), filtered, and purified by preparative reverse-phase HPLC to provide N-[1-(1,3-benzodioxol-5-ylmethyl)piperidin-4-yl]-7-chloro-6-cyano-4-oxo-4H-chromene-2-carboxamide. ¹H NMR (300 MHz, DMSO-d₆) δ ppm 1.80 (m, 2H), 2.10 (m, 2H), 3.10 (m, 2H), 3.40 (m, 2H), 4.00 (m, 1H), 4.20 (s, 2H), 6.10 (s, 2H), 6.90-7.10 (m, 4H), 8.20 (s, 1H), 8.60 (s, 1H), 9.10 (d, 1H); MS (ESI) m/z 466 [M+H]⁺.

### Example 50

### N-[1-(1,3-benzothiazol-6-ylmethyl)piperidin-4-yl]-6,7-dichloro-4-oxo-4H-chromene-2-carboxamide

### Example 50A

### 2-(3,4-Dichloro-phenoxy)-but-2-enedioic acid

Benzyltrimethylammonium hydroxide (1.5 mL of a 40% weight aqueous solution, 1.2%) was added to a solution of 3,4-dichlorophenol (20.0 g, 123 mmol), dimethyl acetylenedicarboxylate (16.6 mL, 135 mmol), and dioxane (230 mL). The dark, homogeneous mixture was heated at 90° C. for 1.5 hour, cooled to ambient temperature, combined with 20% aqueous NaOH (100 mL), and warmed to 90° C. After an hour, the mixture was cooled to ambient temperature and combined with 2N aqueous HCl until the pH of the solution was pH=7. White sediment was removed by filtration and the filtrate was combined with 2N aqueous HCl until acidic (pH=2). The resulting precipitate was collected by filtration, washed with water, and air-dried to provide the product as a solid. ¹H NMR (300 MHz, DMSO-d₆) δ ppm 6.64 (s, 1H), 6.95 (dd, J=8.82, 2.71 Hz, 1H), 7.25 (d, J=3.05 Hz, 1H), 7.57 (d, J=8.82 Hz, 1H); MS (APCI) m/z 275 [M−H]⁺.

### Example 50B

### 6,7-Dichloro-4-oxo-4H-chromene-2-carboxylic acid

Example 50A (17.98 g, 64.9 mmol) was combined with Eaton's Reagent (140 ml). The mixture was heated a 70° C. overnight. After 14 hours the mixture was cooled to ambient temperature and added to ice (600 g). The resulting white precipitate was collected by filtration, washed with water, and air-dried. The white solid (16.8 g) was dissolved in hot dimethyl sulfoxide and cooled slowly to ambient temperature. The supernatant was recovered and concentrated, and the process was repeated to provide the title compound. The crystalline material proved to be the undesirable 5,6-dichloro isomer. ¹H NMR (300 MHz, DMSO-d₆) δ ppm 6.96 (s, 1H), 8.16 (s, 1H), 8.26 (s, 1H); MS (APCI) m/z 259 [+H]⁺.

### Example 50C

### 4-[(6,7-Dichloro-4-oxo-4H-chromene-2-carbonyl)-amino]-piperidine

To a solution of Example 50B (6,7-dichloro-4-oxo-4H-chromene-2-carboxylic acid, 2.5 g, 9.7 mmol) in 30 mL of dimethylformamide was added 4-amino-1-BOC-piperidine (1.94 g, 9.7 mmol), hydroxybenzotriazole (1.31 g, 9.7 mmol), diisopropylethylamine (1.29 g, 10 mmol), under an atmosphere of nitrogen followed by the addition of ethyldimethylaminocarbodiimide hydrochloride (1.90 g, 10 mmol). The mixture was stirred for 14 hours, diluted with ethyl acetate, washed with 1M H₂SO4, 1M K₂CO₃, brine, dried (Na₂SO₄), filtered and concentrated under reduced pressure to provide an oil. This residue was dissolved in 50 mL dichloromethane, under nitrogen, cooled to 0° C., after which 45 mL of trifluoroacetic acid in was added three portions. After 15 minutes at 0° C., the reaction was allowed to warm to room temperature, stirred for 1.5 hours and then concentrated to a brown oil. This oil was dissolved in dichloromethane (300 mL) and washed with 1M K₂CO₃, aqueous extracted with 95/5 dichloromethane/methanol (3×100 mL), organic extracts combined, dried (Na₂SO₄), filtered and concentrated to provide the title compound. [M+H], 342.

### Example 50D

### benzothiazole-6-carboxylic acid methyl ester

To a 250 mL round bottom flask was added benzothiazole-6-carboxylic acid (5.0 g, 27.9 mmol), 100 mL methanol, and 10 mL thionyl chloride. The solution was refluxed 2 hours, cooled to ambient temperature, and concentrated under reduced pressure to provide the title compound. ¹HNMR (300 MHz, DMSO-d₆) δ ppm 3.91 (s, 3H), 8.10 (dd, 1H), 8.19 (d, 1H), 8.86 (d, 1H), 9.60 (s, 1H); MS (ESI) m/z 194 [M+H]⁺.

### Example 50E

### benzothiazole-6-methanol

To a 500 mL round bottom flask containing benzothiazole-6-carboxylic acid methyl ester (3.0 g, 15.5 mmol) and 100 mL dichloromethane at 0° C. was added diisobutylaluminum hydride (50 mL of 1.0 M solution in heptane). The solution was allowed to warm to ambient temperature and stirred for 4 hours. The mixture was diluted with ethyl acetate (100 mL), followed by stirring with sat. potassium sodium tartrate (100 mL) for 2 hours. The layers were separated, and the organic layer was washed with brine (100 mL), dried (Na₂SO₄), filtered and solvents were removed under reduced pressure. Purification by silica gel chromatography (25% Ethyl acetate/hexanes) provided the title compound as an oil. MS (ESI) m/z 166 [M+H]⁺.

### Example 50F

### 6-bromomethylbenzothiazole

To a 50 mL round bottom flask was added benzothiazole-6-methanol (1.0 g, 6 mmol) and 10 mL of 30 HBr in acetic acid, 50 mmol and the solution was heated to 60° C. for 2 hours. The solution was cooled to ambient and purged with nitrogen. Toluene (30 mL) was added and the solution was concentrated under vacuum (repeat 2×). The solid obtained was dried under reduced pressure at 25° C. for 16 hours to provide the title compound. ¹H NMR (300 MHz, DMSO-D6) δ ppm 4.89 (s, 2H), 7.62 (dd, 1H), 8.08 (d, 1H), 8.27 (d, 1H), 9.43 (s, 1H); MS (ESI) 229 (M+H)⁺.

### Example 50

### N-[1-(1,3-benzothiazol-6-ylmethyl)piperidin-4-yl]-6,7-dichloro-4-oxo-4H-chromene-2-carboxamide

Example 50F and Example 50C were processed according to the procedure described in Example 3 to prepare the title compound. ¹H NMR (300 MHz, DMSO-D6) δ ppm 1.64 (m, 2H), 1.81 (m, 2H), 2.10 (m, 2H), 2.87 (m, 2H), 3.65 (s, 2H), 3.80 (m, 1H), 6.85 (s, 1H), 7.51 (dd, 1H), 8.05 (d, 1H), 8.08 (d, 1H), 8.13 (s, 1H), 8.16 (s, 1H), 8.83 (d, 1H), 9.35 (s, 1H); MS (ESI) 488 (M+H)⁺.

### Example 51

### N-[1-(1,3-benzoxazol-5-ylmethyl)piperidin-4-yl]-6,7-dichloro-4-oxo-4H-chromene-2-carboxamide

### Example 51A

### 5-bromomethylbenzoxazole

A mixture of 5-Methyl-benzoxazole (3 g, 22 mmol), N-bromosuccinimide (4 g, 22 mmol), and Benzoyl peroxide (5 mg, 0.02 mmol) was heated to reflux for 5 hours in CCl₄ (100 mL). The mixture was cooled to room temperature, and resulting precipitate was removed by passing through a plug of silica gel. Filtrate was concentrated under reduced pressure and the residue was crystalized from ethyl acetate and hexane (1/1) to provide a pale yellow solid. MS (ESI) 213 (M+H)⁺.

### Example 51

### N-[1-(1,3-benzoxazol-5-ylmethyl)piperidin-4-yl]-6,7-dichloro-4-oxo-4H-chromene-2-carboxamide

Example 51A and Example 50C were processed according to the procedure described in Example 3 to provide the title compound. ¹H NMR (300 MHz, DMSO-D6) δ ppm 1.64 (m, 2H), 1.82 (m, 2H), 2.08 (m, 2H), 2.87 (m, 2H), 3.62 (s, 2H), 3.81 (m, 1H), 6.85 (s, 1H), 7.40(dd, 1H), 7.71 (m, 2H), 8.13 (s, 1H), 8.16 (s, 1H), 8.72 (s, 1H), 8.83 (d, 1H); MS (ESI) 473 (M+H)⁺.

### Example 52

### N-[1-(1,3-benzodioxol-5-ylmethyl)piperidin-4-yl]-6-chloro-7-methoxy-4-oxo-4H-chromene-2-carboxamide

### Example 52A 2-Hydroxy-3-iodo-4-methoxyacetophenone

To a stirred solution of 2-hydroxy-4-methoxyacetophenone (2.0 g, 12.1 mmol) in ethanol (40 mL) was added iodine (1.22 g, 4.82 mmol) followed by a solution of HIO₃ (0.42 g, 2.41 mmol) in H₂O (11 mL). The mixture was stirred vigorously for 2 hours and was then diluted with H₂O (10 mL). The solid was filtered, air-dried, and crystallized from ethanol to provide the title compound. ¹H NMR (300 MHz, DMSO-d₆) δ ppm 2.63 (s, 3H), 3.94 (s, 3H), 6.70 (d, J=8.82 Hz, 1H), 8.01 (d, J=8.82 Hz, 1H), 13.49 (s, 1H); MS (ESI) m/z 413 [M+H]⁺.

### Example 52B

### 2-Hydroxy-3-iodo-4-methoxy-5-chloro-acetophenone

To a stirred solution of 2-hydroxy-3-iodo-4-methoxyacetophenone (1.50 g, 5.14 mmol in 1:1 ethyl acetate/acetic acid (20 mL) was added N-chlorosuccinimide (0.717 g, 5.39 mmol). The mixture was heated to 45° C. for 16 hours, and concentrated under reduced pressure to a volume of ˜10 mL. The mixture was diluted with H₂O (20 mL) and diethyl ether (15 mL). The layers were separated, and the aqueous was extracted with additional diethyl ether (3×15 mL). The combined organic layers were dried with anhydrous Na₂SO₄, filtered, and concentrated under reduced pressure to provide 2-hydroxy-3-iodo-4-methoxy-5-chloro-acetophenone. ¹H NMR (300 MHz, DMSO-d₆) δ ppm 2.68 (m, 3H), 2.68 (s, 3H), 8.16 (s, 1H), 13.25 (s, 1H); MS (ESI) m/z 325 [M−H]⁻.

### Example 52C

### 2-Hydroxy-4-methoxy-5-chloro-acetophenone

To a stirred solution of 2-hydroxy-3-iodo-4-methoxy-5-chloro-acetophenone (1.67 g, 5.14 mmol) in DMF (20 mL) under an atmosphere of N₂ was added sodium formate (1.74 g, 25.7 mmol), and Pd(Ph₃)₄ (0.178 g, 0.153 mmol), and the mixture was heated to 120° C. for 0.5 hour. The mixture was cooled to room temperature and diluted with H₂O (20 mL). The solid was filtered and dried under reduced pressure. Purification of the residue by MPLC in 95:5 dichloromethane/methanol to provide 2-hydroxy-4-methoxy-5-chloro-acetophenone as a solid. ¹H NMR (300 MHz, DMSO-d₆) δ ppm 2.59 (s, 3H), 3.91 (s, 3H), 6.70 (s, 1H), 7.95 (s, 1H), 12.53 (s, 1H); MS (ESI) m/z 413 [M+H]⁺.

### Example 52D

### 6-Chloro-7-methoxy-4-oxo-4H-chromene-2-carboxylic acid

To a solution of 2-hydroxy-4-methoxy-5-chloro-acetophenone (440 mg, 2.20 mmol) in diethylglyoxalate (1.7 mL) was added NaOEt (3.44 mL, 8.80 mmol, 20 weight % in EtOH), and the mixture was heated to 50° C. for 0.5 hour. The mixture was cooled to ambient temperature, diluted with diethyl ether (5 mL), and filtered. The yellow solid was suspended in a solution of concentrated HCl (12 M, 0.30 mL) in acetic acid (2.20 mL), and heated to reflux (bath temperature=115° C.) for 1.5 hours. 6 N HCl (0.70 mL) was added to the reaction, and it was heated to reflux for an additional 16 hours. The mixture was cooled to ambient temperature, diluted with H₂O (5 mL), and filtered. The solid was air-dried to provide the title compound as a solid. ¹H NMR (300 MHz, DMSO-d₆) δ ppm 4.03 (s, 3H), 6.89 (s, 1H), 7.48 (s, 1H), 7.99 (s, 1H); MS (ESI) m/z 255 [M+H]⁺.

### Example 52

### N-[1-(1,3-benzodioxol-5-ylmethyl)piperidin-4-yl]-6-chloro-7-methoxy-4-oxo-4H-chromene-2-carboxamide

To a stirred solution of 6-chloro-7-methoxy-4-oxo-4H-chromene-2-carboxylic acid (20 mg, 0.078 mmol), 1-benzo[1,3]dioxol-5-ylmethyl-piperidin-4-ylamine bis-hydrochloride salt (24 mg, 0.078 mmol), NMM (0.041 mL, 0.38 mmol), and HOBt (13 mg, 0.094 mmol) in DMF (1 mL) was added EDCI (18 mg, 0.094 mmol). The mixture was heated to 55° C. for 12 hours, and concentrated under reduced pressure. Purification of the residue by RP-HPLC provided the titled compound has yellow solid. ¹H NMR (300 MHz, DMSO-d₆) δ ppm 1.65 (m, 2H), 1.78 (s, 2H), 2.01 (m, 2H), 2.84 (d, J=11.87 Hz, 2H), 3.38 (d, J=7.80 Hz, 2H), 3.79 (m, 1H), 4.02 (m, 3H), 5.99 (s, 2H), 6.78 (m, 4H), 7.43 (s, 1H), 7.98 (s, 1H), 8.83 (d, J=8.14 Hz, 1H); MS (ESI) m/z 471 [M+H]⁺.

### Example 53

### 6-chloro-N-{1-[(2,2-difluoro-1,3-benzodioxol-5-yl)methyl]piperidin-4-yl}-7-methoxy-4-oxo-4H-chromene-2-carboxamide

Example 53 was prepared according to the procedure outlined in Example 52, substituting 1-[(2,2-difluoro-1,3-benzodioxol-5-yl)methyl]piperidin-4-ylamine bis-hydrochloride salt for 1-benzo[1,3]dioxol-5-ylmethyl-piperidin-4-ylamine bis-hydrochloride salt. ¹H NMR (300 MHz, DMSO-d₆) δ ppm 1.66 (m, 2H), 1.80 (m, 2H), 2.06 (m, 2H), 2.84 (d, J=11.53 Hz, 2H), 3.50 (s, 2H), 3.80 (m, 1H), 4.03 (d, J=6.78 Hz, 3H), 6.79 (s, 1H), 7.14 (d, J=8.14 Hz, 1H), 7.35 (m, 2H, 7.44 (m, 1H), 7.98 (s, 1H), 8.84 (d, J=8.14 Hz, 1H).

### Example 54

### N-[1-(1,3-benzodioxol-5-ylmethyl)piperidin-4-yl]-7-chloro-6-fluoro-4-oxo-4H-chromene-2-carboxamide

### Example 54A

### Acetic acid 3-chloro-4-fluoro-phenyl ester

To a 100 mL round bottom flask was added 3-Chloro-4-fluoro-phenol (5 g, 34.1 mmol), pyridine (8.3 mL, 102.6 mmol), dichloromethane (10 mL), and acetic anhydride (3.54 mL, 37.4 mmol). The mixture was stirred at room temperature for 16 hours. The mixture was washed with 10% acetic acid/water. The organic layer was concentrated to dryness to provide acetic acid 3-chloro-4-fluoro-phenyl ester as a white solid. ¹H NMR (300 MHz, DMSO-d₆) δ ppm 2.25 (s, 3H), 7.20 (m, 1H), 7.50 (m, 2H).

### Example 54B

### 1-(4-Chloro-5-fluoro-2-hydroxy-phenyl)-ethanone

To a 100 mL round bottom flask was added acetic acid 3-chloro-4-fluoro-phenyl ester (2 g, 10.6 mmol), aluminum chloride (4 g, 30 mmol), and dichloromethane (10 mL). The mixture was stirred at room temperature for 1 hour after which the dichloromethane was then distilled off using a short-path distillation (oil bath temperature=70° C.). The mixture was slowly heated to 120° C. and HCl gas evolved. After 10 minutes, the bath temperature was increased to 140° C. and the mixture was stirred for 2 hours. The mixture was allowed to cool to room temperature and then diluted with 50% HCl and water. Solid was filtered and washed with water to provide 1-(4-Chloro-5-fluoro-2-hydroxy-phenyl)-ethanone as a solid. ¹H NMR (300 MHz, DMSO-d₆) δ ppm 2.60 (s, 3H), 7.2 (d, 1H), 7.85 (d, 1H), 11.70 (s, 1H).

### Example 54C

### 7-Chloro-6-fluoro-4-oxo-4H-chromene-2-carboxylic acid

To a solution of 1-(4-Chloro-5-fluoro-2-hydroxy-phenyl)-ethanone (1.25 g, 6.63 mmol) in diethyl oxalate (5.4 mL) was added NaOEt (9.0 mL, 23.0 mmol, 20 weight % in EtOH), and the mixture heated to 50° C. for 0.5 hour. The mixture was cooled to ambient temperature, diluted with diethyl ether (25 mL), and filtered. The yellow solid was suspended in a solution of concentrated HCl (1 mL) in acetic acid (7 mL), and heated to reflux for 1.5 hour. 6 N HCl (4 mL) was added to the mixture, and it was heated to reflux for an additional 16 hour. The mixture was cooled to ambient temperature, diluted with H₂O (25 mL), and filtered. The solid was air-dried to provide the title compound as a solid. ¹H NMR (300 MHz, DMSO-d₆) δ ppm 3.5 (s, 1H), 6.9 (s, 1H), 7.90 (d, 1H), 8.25 (d, 1H); MS (ESI) m/z 241 [M−H]⁻.

### Example 54

### N-[1-(1,3-benzodioxol-5-ylmethyl)piperidin-4-yl]-7-chloro-6-fluoro-4-oxo-4H-chromene-2-carboxamide

To a 4 mL vial with screw cap was added 7-Chloro-6-fluoro-4-oxo-4H-chromene-2-carboxylic acid (306 mg, 1.26 mmol), 1-benzo[1,3]dioxol-5-ylmethyl-piperidin-4ylamine dihydrochloric acid salt (407 mg, 1.32 mmol), EDCI (242 mg, 1.26 mmol), HOBT (170 mg, 1.26 mmol), NMM (509 mg, 5.04 mmol) and 1.5 mL of DMF. The mixture was placed on a shaker at 55° C. for 16 hourrs. The mixture was diluted with water (1 mL), extracted 3 times dichloromethane (5 mL), combined organics were concentrated to dryness. The residue was purified on a FlashMaster II silica column using gradient conditions from 100% dichloromethane to 5% methanol/dichloromethane. Product fractions were combined and concentrated to dryness to provide 7-Chloro-6-fluoro-4-oxo-4H-chromene-2-carboxylic acid (1-benzol[1,3]dioxol-5ylmethyl-piperidin-4-yl)-amide. ¹H NMR (300 MHz, DMSO-d₆) δ ppm 1.60 (m, 2H), 1.80 (m, 2H), 2.00 (m, 2H), 2.80(m, 2H), 3.40 (s, 2H), 3.80 (m, 1H), 6.00 (s, 2H), 6.70-6.90 (m, 4H), 7.95 (d, 1H), 8.10 (d, 1H), 8.80 (d, 1H); MS (ESI) m/z 459[M+H]⁺.

### Example 55

### N-[1-(1,3-benzodioxol-5-ylmethyl)piperidin-4-yl]-6,7-difluoro-4-oxo-4H-chromene-2-carboxamide

### Example 55A

### 3,4-difluorophenyl acetate

Acetyl chloride (3.5 mL, 48.1 mmol) was added slowly to 3,4-difluorophenol (5.00 g, 38.5 mmol), triethylamine (6.70 mL, 48.1 mmol), and CH₂Cl₂ at 0° C. The slurry was permitted to warm to room temperature as it was stirred overnight. After 15 hours the heterogeneous solution was concentrated, diluted with Et₂O, filtered through a 5 gram sep pack (Altech), and rinsed with Et₂O (100 mL). The filtrate was concentrated under reduced pressure to provide the title product. ¹H NMR (300 MHz, DMSO-d6): δ ppm 2.26 (s, 3H) 7.00-7.06 (m, 1H) 7.38 (ddd, J=11.36, 6.95, 2.71 Hz, 1H) 7.49 (dt, J=10.59, 9.11 Hz, 1H).

### Example 55B

### 1-(4,5-difluoro-2-hydroxyphenyl)ethanone

Example 55A (6.78 g, 39.4 mmol) was added to a slurry of aluminum trichloride (16 g, 118 mmol) in CH₂Cl₂ via pipette. The mixture turned violet immediately, and gas evolution was observed during the addition. After the solution was stirred for 1 hour at room temperature the mixture was warmed to 60-70° C. to distil off CH₂Cl₂. Once the solvent was gone, the mixture was warmed to 80° C. then to 140° C. After 1 hour 40 minutes at 140° C. the solution was cooled to ambient temperature and combined with 50% aqueous HCl (30 mL) dropwise by pipette. The misture was cooled on ice and the resulting brown solid collected by filtration, washed with distilled water, and air-dried overnight to provide the title compound as a brown solid. ¹H NMR (300 MHz, DMSO-d6): δ ppm 2.62 (s, 3H) 7.07 (dd, J=12.21, 6.78 Hz, 1H) 7.97 (dd, J=1.36, 9.32 Hz, 1H) 12.01 (s, 1H); MS (ESI, MeOH/NH4OH) m/z 171 [M]⁺.

### Example 55C

### 6,7-difluoro-4-oxo-4H-chromene-2-carboxylic acid

Example 55B (1.39 g, 8.07 mmol) and diethyl oxalate (5.5 mL, 40 mmol) were added to sodium ethoxide (10.5 mL of a 21% by weight solution in EtOH, 32.3 mmol), and the system was heated to 40° C. for 2 hours, cooled to room temperature, and filtered. The resulting solid was washed with Et₂O (25 mL). The solid was dissolved in CH₂Cl₂ (30 mL), washed with 10% aqueous acetic acid (1×40 mL), washed with brine (1×40 mL), and concentrated to a dark solid. The solid was dissolved in glacial acetic acid (8 mL) and 6N aqueous HCl (4 mL) and heated to 80° C. for 16 hours. The solution was cooled to room temperature, combined with distilled water (12 mL), the resulting precipitate collected by filtration, rinsed with distilled water (15 mL) and air-dried to provide the title product as a beige solid. ¹H NMR (300 MHz, DMSO-d6): δ ppm 6.93 (s, 1H), 8.00 (dd, J=10.85, 6.44 Hz, 1H), 8.09 (dd, J=10.85, 6.44 Hz, 1H), 12 (br. s, 1H); MS (ESI, MeOH/NH4OH) m/z 225 [M−H]⁺.

### Example 55D

### tert-butyl 4-{[(6,7-difluoro-4-oxo-4H-chromen-2-yl)carbonyl]amino}piperidine-1-carboxylate

Example 55C (1.03 g, 4.55 mmol), 4-amino-1-N-Boc-piperidine (1.00 g, 5.01 mmol), HOBt (769 mg, 5.69 mmol), EDC (1.09 g, 5.69 mmol), diisopropylethyl amine (1.60 mL, 9.10 mmol) were processed as described in Example 1F to provide the title compound as a white solid. ¹H NMR (300 MHz, DMSO-d6): δ ppm 1.38-1.51 (m, 11H), 1.81 (dd, J=12.71, 2.88 Hz, 2H), 2.78-2.94 (m, 2H), 3.91-4.06 (m, 3H), 6.85 (s, 1H), 7.89 (dd, J=10.85, 6.44 Hz, 1H), 8.00 (dd, J=10.00, 8.65 Hz, 1H), 8.85 (d, J=8.14 Hz, 1H); MS (ESI, MeOH/NH4OH) m/z 309 [M+H-Boc]⁺, 307 [M-Boc-H]⁺.

### Example 55E

### 6,7-difluoro-4-oxo-N-piperidin-4-yl-4H-chromene-2-carboxamide

Example 55D (856 mg, 2.10 mmol), trifluoroacetic acid (3 mL), and CH₂Cl₂ (7 mL) were processed as described in Example 1G to provide the title compound as a yellow solid. ¹H NMR (300 MHz, DMSO-d6): δ ppm 1.39-1.54 (m, 2H), 1.71-1.78 (m, 2H), 2.46-2.55 (m, 2H), 2.93-3.01 (m, 2H), 3.82 (dd, J=7.63, 3.56 Hz, 1H), 6.84 (s, 1H), 7.92 (dd, J=10.68, 6.61 Hz, 1H), 8.00 (dd, J=10.17, 8.82 Hz, 1H), 8.82 (d, J=8.14 Hz, 1H); MS (ESI, MeOH/NH4OH) m/z 309 [M+H]⁺, 307 [M−H]⁺.

### Example 55

### N-[1-(1,3-benzodioxol-5-ylmethyl)piperidin-4-yl]-6,7-difluoro-4-oxo-4H-chromene-2-carboxamide

Example 55E (35 mg, 0.113 mmol), piperonal (18 mg, 0.119 mmol), sodium triacetoxyborohydride (48 mg, 0.226 mmol), Na₂SO₄ (32 mg, 0.226 mmol), acetic acid (20 μL), and THF (0.5 mL) were processed as described in Example 1 to provide the title compound as an off-white solid. ¹H NMR (300 MHz, DMSO-d6): δ ppm 1.52-1.71 (m, 2H), 1.73-1.87 (m, 2H), 1.94-2.10 (m, 2H), 2.76-2.92 (m, 2H), 3.35-3.46 (m, 2H), 3.67-3.82 (m, 1H), 5.99 (s, 2H), 6.77 (s, 1H), 6.81-6.92 (m, 3H), 7.91 (dd, J=10.51, 6.44 Hz, 1H), 7.95-8.05 (m, 1H), 8.84 (d, J=8.14 Hz, 1H); MS (ESI, MeOH/NH4OH) m/z 443 [+H]⁺, 441 [M−H]⁺.

### Example 56

### N-[1-(1,3-benzodioxol-5-ylmethyl)piperidin-4-yl]-6-fluoro-7-methoxy-4-oxo-4H-chromene-2-carboxamide

### Example 56A

### 1-(4,5-difluoro-2-methoxymethoxy-phenyl)-ethanone

A mixture of 1-(4,5-difluoro-2-hydroxy-phenyl)-ethanone (4.00 g, 23.2 mmol), K₂CO₃ (3.84 g, 27.8 mmol), and MOM-Cl (2.12 mL, 27.8 mmol) in acetone (30 mL) was stirred at room temperature for 16 hours. The mixture was then diluted by the addition of H₂O (20 mL) and Et₂O (20 mL). The layers were separated, and the aqueous was extracted with additional Et₂O (3×20 mL). The combined organic layers were dried with anhydrous Na₂SO₄, filtered, and concentrated under reduced pressure. The residue was purified by MPLC (10% EtOAc in hexane to 35% EtOAc in hexane)) to provide the title compound as a colorless oil. ¹H NMR (300 MHz, DMSO-d6) δ ppm 2.57 (s, 3H), 3.42 (s, 3H), 5.43 (s, 2H), 7.35 (dd, 1H, J=6.4 and 12.6 Hz), and 7.64 (dd, 1H, J=11.2 and 9.5 Hz).

### Example 56B

### 1-(5-fluoro-4-methoxy-2-methoxymethoxy-phenyl)-ethanone

To a solution of 1-(4,5-difluoro-2-methoxymethoxy-phenyl)-ethanone (3.00 g, 13.9 mmol) in MeOH (30 mL) was added solid NaOMe (11.3 g, 209 mmol) in three portions. The resulting solution was heated to 40° C. for 8 hours. The cooled mixture was then quenched by addition of H₂O (20 mL) and EtOAc (100 mL).). The layers were separated, and the aqueous was extracted with additional EtOAc (3×20 mL). The combined organic layers were dried with anhydrous Na₂SO₄, filtered, and concentrated under reduced pressure to provide the title compound as an oil that was used without further purification. ¹H NMR (300 MHz, DMSO-d6) δ ppm 2.55 (s, 3H), 3.45 (s, 3H), 3.91 (s, 3H), 5.37 (s, 2H), 6.94 (d, 1H, J=7.1 Hz), and 7.47 (d, 1H, J=12.2 Hz).

### Example 56C

### 1-(5-fluoro-4-methoxy-2-hydroxy-phenyl)-ethanone

To an ambient solution of 1-(5-fluoro-4-methoxy-2-methoxymethoxy-phenyl)-ethanone (2.97 g, 13.03 mmol) in MeOH (11 mL) and H₂O (11 mL) was added TFA (7.28 mL). The mixture was stirred at room temperature for 16 hours, and was then diluted with H₂O (25 mL) and Et₂O (25 mL). The layers were separated, and the aqueous was extracted with additional Et₂O (3×25 mL). The combined organic layers were dried with anhydrous Na₂SO₄, filtered, and concentrated under reduced pressure. The residue was purified on SiO₂ gel by MPLC (15% EtOAc in hexane to 50% EtOAc in hexane) to provide the title compound. MS (ESI) m/z 183 (M−H)⁻.

### Example 56D

### 6-fluoro-7-methoxy-4-oxo-4H-chromene-2-carboxylic acid

Example 56D was prepared according to the procedure outlined in Example 52D, substituting 2-hydroxy-4-methoxy-5-fluoro-acetophenone for 2-hydroxy-4-methoxy-5-chloroacetophenone. MS (ESI) m/z 237 (M−H)⁻.

### Example 56E

### 4-[(6-fluoro-7-methoxy-4-oxo-4H-chromene-2-carbonyl)-amino]-piperidine

Example 56E was prepared according to the procedure outlined in Example 50C, substituting 6-fluoro-7-methoxy-4-oxo-4H-chromene-2-carboxylic acid for 6,7-dichloro-4-oxo-4H-chromene-2-carboxylic acid. ¹H NMR (300 MHz, DMSO-d6) δ ppm 1.77 (dq, 2H, J=4.4 and 13.5 Hz), 2.00 (br dd, 2H, J=2.0 and 13.6 Hz), 3.05 (q, 2H, J=11.5 Hz), 3.36 (br d, 2H, J=11.5 Hz), 4.03 (s, 3H), 4.04-4.15 (m, 1H), 6.83 (s, 1H), 7.45 (d, 1H, J=7.1 Hz), 7.75 (d, 1H, J=11.2 Hz), 8.37 (s, 1H), and 9.02 (d, 1H, J=7.5 Hz); MS (ESI) m/z 319 (M−H)⁻.

### Example 56

### N-[1-(1,3-benzodioxol-5-ylmethyl)piperidin-4-yl]-6-fluoro-7-methoxy-4-oxo-4H-chromene-2-carboxamide

To a mixture of 4-[(6-fluoro-7-chloro-4-oxo-4H-chromene-2-carbonyl)-amino]-piperidine (400 mg, 1.25 mmol), piperonal (206 mg, 1.38 mmol), and Na₂SO₄ (355 mg, 2.50 mmol) in THF (5 mL) was added AcOH (0.200 mL), and the mixture stirred for 0.5 hours. NaBH(OAc)₃ (530 mg, 2.50 mmol) was added, and the mixture was stirred for an additional 16 hours at ambient temperature. The mixture was then quenched by the addition of EtOAc (10 mL) and H₂O (10 mL). The layers were separated, and the aqueous was extracted with additional EtOAc (3×10 mL). The combined organics were dried with anhydrous Na₂SO₄, filtered, and concentrated under reduced pressure. The solid residue was recrystallized from hot CH₃CN to provide the title product as a white solid. ¹H NMR (300 MHz, DMSO-d6) δ ppm 1.53-1.72 (m, 2H), 1.72-1.87 (m, 2H), 1.92-2.10 (m, 2H), 2.84 (d, 2H, J=11.2 Hz), 3.39 (s, 2H), 3.68-3.87 (m, 1H), 4.02 (s, 3H), 5.99 (s, 2H), 6.75 (dd, 1H, J=7.9, 1.6 Hz), 6.78 (s, 1H), 6.83-6.87 (m, 2H), 7.46 (d, 1H, J=7.0 Hz), 7.73 (d, 1H, J=10.9 Hz), 8.82 (d, 1H, J=8.0 Hz); MS (ESI) m/z 455 (M+H)⁺.

### Example 57

### N-{1-[(1-ethyl-2-oxo-1,2-dihydroquinolin-7-yl)methyl]piperidin-4-yl}-7-fluoro-4-oxo-4H-chromene-2-carboxamide

### Example 57A

### 1-Ethyl-7-methyl-1H-quinolin-2-one

Example 57A was prepared using a similar procedure as described for Example 69A substituting ethyl iodide for methyl iodide in the step described in Example 69A. ¹H NMR (300 MHz, DMSO-d6) δ ppm 1.21 (t, 3H, J=10 Hz), 2.47 (s, 3H), 4.26 (q, 2H, J=10 Hz), 6.52 (d, 1H, J=9 Hz), 7.10 (d, 1H, J=6 Hz), 7.40 (s, 1H), 7.59 (d, 1H, J=9 Hz), 7.84 (d, 1H, J=9 Hz).

### Example 57

### N-{1-[(1-ethyl-2-oxo-1,2-dihydroquinolin-7-yl)methyl]piperidin-4-yl}-7-fluoro-4-oxo-4H-chromene-2-carboxamide

Example 57 was prepared according to the conditions described for Example 87 substituting 1-ethyl-7-methyl-1H-quinolin-2-one for 7-methyl-1H-quinolin-2-one to provide the target compound as a tan solid. ¹H NMR (500 MHz, DMSO-d6) δ ppm 1.23 (t, 3H, J=10 Hz), 1.63-1.73 (m, 2H), 1.80-1.88 (m, 2H), 2.07-2.17 (m, 2H), 2.85-2.93 (m, 2H), 3.66 (s, 2H), 3.77-3.87 (m, 1H), 4.27 (q, 2H, J=10 Hz), 6.56 (d, 1H, J=12 Hz), 6.83 (s, 1H), 7.22 (d, 1H, J=10 Hz), 7.43 (dt, 1H, J=10, 5 Hz), 7.49 (s, 1H), 7.60 (dd, 1H, J=10, 5 Hz), 7.68 (d, 1H, J=12 Hz), 7.88 (d, 1H, J=12 Hz), 8.11 (dd, 1H, J=10, 5 Hz), 8.84 (d, 1H, J=10 Hz); MS (ESI) m/z 476 [M+H]⁺.

### Example 58

### N-{1-[(5R)-6,7-dihydro-5H-indeno[5,6-d][1,3]dioxol-5-yl]piperidin-4-yl}-7-fluoro-4-oxo-4H-chromene-2-carboxamide

### Example 58A

### [1-(6,7-Dihydro-5H-indeno[5,6-d][1,3]dioxol-5-yl)-piperidin-4-yl]-carbamic acid tert-butyl ester

A solution of 4-N-boc-aminopiperidine (2.00 g, 10.0 mmol) and 6,7-Dihydro-indeno[5,6-d][1,3]dioxol-5-one (1.76 g, 10.0 mmol) in THF (30 mL) was placed under N₂ and Ti(ⁱOPr)₄ (3.66 mL, 13.3 mmol) was added slowly via syringe. The mixture was then heated to reflux for 8 hours. After this time, the mixture was cooled to room temperature, and NaBH₃CN (0.415 g, 6.69 mmol) was added in portions with stirring. The mixture was then slowly heated to 50° C. and allowed to stir at this temperature overnight. After 14 hours, the mixture was first cooled to room temperature, and then placed in an ice bath prior to the addition of 2 mL of sat. NaHCO₃. The resulting quenched mixture was stirred for 30 min, and then filtered over a plug of celite, and the cake rinsed with EtOAc (50 mL). Following concentration of the eluant, the residue was then taken up in CH₂Cl₂ and purified via silica gel chromatography (10-25% EtOAc (1% Et₃N) in hexanes) to provide the titled compound. MS (ESI) m/z 361 [M+H]⁺.

### Example 58B

### 1-(6,7-Dihydro-5H-indeno[5,6-d][1,3]dioxol-5-yl)-piperidin-4-ylamine

A portion of 58A (0.400 g, 1.11 mmol) was dissolved in CH₂Cl₂ (2 mL) and 1 mL of HCl/ether (1 M) was added via syringe. The solution was allowed to stand at room temperature for 6 hours, within which time a white precipitate formed. This precipitate was collected via suction filtration and rinsed with ether to provide the title product as the bis-HCl salt. MS (ESI) m/z 261 [M+H]⁺.

### Example 58

### N-{1-[(5R)-6,7-dihydro-5H-indeno[5,6-d][13]dioxol-5-yl]piperidin-4-yl}-7-fluoro-4-oxo-4H-chromene-2-carboxamide

After drying in a heated oven for 2 hours, 58B was dissolved in 4.4 mL of DMF and 0.243 g (1.17 mmol) of 7-methoxy-4-oxo-4H-chromene-2-carboxylic acid, 0.473 g of HATU (1.24 mmol), and 0.773 mL of DIPEA (4.44 mmol) was added. The solution was stirred for 4 hours, after which time the solvents evaporated. The residue was then dissolved in EtOAc and purified via column chromatography to provide the title compound in racemic form. Chromatography on a Chiralcell OJ column using hexanes/EtOH/MeOH (70/15/15) as the mobile phase provide the titled enantiomer. ¹H NMR (300 MHz, DMSO-d6) δ ppm 1.45-1.61 (m, 1H), 1.65-1.89 (m, 3H), 1.92-2.09 (m, 2H), 2.11-2.22 (m, 1H), 2.31-2.41 (m, 1H), 2.45-2.52 (m, 1H), 2.57-2.83 (m, 2H), 2.83-2.95 (m, 1H), 3.69-3.83 (m, 1H), 4.14-4.29 (m, 1H), 5.98 (d, 2H, J=3.73 Hz), 6.73 (s, 1H), 6.77 (s, 1H), 6.82 (s, 1H), 7.43 (td, 1H, J=8.69, 2.49 Hz), 7.62 (dd, 1H, J=9.47, 2.46 Hz), 8.11 (dd, 1H, J=8.89, 6.37 Hz), 8.84 (d, 1H, J=7.90 Hz); MS (ESI) m/z 451 [M+H]⁺.

### Example 59

### N-[(3S,4S)-1-(1,3-benzodioxol-5-ylmethyl)-3-methylpiperidin-4-yl]-7-methoxy-4-oxo-4H-chromene-2-carboxamide

### Example 59A

### 1,3-benzodioxol-5-ylmethyl methanesulfonate

Methanesulfonyl chloride (15.3 mL, 197 mmol) was added to a mixture of piperonyl alcohol (20.0 g, 131 mmol), triethylamine (36.5 mL, 26.2 mmol), and CH₂Cl₂ (210 mL) at 0° C. The solution was allowed to warm to room temperature as it was stirred overnight. After 13 hours, methanol (10 mL) was added, and the amber solution was stirred for 0.5 hours before it was diluted with CH₂Cl₂ (200 mL), washed with 0.5 N aqueous HCl (3×200 mL), washed with brine (1×200 mL), dried (MgSO₄), filtered, and concentrated to a dark oil (16.3 g, 53%). ¹H NMR (300 MHz, DMSO-d₆): δ ppm 3.32 (s, 3H) 4.69 (s, 2H) 6.03 (s, 2H) 6.83-6.95 (m, 2H) 7.00 (d, J=1.36 Hz, 1H).

### Example 59B

### 1-(1,3-benzodioxol-5-ylmethyl)piperidin-4-one

Potassium carbonate (24.5 g, 177 mmol) was added to Example 59A (16.3 g, 20.8 mmol), 4-piperidone monohydrate hydrochloride (16.3 g, 106 mmol), and DMF (140 mL). The dark, heterogeneous solution was vigorously stirred over the weekend. The mixture was diluted with Et₂O (350 mL), washed with distilled water (3×200 mL), washed with brine (1×200 mL), dried (MgSO₄), filtered, and concentrated under reduced pressure overnight to provide crystals of the title compound (16.9 g, 100%). ¹H NMR (300 MHz, DMSO-D6): δ ppm 2.33 (t, J=6.10 Hz, 4H) 2.65 (t, J=6.10 Hz, 4H) 3.51 (s, 2H) 5.98 (s, 2H) 6.80 (m, 2H) 6.92 (d, J=1.70 Hz, 1H).

### Example 59C

### 1-(1,3-benzodioxol-5-ylmethyl)-3-methylpiperidin-4-one

A solution of Example 59B (1 gram, 4.29 mmol) in 15 mL of THF under nitrogen at −78° C. was charged with 2.5 mL (4.5 mmol) of lithium diisopropylamide (1.8 M in heptane/THF) dropwise via syringe. After one hour, iodomethane (0.28 mL, 4.5 mmol) was added by syringe. After an additional four hours, the was allowed to warm to room temperature, diluted with CH₂Cl₂, washed with 1 M aqueous potassium carbonate, dried (Na₂SO₄), filtered and concentrated to provide a brown oil. Purification by flash silica gel chromatography (gradient elution of 0 to 30% ethyl acetate/hexane) to provide the title compound as a clear oil. MS (ESI, MeOH/NH₄OH) m/z 248 [M+H].

### Example 59D

### 1-(1,3-benzodioxol-5-ylmethyl)-3-methylpiperidin-4-amine

A solution of Example 59C (105 mg, 0.43 mmol) was charged with ammonium acetate (330 mg, 4.3 mmol) and NaCNBH₃ (20 mg, 0.32 mmol). The heterogeneous mixture was stirred for 24 hours, diluted with 95/5 CH₂Cl₂/MeOH, washed with 1M aqueous K₂CO₃, aqueous extracted with additional 95/5 CH₂Cl₂/MeOH, organic extracts combined, dried (Na₂SO₄), filtered and concentrated to provide the title compound as a 2:1 mixture of diastereomers. MS (ESI, MeOH/NH4OH) m/z 249 [M+H].

### Example 59

### N-[(3 S,4S)-1-(1,3-benzodioxol-5-ylmethyl)-3-methylpiperidin-4-yl]-7-methoxy-4-oxo-4H-chromene-2-carboxamide

A solution of Example 59D (33 mg, 0.13 mmol) in 0.6 mL dimethylformamide was charged with hydroxybenzotriazole (20 mg, 0.15 mmol), ethyldimethylaminocarbodiimde (28 mg, 0.15 mmol) and diisopropylethylamine (30 uL, 0.16 mmol). The was shaken for ten days, diluted with CH₂Cl₂, washed with 1M K₂CO₃, dried (Na₂SO₄), filtered through a plug of silica (2 g SepPak) rinsed through with 95/5 CH₂Cl₂/MeOH and concentrated. Purification by flash silica gel chromatography (0 to 100% ethyl acetate/hexane) produced two clean diastereomers. The title compound was the more polar on normal phase TLC (Rf=0.69 90/10 CH₂Cl₂/MeOH). MS (ESI, MeOH/NH₄OH) m/z 451 [M+H], 449 [M−H]. ¹H NMR (300 MHz, DMSO-d6) δ ppm 0.81 (d, 3H, J=6.10 Hz), 1.62-2.06 (m, 5H), 2.80-2.89 (m, 2H), 3.40 (s, 2H), 3.44-3.60 (m, 1H), 3.93 (s, 3H), 5.99 (s, 2H), 6.73-6.77 (m, 2H), 6.82-6.86 (m, 2H), 7.10 (dd, 1H, J=8.81, 2.42 Hz), 7.23 (d, 1H, J=2.38 Hz), 7.95 (d, 1H, J=8.82 Hz), 8.76 (d, 1H, J=8.96 Hz).

### Example 60

### N-[1-(1,3-benzodioxol-5-ylmethyl)piperidin-4-yl]-7-fluoro-4-oxo-4H-chromene-2-carboxamide

### Example 60A

### ethyl 7-fluoro-4-oxo-4H-chromene-2-carboxylate

4′-Fluoro-2′-hydroxyacetophenone (5.00 g, 32.4 mmol), diethyl oxalate (22 mL, 162 mmol), and sodium ethoxide (42 mL of a 21% by weight solution in ethanol, 130 mmol) was processed as described in Example 1D to provide the title compound as a one to one mixture of carboxylic acid and ethyl ester. ¹H NMR (300 MHz, DMSO-d6): δ ppm 1.35 (t, J=7.12 Hz, 1.5H), 4.40 (q, J=7.12 Hz, 1H), 6.92 (s, 0.5H), 6.97 (s, 0.5H), 7.40-7.48 (m, J=8.69, 8.69, 3.81, 2.54 Hz, 1H), 7.75 (ddd, J=12.38, 9.66, 2.37 Hz, 1H), 8.12 (ddd, J=8.65, 6.61, 1.70 Hz, 1H); MS (APCI) m/z 237 [M+H]⁺.

### Example 60B

### 7-fluoro-4-oxo-4H-chromene-2-carboxylic acid

Example 60A (4.52 g, 19.1 mmol), glacial acetic acid (80 mL), and 6N aqueous HCl (40 mL) were processed as described in Example 1E to provide the title compound as a white powder. ¹H NMR (300 MHz, DMSO-d6): δ ppm 6.92 (s, 1H), 7.43 (td, J=8.82, 2.37 Hz, 1H), 7.73 (dd, J=9.49, 2.37 Hz, 1H), 8.12 (dd, J=9.16, 6.44 Hz, 1H); MS (APCI) m/z 209 [M+H]⁺, 208 [M−H]⁺.

### Example 60C

### tert-butyl 4-{[(7-fluoro-4-oxo-4H-chromen-2-yl)carbonyl]amino}piperidine-1-carboxylate

Example 60B (2.00 g, 8.47 mmol),), 4-amino-1-N-Boc-piperidine (1.87 g, 9.32 mmol), HOBt (1.43 g, 10.6 mmol), EDC (2.03 g, 10.6 mmol), diisopropylethyl amine (3.00 mL, 16.9 mmol) were processed as described in Example 1F to provide the title compound as a pale yellow solid. ¹H NMR (300 MHz, DMSO-d6): δ ppm 1.38-1.52 (m, 11H), 1.78 (d, J=3.73 Hz, 2H), 2.78-2.94 (m, 2H), 3.91-4.05 (m, 3H), 6.84 (s, 1H), 7.43 (td, J=8.73, 2.54 Hz, 1H), 7.60 (dd, J=9.49, 2.37 Hz, 1H), 8.12 (dd, J=8.99, 6.27 Hz, 1H), 8.85 (d, J=8.14 Hz, 1H); MS (APCI) m/z 391 [M+H]⁺, 389 [M−H]⁺.

### Example 60D

### 7-fluoro-4-oxo-N-piperidin-4-yl-4H-chromene-2-carboxamide

Example 60C (2.90 g, 7.43 mmol), trifluoroacetic acid (7.4 mL), and CH₂Cl₂ (16 mL) were processed as described in Example 1G to provide the title compound as a white solid. ¹H NMR (300 MHz, DMSO-d6): δ ppm 1.49 (dd, J=11.53, 4.07 Hz, 2H), 1.74-1.81 (m, 2H), 2.51-2.61 (m, 2H), 2.98-3.05 (m, 2H), 3.79-3.91 (m, J=7.46 Hz, 1H), 6.83 (s, 1H), 7.43 (td, J=8.73, 2.54 Hz, 1H), 7.63 (dd, J=9.49, 2.37 Hz, 1H), 8.12 (dd, J=8.98, 6.27 Hz, 1H), 8.84 (d, J=8.14 Hz, 1H); MS (APCI) m/z 291 [M+H]⁺, 289 [M−H]⁺.

### Example 60

### N-[1-(1,3-benzodioxol-5-ylmethyl)piperidin-4-yl]-7-fluoro-4-oxo-4H-chromene-2-carboxamide

Example 60C (500 mg, 1.55 mmol), piperonal (244 mg, 1.63 mmol), sodium triacetoxyborohydride (657 mg, 3.10 mmol), Na₂SO₄ (440 mg, 3.10 mmol), acetic acid (250 μL), and THF (6.2 mL) were processed as described in Example 1 to provide the title compound as a white solid. ¹H NMR (300 MHz, DMSO-d6): δ ppm 1.51-1.72 (m, 2H), 1.72-1.87 (m, 2H), 1.92-2.10 (m, 2H), 2.83 (d, 2H, J=11.78 Hz), 3.39 (s, 2H), 3.70-3.85 (m, 1H), 5.99 (s, 2H), 6.75 (dd, 1H, J=7.93, 1.40 Hz), 6.81-6.87 (m, 3H), 7.43 (td, 1H, J=8.69, 2.49 Hz), 7.62 (dd, 1H, J=9.49, 2.46 Hz), 8.11 (dd, 1H, J=8.94, 6.40 Hz), 8.83 (d, 1H, J=8.14 Hz); MS (APCI) m/z 425 [M+H]⁺.

### Example 61

### N-{1-[(1R)-1-(2,2-difluoro-1,3-benzodioxol-5 yl)ethyl]piperidin-4-yl}-7-fluoro-4-oxo-4H-chromene-2-carboxamide

### Example 61A

### 1-(2,2-difluoro-benzo[1,3]dioxol-5-yl)-ethanone

Example 61A (142 mg) was made according to the procedure described in Example 3, substituting 2,2-difluoro-1,3-benzodioxole-5-carboxaldehyde for Example 3A. MS (ESI) m/z 265 [M+H]⁺.

### Example 61B

### N-{1-[1-(2,2-difluoro-1,3-benzodioxol-5-yl)ethyl]piperidin-4-yl}-7-fluoro-4-oxo-4H-chromene-2-carboxamide

Example 60D and Example 61A were processed according to the procedure described in Example 3 to prepare example 61B.

### Example 61

### N-{1-[(1R)-1-(2,2-difluoro-1,3-benzodioxol-5-yl)ethyl]piperidin-4-yl}-7-fluoro-4-oxo-4H-chromene-2-carboxamide

Example 61B was separated into its enantiomers by HPLC chromatography using a Chiralcel OJ column eluting with a mobile phase of hex/EtOH/MeOH (80/10/10). Example 61 was the second enantiomer to elute from the column. ¹H NMR (300 MHz, DMSO-d6) δ ppm 1.30 (d, 3H, J=6.61 Hz), 1.52-1.69 (m, 2H), 1.71-1.89 (m, 2H), 1.88-2.11 (m, 2H), 2.71-2.86 (m, 1H), 2.88-3.03 (m, 1H), 3.48-3.62 (m, 1H), 3.67-3.79 (m, 1H), 6.81 (s, 1H), 7.16 (t, 1H, J=8.18 Hz), 7.32-7.37 (m, 2H), 7.43 (td, 1H, J=8.71, 2.51 Hz), 7.61 (dd, 1H, J=9.46, 2.45 Hz), 8.11 (dd, 1H, J=8.89, 6.36 Hz), 8.78-8.84 (m, 1H); MS (ESI) m/z 475 [M+H]⁺.

### Example 62

### N-[1-(1-benzofuran-5-ylmethyl)piperidin-4-yl]-7-fluoro-4-oxo-4H-chromene-2-carboxamide

A solution of Example 60D (29 mg, 0.1 mmol) and benzofuran-5-carboxaldehyde (15 mg, 0.1 mmol) in THF/2% acetic acid (0.5 mL) was charged with NaBH(OAc)₃ (42 mg, 0.2 mmol) and shaken for five days. The heterogeneous mixture was quenched by addition of 1M K₂CO₃ (0.5 mL), diluted with CH₂Cl₂ (2 mL) dried (Na₂SO₄), placed onto a plug of silica (2 g sep Pak), rinsed with ethyl acetate (10 mL), collection tube changed, rinsed with 95/5 CH₂Cl₂/MeOH and concentrated to provide the title compound as a light orange solid. MS (ESI, MeOH/NH₄OH) m/z 421 [M+H], 419 [M−H]. ¹H NMR (300 MHz, DMSO-d6) δ ppm 8.80-8.84 (m, 1H), 8.11 (dd, 1H, J=8.89, 6.36 Hz), 7.97 (d, 1H, J=2.19 Hz), 7.62 (dd, 1H, J=9.47, 2.45 Hz), 7.57 (d, 1H, J=1.67 Hz), 7.53 (d, 1H, J=8.43 Hz), 7.43 (td, 1H, J=8.68, 2.49 Hz), 7.27 (dd, 1H, J=8.45, 1.72 Hz), 6.93 (dd, 1H, J=2.19, 0.93 Hz), 6.82 (s, 1H), 3.69-3.85 (m, 1H), 3.57 (s, 2H), 2.86 (d, 2H, J=11.97 Hz), 1.94-2.15 (m, 2H), 1.73-1.85 (m, 2H), 1.65 (qd, 2H, J=11.88, 3.78 Hz).

### Example 63

### N-{1-[(6-chloro-1,3-benzodioxol-5-yl)methyl]piperidin-4-yl}-7-fluoro-4-oxo-4H-chromene-2-carboxamide

Example 60C (19 mg, 0.065 mmol) and 6-chloropiperonal were processed in a manner similar to Example 40 to provide the title compound as the trifluoroacetic acid salt. ¹H NMR (300 MHz, DMSO-d6) δ ppm 1.57-1.72 (m, 2H), 1.76-1.86 (m, 2H), 2.07-2.19 (m, 2H), 2:85 (d, 2H, J=11.46 Hz), 3.48 (s, 2H), 3.74-3.88 (m, 1H), 6.06 (s, 2H), 6.83 (s, 1H), 7.01 (s, 1H), 7.05 (s, 1H), 7.43 (td, 1H, J=8.70, 2.47 Hz), 7.61 (dd, 1H, J=9.48, 2.46 Hz), 8.12 (dd, 1H, J=8.90, 6.36 Hz), 8.83 (d, 1H, J=7.94 Hz); MS (ESI, MeOH/NH4OH) m/z 459 [M+H]⁺, 457 [M−H]⁺.

### Example 64

### N-{1-[(1R)-6-(acetylamino)-2,3-dihydro-1H-inden-1-yl]piperidin-4-yl}-7-fluoro-4-oxo-4H-chromene-2-carboxamide

rac-Example 67 was separated into its enantiomers using a Chiralcel OJ column with 70:30 hexanes:ethanol mixture as the eluent to provide the title compound. ¹H NMR (500 MHz, DMSO-d6) δ ppm 1.50-1.59 (m, 1H), 1.67-1.76 (m, 2H), 1.80-1.85 (m, 1H), 1.95-2.05 (m, 5 H), 2.15-2.22 (m, 1H), 2.33-2.40 (m, 1H), 2.50-2.52 (m, 1H), 2.65-2.72 (m, 1H), 2.77-2.85 (m, 1H), 2.89-2.93 (m, 1H), 3.70-3.80 (m, 1H), 4.28 (t, 1H, J=7 Hz), 6.81 (s, 1H), 7.10 (d, 1H, J=5 Hz), 7.28 (d, 1H, J=5 Hz), 7.42 (dt, 1H, J=10, 3 Hz), 7.61 (dd, 1H, J=10, 5 Hz), 7.64 (s, 1H), 8.11 (dd, 1H, J=10, 5 Hz), 8.85 (d, 1H, J=10 Hz), 9.83 (s, 1H).

### Example 65

### N-[1-(1,3-benzodioxol-5-ylmethyl)piperidin-4-yl]-7-bromo-6-fluoro-4-oxo-4H-chromene-2-carboxamide

### Example 65A

### Acetic acid 3-bromo-4-fluoro-phenyl ester

A flask was charged with 3-Bromo-4-fluoro-phenol (0.5 g, 2.62 mmol), pyridine (0.53 mL, 6.55 mmol), CH₂Cl₂ (5 mL), and acetic anhydride (0.273 mL, 2.89 mmol). The solution was stirred at room temperature for 16 hours. The solution was washed with 10% acetic acid/water. The organic layer was concentrated to dryness to provide acetic acid 3-bromo-4-fluoro-phenyl ester. Product was used without further purification. MS (ESI) m/z 233 [M+H]⁺.

### Example 65B

### 1-(4-Bromo-5-fluoro-2-hydroxy-phenyl)-ethanone

A flask was charged with acetic acid 3-bromo-4-fluoro-phenyl ester (2.62 mmol) and boron trifluoride-acetic acid complex (10 mL, mmol). The solution was stirred and heated to reflux. After 2 hours, solution was cooled to room temperature. Solid formed. Solid was filtered, washed with water, and air dried to provide 1-(4-Bromo-5-fluoro-2-hydroxy-phenyl)-ethanone as a solid. MS (ESI) m/z 233 [M+H]⁺.

### Example 65C

### 7-Bromo-6-fluoro-4-oxo-4H-chromene-2-carboxylic acid

To a solution of 1-(4-Bromo-5-fluoro-2-hydroxy-phenyl)-ethanone (2.62 mmol) in diethyl oxalate (2.1 mL) was added NaOEt (4.1 mL, 10.4 mmol, 20 weight % in EtOH), and the mixture was heated to 50° C. for 0.5 hours. The mixture was cooled to room temperature, diluted with Et₂O (25 mL), and filtered. The yellow solid was suspended in a solution of concentrated HCl (1 mL) in AcOH (10 mL) and heated to reflux for 1.5 hours. 6 N HCl (4 mL) was added to the reaction, and it was heated to reflux for an additional 16 hours. The mixture was then cooled to room temperature, diluted with H₂O (25 mL), and filtered. The solid was air-dried to provide the title compound as a solid. ¹H NMR (300 MHz, DMSO-d₆) δ ppm 3.5 (s, 1H), 6.95 (s, 1H), 7.88 (d, 1H), 8.35 (d, 1H); MS (ESI) m/z 287 [M+H]⁺.

### Example 65

### N-[1-(1,3-benzodioxol-5-ylmethyl)piperidin-4-yl]-7-bromo-6-fluoro-4-oxo-4H-chromene-2-carboxamide

A 4 mL vial with screw cap was charged with 7-Bromo-6-fluoro-4-oxo-4H-chromene-2-carboxylic acid (32 mg, 0.11 mmol), 1-benzo[1,3]dioxol-5-ylmethyl-piperidin-4ylamine dihydrochloric acid salt (35 mg, 0.11 mmol), EDCI (22 mg, 0.11 mmol), HOBT (15 mg, 0.11 mmol), NMM (0.05 mL, 0.46 mmol) and 0.4 mL of DMF. The mixture vessel was placed on a shaker at 55 C for 16 hours. The solution was diluted with water (1 mL), extracted 3 times CH₂Cl₂ (5 mL), combined organics were concentrated to dryness. The crude product was purified on a FlashMaster II silica column using gradient conditions from 100% CH₂Cl₂ to 5% methanol/CH₂Cl₂. Product fractions were combined and concentrated to dryness to provide N-[1-(1,3-benzodioxol-5-ylmethyl)piperidin-4-yl]-7-bromo-6-fluoro-4-oxo-4H-chromene-2-carboxamide. ¹H NMR (300 MHz, Methanol-d4) δ ppm 1.67-1.82 (m, 2H), 1.90-1.99 (m, 2H), 2.13-2.24 (m, 2H), 2.93-3.02 (m, 2H), 3.50 (s, 2H), 3.84-3.97 (m, 1H), 5.93 (s, 2H), 6.72-6.83 (m, 2H), 6.88 (d, 1H, J=1.44 Hz), 6.97 (s, 1H), 7.87 (d, 1H, J=8.08 Hz), 8.14 (d, 1H, J=5.48 Hz) MS (ESI) m/z 503 [M+H]⁺.

### Example 66

### 7-fluoro-4-oxo-N-{1-[(1S)-1-(2-oxo-2H-chromen-7-yl)ethyl]piperidin-4-yl}-4H-chromene-2-carboxamide

Example 75 was placed on a Chiralcell OJ column and eluted using hexanes/EtOH/MeOH (50/25/25) as the mobile phase to provide the titled enantiomer. ¹H NMR (400 MHz, DMSO-D6) δ ppm 1.34 (d, J=6.44 Hz, 3H), 1.55-1.72 (m, 2H), 1.74-1.87 (m, 2H), 1.96-2.13 (m, J=11.97 Hz, 2H), 2.74-2.84 (m, 1H), 2.93-3.02 (m, 1H), 3.58-3.68 (m, J=6.75 Hz, 1H), 3.68-3.79 (m, 1H), 6.45 (d, J=9.51 Hz, 1H), 6.81 (s, 1H), 7.31-7.37 (m, 2H), 7.38-7.47 (m, 1H), 7.68 (d, J=7.98 Hz, 2H), 8.05 (d, J=9.82 Hz, 1H), 8.11 (dd, J=8.75, 6.29 Hz, 1H), 8.80 (d, J=7.98 Hz, 1H); MS (ESI) m/z 463 [M+H]⁺.

### Example 67

### N-{1-[6-(acetylamino)-2,3-dihydro-1H-inden-1-yl]piperidin-4-yl}-7-fluoro-4-oxo-4H-chromene-2-carboxamide

6-nitroindan-1-one (0.27 g, 1.52 mmol), iron powder (0.34 g, 6.08 mmol) and ammonium chloride (0.08 g, 1.52 mmol) were placed together in an ethanol (8 mL)/water (3 mL) mixture and heated at reflux for 6 hours. The mixture was cooled, filtered over wet celite, extracted with ethyl acetate (3×50 mL), dried (MgSO₄) and used without further purification. The crude amine (0.22 g, 1.52 mmol) was placed along with acetic acid (0.1 g, 1.6 mmol), EDCI (0.37 g, 1.9 mmol), HOBt (0.26 g, 1.9 mmol) and N-methyl morpholine (0.62 g, 6.1 mmol) in DMF (10 mL) and stirred at room temperature for 12 hours. The mixture was diluted with water, extracted with ethyl acetate (3×30 mL), organics washed with water, brine, dried (MgSO₄) and concentrated to a yellow power that was used without purification in the next step. N-(3-Oxo-indan-5-yl) acetamide (0.3 g, 1.59 mmol) and piperidin-4-yl-carbamic acid tert-butyl ester (0.38 g, 1.9 mmol) were placed with titanium tetraisopropoxide (1.36 g, 4.8 mmol) in THF (15 mL) and heated at reflux for 24 hours. The mixture was cooled to room temperature, sodium triacetoxyborohydride (1.2 g, 5.6 mmol) and ethanol (4 mL) added to the mixture and heated at 50° C. After 24 hours, the mixture was cooled, quenched with isopropanol (10 mL), saturated sodium bicarbonate (30 mL) and filtered over wet celite. The crude mixture was extracted with ethyl acetate (5×30 mL), organic extracts washed with water, brine, dried (MgSO₄) and concentrated to a brown foam. The crude product was dissolved in dichloromethane (6 mL) and treated with a 2M solution of HCl in Et₂O (2 mL) and stirred at room temperature for 6 hours. The precipitated solid was filtered and washed with ether, dried and used as is in the next step. N-[3-(4-amino-piperidin-1-yl)-indan-5-yl]-acetamide (0.8 g, 2.32 mmol) was placed along with 7-fluoro-4-oxo-4H-chromene-2-carboxylic acid (0.54 g, 2.6 mmol), EDCI (0.60 g, 3.1 mmol), HOBt (0.42 g, 3.1 mmol) and N-methyl morpholine (1.04 g, 10.4 mmol) in DMF (10 mL) and stirred at room temperature for hours: The mixture was diluted with water, extracted with ethyl acetate (6×50 mL), organics washed with saturated sodium bicarbonate, water, brine, dried (MgSO₄) and concentrated to one-half the volume when thick precipitate fell out of solution. The precipitate was filtered, washed with ethyl acetate and dried to provide a racemic mixture of the target compound as a white solid. ¹H NMR (500 MHz, DMSO-d6) δ ppm 1.50-1.59 (m, 1H), 1.67-1.76 (m, 2H), 1.80-1.85 (m, 1H), 1.95-2.05 (m, 5H), 2.15-2.22 (m, 1H), 2.33-2.40 (m, 1H), 2.50-2.52 (m, 1H), 2.65-2.72 (m, 1H), 2.77-2.85 (m, 1H), 2.89-2.93 (m, 1H), 3.70-3.80 (m, 1H), 4.28 (t, 1H, J=7 Hz), 6.81 (s, 1H), 7.10 (d, 1H, J=5 Hz), 7.28 (d, 1H, J=5 Hz), 7.42 (dt, 1H, J=10, 3 Hz), 7.61 (dd, 1H, J=10, 5 Hz), 7.64 (s, 1H), 8.11 (dd, 1H, J=10, 5 Hz), 8.85 (d, 1H, J=10 Hz), 9.83 (s, 1H); MS (ESI) m/z 464 [M+H]⁺.

### Example 68

### N-[1-(1,3-benzodioxol-5-ylmethyl)piperidin-4-yl]-6-fluoro-7-methyl-4-oxo-4H-chromene-2-carboxamide

The Example 65 procedure was applied to 3-methyl-4-fluoro-phenol (3 mL, 27 mmol) to provide N-[1-(1,3-benzodioxol-5-ylmethyl)piperidin-4-yl]-6-fluoro-7-methyl-4-oxo-4H-chromene-2-carboxamide. ¹H NMR (300 MHz, Methanol-d4) δ ppm 1.75 (qd, 2H, J=12.12, 3.70 Hz), 1.87-2.01 (m, 2H), 2.15 (td, 2H, J=12.05, 2.53 Hz), 2.46 (d, 3H, J=2.06 Hz), 2.92-3.00 (m, 2H), 3.47 (s, 2H), 3.90 (tt, 1H, J=11.37, 4.33 Hz), 5.93 (s, 2H), 6.72-6.83 (m, 2H), 6.87-6.88 (m, 1H), 6.94 (s, 1H), 7.66-7.72 (m, 2H) MS (ESI) m/z 439 [M+H]⁺.

### Example 69

### 7-fluoro-N-{1-[(1-methyl-2-oxo-1,2-dihydroquinolin-7-yl)methyl]piperidin-4-yl}-4-oxo-4H-chromene-2-carboxamide

### Example 69A

### 1,7-dimethyl-1H-quinolin-2-one

Example 87B (1.76 g, 11 mmol) was added to a suspension of sodium hydride (1 g, 24 mmol) in THF (30 mL) at 0° C. and allowed to warm up to room temperature over 2 hours. Methyl iodide was then added to the slurry and stirred at room temperature for 15 hours. The mixture was quenched with water, extracted with ethyl acetate (3×50 mL), organic extracts washed with water, brine, dried (Na₂SO₄) and purified by flash chromatography to provide the target compound as a yellow solid. ¹H NMR (400 MHz, DMSO-d6) δ ppm 2.47 (s, 3H), 3.60 (s, 3H), 6.54 (d, 1H, J=8 Hz), 7.10 (m, 1H), 7.35 (s, 1H), 7.59 (d, 1H, J=12 Hz), 7.84 (d, 1H, J=12 Hz).

### Example 69

### 7-fluoro-N-{1-[(1-methyl-2-oxo-1,2-dihydroquinolin-7-yl)methyl]piperidin-4-yl}-4-oxo-4H-chromene-2-carboxamide

Example 69 was prepared according to the conditions described for Example 87 substituting 1,7-dimethyl-1H-quinolin-2-one for 7-methyl-1H-quinolin-2-one to provide the target compound as a white solid. ¹H NMR (500 MHz, DMSO-d6) δ ppm 1.65-1.73 (m, 2H), 1.80-1.88 (m, 2H), 2.07-2.15 (m, 2H), 2.85-2.95 (m, 2H), 3.63 (s, 3H), 3.65 (s, 2H), 3.77-3.87 (m, 1H), 6.58 (d, 1H, J=10 Hz), 6.83 (s, 1H), 7.25 (d, 1H, J=5 Hz), 7.43 (dt, 1H, J=10, 5 Hz), 7.46 (s, 1H), 7.60 (dd, 1H, J=10, 5 Hz), 7.68 (d, 1H, J=10 Hz), 7.89 (d, 1H, J=10 Hz), 8.11 (dd, 1H, J=10, 5 Hz), 8.87 (d, 1H, J=10 Hz); MS (ESI) m/z 462 [M+H]⁺.

### Example 70

### 6,7-difluoro-N-{1-[(1-methyl-1H-indol-5-yl methyl]piperidin-4-yl}-4-oxo-4H-chromene-2-carboxamide

Example 55E (31 mg, 0.100 mmol), Example 17A, N-methylindole-5-carboxaldehyde, (16 mg, 0.100 mmol), macroporous-cyanoborohydride (101 mg, 0.250 mmol of 2.47 mmol/gram resin, Argonaut Technologies), acetic acid (30 μL), CH₂Cl₂ (0.5 mL) and THF (0.5 mL) were combined and agitated overnight. The mixture was added to a MP-TsOH cartridge (Argonaut Technologies), washed with THF, and rinsed with 2N ammonia in methanol. The ammonia in methanol fraction was concentrated and purified by a sep pack (Altech, 1 gram) eluting with 100% EtOAc to provide the title compound as a white solid. ¹H NMR (300 MHz, DMSO-d6): δ ppm 1.55-1.70 (m, 2H), 1.71-1.86 (m, 2H), 1.96-2.10 (m, 2H), 2.82-2.93 (m, 2H), 3.50-3.56 (m, 2H), 3.67-3.86 (m, 1H), 3.77 (s, 3H), 6.37 (d, 1H, J=3.04 Hz), 6.84 (s, 1H), 7.12 (dd, 1H, J=8.36, 1.49 Hz), 7.29 (d, 1H, J=3.08 Hz), 7.37 (d, 1H, J=8.39 Hz), 7.44 (s, 1H), 7.90 (dd, 1H, J=10.68, 6.44 Hz), 8.00 (dd, 1H, J=9.96, 8.86 Hz), 8.83 (d, 1H, J=7.84 Hz); MS (ESI, MeOH/NH4OH) m/z 452 [M+H]⁺, 450 [M−H]⁺.

### Example 71

### 7-fluoro-4-oxo-N-{1-[(2-oxo-2H-chromen-7-yl)methyl]piperidin-4-yl}-4H-chromene-2-carboxamide

### Example 71A

### 7-bromomethylcoumarin

A suspension of 7-methylcoumarin (1.49 g, 9.36 mmol) and N-bromosuccinimide (1.67 g, 9.36 mmol) in carbontetrachloride (200 mL) was treated with 2,2′-azobisisobutyronitril (0.050 g, 0.30 mmol) and heated at reflux for 24 hours. The mixture was cooled to room temperature, filtered through a pad of celite, and the filtrate was concentration to provide a white solid. The solid was recrystallized from hot acetonitrile to provide example 71A as a white solid. MS (ESI) m/z 239 [M+H]⁺.

### Example 71

### 7-fluoro-4-oxo-N-{1-[(2-oxo-2H-chromen-7-yl)methyl]piperidin-4-yl}4H-chromene-2-carboxamide

A solution of Example 60D (0.300 g, 1.03 mmol) and 7-bromomethylcoumarin (0.245 g, 1.03 mmol) in DMF (5 mL) was heated to 50° C. and stirred for 4 hours. The mixture was cooled to 0° C., and the white precipitate was collected by filtration, washed with diethylether (3×20 mL), and dried in a vacuum oven to provide Example 71. ¹H NMR (300 MHz, DMSO-d6) δ ppm 1.60-1.95 (m, 2H), 2.05-2.12 (m, 2H), 3.10-3.25 (m, 2H), 3.40-3.55 (m, 2H), 3.95-4.10 (m, 1H), 4.41 (d, 2H, J=4.7 Hz), 6.60 (d, 1H, J=9.6 Hz), 6.85 (s, 1H), 7.42-7.46 (m, 1H), 7.49 (d, 1H, J=9.6 Hz), 7.61 (m, 1H), 7.85 (d, 1H, J=8.1 Hz), 8.10-8.16 (m, 2H), 9.07 (d, 1H, J=7.5 Hz), 9.54-9.63 (m, 1H); MS (ESI) m/z 449 [M+H]⁺.

### Example 72

### 7-fluoro-N-[1-(4-methylbenzyl)piperidin-4-yl]-4-oxo-4H-chromene-2-carboxamide

The title compound was prepared according to the procedure for Example 79 substituting 4-methylbenzaldehyde for 3-fluoro-4-(3-piperidin-1-yl-propoxy)-benzaldehyde. 1H NMR (500 MHz, Solvent) δ ppm 2.23-2.27 (m, 3H), 2.27-2.37 (m, J=13.12 Hz, 2H), 2.39-2.53 (m, 2H), 2.98 (t, J=11.29 Hz, 2H), 3.43-3.56 (m, J=12.21 Hz, 2H), 4.24 (s, 2H), 4.44-4.58 (m, 1H), 7.14-7.22 (m, J=7.63 Hz, 2H), 7.23-7.27 (m, 2H), 7.37 (s, 1H), 7.47 (dd, J=9.31, 2.29 Hz, 1H), 7.50-7.59 (m, 2H), 8.31 (dd, J=8.85, 6.41 Hz, 1H); MS (ESI) m/z 395 [M+H]⁺.

### Example 73

### N-[1-(1-benzothien-5-ylmethyl)piperidin-4-yl]-7-fluoro-4-oxo-4H-chromene-2-carboxamide

### Example 73A

### 1-benzothien-5-ylmethyl methanesulfonate

A solution of benzo[b]thiophen-yl-methanol (500 mg, 3 mmol) in 12 mL CH₂Cl₂ was charged with diisopropylamine (594 uL, 3.3 mmol) followed by methanesulfonylchloride (236 uL, 3 mmol). After 24 hours, an additional aliquot of diisopropyl amine (120 uL, 0.7 mmol) and methanesulfonyl chloride (50 uL, 0.6 mmol) was added. After another 24 hours, the mixture was diluted with ethyl acetate, washed with 1M H₂SO₄, saturated aqueous NaHCO₃, brine, dried (Na₂SO₄) and concentrated to provide the title compound as a yellow oil. 1H NMR (300 MHz, DMSO-D6) δ ppm 3.32 (s, 3H) 4.90 (s, 2H) 7.40-7.45 (m, 1H) 7.48 (d, J=5.43 Hz, 1H) 7.81 (d, J=5.43 Hz, 1H) 7.96 (d, J=1.36 Hz, 1H) 8.02 (d, J=8.48 Hz, 1H).

### Example 73

### N-[1-(1-benzothien-5-ylmethyl)piperidin-4-yl]-7-fluoro-4-oxo-4H-chromene-2-carboxamide

A solution of Example 60D (58 mg, 0.2 mmol) in dimethylformamide (0.4 mL) was charged with K₂CO₃ (41 mg, 0.3 mmol) followed by example 73A (51 mg, 0.21 mmol). Stirred for 28 hours, diluted with CH₂Cl₂, washed with distilled water, dried (Na₂SO₄), filtered and concentrated. Purification by flash silica gel chromatography (0 to 100% ethyl acetate/hexane) provided the title compound as a white solid. MS (ESI, MeOH/NH4OH) m/z 437 [M+H], 435 [M−H]. 1H NMR (300 MHz, DMSO-D6) δ ppm 1.56-1.73 (m, 2H) 1.75-1.88 (m, 2H) 2.01-2.15 (m, 2H) 2.81-2.94 (m, 2H) 3.57-3.64 (m, 2H) 3.70-3.88 (m, 1H) 6.82 (s, 1H) 7.33 (s, 1H) 7.38-7.48 (m, 2H) 7.62 (dd, J=9.49, 2.37 Hz, 1H) 7.75 (d, J=5.43 Hz, 1H) 7.80 (s, 1H) 7.95 (d, J=8.48 Hz, 1H) 8.11 (dd, J=8.99, 6.27 Hz, 1H) 8.83 (d, J=7.80 Hz, 1H).

### Example 74

### methyl 5-[(4-{[(7-fluoro-4-oxo-4H-chromen-2-yl)carbonyl]amino}piperidin-1-yl)methyl]-1H-indole-1-carboxylate

### Example 74A

### methyl 5-formyl-1H-indole-1-carboxylate

A solution of indole-5-carboxaldehyde in 14 mL dry tetrahydrofuran was cooled to 0° C. To this solution was added sodium hydride (60% dispersion in mineral oil, 173 mg, 4.3 mmol) (vigorous gas evolution). This yellow heterogeneous mixture was stirred for 45 minutes and then methyl chloroformate (278 uL, 3.6 mmol) was added. The mixture was allowed to warm to room temperature, stirred two hours, then quenched by slow addition of 3M H₂SO₄ (0.5 mL) (vigorous gas evolution). After gas evolution ceased, the off-white precipitate was filtered off, washed with distilled water and then dried overnight in a vacuum oven to provide the title compound as a white powder. 1H NMR (300 MHz, DMSO-D6) δ ppm 4.03 (s, 3H) 6.94 (d, J=3.73 Hz, 1H) 7.88 (s, 2H) 8.26 (s, 2H) 10.06 (s, 1H).

### Example 74

### methyl 5-[(4-{[(7-fluoro-4-oxo-4H-chromen-2-yl)carbonyl]amino}piperidin-1-yl)methyl]-1H-indole-1-carboxylate

A solution of example 60D (58 mg, 0.2 mmol) and example 74A (41 mg, 0.2 mmol) in THF/2% AcOH (0.8 mL) was charged with NaBH(OAc)₃ (168 mg, 0.4 mmol). The mixture was shaken for 24 hours then quenched by slow addition of aqueous K₂CO₃ (1M, 250 uL), shaken for 10 minutes, diluted with CH₂Cl₂ (2 mL), dried (Na₂SO₄), filtered through a plug of silica (2 g SepPak), rinsed through with 50/50 heaxnae/ethyl acetate, collection tube changed, then rinsed through with 95/5 CH₂Cl₂/MeOH and concentrated. Further purification by flash silica gel chromatography (gradient elution of 0 to 100% ethyl acetate/hexane) provided the title compound as a white solid. MS (ESI, MeOH/NH4OH) m/z 478 [M+H], 476 [M−H]. 1H NMR (300 MHz, DMSO-D6) δ ppm 1.65 (dd, J=11.87, 3.05 Hz, 2H) 1.80 (s, 2H) 2.03 (s, 2H) 2.87 (d, J=11.87 Hz, 2H) 3.71-3.88 (m, 1H) 4.01 (s, 3H) 6.73 (d, J=3.73 Hz, 1H) 6.82 (s, 1H) 7.30 (s, 1H) 7.37-7.48 (m, 3H) 7.55 (s, 1H) 7.61 (dd, J=9.49, 2.71 Hz, 1H) 7.70 (d, J=3.73 Hz, 1H) 8.05 (d, J=8.48 Hz, 1H) 8.11 (dd, J=8.99, 6.27 Hz, 1H) 8.83 (d, J=7.80 Hz, 1H).

### Example 75

### 7-fluoro-4-oxo-N-{1-[1-(2-oxo-2H-chromen-7-yl)ethyl]piperidin-4-yl}-4H-chromene-2-carboxamide

### Example 75A

### 2-Oxo-2H-chromene-7-carbaldehyde

A 1-liter flask was charged with 4.00 grams of 7-methyl-chromen-2-one (25.0 mmol), 333 mL of CCl₄, 5.37 grams (30.0 mmol) of NBS, and 0.410 grams (2.49 mmol) of AIBN. The solution was heated to reflux and allowed to stir at this temperature for 5 hours. After this time, the solution was cooled to room temperature and 4.03 grams of NBS was added, followed by an additional 0.410 grams of AIBN, and the solution heated to reflux. After an additional 12 hours, this procedure was repeated, followed by another 12 hours of stirring at reflux. After this time, the vessel was cooled to room temperature and filtered. The eluant was then evaporated to provide an off-white solid. This material was then dissolved in 333 mL of EtOH and 60 mL of DI H₂O was added. The flask was then equipped with a reflux condenser and the entire apparatus covered with aluminum foil prior to the addition of AgNO₃ (10.9 g, 64.2 mmol). The mixture was heated to reflux for 16 hours, cooled to room temperature, and then filtered to remove the salts. The eluant was then concentrated to about 50 mL on a rotary evaporator, and then removed and allowed to stand at room temperature for 15 minutes. Following this time, the precipitate was collected on a Buchner funnel via suction filtration to provide the titled compound. 1H NMR (300 MHz, DMSO-D6) δ ppm 6.67 (d, J=9.49 Hz, 1H), 7.77-8.00 (m, 3H), 8.16 (d, J=9.15 Hz, 1H), 10.09 (s, 1H); MS (ESI) m/z 192 [+H]⁺.

### Example 75B

### 7-(1-Chloro-ethyl)-chromen-2-one

To a 1 liter flask was added 6.40 grams of 2-oxo-2H-chromene-7-carbaldehyde (36.6 mmol) and 300 mL of dry ether. The solution was then cooled to −78° C. in a dry ice bath. A 3M solution of methylmagnesium bromide (15.0 mL, 45.0 mmol) was then added via addition funnel over 15-20 minutes, and the mixture allowed to warm slowly to room temperature. Upon consumption of the starting material by TLC (5% EtOAc in hexanes), the mixture was quenched by the slow addition of a mixture of AcOH/MeOH (1:1) via addition funnel. The resultant mixture was allowed to warm to room temperature and then concentrated in vacuo. The residue was taken up in EtOAc and purified via column chromatography (15-50% EtOAc in hexanes) to provide the corresponding secondary alcohol. 1H NMR (300 MHz, DMSO-D6) δ ppm 1.35 (d, J=6.44 Hz, 3H), 4.67-4.89 (m, 1H), 5.40 (d, J=4.41 Hz, 1H), 6.44 (d, J=9.49 Hz, 1H), 7.22-7.42 (m, 2H), 7.57-7.73 (m, 1H), 7.97-8.11 (m, J=9.49 Hz, 1H); MS (ESI) m/z 191 [M+H]⁺. This material (8.88 g, 44.0 mmol) was then dissolved in 16 mL of heptane and 3.30 mL of pyridine (40.8 mmol), 1.80 mL of DMF (23.3 mmol), and 3.10 mL of methanesulfonyl chloride (40.0 mmol) was added. The mixture was allowed to stir for 12 hours at room temperature after which time it was diluted with 3:1 EtOAc/hexanes and washed with 0.1 N HCl (×2), sat NaHCO₃ (×2), H₂O, dried, and filtered. Evaporation of the solvents provided the titled product. 1H NMR (300 MHz, DMSO-D6) δ ppm 1.82 (d, J=6.78 Hz, 3H), 5.45 (q, J=6.67 Hz, 1H), 6.51 (d, J=9.49 Hz, 1H), 7.42-7.57 (m, 2H), 7.67-7.81 (m, J=7.80 Hz, 1H), 8.07 (d, J=9.49 Hz, 1H); MS (ESI) m/z 209 [M+H]⁺.

### Example 75

### 7-fluoro-4-oxo-N-{1-[1-(2-oxo-2H-chromen-7-yl)ethyl]piperidin-4-yl}-4H-chromene-2-carboxamide

A 100 mL flask was charged with 1.50 gram of 7-(1-chloro-ethyl)-chromen-2-one (7.21 mmol), 2.47 grams of 7-Fluoro-4-oxo-4H-chromene-2-carboxylic acid piperidin-4-ylamide hydrochloride (7.58 mmol), 2.29 grams of Na₂CO₃ (21.6 mmol, pulverized and then dried via vacuum oven at 200° C.), and 15 mL of CH₃CN, and the suspension slowly heated to 70° C. After 16 hours, the suspension was cooled to room temperature and diluted with 20 mL of EtOAc, passed over celite, and the cake rinsed with 5% Et₃N in EtOAc (20 mL×2). The eluant was evaporated and the residue taken up in 5% Et₃N in EtOAc and purified via column chromatography (2-5% Et₃N in EtOAc) to provide the racemic product. ¹H NMR (400 MHz, DMSO-D6) δ ppm 1.34 (d, J=6.44 Hz, 3H), 1.55-1.72 (m, 2H), 1.74-1.87 (m, 2H), 1.96-2.13 (m, J=11.97 Hz, 2H), 2.74-2.84 (m, 1H), 2.93-3.02 (m, 1H), 3.58-3.68 (m, J=6.75 Hz, 1H), 3.68-3.79 (m, 1H), 6.45 (d, J=9.51 Hz, 1H), 6.81 (s, 1H), 7.31-7.37 (m, 2H), 7.38-7.47 (m, 1H), 7.68 (d, J=7.98 Hz, 2H), 8.05 (d, J=9.82 Hz, 1H), 8.11 (dd, J=8.75, 6.29 Hz, 1H), 8.80 (d, J=7.98 Hz, 1H); MS (ESI) m/z 463 [M+H]⁺.

### Example 76

### N-[1-(1,3-benzodioxol-5-ylmethyl)piperidin-4-yl]-7-bromo-4-oxo-4H-chromene-2-carboxamide

The Example 65 procedure was applied to 3-Bromo-phenol (5 g, 28.9 mmol) to give N-[1-(1,3-benzodioxol-5-ylmethyl)piperidin-4-yl]-7-bromo-4-oxo-4H-chromene-2-carboxamide. ¹H NMR (300 MHz, DMSO-d₆) δ ppm 1.63 (qd, 2H, J=11.86, 3.65 Hz), 1.71-1.87 (m, 2H), 1.96-2.07 (m, 2H), 2.83 (d, 2H, J=11.19 Hz), 3.39 (s, 2H), 3.67-3.86 (m, 1H), 5.99 (s, 2H), 6.75 (dd, 1H, J=7.88, 1.53 Hz), 6.82 (s, 1H), 6.85 (d, 1H, J=7.71 Hz), 6.86 (s, 1H), 7.72 (dd, 1H, J=8.45, 1.84 Hz), 7.96 (d, 1H, J=8.43 Hz), 8.06 (d, 1H, J=1.81 Hz), 8.80 (d, 1H, J=7.88 Hz) MS (ESI) m/z 485 [M+H]⁺.

### Example 77

### 7-fluoro-4-oxo-N-[1-(1-quinolin-6-ylethyl)piperidin-4-yl-4H-chromene-2-carboxamide

Example 55E (31 mg, 0.100 mmol), Example 17A, N-methylindole-5-carboxaldehyde, (16 mg, 0.100 mmol), macroporous-cyanoborohydride (101 mg, 0.250 mmol of 2.47 mmol/gram resin, Argonaut Technologies), acetic acid (30 μL), CH₂Cl₂ (0.5 mL) and THF (0.5 mL) were combined and agitated overnight. The mixture was added to a MP-TsOH cartridge (Argonaut Technologies), washed with THF, and rinsed with 2N ammonia in methanol. The ammonia in methanol fraction was concentrated and purified by a sep pack (Altech, 1 gram) eluting with 100% EtOAc to provide the title compound as a white solid. ¹H NMR (300 MHz, DMSO-d6): δ ppm 1.55-1.70 (m, 2H), 1.71-1.86 (m, 2H), 1.96-2.10 (m, 2H), 2.82-2.93 (m, 2H), 3.50-3.56 (m, 2H), 3.67-3.86 (m, 1H), 3.77 (s, 3H), 6.37 (d, 1H, J=3.04 Hz), 6.84 (s, 1H), 7.12 (dd, 1H, J=8.36, 1.49 Hz), 7.29 (d, 1H, J=3.08 Hz), 7.37 (d, 1H, J=8.39 Hz), 7.44 (s, 1H), 7.90 (dd, 1H, J=10.68, 6.44 Hz), 8.00 (dd, 1H, J=9.96, 8.86 Hz), 8.83 (d, 1H, J=7.84 Hz); MS (ESI, MeOH/NH4OH) m/z 452 [M+H]⁺, 450 [M−H]⁺.

### Example 78

### 2-({2-fluoro-4-[(4-{[(7-fluoro-4-oxo-4H-chromen-2-yl)carbonyl]amino}piperidin-1-yl)methyl]phenyl}amino)-2-oxoethyl acetate

A vial was charged with N-[1-(4-amino-3-fluorobenzyl)piperidin-4-yl]-7-fluoro-4-oxo-4H-chromene-2-carboxamide prepared as described in Example 90 (25 mg, 0.04 mmol), acetoxy acetic acid (4.6 mg, 0.04 mmol), EDCI (17.5 mg, 0.04 mmol), HOBT (5.3 mg, 0.04 mmol), NMM (0.017 mL, 0.16 mmol), and DMF (0.4 mL). Solution was shaken at 55° C. for 16 hours. Sample was purified on a RP-HPLC system to provide 2-({2-fluoro-4-[(4-{[(7-fluoro-4-oxo-4H-chromen-2-yl)carbonyl]amino}piperidin-1-yl)methyl]phenyl}amino)-2-oxoethyl acetate. ¹H NMR (300 MHz, DMSO-D6) δ ppm 1.73-1.87 (m, 2H) 2.00-2.12 (m, 4H) 3.04 (d, J=11.87 Hz, 2H) 3.17 (s, 1H) 3.43 (s, 3H) 4.12-4.14 (m, 2H) 4.69-4.78 (m, 1H) 6.76-6.84 (m, 1H) 6.85 (s, 1H) 6.93-7.02 (m, 1H) 7.15 (dd, J=12.38, 1.86 Hz, 1H) 7.40-7.50 (m, 2H) 7.61 (dd, J=9.49, 2.37 Hz, 1H) 8.09-8.17 (m, 1H) 9.06 (d, J=7.46 Hz, 1H) 9.31 (s, 1H) MS (ESI) m/z 514 [M+H]⁺.

### Example 79

### 7-chloro-6-fluoro-N-{1-[3-fluoro-4-(3-piperidin-1-ylpropoxy)benzyl]piperidin-4-yl}-4-oxo-4H-chromene-2-carboxamide

### Example 79A

### 3-fluoro-4-(3-piperidin-1-yl-propoxy]-benzaldehyde

To a cold (0° C.) solution of 3-fluoro-4-hydroxy-benzaldheyde (500 mg, 3.57 mmol), 3-piperidin-1-yl-propan-1-ol (0.541 mL, 3.57 mmol), and Ph₃P (935 mg, 3.57 mmol) in THF (12 mL) was added DEAD (0.561 mL, 3.57 mmol). The ice bath was removed, and the mixture was stirred at room temperature for 8 hours. The mixture was concentrated under reduced pressure, and the residue was purified by MPLC (0% to 5% MeOH in DCM) to provide the titled compound as an oil. MS (ESI) m/z 266 (M+H)⁺.

### Example 79

### 7-chloro-6-fluoro-N-{1-[3-fluoro-4-(3-piperidin-1-ylpropoxy)benzyl]piperidin-4-yl}-4-oxo-4H-chromene-2-carboxamide

To a solution of 3-fluoro-4-(3-piperidin-1-yl-propoxy)-benzaldehyde (18.0 mg, 0.069 mmol), and 4-[(6-fluoro-7-chloro-4-oxo-4H-chromene-2-carbonyl)-amino]-piperidine (22.0 mg, 0.069 mmol) in methanol containing 2% v/v AcOH (1 mL) was added MP-CNBH₃ (30 mg, 0.090 mmol, ˜3.0 mmol/g loading). The mixture was shaken vigorously for 18 hours, was filtered, and the methanol solution directly purified by RP-HPLC to provide the title compound as its bis-TFA salt. ¹H NMR (300 MHz, DMSO-d6) δ ppm 1.32-1.49 (m, 1H), 1.55-1.91 (m, 7H), 1.97-2.26 (m, 4H), 2.81-3.00 (m, 2H), 3.00-3.17 (m, 2H), 3.16-3.25 (m, 2H), 3.38-3.56 (m, 4H), 3.94-4.07 (m, 1H), 4.13-4.19 (m, 2H), 4.22-4.28 (m, 2H), 6.87 (s, 1H), 7.27-7.29 (m, 2H), 7.36-7.43 (m, 1H), 7.95 (d, 1H, J=8.67 Hz), 8.09 (d, 1H, J=5.97 Hz), 9.08 (d, 1H, J=7.43 Hz), 9.11-9.20 (bs, 1H), 9.73-9.84 (bs, 1H); MS (ESI) m/z 574 (M+H)⁺.

### Example 80

### N-[1-(2-chloro-4-{[(2-pyrrolidin-1-ylethyl)amino]carbonyl}benzyl)piperidin-4-yl]-7-fluoro-4-oxo-4H-chromene-2-carboxamide

### Example 80A

### 4-Bromomethyl-3-chloro-benzoic acid

A 50 mL round bottom flask was charged with 3-chloro-4-methyl-benzoic acid (1 g, 5.9 mmol), NBS (1.043 g, 5.9 mmol), AIBN (97 mg, 0.59 mmol) and CCl₄ (20 mL). The mixture was completed after refluxing at 80° C. for 3 hours, then quenched with water, diluted with CH₂Cl₂. The aqueous layer was extracted with CH₂Cl₂. The combined organic layer was washed with brine, dried over Na₂SO₄, concentrated to provide the crude mixtures. Then preparative reverse-phase HPLC was used to provide the pure title compound. ¹H NMR (500 MHz, DMSO-d₆) δ ppm 4.79(s, 2H), 7.75(d, 1H, J=7.93), 7.89(d, 1H, J=7.93), 7.94 (s, 1H), 13.35(s, br, 1H); MS (ESI) m/e 246.6 (M−H)⁺.

### Example 80B

### 3-chloro-4-{4-[(7-fluoro-4-oxo-4H-chromene-2-carbonyl)-amino]-piperidin-1-ylmethyl}-benzoic acid

A 100 mL round bottom flask was charged with 4-bromomethyl-3-chloro-benzoic acid (440 mg, 1.76 mmol), example 7B (510 mg, 1.76 mmol), K₂CO₃ (973 mg, 7.04 mmol) and EtOH (60 mL). The mixture was stirred at room temperature overnight, then quenched with 1 N HCl to adjust pH to about 2. EtOAc (150 mL) was added and the two layers were separated. The aqueous layer was extracted with EtOAc. The combined organic layer was washed with brine, dried over Na₂SO₄, concentrated to give the crude mixtures. Preparative reverse-phase HPLC was used to provide the title compound. ¹H NMR (400 MHz, DMSO-d₆) δ ppm 2.09-2.12(m, 4H), 3.42(m, 4H), 4.06(m, 1H), 4.46(s, 2H), 6.84(s, 1H), 7.41(m, 1H), 7.65(d, 1H, J=8.6), 7.95(d, 1H, J=9.21), 7.99(s, 1H), 8.08-8.13(m, 2H), 9.20(d, 1H, J=6.75), 11.26(s, br, 1H); MS (ESI) m/e 456.9 (M−H)⁺.

### Example 80

### N-[1-(2-chloro-4-{[(2-pyrrolidin-1-ylethyl)amino]carbonyl}benzyl)piperidin-4-yl]-7-fluoro-4-oxo-4H-chromene-2-carboxamide

A 50 mL round bottom flask was charged with 3-chloro-4-{4-[(7-fluoro-4-oxo-4H-chromene-2-carbonyl)-amino]-piperidin-1-ylmethyl}-benzoic acid (100 mg, 0.22 mmol), CH₂Cl₂ (10 mL) and DMF (2 mL). Cooled to 0° C., then (COCl)₂ (0.14 mL, 0.264 mmol) was added into it. The mixture was slowly warm to room temperature and stirred at room temperature for 1 hour. CH₂Cl₂ was removed under reduced pressure and 2 mL of DMF was added. Then 2-pyrrolidin 1-yl-ethylamine (30 mg, 0.264 mmol) was added. The mixture was stirred at room temperature for 3 hours, the DMF was removed under reduced pressure. The residue was dissolved in 1.5 mL of a 1:1 mixture of DMSO/MeOH and purified by preparative reverse-phase HPLC. ¹H NMR (400 MHz, DMSO-d₆) δ ppm 1.64-1.83(m, 8H), 2.19(t, 2H), 2.47(m, 4H), 2.56(t, 2H), 2.86 (d, 2H, J=11.36), 3.36-3.40(m, 2H), 3.62(s, 2H), 3.81-3.84(m, 1H), 6.83(s, 1H), 7.40-7.45(m, 1H), 7.57-7.62(m, 2H), 7.81(d, 1H, J=9.52), 7.90(s, 1H), 8.10-8.13 (m, 1H), 8.51(t, 1H), 8.83(d, 1H, J=7.97); MS (ESI) m/e 553.2 (M−H)⁺.

### Example 81

### 7-fluoro-N-(1-{3-fluoro-4-[(3-pyrrolidin-1-ylpropyl)amino]benzyl}piperidin-4-yl)-4-oxo-4H-chromene-2-carboxamide

### Example 81A

### 3-Fluoro-4-(3-pyrrolidin-1-yl-propylamino)-benzonitrile

A flask was charged with 3,4-difluoro-benzonitrile (2.5 g, 18.0 mmol), 3-Pyrrolidin-1-yl-propylamine (6.9 g, 54 mmol), and MeOH (10 mL). Solution was stirred at 40 C for 16 hr. Solution was concentrated and purified on a FlashMaster II silica column using gradient conditions from 100% CH₂Cl₂ to 5% methanol/CH₂Cl₂ to provide 3-Fluoro-4-(3-pyrrolidin-1-yl-propylamino)-benzonitrile. MS (APCI) m/z 248 [M+H]⁺.

### Example 81B

### 3-Fluoro-4-(3-pyrrolidin-1-yl-propylamino)-benzaldehyde

A flask was charged with 3-Fluoro-4-(3-pyrrolidin-1-yl-propylamino)-benzonitrile (3.5 g, 14.2 mmol), sodium hypophosphite hydrate (10 g, 114 mmol), pyridine (20 mL), acetic acid (10 mL), and water (10 mL). Solution was cooled to 0 C and Raney-Ni 2800 (2 mL) was added. Mixture was slowly heated to 40 C and stirred for 16 hours. Solution was filtered, concentrated, taken up in EA, washed with 1× saturated sodium bicarbonate, 1× brine, and dried over Na₂SO₄ to provide 3-Fluoro-4-(3-pyrrolidin-1-yl-propylamino)-benzaldehyde. MS (APCI) m/z 251 [M+H]⁺.

### Example 81

### 7-fluoro-N-(1-{3-fluoro-4-[(3-pyrrolidin-1-ylpropyl)amino]benzyl}piperidin-4-yl)-4-oxo-4H-chromene-2-carboxamide

A flask was charged with 3-Fluoro-4-(3-pyrrolidin-1-yl-propylamino)-benzaldehyde (0.7 g, 2.8 mmol), 7-Fluoro-4-oxo-4H-chromene-2-carboxylic acid piperidin-4-ylamide (1 g, 3.1 mmol), sodium triacetoxyborohydride (2 g, 9.4 mmol), THF (25 mL), and acetic acid (0.2 mL). Solution was stirred at room temperature for 16 hr. Solution was concentrated, taken up in EA, washed with 1× saturated sodium bicarbonate, 1× brine, dried over Na₂SO₄, and purified on a FlashMaster II silica column using gradient conditions from 100% CH₂Cl₂ to 5% methanol/CH₂Cl₂ to provide 7-fluoro-N-(1-{3-fluoro-4-[(3-pyrrolidin-1-ylpropyl)amino]benzyl}piperidin-4-yl)-4-oxo-4H-chromene-2-carboxamide. 1H NMR (300 MHz, DMSO-D6) δ ppm 1.84 (s, 2H) 1.87 (d, J=3.05 Hz, 2H) 1.95 (s, 1H) 1.96-2.11 (m, 4H) 3.02 (s, 4H) 3.14-3.25 (m, 6H) 3.40 (s, 2H) 3.54 (td, J=10.68, 4.75 Hz, 2H) 4.16 (m, 1H) 6.76-6.87 (m, 2H) 7.08-7.14 (m, 1H) 7.19 (dd, J=12.54, 1.70 Hz, 1H) 7.44 (td, J=8.65, 2.37 Hz, 1H) 7.60 (dd, J=9.49, 2.37 Hz, 1H) 8.12 (dd, J=8.82, 6.44 Hz, 1H) 9.07 (d, J=7.46 Hz, 1H) 9.50 (s, 2H) MS (ESI) m/z 525 [M+H]⁺.

### Example 82

### 7-fluoro-N-[1-(1H-indol-6-ylmethyl)piperidin-4-yl]-4-oxo-4H-chromene-2-carboxamide

1H-Indole-6-carbaldehyde (0.11 g, 0.75 mmol), Example 60D (0.2 g, 0.7 mmol), sodium cyanoborohydride (1M in THF, 2.1 mL) and acetic acid (0.01 mL) were taken up in methanol (8 mL) and dichloromethane (8 mL) and stirred for 24 h at room temperature. The mixture was quenched with saturated sodium bicarbonate and 20% sodium hydroxide solution (˜pH 10) and extracted with ethyl acetate (4×20 mL). The organic extracts were washed with water, brine, dried (MgSO₄), concentrated and purified using flash chromatography to provide the title product as a white foam. ¹H NMR (300 MHz, DMSO-d6) δ ppm 1.57-1.72 (m, 2H), 1.75-1.85 (m, 2H), 1.99-2.08 (m, 2H), 2.83-2.93 (m, 2H), 3.55 (s, 2H), 3.72-3.87 (m, 1H), 6.37-6.40 (m, 1H), 6.82 (s, 1H), 6.96 (dd, 1H, J=8.06, 1.40 Hz), 7.28-7.31 (m, 2H), 7.38-7.49 (m, 2H), 7.62 (dd, 1H, J=9.47, 2.46 Hz), 8.11 (d, 1H, J=2.51 Hz), 8.83 (d, 1H, J=7.85 Hz), 10.99 (s, 1H); MS (ESI) m/z 420 [M+H]⁺.

### Example 83

### N-[1-(1,3-benzoxazol-6-ylmethyl)piperidin-4-yl]-7-fluoro-4-oxo-4H-chromene-2-carboxamide

The title compound was prepared according to the procedure for example 79 substituting benzooxazole-6-carbaldehyde for 3-fluoro-4-(3-piperidin-1-yl-propoxy)-benzaldehyde. ¹H NMR (300 MHz, DMSO-D6) δ ppm 1.56-1.75 (m, 2H), 1.76-1.88 (m, 2H), 1.96-2.17 (m, 2H), 2.82-2.88 (m, 2H), 3.64 (s, 2H), 3.71-3.88 (m, 1H), 6.82 (s, 1H), 7.33-7.47 (m, 2H), 7.62 (dd, J=9.49, 2.37 Hz, 1H), 7.69 (s, 1H), 7.72-7.78 (m, J=8.14 Hz, 1H), 8.11 (dd, J=8.82, 6.44 Hz, 1H), 8.70 (s, 1H), 8.84 (d, J=7.80 Hz, 1H); MS (ESI) m/z 422 [+H]⁺.

### Example 84

### 7-fluoro-N-{1-[3-fluoro-4-(3-piperidin-1-ylpropoxy)benzyl]piperidin-4-yl}-4-oxo-4H-chromene-2-carboxamide

Example 84 was prepared according to the procedure described in Example 79, substituting 4-[(7-fluoro-4-oxo-4H-chromene-2-carbonyl)-amino]-piperidine for 4-[(6-fluoro-7-chloro-4-oxo-4H-chromene-2-carbonyl)-amino]-piperidine. The crude residue was purified by MPLC (30% Et₃N in EtOAc) to provide the title compound as a white solid. ¹H NMR (300 MHz, DMSO-d6) δ ppm 1.32-1.42 (m, 2H), 1.43-1.56 (m, 4H), 1.56-1.73 (m, 2H), 1.73-1.92 (m, 4H), 1.95-2.12 (m, 2H), 2.26-2.43 (m, 6H), 2.76-2.88 (m, 2H), 3.42 (s, 2H), 3.72-3.85 (m, 1H), 4.05 (t, 2H, J=6.37 Hz), 6.82 (s, 1H), 7.04 (dd, 1H, J=8.57, 1.90 Hz), 7.06-7.19 (m, 2H), 7.43 (td, 1H, J=8.71, 2.43 Hz), 7.62 (dd, 1H, J=9.54, 2.42 Hz), 8.12 (dd, 1H, J=8.94, 6.40 Hz), and 8.83 (d, 1H, J=7.80 Hz); MS (ESI) m/z 540 (M+H)⁺.

### Example 85

### 7-fluoro-4-oxo-N-[1-(3-oxo-2,3,4,6,7,8-hexahydroindeno[5,6-b][1,4]oxazin-6-yl)piperidin-4-yl]-4H-chromene-2-carboxamide

### Example 85A

### 3-(3-hydroxy-4-nitrophenyl)-acrylic acid methyl ester

To an ambient solution of 3-hydroxy-4-nitrobenzaldehyde (1.00 g, 5.99 mmol) and trimethyl phosphonoacetate (0.950 mL, 6.59 mmol) in DMF (8 mL) was added NaOMe (356 mg, 6.59 mmol). The mixture was stirred at room temperature for 16 h and was then slowly quenched by the addition of 1 N HCl (15 mL). The mixture was acidified to pH˜1 with concentrated HCl, and the solid was collected and air-dried to provide the title compound. MS (ESI) m/z 224 (M+H)⁺.

### Example 85B

### 3-(4-amino-3-hydroxy-phenyl)-acrylic acid methyl ester

To a mixture of 3-(3-hydroxy-4-nitrophenyl)-acrylic acid methyl ester (700 mg, 3.14 mmol) and ammonium chloride (141 mg, 2.51 mmol) in EtOH (20 mL) and H₂O (10 mL) was added Fe powder (424 mg, 7.85 mmol). The mixture was heated to reflux for 6 hours, cooled to room temperature, and filtered through celite, eluting with MeOH. The mixture was concentrated under reduced pressure to give a dark solid. The residue was triturated with EtOAc, filtered, and the eluent concentrated to provide the title compound as a solid, which was used without further purification. MS (ESI) m/z 194 (M+H)⁺.

### Example 85C

### 3-(3-Oxo-3,4-dihydro-2H-benzo[1,4]oxazin-7-yl)-acrylic acid methyl ester

To a mixture of 3-(4-amino-3-hydroxy-phenyl)-acrylic acid methyl ester (266 mg, 1.38 mmol) and K₂CO₃ (571 mg, 4.14 mmol) in acetone (4.6 mL) was added 2-bromoacetyl bromide (0.120 mL, 1.38 mmol). The mixture was heated to 50° C. for 2 hours, was cooled to room temperature, and then quenched by the addition of saturated aqueous NaHCO₃ (10 mL) and EtOAc (10 mL). The layers were separated, and the aqueous was extracted with additional EtOAc (3×10 mL). The combined organics were dried with anhydrous Na₂SO₄, filtered, and concentrated under reduced pressure. The residue was passed through a small plug of SiO₂ gel, eluting with EtOAc. The eluent was concentrated to provide the title compound as a solid. MS (ESI) m/z 234 (M+H)⁺.

### Example 85D

### 3-(3-Oxo-3,4-dihydro-2H-benzo[1,4]oxazin-7-yl)-propionic acid methyl ester

A mixture of 3-(3-oxo-3,4-dihydro-2H-benzo[1,4]oxazin-7-yl)-acrylic acid methyl ester (270 mg, 1.16 mmol) and 10% Pd/C (27 mg) in EtOAc (10 mL) and MeOH (10 mL) was stirred over an atmosphere of H₂ (1 atm, balloon) for 16 hours. The mixture was then filtered through celite, eluting with MeOH. The eluent was concentrated under reduced pressure to provide the title compound as a solid. MS (ESI) m/z 236 (M+H)⁺.

### Example 85E

### 3-(3-Oxo-3,4-dihydro-2H-benzo[1,4]oxazin-7-yl)-propionic acid

An ambient solution of 3-(3-oxo-3,4-dihydro-2H-benzo[1,4]oxazin-7-yl)-propionic acid methyl ester (215 mg, 0.915 mmol) and LiOH (46.0 mg, 1.92 mmol) in THF (4.5 mL) and H₂O (1.5 mL) was stirred for 8 hours. The THF was removed under reduced pressure, and the remaining aqueous layer was acidified to pH˜1 with 1N HCl. The solid was filtered and air-dried to provide the title compound. MS (ESI) m/z 222 (M+H)⁺.

### Example 85F

### 3,8-Dihydro-2H-5-oxa-8-aza-cyclopenta[b]naphthalene-1,7-dione

A mixture of 3-(3-oxo-3,4-dihydro-2H-benzo[1,4]oxazin-7-yl)-propionic acid (125 mg, 0.566 mmol), AlCl₃ (1.0 g, 7.5 mmol), and LiCl (160 mg, 2.76 mmol) was heated to 140° C. for 2 h with vigorous stirring. The resulting melt was cooled to room temperature. Water (1 mL) was slowly added to the cooled (0° C.) glass, followed by 1 N HCl (2 mL). The mixture was stirred vigorously for 1 hours, and the solid was filtered and air-dried to provide the title compound. MS (ESI) m/z 204 (M+H)⁺.

### Example 85

### 7-fluoro-4-oxo-N-[1-(3-oxo-2,3,4,6,7,8-hexahydroindeno[5,6-b][1,4]oxazin-6-yl)piperidin-4-yl]-4H-chromene-2-carboxamide

Example 85 was prepared according to the procedure described in Example 92, substituting 3,8-dihydro-2H-5-oxa-8-aza-cyclopenta[b]naphthalene-1,7-dione for 6-acetyl-3-ethyl-3H-benzothiazole-2-one. The crude residue was purified by RP-HPLC to provide the title compound as a solid. ¹H NMR (300 MHz, DMSO-d6) δ ppm 1.94-2.05 (m, 2H), 2.24-2.28 (m, 2H), 2.49-2.57 (m 1H), 2.96-3.02 (m, 1H), 3.06-3.11 (m, 2H), 3.34 (m, 2H), 3.35-3.45 (m, 2H), 3.51-3.59 (m, 2H), 4.09-4.20 (m, 1H), 4.85-4.90 (m, 1H), 6.97 (s, 1H), 7.00 (s, 1H), 7.11 (s, 1H), 7.34 (dt, 1H, J=2.4 and 8.5 Hz), 7.48 (dd, 1H, J=2.4 and 9.1 Hz), 8.21 (dd, 1H, J=6.4 and 9.1 Hz), 9.04 d(1H, J=7.5 Hz), and 11.80 (s, 1H); MS (ESI) m/z 478 (M+H)⁺.

### Example 86

### N-[1-(2,3-dihydro-1-benzofuran-5-ylmethyl)piperidin-4-yl]-7-fluoro-4-oxo-4H-chromene-2-carboxamide

A solution of Example 60D (29 mg, 0.1 mmol) and 2,3-dihydrobenzofuran-5-carboxaldehyde (15 mg, 0.1 mmol) in THF/2% acetic acid (0.5 mL) was charged with 42 mg of NaBH(OAc)₃ and shaken for five days. The heterogeneous mixture was quenched by addition of 1M K₂CO₃ (0.5 mL), diluted with CH₂Cl₂ (2 mL) dried (Na₂SO₄), placed onto a plug of silica (2 g sep Pak), rinsed with ethyl acetate (10 mL), collection tube changed, rinsed with 95/5 CH₂Cl₂/MeOH and concentrated to provide the title compound as a light orange solid (20 mg). MS (ESI, MeOH/NH4OH) m/z 423 [M+H], 421 [M−H]. ¹H NMR (300 MHz, DMSO-d6) δ ppm 1.51-1.69 (m, 2H), 1.70-1.87 (m, 2H), 1.94-2.06 (m, 2H), 2.83 (d, 2H, J=11.55 Hz), 3.16 (t, 2H, J=8.63 Hz), 3.38 (s, 2H), 3.69-3.84 (m, 1H), 4.50 (t, 2H, J=8.66 Hz), 6.69 (d, 1H, J=8.04 Hz), 6.82 (s, 1H), 6.99 (dd, 1H, J=8.06, 1.83 Hz), 7.15 (s, 1H), 7.43 (td, 1H, J=8.68, 2.50 Hz), 7.61 (dd, 1H, J=9.47, 2.47 Hz), 8.11 (dd, 1H, J=8.89, 6.38 Hz), 8.81 (d, 1H, J=7.91 Hz).

### Example 87

### 7-fluoro-4-oxo-N-{1-[(2-oxo-1,2-dihydroquinolin-7-yl)methyl]piperidin-4-yl}-4H-chromene-2-carboxamide

### Example 87A

### 7-Methyl quinoline-1-oxide

m-Chloroperbenzoic acid (10.95 g, 77%, 49 mmol) was added to a solution of 7-methyl quinoline (7.0 g, 49 mmol) in ethyl acetate (500 mL) and stirred at room temperature for 5 hours. The solution was then concentrated to a tenth of the total volume and purified by flash chromatography to provide 7-Methyl quinoline-1-oxide as a white solid. ¹H NMR (300 MHz, DMSO-d6) δ ppm 2.56 (s, 3H), 7.39 (dd, 1H, J=6, 3 Hz), 7.58 (dd, 1H, J=9, 3 Hz), 7.89 (d, 1H, J=9 Hz), 7.98 (d, 1H, J=9 Hz), 8.35 (br s, 1H), 8.54 (dd, 1H, J=9, 1 Hz).

### Example 87B

### 7-methyl-1H-quinolin-2-one

7-Methyl quinoline-1-oxide was placed in acetic anhydride and heated at 140° C. for 4 hours, cooled to room temperature, poured over ice (1 L) and extracted with dichloromethane (3×200 mL). The organic extracts were washed with water, brine, dried (Na₂SO₄), concentrated and purified by flash chromatography to provide the title compound as a brown solid. ¹H NMR (300 MHz, DMSO-d6) δ ppm 2.37 (s, 3H), 6.40 (dd, 1H, J=9, 1 Hz), 7.00 (m, 1H), 7.08 (m, 1H), 7.53 (d, 1H, J=9 Hz), 7.84 (d, 1H, J=9 Hz), 11.65 (br s, 1H).

### Example 87

### 7-fluoro-4-oxo-N-{1-[(2-oxo-1,2-dihydroquinolin-7 yl)methyl]piperidin-4-yl 1-4H-chromene-2-carboxamide

A solution of 7-methyl-1H-quinolin-2-one (0.26 g, 1.66 mmol), N-bromosuccinamide (0.3 g, 1.67 mmol), azoisobutyronitrile (0.03 g, 0.2 mmol) in carbon tetrachloride (10 mL) was heated at 80° C. for 5 hours. The mixture was cooled, filtered and the filtrate concentrated to a yellow paste. The residue was diluted with ethyl acetate, filtered and the isolated solid used without further purification in the next step. 7-bromomethyl-1H-quinolin-2-one (0.28 g, 1.17 mmol) was placed along with Example 60D (0.34 g, 1.17 mmol), potassium carbonate (0.24 g, 1.75 mmol), and potassium iodide (0.2 g, 1.17 mmol) in THF (5 mL) and DMF (2 mL). The mixture was stirred at room temperature for 2.5 hours, filtered through a plug of cotton, concentrated and stirred in ethyl acetate (40 mL). The slurry was filtered and the filtrate purified further by RP-HPLC. The resultant TFA salt was taken up in 10% methanol/ethyl acetate, washed with saturated sodium bicarbonate, dried (Na₂SO₄) and concentrated to provide the target compound as a brown solid. ¹H NMR (500 MHz, DMSO-d6) δ ppm 1.61-1.72 (m, 2H), 1.77-1.85 (m, 2H), 2.03-2.12 (m, 2H), 2.81-2.89 (m, 2H), 3.54 (s, 2H), 3.75-3.85 (m, 1H), 6.45 (d, 1H, J=10 Hz), 6.83 (s, 1H), 7.13 (d, 1H, J=5 Hz), 7.26 (s, 1H), 7.43 (dt, 1H, J=10, 5 Hz), 7.59-7.65 (m, 2H), 7.87 (d, 1H, J=10 Hz), 8.11 (dd, 1H, J=10, 5 Hz), 8.86 (d, 1H, J=10 Hz), 11.71 (s, 1H); MS (ESI) m/z 448 [M+H]⁺.

### Example 88

### N-{1-[6-(acetylamino)-4-methyl-2,3-dihydro-1H-inden-1-yl]piperidin-4-yl}-7-fluoro-4-oxo-4H-chromene-2-carboxamide

Example 88 was prepared according to the procedure outlined in Example 67, substituting 4-methyl-6-nitro-indan-1-one for 6-nitro-indan-1-one. ¹H NMR (400 MHz, DMSO-d6) δ ppm 1.75-1.95(m, 2H), 2.04(s, 3H), 2.07(m, 2H), 2.21(s, 3H), 2.28-2.53(m, 2H), 2.79-3.11(m, 6H), 3.46(m, 1H), 4.04(m, 1H), 6.84(s, 1H), 7.23(s, 1H), 7.42-7.44(m, 1H), 7.58-7.61(m, 1H), 7.89(s, 1H), 8.09-8.13(m, 1H), 9.04(d, 1H, J=7.36), 9.60(s, br, 1H); MS (ESI) m/e 476.2 (M−H)⁺.

### Example 89

### N-[1-(1,3-benzodioxol-5-ylmethyl)piperidin-4-yl]-7-ethyl-6-fluoro-4-oxo-4H-chromene-2 carboxamide

### Example 89A

### 7-Ethyl-6-fluoro-4-oxo-4H-chromene-2-carboxylic acid

A flask was charged with 7-Bromo-6-fluoro-4-oxo-4H-chromene-2-carboxylic acid prepared as described in Example 65C (60 mg, 0.21 mmol), 1M diethylzinc in heptane (0.418 mL, 0.418 mmol), Pd(dppf) Cl2 (2 mg), and Benzene (5 mL). Mixture was refluxed for 2 hr, then quenched with MeOH, diluted with diethyl ether, washed with 1M HCl and brine. Organics were filtered through a bed of celite and purified on a RP-HPLC to provide 7-Ethyl-6-fluoro-4-oxo-4H-chromene-2-carboxylic acid. 1H NMR (300 MHz, DMSO-D6) δ ppm 1.25 (t, J=7.63 Hz, 3H) 2.79 (q, J=7.80 Hz, 2H) 6.89 (s, 1H) 7.68 (d, J=9.49 Hz, 1H) 7.76 (d, J=5.76 Hz, 1H).

### Example 89

### N-[1-(1,3-benzodioxol-5-ylmethyl)piperidin-4-yl]-7-ethyl-6-fluoro-4-oxo-4H-chromene-2 carboxamide

A vial was charged with 7-Ethyl-6-fluoro-4-oxo-4H-chromene-2-carboxylic acid (4 mg, 0.017 mmol), 1-benzo[1,3]dioxol-5-ylmethyl-piperidin-4-ylamine dihydrochloric acid salt (5 mg, 0.017 mmol), EDCI (3 mg, 0.017 mmol), HOBT (2 mg, 0.017 mmol), NMM (0.008 mL, 0.07 mmol), and DMF (0.4 mL). Solution was shaken at 55 C for 16 h then purified on a RP-HPLC to give N-[1-(1,3-benzodioxol-5-ylmethyl)piperidin-4-yl]-7-ethyl-6-fluoro-4-oxo-4H-chromene-2-carboxamide. ¹H NMR (400 MHz, DMSO-D6) δ ppm 1.23-1.30 (m, 3H) 1.86 (m, 2H) 2.05 (d, J=10.74 Hz 2H) 2.75-2.84 (m, 3H) 2.99-3.11 (m, 3H) 4.01 (m, 1H) 4.21 (m, 2H) 6.07 (s, 1H) 6.08 (s, 2H) 7.01 (t, J=7.83 Hz, 3H) 7.07 (s, 1H) 7.70 (dd, J=17.49, 7.67 Hz, 2H) MS (APCI) m/z 453 [M+H]⁺.

### Example 90

### N-[1-(4-amino-3-fluorobenzyl)piperidin-4-yl]-7-fluoro-4-oxo-4H-chromene-2-carboxamide

A flask was charged with 7-Fluoro-4-oxo-4H-chromene-2-carboxylic acid piperidin-4-ylamide hydrochloride salt (0.5 g, 1.53 mmol), (2-Fluoro-4-formyl-phenyl)-carbamic acid tert-butyl ester (0.37 g, 1.53 mmol), sodium triacetoxyborohydride (0.65 g, 3.1 mmol), THF (10 mL) and acetic acid (0.25 mL). Solution was stirred at room temperature for 16 hr. Solution was concentrated to dryness and purified on a FlashMaster II silica column using gradient conditions from 100% CH₂Cl₂ to 5% methanol/CH₂Cl₂. Product fractions were combined and concentrated to dryness. The compound was taken up in CH₂Cl₂ (20 mL) and treated with trifluoroacetic acid (4 mL) for 16 hr. Solution was concentrated to dryness and then diluted with diethyl ether (20 mL). The resulting solid was filtered and washed with diethyl ether to provide N-[1-(4-amino-3-fluorobenzyl)piperidin-4-yl]-7-fluoro-4-oxo-4H-chromene-2-carboxamide as the di-TFA salt. ¹H NMR (300 MHz, DMSO-d6) δ ppm 1.75-1.91 (m, 2H), 2.00-2.13 (m, 2H), 2.96-3.13 (m, 2H), 3.37-3.45 (m, 2H), 3.91-4.07 (m, 1H), 4.12 (d, 2H, J=4.69 Hz), 5.45 (s, 3H), 6.80 (dd, 1H, J=9.16, 8.39 Hz), 6.85 (s, 1H), 6.99 (dd, 1H, J=8.12, 1.94 Hz), 7.16 (dd, 1H, J=12.28, 1.85 Hz), 7.44 (td, 1H, J=8.71, 2.40 Hz), 7.62 (ddd, 1H, J=17.52, 9.47, 2.45 Hz), 8.12 (dd, 1H, J=8.90, 6.44 Hz), 9.06 (d, 1H, J=7.42 Hz), 9.34-9.43 (bs, 1H) MS (ESI) m/z 414 [M+H]⁺.

### Example 91

### N-{1-[1-(2,2-difluoro-1,3-benzodioxol-5-yl)ethyl]piperidin-4-yl}-7-fluoro-4-oxo-4H-chromene-2-carboxamide

### Example 91A

### 5-(1-Chloro-ethyl)-2,2-difluoro-benzo[1,3]dioxole

To a 500 mL rb flask was added 10.0 grams of 2,2-Difluoro-benzo[1,3]dioxole-5-carbaldehyde (53.8 mmol) and 100 mL of dry ether. The solution was cooled to 0° C. in an ice bath, a 3M solution of methylmagnesium bromide (27.0 mL, 81.0 mmol) was added via addition funnel over 15-20 minutes, and the mixture allowed to warm slowly to room temperature. Upon consumption of the starting material by TLC (5% EtOAc in hexanes), the mixture was cooled to 0° C. and slowly quenched with sat. NaCl (5 mL), the resultant precipitate filtered over silica gel, and the cake rinsed with ether. The eluant was then concentrated in vacuo and the residue taken up in a biphasic mixture of 3:1 EtOAc/sat NaCl, partitioned in a separatory funnel, and the organic layer isolated. The organic layer was then washed with sat NaCl, H₂O, dried, and then filtered. Evaporation of the solvents provided the secondary alcohol in 83.5% yield. A portion of this material (8.88 g, 44.0 mmol) was then dissolved in 19 mL of heptane and pyridine (3.91 mL, 48.3 mmol), DMF (2.18 mL, 28.2 mmol), and methanesulfonyl chloride were added (3.74 mL, 48.3 mmol). The mixture was allowed to stir for 12 hours at room temperature after which time it was diluted with 3:1 EtOAc/hexanes and washed with 0.1 N HCl (×2), sat NaHCO₃ (×2), H₂O, dried, and filtered. Evaporation of the solvents provided the titled product in 67% yield. ¹H NMR (300 MHz, CHLOROFORM-D) δ ppm 1.83 (d, J=6.78 Hz, 3H), 5.06 (q, J=7.01 Hz, 1H), 6.97-7.04 (m, 1H), 7.08-7.13 (m, 1H), 7.18 (d, J=1.70 Hz, 1H).

### Example 91

### N-{1-[1-(2,2-difluoro-1,3-benzodioxol-5-yl)ethyl]piperidin-4-yl}-7-fluoro-4-oxo-4H-chromene-2-carboxamide

A 100 mL flask was charged with 5-(1-Chloro-ethyl)-2,2-difluoro-benzo[1,3]dioxole (1.00 g, 4.55 mmol), 7-fluoro-4-oxo-4H-chromene-2-carboxylic acid piperidin-4-ylamide hydrochloride (1.53 g, 4.78 mmol), Na₂CO₃ (1.45 g, 13.7 mmol, pulverized and then dried via vacuum oven at 200° C.), and 10 mL of CH₃CN, and the suspension slowly heated to 70° C. After 16 hours, the suspension was cooled to room temperature and diluted with 20 mL of EtOAc, passed over celite, and the cake rinsed with 5% Et₃N in EtOAc (20 mL×2). The eluant was evaporated and the residue taken up in 5% Et₃N in EtOAc and purified via column chromatography (2-5% Et₃N in EtOAc) to provide the racemic title product. ¹H NMR (300 MHz, DMSO-d6) δ ppm 1.30 (d, 3H, J=6.61 Hz), 1.52-1.69 (m, 2H), 1.71-1.89 (m, 2H), 1.88-2.11 (m, 2H), 2.71-2.86 (m, 1H), 2.88-3.03 (m, 1H), 3.48-3.62 (m, 1H), 3.67-3.79 (m, 1H), 6.81 (s, 1H), 7.16 (t, 1H, J=8.18 Hz), 7.32-7.37 (m, 2H), 7.43 (td, 1H, J=8.71, 2.51 Hz), 7.61 (dd, 1H, J=9.46, 2.45 Hz), 8.11 (dd, 1H, J=8.89, 6.36 Hz), 8.78-8.84 (m, 1H); MS (ESI) m/z 475 [M+H]⁺.

### Example 92

### N-{1-[1-(3-ethyl-2-oxo-2,3-dihydro-1,3-benzothiazol-6-yl)ethyl]piperidin-4-yl}-7-fluoro-4-oxo-4H-chromene-2-carboxamide

### Example 92A

### 6-acetyl-3-ethyl-3H-benzothiazole-2-one

A suspension of 6-acetyl-3H-benzthiazole-2-one (300 mg, 1.55 mmol), K₂CO₃ (215 mg, 1.55 mmol), and EtI (0.163 mL, 2.03 mmol) in DMF (2 mL) was heated to 130° C. for 2 hours. After cooling to room temperature, the mixture was diluted with MeOH (3 mL) and filtered, eluting with additional MeOH (3 mL). The solution was concentrated under reduced pressure to provide the title compound as a beige solid that was used without further purification. MS (ESI) 222 (M+H)⁺.

### Example 92

### N-{1-[1-(3-ethyl-2-oxo-2,3-dihydro-1,3-benzothiazol-6-yl)ethyl]piperidin-4-yl}-7-fluoro-4-oxo-4H-chromene-2-carboxamide

A mixture of 7-fluoro-4-oxo-4H-chromene-2-carboxylic acid piperidin-4-ylamide (0.284 g, 0.976 mmol), 6-acetyl-3-ethyl-3H-benzothiazole-2-one (216 mg, 0.976 mmol), and Ti(OⁱPr)₄ (1.14 mL, 3.90 mmol) in THF (5 mL) was heated to 70° C. for 5 hours. EtOH (1.0 mL) and NaBH(OAc)₃ (0.412 g, 1.95 mmol) were added to the mixture, and the mixture was heated to 50° C. for an additional 2 hours. The mixture was quenched by the addition of saturated aqueous NaHCO₃ (10 mL) and EtOAc (10 mL). The layers were separated, and the aqueous was extracted with additional EtOAc (3×10 mL). The combined organic layers were dried with anhydrous Na₂SO₄, filtered, and concentrated under reduced pressure. The residue was purified by MPLC (5% MeOH in EtOAc) to provide the title compound as a white solid. ¹H NMR (300 MHz, DMSO-d6) δ ppm 1.22 (t, 3H, J=7.1 Hz), 1.32 (d, 3H, J=6.8 Hz), 1.50-1.70 (m, 2H), 1.79 (br t, 2H, J=14.2 Hz), 1.90-2.07 (m, 2H), 2.81 (br d, 1H, J=11.5 Hz), 2.97 (br d, 1H, J=10.2 Hz), 3.54 (q, 1H, J=6.8 Hz), 3.62-3.80 (m, 1H), 3.97 (q, 2H, J=7.1 Hz), 6.81 (s, 1H), 7.33 (m, 2H), 7.42 (dt, 1H, J=2.4 and 8.5 Hz), 7.59-7.63 (m, 2H), 8.11 (dd, 1H, J=6.1 and 8.8 Hz), and 8.81 (d, 1H, J=7.8 Hz); MS (ESI) m/z 496 (M+H)⁺.

### Example 93

### 7-fluoro-4-oxo-N-1-[6-(propionylamino)-2,3-dihydro-1H-inden-1-yl]piperidin-4-yl}-4H-chromene-2-carboxamide

Example 93 was prepared according to the procedure outlined in Example 88 substituting propionic acid for acetic acid. ¹H NMR (400 MHz, DMSO-d₆) δ ppm 1.08(t, 3H), 1.54-1.83(m, 4H), 2.00-2.03(m, 2H), 2.19-2.40(m, 4H), 2.70-2.93(m, 4H), 3.74-3.78(m, 1H), 4.30(t, 1H), 6.83(s, 1H), 7.12(d, 1H, J=7.94), 7.32(d, 1H, J=7.94), 7.43(t, 1H), 7.64(dd, 1H, J=2.44, 9.46), 7.69(s, 1H), 8.12(dd, 1H J=2.44, 8.85), 8.87(d, 1H, J=7.93), 9.79(s, 1H); MS (ESI) m/e 476.2 (M−H)⁺.

### Example 94

### 7-fluoro-N-{1-[1-(3-methyl-2-oxo-2,3-dihydro-1,3-benzoxazol-6-yl)ethyl]piperidin-4-yl}-4-oxo-4H-chromene-2-carboxamide

### Example 94A

### 6-acetyl-3-methyl-3H-benzoxazole-2-one

A suspension of 6-acetyl-3H-benzoxazole-2-one (100 mg, 0.565 mmol), K₂CO₃ (94 mg, 0.678 mmol), and MeI (0.042 mL, 0.678 mmol) in DMF (1 mL) was heated to 130° C. for 2 hours. After cooling to room temperature, the mixture was diluted with MeOH (2 mL) and filtered, eluting with additional MeOH (3 mL). The solution was concentrated under reduced pressure to provide the title compound as a beige solid that was used without further purification. MS (ESI) 192 (M+H)⁺.

### Example 94

### 7-fluoro-N-{1-[1-(3-methyl-2-oxo-2,3-dihydro-1,3-benzoxazol-6-yl)ethyl]piperidin-4-yl}-4-oxo-4H-chromene-2-carboxamide

A mixture of 7-fluoro-4-oxo-4H-chromene-2-carboxylic acid piperidin-4-ylamide (1.00 g, 3.44 mmol), 6-acetyl-3-methyl-3H-benzoxazole-2-one (656 mg, 3.44 mmol), and Ti(OⁱPr)₄ (4.02 mL, 13.76 mmol) in THF (10 mL) was heated to 70° C. for 5 hours. EtOH (2.0 mL) and NaBH(OAc)₃ (1.45 g, 6.88 mmol) were added to the mixture, and the mixture was heated to 50° C. for an additional 2 hours. The mixture was quenched by the addition of saturated aqueous NaHCO₃ (25 mL) and EtOAc (25 mL). The layers were separated, and the aqueous was extracted with additional EtOAc (3×25 mL). The combined organic layers were dried with anhydrous Na₂SO₄, filtered, and concentrated under reduced pressure. The residue was purified by MPLC (5% MeOH in EtOAc) to provide the title compound as a white solid. ¹H NMR (300 MHz, DMSO-d6) δ ppm 1.69 (d, 3H, J=6.8 Hz), 1.76-1.94 (m, 2H), 2.32 (br t, 2H, J=13.2 Hz), 2.85-3.00 (m, 2H), 3.31 (br d, 2H, J=13.0 Hz), 3.38 (s, 3H), 3.87-3.98 (m, 1H), 4.52-4.62 (m, 1H), 6.84 (s, 1H), 7.39 (m, 2H), 7.43 (dt, 1H, J=2.7 and 8.8 Hz), 7.57 (s, 1H), 7.61 (dd, 1H, J=2.4 and 9.5 Hz), 8.13 (dd, 1H, J=6.4 and 8.8 Hz), and 9.03 (d, 1H, J=7.5 Hz); MS (ESI) m/z 466 (M+H)⁺.

### Example 95

### 7-fluoro-N-(1-{3-fluoro-4-[(3-piperidin-1-ylpropanoyl)amino]benzyl}piperidin-4-yl)-4-oxo-4H-chromene-2-carboxamide

A vial was charged with N-[1-(4-amino-3-fluorobenzyl)piperidin-4-yl]-7-fluoro-4-oxo-4H-chromene-2-carboxamide prepared as described in Example 90 (25 mg, 0.04 mmol), 1-piperidinepropionic acid (6.1 mg, 0.04 mmol), EDCI (17.5 mg, 0.04 mmol), HOBT (5.3 mg, 0.04 mmol), NMM (0.017 mL, 0.16 mmol), and DMF (0.4 mL). Solution was shaken at 55° C. for 16 hours. Sample was purified on a FlashMaster II silica column using gradient conditions from 100% CH₂Cl₂ to 5% methanol/CH₂Cl₂ to provide 7-fluoro-N-(1-{3-fluoro-4-[(3-piperidin-1-ylpropanoyl)amino]benzyl}piperidin-4-yl)-4-oxo-4H-chromene-2-carboxamide. ¹H NMR (300 MHz, DMSO-D6) δ ppm 1.31-1.39 (m, 2H) 1.40-1.46 (m, 2H) 1.49-1.58 (m, 2H) 1.59-1.72 (m, 3H) 1.77-1.91 (m, 3H) 2.95-3.03 (m, 2H) 3.42-3.50 (m, 1H) 3.74-3.88 (m, 1H) 4.66 (ddd, J=10.60, 6.70, 4.41 Hz, 2H) 6.72 (t, J=8.82 Hz, 1H) 6.84 (d, J=3.73 Hz, 2H) 6.88-7.01 (m, 1H) 7.39-7.46 (m, 3H) 7.58-7.70 (m, 3H) 7.84 (d, J=8.14 Hz, 1H) 7.93-7.98 (m, 1H) 8.12 (dd, J=8.82, 6.10 Hz, 2H) 8.87 (d, J=7.80 Hz, 2H) MS (ESI) m/z 553 [M+H]⁺.

### Example 96

### N-{1-[5-(acetylamino)-2-chlorobenzyl]piperidin-4-yl}-7-fluoro-4-oxo-4H-chromene-2-carboxamide

### Example 96A

### 7-Flouro-4-oxo-4H-chromene-2-carboxylic acid[1-(2-chloro-5-nitro-benzyl)-piperidin-4-yl]-amide

Example 60D (0.1 g, 0.34 mmol), 2-chloro-5-nitro-benzaldehyde (0.075 g, 0.4 mmol), sodium triacetoxyborohydride (0.144 g, 0.7 mmol), acetic acid (0.25 mL) and THF (5 mL) were processed as in Example 1 to provide the title compound as a white solid. MS (ESI) m/z 460 [M+H]⁺.

### Example 96

### N-{1-[5-(acetylamino)-2-chlorobenzyl]piperidin-4-yl}-7-fluoro-4-oxo-4H-chromene-2-carboxamide

Example 96A (0.14 g, 0.3 mmol), iron powder (0.08 g, 1.5 mmol) and ammonium chloride (0.02 g, 0.3 mmol) were placed together in ethanol (10 mL) and water (2 mL) and heated at reflux for 4 hours. The mixture was cooled, filtered over wet celite, extracted with ethyl acetate (3×20 mL), dried (MgSO₄) and used without further purification. The crude amine (0.05 g, 0.12 mmol) was placed along with acetic acid (0.01 g, 0.25 mmol), EDCI (0.03 g, 0.14 mmol), HOBt (0.02 g, 0.14 mmol) and N-methyl morpholine (0.05 g, 0.5 mmol) in DMF (4 mL) and stirred at room temperature for 15 hours. The mixture was diluted with water, extracted with ethyl acetate (3×20 mL), organics washed with water, brine, dried (MgSO₄) and purified using RP-HPLC to provide the title compound as a tan solid. ¹H NMR (300 MHz, DMSO-d6) δ ppm 1.60-1.76 (m, 2H), 1.77-1.87 (m, 2H), 2.04 (s, 3H), 2.10-2.22 (m, 2H), 2.85-2.95 (m, 2H), 3.53 (s, 2H), 3.73-3.89 (m, 1H), 6.83 (s, 1H), 7.33 (d, 1H, J=8.63 Hz), 7.44 (td, 1H, J=8.77, 2.46 Hz), 7.47 (dd, 1H, J=8.86, 2.50 Hz), 7.62 (dd, 1H, J=9.49, 2.46 Hz), 7.79 (d, 1H, J=2.58 Hz), 8.12 (dd, 1H, J=8.89, 6.36 Hz), 8.87 (d, 1H, J=7.89 Hz), 10.04 (s, 1H). MS (ESI) m/z 472 [M+H]⁺.

### Example 97

### ethyl 3-(4-{[(7-fluoro-4-oxo-4H-chromen-2-yl)carbonyl]amino}piperidin-1-yl)-2,3-dihydro-1H-inden-5-ylcarbamate

Example 97 was prepared according to the procedure outlined in Example 88, substituting ethyl chloroformate for acetic acid. ¹H NMR (400 MHz, DMSO-d₆) δ ppm 1.24(t, 3H), 1.59-1.85(m, 4H), 1.97-2.03(m, 2H), 2.17-2.40(m, 2H), 2.52-2.92(m, 4H), 3.75-3.78(m, 1H), 4.12(q, 2H, J=7.32), 4.29(t, 1H), 6.82(s, 1H), 7.10(d, 1H, J=8.24), 7.19(d, 1H, J=7.93), 7.43(t, 1H), 7.53(s, 1H), 7.62(dd, 1H, J=2.44, 9.46), 8.12(dd, 1H J=2.44, 8.85), 8.86(d, 1H, J=8.24), 9.50(s, 1H); MS (ESI) m/e 494.1 (M+H)⁺.

### Example 98

### 7-fluoro-N-[1-(1H-indol-5-ylmethyl)piperidin-4-yl]-4-oxo-4H-chromene-2-carboxamide

Example 60C (29 mg, 0.100 mmol), indole-5-carboxaldehyde (15 mg, 0.100 mmol), sodium triacetoxyborohydride (42 mg, 0.200 mmol), Na₂SO₄ (28 mg, 0.200 mmol), acetic acid (20 μL), and THF (1 mL) were processed as described in Example 1 to provide the title compound (28 mg, 67%) as a white solid. ¹H NMR (300 MHz, DMSO-d6): δ ppm 1.53-1.71 (m, 2H), 1.71-1.85 (m, 2H), 1.97-2.07 (m, 2H), 2.87 (d, 2H, J=11.22 Hz), 3.53 (s, 2H), 3.72-3.84 (m, 1H), 6.31-6.44 (m, 1H), 6.82 (s, 1H), 7.05 (dd, 1H, J=8.27, 1.56 Hz), 7.24-7.51 (m, 4H), 7.62 (dd, 1H, J=9.43, 2.44 Hz), 8.11 (dd, 1H, J=8.84, 6.35 Hz), 8.81 (d, 1H, J=7.87 Hz), 11.00 (s, 1H); MS (APCI) m/z 420 [M+H]⁺, 418 [M+H]⁺.

### Example 99

### 7-fluoro-4-oxo-N-[1-(5-{3-[(trifluoroacetyl)amino]propoxy}-2,3-dihydro-1H-inden-1-yl)piperidin-4-yl]-4H-chromene-2-carboxamide

### Example 99A

### 2,2,2-Trifluoro-N-[3-(1-oxo-indan-5-yloxy)-propyl]-acetamide

A 20 mL scintillation vial with a septum cap was charged with PS-PPh₃ resin (Aldrich Chemical Co., Inc, 100 mg, 2.2 equiv), 5-hydroxy-indan-1-one (0.300 g, 2.03 mmol), DBAD (0.746 g, 3.20 mmol) and purged by passing a stream of N2 for 45 seconds. Anhydr. THF (15 mL) was added and the contents of the vial were shaken for 5 min. Following this, a solution of N-(3-hydroxypropyl)-2,2,2-trifluoroacetamide (0.430 g, 2.51 mmol) in anhydr. THF (1 mL) was added and the resulting suspension was shaken at room temperature for 8 hours. The suspension was filtered, and the resin washed with THF. Evaporation of the filtrate and purification via RP-HPLC provided the title product. 1H NMR (300 MHz, CHLOROFORM-D) δ ppm 2.06-2.25 (m, 2H), 2.57-2.79 (m, 2H), 2.99-3.19 (m, 2H), 3.63 (q, J=6.10 Hz, 2H), 4.09-4.24 (m, 2H), 6.72-6.94 (m, 2H), 7.12-7.34 (m, 1H), 7.70 (d, J=9.15 Hz, 1H); MS (ESI) m/z 302 [M+H]⁺.

### Example 99

### 7-fluoro-4-oxo-N-[1-(5-{3-[(trifluoroacetyl)amino]propoxy}-2,3-dihydro-1H-inden-1-yl)piperidin-4-yl]-4H-chromene-2-carboxamide

A solution of 2,2,2-Trifluoro-N-[3-(1-oxo-indan-5-yloxy)-propyl]-acetamide (0.150 g, 0.498 mmol) and 7-Fluoro-4-oxo-4H-chromene-2-carboxylic acid piperidin-4-ylamide (0.217 g, 0.748 mmol) in THF (5 mL) was placed under N₂ and Ti(OPr)₄ (0.146 mL, 0.748 mmol) was added slowly via syringe. The mixture was then heated to reflux for 8 hours. After this time, the mixture was cooled to room temperature, and NaBH₃CN (0.046 g, 0.730 mmol) was added in portions with stirring. The mixture was then slowly heated to 50° C. and allowed to stir at this temperature overnight. After 14 hours, the mixture was first cooled to room temperature, and then placed in an ice bath prior to the addition of 2 mL of sat. NaHCO₃. The resulting quenched mixture was stirred for 30 minutes, and then filtered over a plug of celite, and the cake rinsed with EtOAc (50 mL). Following concentration of the eluant, the residue was then taken up in EtOAc and purified via silica gel chromatography (2-5% Et₃N in EtOAc) to provide the title compound. 1H NMR (300 MHz, DMSO-D6) δ ppm 1.49-1.68 (m, 2H), 1.70-1.86 (m, 2H), 1.87-2.05 (m, 4H), 2.10-2.23 (m, 1H), 2.24-2.38 (m, 1H), 2.65-2.95 (m, 4H), 3.66-3.81 (m, 1H), 3.93-4.01 (m, 2H), 4.03-4.13 (m, 2H), 4.17-4.30 (m, 1H), 6.70-6.81 (m, 2H), 6.82 (s, 1H), 7.08-7.22 (m, J=8.14 Hz, 1H), 7.35-7.49 (m, 1H), 7.62 (dd, J=9.49, 2.37 Hz, 1H), 8.11 (dd, J=8.82, 6.44 Hz, 1H), 8.76-8.88 (m, J=7.80 Hz, 1H), 9.41-9.55 (m, 1H); MS (ESI) m/z 576 [M+H]⁺.

### Example 100

### N-[1-(2,3-dihydro-1,4-benzodioxin-6-ylmethyl)piperidin-4-yl]-6-fluoro-7-methoxy-4-oxo-4H-chromene-2-carboxamide

A vial was charged with 6-Fluoro-7-methoxy-4-oxo-4H-chromene-2-carboxylic acid piperidin-4-ylamide as prepared in example 56 (20 mg, 0.06 mmol), 2,3-Dihydro-benzo[1,4]dioxine-6-carbaldehyde (10.3 mg, 0.06 mmol), PS-cyanoborohydride (40 mg, 0.12 mmol), THF (0.4 mL), and MeOH (0.2 mL). Mixture was shaken at 40° C. for 16 hours. Solution was filtered and purified on a RP-HPLC to provide N-[1-(2,3-dihydro-1,4-benzodioxin-6-ylmethyl)piperidin-4-yl]-6-fluoro-7-methoxy-4-oxo-4H-chromene-2-carboxamide. ¹H NMR (300 MHz, DMSO-d6) δ ppm 1.75-1.91 (m, 2H), 2.02-2.12 (m, 2H), 3.00-3.15 (m, 2H), 3.38-3.47 (m, 2H), 4.02 (s, 3H), 4.16-4.22 (m, 3H), 4.27 (s, 2H), 6.82 (s, 1H), 6.91-6.99 (m, 3H), 7.02-7.07 (m, 2H), 7.44 (d, 1H, J=6.95 Hz), 7.74 (d, 1H, J=10.85 Hz), 9.07 (d, 1H, J=7.46 Hz), 9.47 (s, 1H) MS (ESI) m/z 469 [M+H]⁺.

### Example 101

### N-{1-[1-(6-chloro-1,3-benzodioxol-5-yl)ethyl]piperidin-4-yl}-7-fluoro-4-oxo-4H-chromene-2-carboxamide

Methyl magnesium bromide (1M solution in butyl ether, 10 mL) was added to a solution of 6-Chloro-benzo[1,3]dioxole-5-carbaldehyde (1.5 g, 8.13 mmol) in THF (25 mL) at 0° C. and stirred for 2 hours. The mixture was quenched by the addition of saturated ammonium chloride, extracted with ethyl acetate (3×20 mL), organic extracts washed with water, brine, dried MgSO₄) and concentrated to a crude oil that was used without further purification. Methanesulfonyl chloride (0.1 g, 0.85 mmol) was added to a solution of 1-(6-Chloro-benzo [1,3]dioxol-5-yl)-ethanol (0.17 g, 0.85 mmol) and triethyl amine (0.13 g, 1.25 mmol) in dichloromethane (5 mL) at 0° C. and was stirred for 2.5 hours. Example 60D was then added to this solution at room temperature and heated at 50° C. for 12 hours. The mixture was cooled, quenched with water, extracted with ethyl acetate, concentrated and purified by RP-HPLC to provide the title compound as a colorless oil. ¹H NMR (500 MHz, DMSO-d6) δ ppm 1.61 (d, 3H, J=10 Hz), 1.77-1.87 (m, 1H), 1.88-1.98 (m, 1H), 2.00-2.15 (m, 2H), 2.95-3.05 (m, 1H), 3.10-3.21 (m, 1H), 3.25-3.30 (m, 1H), 3.73-3.84 (m, 1H), 3.98-4.07 (m, 1H), 4.73-4.81 (m, 1H), 6.15 (s, 2H), 6.85 (s, 1H), 7.22 (s, 1H), 7.34 (s, 1H), 7.44 (td, 1H, J=10, 4 Hz), 7.61 (dd, 1H, J=10, 4 Hz), 8.12 (dd, 1H, J=10, 5 Hz), 9.05 (d, 1H, J=10 Hz). MS (ESI) m/z 473 [M+H]⁺.

### Example 102

### N-[1-(1,3-benzodioxol-5-ylmethyl)piperidin-4-yl]-6-fluoro-7-(isobutylamino)-4-oxo-4H-chromene-2-carboxamide

A 4 mL vial with screw cap was charged with example 55 (30 mg, 0.068 mmol), DMF (3 mL), isobutylamine (7.5 mg, 0.102 mmol) and K₂CO₃ (19 mg, 0.136 mmol).). The mixture vessel was placed on a shaker at 40° C. for 16 hrs. After this time, the DMF was removed under reduced pressure. EtOAc was added into the residue, washed with water. The layers were separated, and the organic layer was washed with brine (100 mL), dried (Na₂SO₄), and solvents were removed under reduced pressure. The residue was dissolved in 1.5 mL of a 1:1 mixture of DMSO/MeOH and purified by preparative reverse-phase HPLC. ¹H NMR (400 MHz, DMSO-d₆) δ ppm 0.94(d, 6H, J=6.75), 1.82-2.05(m, 5H), 3.00-3.17(m, 6H), 3.97(m, 1H), 4.20(s, 2H), 6.08(s, 2H), 6.68(s, 1H), 6.75(d, 1H, J=7.05), 6.97-7.03(m, 3H), 7.09(s, 1H), 7.49(d, 1H, J=11.36), 8.95(d, 1H, J=7.67); MS (ESI) m/e 494.1 (M−H)⁺.

### Example 103

### N-[1-(4-{[(2-anilinoethyl)amino]carbonyl}benzyl)piperidin-4-yl]-7-fluoro-4-oxo-4H-chromene-2-carboxamide

### Example 103A

### 4-{4-[(7-Fluoro-4-oxo-4H-chromene-2-carbonyl)-amino]-piperidin-1-ylmethyl}-benzoic acid

A scintillation vial was charged with 7-fluoro-4-oxo-4H-chromene-2-carboxylic acid piperidin-4-ylamide hydrochloride (375 mg, 1.2 mmol), 4-Formyl-benzoic acid (0.220 g, 1.47 mmol), 5.5 mL of a solution of DCE/MeOH (1% AcOH). Macroporous BH₃CN was then added (0.80 g, 2.0 equiv, 3.0 mmol/g loading) and the mixture placed in a heated shaker (50 C) for 8 hours. After this time an additional 2 equiv of solid supported reagent was added, and the mixture was replaced onto the heated shaker. After 8 hours, the mixture was rapidly poured onto a Buchner funnel and rinsed with MeOH (20 mL). The eluant was evaporated to dryness to provide the title product. 1H NMR (500 MHz, Solvent) δ ppm 1.86-1.99 (m, 2H), 2.05-2.22 (m, 4H), 2.89-3.00 (m, J=11.85 Hz, 2H), 3.57 (s, 2H), 4.18-4.34 (m, 1H), 6.44 (dd, J=9.36, 2.18 Hz, 1H), 7.10-7.17 (m, 2H), 7.48-7.54 (m, J=8.11 Hz, 2H), 8.28 (dd, J=8.73, 6.55 Hz, 1H), 8.36-8.44 (m, J=8.11 Hz, 2H), 9.53-9.67 (m, J=8.11 Hz, 1H); MS (ESI) m/z 543 [M+H]⁺.

### Example 103

### N-[1-(4-{[(2-anilinoethyl)amino]carbonyl}benzyl)piperidin-4-yl]-7-fluoro-4-oxo-4H-chromene-2-carboxamide

A portion of 103A (0.030 g, 0.071 mmol) was then dissolved in 2 mL of DMF in a scintillation flask and N1-Phenyl-ethane-1,2-diamine (11.6 mg, 0.085 mmol), HOBt (14.4 mg, 0.107 mmol), and EDCI (17.9 mg, 0.071 mmol) were added. The flask was placed on a heated shaker for 6 hours, and then cooled to room temperature. Evaporation of the solvents and purification via RP-HPLC provided the desired product. 1H NMR (500 MHz, PYRIDINE-D5) δ ppm 1.91-2.04 (m, 2H), 2.06-2.16 (m, J=10.07 Hz, 2H), 2.22 (t, J=11.14 Hz, 2H), 2.93-3.07 (m, J=11.60 Hz, 2H), 3.54-3.68 (m, 4H), 3.84-3.97 (m, J=6.10, 6.10, 6.10 Hz, 2H), 4.24-4.37 (m, 1H), 6.45 (dd, J=9.31, 2.29 Hz, 1H), 6.76 (t, J=7.32 Hz, 1H), 6.88 (d, J=7.93 Hz, 2H), 7.09-7.19 (m, 1H), 7.23-7.27 (m, 3H), 7.42-7.52 (m, 3H), 8.17-8.26 (m, J=8.24 Hz, 2H), 8.29 (dd, J=8.85, 6.41 Hz, 1H), 9.30 (t, J=5.80 Hz, 1H), 9.73 (d, J=7.63 Hz, 1H); MS (ESI) m/z 543 [M+H]⁺.

### Example 104

### 7-fluoro-4-oxo-N-{1-[(1S)-5-(3-piperidin-1-ylpropoxy)-2,3-dihydro-1H-inden-1-yl]piperidin-4-yl}-4H-chromene-2-carboxamide

### Example 104A

### 5-(3-piperidin-1-ylpropoxy)indan-1-one

To a solution of 5-hydroxyindanone (0.5 g, 3.37 mmol), 3-hydroxypropylpiperidine (0.603 g, 4.21 mmol), and di-tert-butyl azodicarboxylate (1.24 g, 5.39 mmol) in anhydrous THF (200 mL) was added PS-PPh₃ resin (Aldrich Chemical Co., Inc, 2.36 g of 3 mmol/g resin). The suspension stirred for 6 hours at room temperature, and was then filtered through a pad of celite, concentrated to dryness, and purified by silica chromatography (5% MeOH/CH₂Cl₂) to provide example 104A as a light yellow oil. MS (ESI) m/z 274 [M+H]⁺.

### Example 104B

### 7-fluoro-4-oxo-N-{1-[5-(3-piperidin-1-ylpropoxy)-2,3-dihydro-1H-inden-1-yl]piperidin-4-yl}-4H-chromene-2-carboxamide

Example 60D and example 104A were processed according to the procedure described in example 58 to provide example 104B. MS (ESI) m/z 548 [M+H]⁺.

### Example 104

### 7-fluoro-4-oxo-N-{1-[(1S)-5-(3-piperidin-1-ylpropoxy)-2,3-dihydro-1H-inden-1-yl]piperidin-4-yl}-4H-chromene-2-carboxamide

Example 104B was separated into its enantiomers by HPLC chromatography using a Chiralcel OJ column eluting with a mobil phase of hex/EtOH/MeOH (85/7.5/7.5). Example 104 was the second enantiomer to elute from the column. ¹H NMR (300 MHz, DMSO-d6) δ ppm 1.30-2.45 (m, 16H), 2.70-3.00 (m, 10H), 3.68-3.82 (m, 1H), 3.96-4.04 (m, 1H), 4.20-4.29 (m, 1H), 6.72-6.82 (m, 2H), 6.82 (s, 1H), 7.17 (t, 1H, J=8.2 Hz), 7.43 (td, 1H, J=8.7, 2.5 Hz), 7.61 (dd, 1H, J=9.5, 2.5 Hz), 8.10 (dd, 1H, J=8.9, 6.4 Hz), 8.78-8.84 (m, 1H); MS (ESI) m/z 548 [M+H]⁺.

### Example 105

### 7-fluoro-N-(1-{3-fluoro-4-[(methoxyacetyl)amino]benzyl}piperidin-4-yl)-4-oxo-4H-chromene-2-carboxamide

A vial was charged with N-[1-(4-amino-3-fluorobenzyl)piperidin-4-yl]-7-fluoro-4-oxo-4H-chromene-2-carboxamide prepared as described in Example 90 (25 mg, 0.04 mmol), methoxy acetic acid (0.003 mL, 0.04 mmol), EDCI (17.5 mg, 0.04 mmol), HOBT (5.3 mg, 0.04 mmol), NMM (0.017 mL, 0.16 mmol), and DMF (0.4 mL). Solution was shaken at 55° C. for 16 hours. Sample was purified on a RP-HPLC system to provide 7-fluoro-N-(1-{3-fluoro-4-[(methoxyacetyl)amino]benzyl}piperidin-4-yl)-4-oxo-4H-chromene-2-carboxamide. 1H NMR (300 MHz, DMSO-D6) δ ppm 1.75-1.91 (m, 2H) 1.99-2.14 (m, 3H) 3.02-3.17 (m, 2H) 3.37-3.50 (m, 4H) 3.94-4.06 (m, 1H) 4.08 (s, 1H) 4.13 (s, 2H) 4.29 (d, J=3.73 Hz, 2H) 6.85 (s, 1H) 7.10-7.20 (m, 1H) 7.40-7.50 (m, 2H) 7.63 (ddd, J=16.53, 9.41, 2.54 Hz, 1H) 8.09-8.18 (m, 1H) 9.06 (dd, J=7.46, 4.07 Hz, 1H) 9.61 (s, 1H) MS (ESI) m/z 486 [M+H]⁺.

### Example 106

### 7-methoxy-4-oxo-N-{1-[(1S)-1-quinolin-6-ylethyl]piperidin-4-yl}-4H-chromene-2-carboxamide

A solution of example 2C (500 mg, 1.66 mmol) and example 3C (526 mg, 1.66 mmol) in dimethylformamide (6.7 mL) was charged with K₂CO₃ (505 mg, 3.65 mmol). The resultant dark orange heterogeneous mixture was stirred for 42 hours, diluted with CH₂Cl₂, washed with distilled water, dried (Na₂SO₄) and concentrated. Purification by silica gel chromatography (0 to 10% methanol/CH₂Cl₂) provided pure racemic compound. Separation of enantiomers was accomplished by using a chiral column (Chiralcel OJ, 4.6×250 mm, 70/15/15 hexane/ethanol/methanol, 0.8 mL/min, 40 C, Rt=11.3 minutes) to provide the title compound as an off-white solid. [α]589/22.9 C=+5.8, c=0.31 CH₂Cl₂. MS (ESI, MeOH/NH₄OH) m/z 456 [M−H]; 1H NMR (300 MHz, CHLOROFORM-D) δ ppm 1.53 (m, 5H), 2.05 (m, 2H), 2.28 (m, 2H), 2.75-3.01 (m, 1H) 3.04-3.26 (m, 1H) 3.60-3.81 (m, 1H) 3.94 (s, 3H) 6.61-6.77 (m, 1H) 6.92 (s, 1H) 7.01 (dd, J=8.82, 2.37 Hz, 1H) 7.09 (s, 1H) 7.26 (s, 2H) 7.42 (dd, J=8.48, 4.07 Hz, 1H) 7.70-7.88 (m, J=9.16 Hz, 2H) 8.12 (s, 3H) 8.91 (d, J=2.71 Hz, 1H).

### Example 107

### 7-fluoro-N-[1-(3-fluoro-4-methoxybenzyl)piperidin-4-yl]-4-oxo-4H-chromene-2-carboxamide

Example 60C (29 mg, 0.100 mmol), 3-fluoro-4-methoxybenzaldehyde (15 mg, 0.100 mmol), sodium triacetoxyborohydride (42 mg, 0.200 mmol), Na₂SO₄ (28 mg, 0.200 mmol), acetic acid (20 μL), and THF (1 mL) were processed as described in Example 1 to provide the title compound (22 mg, 51%) as a white solid. ¹H NMR (300 MHz, DMSO-d6) δ ppm 1.52-1.72 (m, 2H), 1.74-1.85 (m, 2H), 1.97-2.09 (m, 2H), 2.82 (d, 2H, J=11.28 Hz), 3.42 (s, 2H), 3.70-3.86 (m, 1H), 3.82 (s, 3H), 6.82 (s, 1H), 6.98-7.20 (m, 3H), 7.43 (td, 1H, J=8.69, 2.49 Hz), 7.61 (dd, 1H, J=9.47, 2.46 Hz), 8.11 (dd, 1H, J=8.90, 6.36 Hz), 8.82 (d, 1H, J=7.95 Hz); MS (APCI) m/z 429 [M+H]⁺, 427 [M+H]⁺.

### Example 108

### N-[1-(1,3-benzodioxol-5-ylmethyl)piperidin-4-yl]-6-fluoro-7-(isopropylamino)-4-oxo-4H-chromene-2-carboxamide

Example 108 was prepared according to the procedure outlined in Example 102, substituting isopropylamine for isobutylamine. ¹H NMR (400 MHz, DMSO-d₆) δ ppm 1.24(d, 6H, J=6.14), 1.82-2.07(m, 4H), 3.05-3.07(m, 2H), 3.43-3.45(m, 2H), 3.72-3.73(m, 1H), 3.97-3.99(m, 1H), 4.21(s, 2H), 6.08(s, 2H), 6.68(s, 1H), 6.78(d, 1H, J=7.05), 6.97-7.01(m, 3H), 7.08(s, 1H), 7.49(d, 1H, J=11.66), 8.95(d, 1H, J=7.36); MS (ESI) m/e 480.2 (M−H)⁺.

### Example 109

### ethyl {4-[(4-{[(7-fluoro-4-oxo-4H-chromen-2-yl)carbonyl]amino}piperidin-1-yl)methyl]phenyl}acetate

A 500 mL rb flask was charged with 7-Fluoro-4-oxo-4H-chromene-2-carboxylic acid piperidin-4-ylamide hydrochloride (3.50 g, 10.9 mmol), (4-bromomethyl-phenyl)-acetic acid (2.50 g, 10.9 mmol), K₂CO₃ (4.55 g, 33.0 mmol), and 100 mL of EtOH. The mixture was heated to reflux for six hours, after which time the contents were cooled to room temperature, and diluted with 100 mL EtOAc. The resultant mixture was filtered over a bed of celite, and the eluant evaporated to dryness. Purification of the resultant residue (1:1 EtOAc/hexanes) provided the desired product. 1H NMR (300 MHz, DMSO-D6) δ ppm 1.05-1.23 (m, 3H), 1.53-1.74 (m, 2H), 1.74-1.85 (m, 2H), 1.96-2.11 (m, 2H), 2.78-2.87 (m, 2H), 3.42-3.51 (m, 2H), 3.59-3.67 (m, 2H), 3.71-3.86 (m, 1H), 4.00-4.11 (m, 2H), 6.82 (s, 1H), 7.18-7.29 (m, 4H), 7.35-7.49 (m, 1H), 7.62 (dd, J=9.49, 2.37 Hz, 1H), 8.11 (dd, J=9.15, 6.44 Hz, 1H), 8.76-8.90 (m, J=7.80 Hz, 1H); MS (ESI) m/z 467 [M+H]⁺.

### Example 110

### N-(1-{4-[3-(dimethylamino)propoxy]benzyl}piperidin-4-yl)-7-fluoro-4-oxo-4H-chromene-2-carboxamide

Example 60C (19 mg, 0.065 mmol) and 4-(3-dimethylaminopropoxy)benzaldehyde were processed in a manner analogous to Example 40 to provide the title compound (22 mg, 48%) as the trifluoroacetic acid salt. ¹H NMR (500 MHz, Pyridine-d5/Deuterium Oxide): δ ppm 2.44 (m, 6H), 3.07 (m, 2H), 3.17-3.22 (m, 6H), 3.53-3.63 (m, 6H), 4.18 (m, 2H), 4.26 (m, 2H), 4.51 (m, 1H), 7.07 (d, J=8.73 Hz, 2H), 7.35 (s, 1H), 7.64-7.68 (m, 2H), 7.97 (d, 1H), 8.32 (m, 1H); MS (ESI, MeOH/NH4OH) m/z 482 [M+H]⁺.

### Example 111

### 7-fluoro-4-oxo-N-[1-(4-{[(2-pyrrolidin-1-ylethyl)amino]carbonyl}benzyl)piperidin-4-yl]-4H-chromene-2-carboxamide

The title product was prepared according to example 103 by substituting 2-Pyrrolidin-1-yl-ethylamine for N1-Phenyl-ethane-1,2-diamine. ¹H NMR (500 MHz, Pyridine-d5) δ ppm 1.82-1.88 (m, 4H), 2.09-2.21 (m, 4H), 2.42-2.49 (m, 2H), 3.15-3.20 (m, 2H), 3.40-3.44 (bs, 4H), 3.58 (t, 2H, J=5.85 Hz), 3.86 (s, 2H), 4.04 (q, 2H, J=5.77 Hz), 4.32-4.41 (m, 1H), 6.55 (dd, 1H, J=9.36, 2.34 Hz), 7.14 (td, 1H, J=8.54, 2.44 Hz), 7.42 (s, 1H), 7.57 (d, 2H, J=8.27 Hz), 8.28 (dd, 1H, J=8.97, 6.32 Hz), 8.29 (d, 2H, J=8.27 Hz), 9.61 (t, 1H, J=5.54 Hz), 9.77 (d, 1H, J=7.49 Hz); MS (ESI) m/z 521 [M+H]⁺.

### Example 112

### N-[(3R,4S)-1-(1,3-benzodioxol-5-ylmethyl)-3-methylpiperidin-4-yl]-7-methoxy-4-oxo-4H-chromene-2-carboxamide

Prepared exactly as example 59 except the title compound was the less polar diastereomer on normal phase TLC (19 mg, Rf=0.75 90/10 CH₂Cl₂/MeOH). MS (ESI, MeOH/NH4OH) m/z 451 [M+H], 135, 449 [M−H]. ¹H NMR (300 MHz, DMSO-d6) δ ppm 0.99 (d, 3H, J=6.70 Hz), 1.57-1.66 (m, 1H), 1.84-1.98 (m, 1H), 2.05-2.24 (m, 3H), 2.69-2.79 (m, 1H), 3.37-3.48 (m, 1H), 3.94 (s, 3H), 3.93-4.04 (m, 1H), 4.01 (s, 2H), 5.99 (s, 2H), 6.76 (s, 1H), 6.72-6.90 (m, 3H), 7.10 (dd, 1H, J=8.86, 2.33 Hz), 7.28 (d, 1H, J=2.37 Hz), 7.95 (d, 1H, J=8.90 Hz), 8.52 (d, 1H, J=7.54 Hz).

### Example 113

### 7-fluoro-N-{1-[4-({[2-(3-fluorophenyl)ethyl]amino}carbonyl)benzyl]piperidin-4-yl}-4-oxo-4H-chromene-2-carboxamide

The title product was prepared according to example 103 by substituting 2-(3-Fluoro-phenyl)-ethylamine for N1-Phenyl-ethane-1,2-diamine. 1H NMR (500 MHz, PYRIDINE-D5) δ ppm 1.75-1.91 (m, 2H), 1.97-2.12 (m, 4H), 2.79-2.90 (m, J=12.21 Hz, 2H), 3.05 (t, J=7.17 Hz, 2H), 3.44 (s, 2H), 3.87 (m, 2H), 4.19-4.30 (m, 1H), 6.40 (dd, J=9.31, 2.29 Hz, 1H), 6.91-7.01 (m, 1H), 7.02-7.11 (m, 2H), 7.11-7.20 (m, 3H), 7.42 (d, J=8.24 Hz, 2H), 8.21 (d, J=7.93 Hz, 2H), 8.29 (dd, J=8.85, 6.41 Hz, 1H), 9.17 (t, J=5.80 Hz, 1H), 9.60 (d, J=7.63 Hz, 1H); MS (ESI) m/z 546 [M+H]⁺.

### Example 114

### 7-fluoro-4-oxo-N-[1-(4-{[(2-piperidin-1-ylethyl)amino]carbonyl}benzyl)piperidin-4-yl]-4H-chromene-2-carboxamide

The title product was prepared according to example 103 by substituting 2-piperidin-1-yl-ethylamine for N1-Phenyl-ethane-1,2-diamine. ¹H NMR (500 MHz, Pyridine-d5) δ ppm 1.23-1.35 (m, 2H), 1.45 (p, 4H, J=5.63 Hz), 1.77-1.92 (m, 2H), 2.00-2.09 (m, 4H), 2.32-2.38 (m, 4H), 2.60 (t, 2H, J=6.61 Hz), 2.84 (d, 2H, J=12.21 Hz), 3.44 (s, 2H), 3.78 (q, 2H, J=6.29 Hz), 4.20-4.29 (m, 1H), 6.45 (dd, 1H, J=9.36, 2.34 Hz), 7.14 (td, 1H, J=8.52, 2.44 Hz), 7.42 (d, 2H, J=8.27 Hz), 7.44 (s, 1H), 8.14-8.22 (m, 2H), 8.28 (dd, 1H, J=8.82, 6.34 Hz), 8.66 (t, 1H, J=5.45 Hz), 9.52-9.54 (m, 1H); MS (ESI) m/z 535 [M+H]⁺.

### Example 115

### N-[1-(3-amino-4-methylbenzyl)piperidin-4-yl]-7-fluoro-4-oxo-4H-chromene-2-carboxamide

### Example 115A

### N-[1-(3-nitro-4-methylbenzyl)piperidin-4-yl]-7-fluoro-4-oxo-4H-chromene-2-carboxamide

Example 115A was prepared according to the procedure outlined in Example 60, substituting 4-methyl-3-nitro-benzaldehyde for piperonal. ¹H NMR (500 MHz, DMSO-d6) δ ppm 1.67-1.70(m, 2H), 1.79-1.81(m, 2H), 2.10(t, 2H), 2.51(s, 3H), 2.83-2.86(m, 2H), 3.57(s, 2H), 3.80-3.82(m, 1H), 6.82(s, 1H), 7.40-7.47(m, 2H), 7.55-7.60(m, 2H), 7.93(s, 1H), 8.12(dd, 1H, J=6.55, 2.50), 8.81(d, 1H, J=8.11); MS (ESI) m/e 438.0 (M−H)⁺.

### Example 115

### N-[1-(3-amino-4-methylbenzyl)piperidin-4-yl]-7-fluoro-4-oxo-4H-chromene-2-carboxamide

A 200 mL round bottom flask was charged with N-[1-(3-nitro-4-methylbenzyl)piperidin-4-yl]-7-fluoro-4-oxo-4H-chromene-2-carboxamide (894 mg, 2.04 mmol), iron powder (1.137 g, 20.4 mmol), NH₄Cl (87.3 mg, 1.63 mmol), EtOH (50 mL) and water (25 mL). The mixture was completed after stirring at 80° C. for 5 hours. Filtered the mixture through a silica gel plug. The filtrate was concentrated to provide the pure title compound (755 mg). Yield 90.5%. ¹H NMR (400 MHz, DMSO-d6) δ ppm 1.62 (qd, 2H, J=11.90, 3.76), 1.76-1.80 (m, 2H), 1.93-1.97 (m, 2H), 2.01 (s, 3H), 2.82 (d, 2H, J=11.24), 3.28 (s, 2H), 3.58-3.85 (m, 1H), 4.55-4.84 (bs, 2H), 6.39 (d, 1H, J=7.41), 6.55 (s, 1H), 6.80-6.85 (m, 2H), 7.41 (td, 1H, J=8.67, 2.49), 7.61 (dd, 1H, J=9.45, 2.46), 8.10 (dd, 1H, J=8.88, 6.33), 8.81 (d, 1H, J=7.84 Hz); MS (ESI) m/e 408.2 (M−H)⁺.

### Example 116

### 7-chloro-6-fluoro-4-oxo-N-{1-[(2E)-3-phenylprop-2-enyl]piperidin-4-yl}-4H-chromene-2-carboxamide

### Example 116A

### Acetic acid 3-chloro-4-fluoro-phenyl ester

A 100 mL round bottom flask was charged with 3-Chloro-4-fluoro-phenol (5 g, 34.1 mmol), pyridine (8.3 mL, 102.6 mmol), CH₂Cl₂ (10 mL), and acetic anhydride (3.54 mL, 37.4 mmol). The solution was stirred at room temperature for 16 hours. The solution was washed with 10% acetic acid/water. The organic layer was concentrated to dryness to provide acetic acid 3-chloro-4-fluoro-phenyl ester as a white solid. ¹H NMR (300 MHz, DMSO-d₆) δ ppm 2.25 (s, 3H), 7.20 (m, 1H), 7.50 (m, 2H).

### Example 116B

### 1-(4-Chloro-5-fluoro-2-hydroxy-phenyl)-ethanone

A 100 mL round bottom flask was charged with acetic acid 3-chloro-4-fluoro-phenyl ester (2 g, 10.6 mmol), aluminum chloride (4 g, 30 mmol), and CH₂Cl₂ (10 mL). The solution was stirred at room temperature for 1 hour. CH₂Cl₂ was then distilled off using a short-path distillation (oil bath temperature=70 C). The mixture was slowly heated to 120 C and HCl gas evolved. After 10 minutes, the bath temperature was increased to 140° C. and the mixture was stirred for 2 hours. The mixture was allowed to cool to room temperature and then diluted with 50% HCl and water. Solid was filtered and washed with water to provide 1-(4-Chloro-5-fluoro-2-hydroxy-phenyl)-ethanone as a solid. ¹H NMR (300 MHz, DMSO-d₆) δ ppm 2.60 (s, 3H), 7.2 (d, 1H), 7.85 (d, 1H), 11.70 (s, 1H).

### Example 116C

### 7-Chloro-6-fluoro-4-oxo-4H-chromene-2-carboxylic acid

To a solution of 1-(4-Chloro-5-fluoro-2-hydroxy-phenyl)-ethanone (1.25 g, 6.63 mmol) in diethyl oxalate (5.4 mL) was added NaOEt (9.0 mL, 23.0 mmol, 20 weight % in EtOH), and the mixture was heated to 50° C. for 0.5 hours. The mixture was cooled to room temperature, diluted with Et₂O (25 mL), and filtered. The yellow solid was suspended in a solution of concentrated HCl (1 mL) in AcOH (7 mL), and heated to reflux for 1.5 hours. 6 N HCl (4 mL) was added to the reaction, and it was heated to reflux for an additional 16 hours. The mixture was then cooled to room temperature, diluted with H₂O (25 mL), and filtered. The solid was air-dried to provide the title compound as a beije solid. ¹H NMR (300 MHz, DMSO-d₆) δ ppm 3.5 (s, 1H), 6.9 (s, 1H), 7.90 (d, 1H), 8.25 (d, 1H); MS (ESI) m/z 241 [M−H]⁻.

### Example 116D

### 6-Fluoro-7-chloro-4-oxo-4H-chromene-2-carboxylic acid piperidin-4-ylamide

A flask was charged with 7-Chloro-6-fluoro-4-oxo-4H-chromene-2-carboxylic acid (0.268 g, 1.10 mmol), 4-Amino-piperidine-1-carboxylic acid tert-butyl ester (0.232 g, 1.10 mmol), EDCI (0.212 g, 1.10 mmol), HOBT (0.149 g, 1.10 mmol), NMM (0.48 mL, 4.40 mmol) and 1 mL of DMF. The solution was stirred at 55° C. for 16 hours. The solution was diluted with water (1 mL), extracted 3 times CH₂Cl₂ (5 mL), combined organics were concentrated to dryness. The crude product was purified on a FlashMaster II silica column using gradient conditions from 100% CH₂Cl₂ to 5% methanol/CH₂Cl₂. Product fractions were combined and concentrated to dryness. The compound was taken up in CH₂Cl₂ (5 mL) and treated with trifluoroacetic acid (1 mL) for 16 hour. Solution was concentrated to dryness and then diluted with diethyl ether (20 mL). The resulting solid was filtered and washed with diethyl ether to provide 6-Fluoro-7-chloro-4-oxo-4H-chromene-2-carboxylic acid piperidin-4-ylamide as the TFA salt. ¹H NMR (300 MHz, DMSO-D6) δ ppm 1.74-1.83 (m, 2H) 1.99 (s, 2H) 3.07 (s, 2H) 3.35 (d, J=10.85 Hz, 2H) 4.09 (td, J=7.37, 3.22 Hz, 1H) 6.88 (s, 1H) 7.95 (d, J=8.81 Hz, 1H) 8.10 (d, J=6.10 Hz, 1H) 8.40 (s, 1H) 8.60 (s, 1H) 9.01 (d, J=7.46 Hz, 1H) MS (ESI) m/z 325 [M+H]⁺.

### Example 116

### 7-chloro-6-fluoro-4-oxo-N-{1-[(2E)-3-phenylprop-2-enyl]piperidin-4-yl}-4H-chromene-2-carboxamide

A vial was charged with 6-Fluoro-7-chloro-4-oxo-4H-chromene-2-carboxylic acid piperidin-4-ylamide (53 mg, 0.12 mmol), cinnamyl chloride (0.026 mL, 0.18 mmol), triethylamine (0.078 mL, 0.56 mmol), and THF (0.4 mL). Solution was shaken at room temperature for 16 hours then purified on a RP-HPLC to provide 7-chloro-6-fluoro-4-oxo-N-{1-[(2E)-3-phenylprop-2-enyl]piperidin-4-yl}-4H-chromene-2-carboxamide. ¹H NMR (300 MHz, DMSO-d6) δ ppm 1.70-1.96 (m, 2H), 1.99-2.18 (m, 2H), 3.08-3.24 (m, 2H), 3.53-3.63 (m, 2H), 3.88-3.95 (m, 2H), 4.02-4.14 (m, 1H), 6.27-6.46 (m, 1H), 6.88 (s, 1H), 7.27-7.45 (m, 4H), 7.53-7.57 (m, 2H), 7.95 (d, 1H, J=8.69 Hz), 8.10 (d, 1H, J=5.97 Hz), 9.07 (d, 1H, J=7.56 Hz), 9.55 (s, 1H) MS (ESI) m/z 441 [M+H]⁺.

### Example 117

### N-[1-(1,3-benzodioxol-5-ylmethyl)piperidin-4-yl]-7-nitro-4-oxo-4H-chromene-2-carboxamide

The title product was prepared according to example by substituting 7-(nitro)-4-oxo-4H-chromene-2-carboxylic acid for 7-(difluoromethoxy)-4-oxo-4H-chromene-2-carboxylic acid. 1H NMR (500 MHz, PYRIDINE-D5) δ ppm 2.20-2.31 (m, 3H), 2.30-2.39 (m, 3H), 2.61-2.71 (m, 2H), 3.31-3.44 (m, 2H), 3.91-4.02 (m, 2H), 4.37-4.53 (m, 1H), 6.87-6.90 (m, 2H), 6.96-6.99 (m, 1H), 7.13-7.19 (m, J=1.22 Hz, 1H), 7.40-7.48 (m, 1H), 8.15 (dd, J=8.85, 2.14 Hz, 1H), 8.34-8.41 (m, J=8.85 Hz, 1H), 10.15-10.30 (m, J=7.63 Hz, 1H); MS (ESI) m/z 452 [M+H]⁺.

### Example 118

### 7-chloro-N-[1-(5,6-difluoro-2,3-dihydro-1H-inden-1-yl)piperidin-4-yl]-6-fluoro-4-oxo-4H-chromene-2-carboxamide

### Example 118A

### 5,6-Difluoro-indan-1-one

A flask was charged with 3,4-difluorohydrocinnamic acid (4 g, 21.5 mmol) and polyphosphoric acid (100 mL). Solution was heated to 80° C. and slowly stirred over 72 hours. Solution was carefully poured over ice. Solid was filtered and dried to provide 5,6-Difluoro-indan-1-one. 1H NMR (300 MHz, DMSO-D6) δ ppm 2.68 (m, 2H) 3.05-3.12 (m, 2H) 7.68 (ddd, J=13.39, 9.66, 7.46 Hz, 2H).

### Example 118

### 7-chloro-N-[1-(5,6-difluoro-2,3-dihydro-1H-inden-1-yl)piperidin-4-yl]-6-fluoro-4-oxo-4H-chromene-2-carboxamide

A vial was charged with 6-Fluoro-7-chloro-4-oxo-4H-chromene-2-carboxylic acid piperidin-4-ylamide as prepared in Example 116D (40 mg, 0.12 mmol), 5,6-Difluoro-indan-1-one (20 mg, 0.12 mmol), titanium isopropoxide (1 mL, 3.4 mmol), and THF (0.4 mL). Solution was shaken at 50° C. for 4 hours then cooled to room temperature. 1M Sodium Cyanoborohydride in THF (0.5 mL, 5 mmol) was added and solution was shaken at room temperature for 16 hours. Solution was treated with water (1 mL), filtered through celite, and purified on a RP-HPLC to provide 7-chloro-N-[1-(5,6-difluoro-2,3-dihydro-1H-inden-1-yl)piperidin-4-yl]-6-fluoro-4-oxo-4H-chromene-2-carboxamide. 1H NMR (300 MHz, DMSO-D6) δ ppm 1.74-1.96 (m, 3H) 1.98-2.17 (m, 2H) 2.84-2.97 (m, 2H) 2.98-3.13 (m, 2H) 3.17 (s, 2H) 4.07 (s, 1H) 4.97 (d, J=7.46 Hz, 1H) 6.88 (s, 1H) 7.51 (dd, J=10.68, 7.63 Hz, 1H) 7.68 (dd, J=10.17, 7.80 Hz, 1H) 7.88-8.01 (d, J=8.82 Hz, 1H) 8.10 (d, J=6.10 Hz, 1H) 9.04 (d, J=7.46 Hz, 1H) 9.68 (s, 1H) MS (ESI) m/z 477 [M+H]⁺.

### Example 119

### N-[1-(2,1,3-benzothiadiazol-5-ylmethyl)piperidin-4-yl]-6,7-difluoro-4-oxo-4H-chromene-2-carboxamide

Example 55E (31 mg, 0.100 mmol), 2,1,3-benzothiadiazole-5-carbaldehyde (16 mg, 0.100 mmol), were processed as described in Example 70 to provide Example 119. ¹H NMR (500 MHz, DMSO-d6) δ ppm 1.64-1.73 (m, 2H), 1.80-1.86 (m, 2H), 2.13-2.19 (m, 2H), 2.87-2.93 (m, 2H), 3.71 (s, 2H), 3.77-3.85 (m, 1H), 6.85 (s, 1H), 7.73 (dd, 1H, J=9.08, 1.45 Hz), 7.91 (dd, 1H, J=10.60, 6.79 Hz), 7.96 (s, 1H), 8.00 (dd, 1H, J=9.54, 8.93 Hz), 8.06 (d, 1H, J 9.00 Hz), 8.86 (d, 1H, J=7.93 Hz); MS (ESI, MeOH/NH4OH) m/z 457 [M+H]⁺, 455 [M−H]⁺.

### Example 120

### 7-fluoro-4-oxo-N-{1-[(3-oxo-3,4-dihydro-2H-1,4-benzoxazin-6-yl)methyl]piperidin-4-yl}-4H-chromene-2-carboxamide

### Example 120A

### 7-Fluoro-4-oxo-4H-chromene-2-carboxylic acid[1-(3-amino-4-hydroxy-benzyl)-piperidin-4-yl]-amide

A flask was charged with 7-Fluoro-4-oxo-4H-chromene-2-carboxylic acid piperidin-4-ylamide hydrochloride salt (1 g, 2.8 mmol), 4-Hydroxy-3-nitro-benzaldehyde (1.02 g, 6.1 mmol), sodium triacetoxyborohydride (2.0 g, 9.4 mmol), and 10 ml THF were stirred at room temperature for 16 hours. Solution was concentrated and purified on a FlashMaster II silica column using gradient conditions from 100% CH₂Cl₂ to 5% methanol/CH₂Cl₂. Product fraction were concentrated to dryness and suspended in 1:1 ethanol/water (25 mL) with Fe powder (1 g) and ammonium chloride (1 g). Solution was refuxed for 16 hours then filtered and concentrated to provide 7-Fluoro-4-oxo-4H-chromene-2-carboxylic acid[1-(3-amino-4-hydroxy-benzyl)-piperidin-4-yl]-amide. MS (ESI) m/z 412 [M+H]⁺.

### Example 120

### 7-fluoro-4-oxo-N-{1-[(3-oxo-3,4-dihydro-2H-1,4-benzoxazin-6-yl)methyl]piperidin-4-yl}-4H-chromene-2-carboxamide

A flask was charged with 7-Fluoro-4-oxo-4H-chromene-2-carboxylic acid[1-(3-amino-4-hydroxy-benzyl)-piperidin-4-yl]-amide (50 mg, 0.12 mmol), potassium carbonate (50 mg, 0.36 mmol), acetone (3 mL), and DMF (0.4 mL). Mixture was cooled to −78° C. and bromoacetylbromide (0.011 mL, 0.13 mmol) was added. Solution was slowly warmed to 60° C. After 3 hours, solution was filtered, concentrated and purified on a RP-HPLC to provide 7-fluoro-4-oxo-N-{1-[(3-oxo-3,4-dihydro-2H-1,4-benzoxazin-6-yl)methyl]piperidin-4-yl}-4H-chromene-2-carboxamide. 1H NMR (300 MHz, DMSO-D6) δ ppm 1.77 (d, J=10.85 Hz, 3H) 2.07 (d, J=8.82 Hz, 3H) 3.14 (d, J=14.58 Hz, 3H) 3.45 (m, 3H) 4.21 (s, 2H) 4.62 (s, 1H) 6.81-6.95 (m, 2H) 7.05 (s, 1H) 7.44 (td, J=8.65, 2.37 Hz, 1H) 7.61 (dq, J=6.10, 3.39 Hz, 1H) 8.12 (dd, J=8.82, 6.44 Hz, 1H) 9.06 (d, J=7.12 Hz, 1H) MS (ESI) m/z 452 [M+H]⁺.

### Example 121

### 7-fluoro-4-oxo-N-{1-[4-({[2-(pyridin-2-ylamino)ethyl]amino}carbonyl)benzyl]piperidin-4-yl}-4H-chromene-2-carboxamide

The title product was prepared according to example 103 by substituting N1-Pyridin-2-yl-ethane-1,2-diamine for N1-Phenyl-ethane-1,2-diamine. 1H NMR (500 MHz, PYRIDINE-D5) δ ppm 1.78-1.93 (m, 2H), 1.99-2.12 (m, 4H), 2.80-2.92 (m, 2H), 3.45 (s, 2H), 3.85-3.99 (m, 4H), 4.14-4.32 (m, 1H), 6.36-6.45 (m, 1H), 6.57 (dd, J=7.02, 5.80 Hz, 1H), 6.64 (d, J=8.24 Hz, 1H), 7.10-7.18 (m, 1H), 7.31-7.39 (m, 2H), 7.39-7.49 (m, 3H), 8.19 (d, J=8.24 Hz, 2H), 8.25-8.34 (m, 2H), 9.40 (t, J=5.04 Hz, 1H), 9.61 (d, J=7.63 Hz, 1H); MS (ESI) m/z 544 [M+H]⁺.

### Example 122

### N-{1-[(1-acetyl-1H-indol-6-yl)methyl]piperidin-4-yl}-7-fluoro-4-oxo-4H-chromene-2-carboxamide

### Example 122A

### 1-Acetyl-1H-indole-6-carbaldehyde

A 50 mL round bottom flask was charged with 1H-Indole-6-carbaldehyde (31 mg, 0.213 mmol), CH₂Cl₂(10 mL), CH₃COCl (20 mg, 0.256 mmol) and Et₃N (43 mg, 0.426 mmol). The mixture was stirred at room temperature overnight, then quenched with saturated NH₄Cl solution. Separated the two layers and the aqueous layer was extracted with CH₂Cl₂. The combined organic layer was washed with brine, dried over Na₂SO₄, concentrated to provide the crude product. Flash silica gel chromatography with EtOAc in Hexanes (0-15%) as eluent provided the title compound (19 mg, 48%). ¹H NMR (400 MHz, DMSO-d₆) δ ppm 2.28(s, 3H), 6.67(d, 1H, J 3.99), 7.60 (s, 2H), 7.93(d, 1H, J=3.68), 8.64(s, 1H), 9.86(s, 1H); MS (DCI) m/e 188.0 (M+H)⁺.

### Example 122

### N-{1-[(1-acetyl-1H-indol-6-yl)methyl]piperidin-4-yl}-7-fluoro-4-oxo-4H-chromene-2-carboxamide

Example 122 was prepared according to the procedure outlined in Example 60, substituting l-acetyl-1H-indole-6-carbaldehyde for piperonal. ¹H NMR (400 MHz, DMSO-d₆) δ ppm 1.77-1.87(m, 2), 2.05-2.08(m, 2H), 2.69(s, 3H), 3.15-3.43(m, 4H), 3.97(s, 2H), 4.44-4.45(m, 1H), 6.85(s, 1H), 7.38-7.45(m, 2H), 7.48-7.61(m, 2H), 7.73(d, 1H, J=7.97), 7.98(d, 1H, J=3.68), 8.12(dd, 1H, J=6.45, 2.45), 8.56(s, 1H), 9.05(d, 1H, J=7.37); MS (ESI) m/e 461.8(M+H)⁺.

### Example 123

### 7-fluoro-N-[1-(1-naphthylmethyl)piperidin-4-yl]-4-oxo-4H-chromene-2-carboxamide

The title compound was prepared according to the procedure for Example 79 substituting naphthalene-1-carbaldehyde for 3-fluoro-4-(3-piperidin-1-yl-propoxy)-benzaldehyde. 1H NMR (500 MHz, Solvent) δ ppm 2.06-2.18 (m, 4H), 2.31-2.46 (m, 2H), 3.07-3.19 (m, J=11.60 Hz, 2H), 4.09 (s, 2H), 4.29-4.41 (m, 1H), 7.22-7.28 (m, 2H), 7.36-7.43 (m, 2H), 7.51-7.65 (m, 4H), 7.91-7.97 (m, J=8.24 Hz, 1H), 7.97-8.01 (m, J=8.24 Hz, 1H), 8.31 (dd, J=8.85, 6.41 Hz, 1H), 8.45 (d, J=8.24 Hz, 1H); MS (ESI) m/z 431 [M+H]⁺.

### Example 124

### N-[1-(1,3-benzodioxol-4-ylmethyl)piperidin-4-yl]-7-[(3,4-dimethoxyphenyl)amino]-6-fluoro-4-oxo-4H-chromene-2-carboxamide

Example 55 (0.025 g, 0.06 mmol) was heated at 50° C. along with 3,4-dimethoxy phenylamine (0.01 g, 0.07 mmol) and potassium carbonate (0.015 g, 0.12 mmol) in DMF (1 mL) for 12 hours. The mixtures were then filtered, concentrated and purified by RP-HPLC to provide the title compound as a white solid. ¹H NMR (500 MHz, DMSO-d6) δ ppm 1.75-1.86 (m, 2H), 1.99-2.05 (m, 2H), 3.00-3.10 (m, 2H), 3.37-3.43 (m, 2H), 3.76 (s, 3H), 3.78 (s, 3H), 3.92-4.00 (m, 1H), 4.18-4.21 (m, 2H), 6.08 (s, 2H), 6.69 (s, 1H), 6.87-7.09 (m, 7H), 7.62 (d, 1H, J=10 Hz), 8.71 (s, 1H), 8.92 (d, 1H, J=5 Hz), 9.32 (br s, 1H); MS (ESI) m/z 576 [M+H]⁺.

### Example 125

### 7-chloro-N-{1-[(4-ethoxy-2-oxo-2H-chromen-7-yl)methyl]piperidin-4-yl}-6-fluoro-4-oxo-4H-chromene-2-carboxamide

### Example 125A

### 4-Ethoxy-7-methyl-chromen-2-one

A suspension of 4-Ethoxy-7-methyl-chromen-2-one (0.250 g, 1.23 mmol) and N-bromosuccinimide (0.241 g, 1.35 mmol) in carbon tetrachloride (17.5 mL) was treated with 2,2′-azobisisobutyronitrile (0.020 g, 0.122 mmol) and heated at reflux for 24 hours. The mixture was cooled to room temperature, filtered through a pad of celite, and the filtrate was concentration to provide a white solid. The solid was recrystallized from hot acetonitrile to provide example 125A as an off-white solid that was sued in the next step with no further purification.

### Example 125

### 7-chloro-N-{1-[(4-ethoxy-2-oxo-2H-chromen-7-yl)methyl]piperidin-4-yl}-6-fluoro-4-oxo-4H-chromene-2-carboxamide

7-chloro-N-{1-[(4-ethoxy-2-oxo-2H-chromen-7-yl)methyl]piperidin-4-yl}-6-fluoro-4-oxo-4H-chromene-2-carboxamide (0.030 g, 92.6 mmol) and 7-Bromomethyl-4-ethoxy-chromen-2-one (0.0280 g, 0.0989 mmol) were stirred in DMF at 50° C. for 5 hours. After this time, the solvent was evaporated and the product purified via RP-HPLC to provide the title product as its TFA salt. 1H NMR (300 MHz, DMSO-D6) δ ppm 1.45 (t, J=6.95 Hz, 3H), 1.68-1.93 (m, 2H), 2.00-2.15 (m, 2H), 3.06-3.24 (m, 2H), 3.47-3.54 (m, 2H), 3.92-4.08 (m, 1H), 4.31 (q, J=6.89 Hz, 2H), 4.39-4.54 (m, J=3.73 Hz, 2H), 5.97 (s, 1H), 6.87 (s, 1H), 7.43-7.53 (m, J=7.80 Hz, 1H), 7.58 (s, 1H), 7.88-8.01 (m, J=8.48, 8.48 Hz, 2H), 8.09 (d, J=6.10 Hz, 1H), 8.95-9.11 (m, J=7.46 Hz, 1H); MS (ESI) m/z 527 [M+H]⁺.

### Example 126

### N-(1-{4-[amino(imino)methyl]benzyl}piperidin-4-yl)-7-fluoro-4-oxo-4H-chromene-2-carboxamide

The title compound was prepared according to the procedure for example 79 substituting 4-Formyl-benzamidine for 3-fluoro-4-(3-piperidin-1-yl-propoxy)-benzaldehyde. MS (ESI) m/z 423 [M+H]⁺.

### Example 127

### 7-fluoro-4-oxo-N-[1-(2-oxo-3,5,6,7-tetrahydro-2H-indeno[5,6-d][1,3]oxazol-5-yl)piperidin-4-yl]-4H-chromene-2-carboxamide

### Example 127A

### 3-(2-Oxo-2,3-dihydro-benzooxazol-6-yl)-acrylic acid methyl ester

Example 127A was prepared according to the procedure described in Example 85C, substituting carbonyl diimidazole (CDI) for 2-bromoacetyl bromide. MS (ESI) m/z 220 (M+H)⁺.

### Example 127B

### 3-(2-Oxo-2,3-dihydro-benzooxazol-6-yl)-propionic acid methyl ester

Example 127A was prepared according to the procedure described in Example 85D, substituting 3-(2-oxo-2,3-dihydro-benzooxazol-6-yl)-acrylic acid methyl ester for 3-(3-oxo-3,4-dihydro-2H-benzo[1,4]oxazin-7-yl)-acrylic acid methyl ester. MS (ESI) m/z 222 (M+H)⁺.

### Example 127C

### 3-(2-Oxo-2,3-dihydro-benzooxazol-6-yl)-propionic acid

Example 127C was prepared according to the procedure described in Example 85E, substituting 3-(2-oxo-2,3-dihydro-benzooxazol-6-yl)-propionic acid methyl ester for 3-(3-oxo-3,4-dihydro-2H-benzo[1,4]oxazin-7-yl)-propionic acid methyl ester. MS (ESI) m/z 208 (M+H)⁺.

### Example 127D

### 6,7-Dihydro-3H-indeno[5,6-d]oxazole-2,5-dione

Example 127D was prepared according to the procedure described in Example 85F, substituting 3-(2-oxo-2,3-dihydro-benzooxazol-6-yl)-propionic acid for 3-(3-oxo-3,4-dihydro-2H-benzo[1,4]oxazin-7-yl)-propionic acid. MS (ESI) m/z 190 (M+H)⁺.

### Example 127

### 7-fluoro-4-oxo-N-[1-(2-oxo-3,5,6,7-tetrahydro-2H-indeno[5,6-d][1,3]oxazol-5-yl)piperidin-4-yl]-4H-chromene-2-carboxamide

Example 127 was prepared according to the procedure described in Example 92, substituting 6,7-dihydro-3H-indeno[5,6-d]oxazole-2,5-dione for for 6-acetyl-3-ethyl-3H-benzothiazole-2-one. The crude residue was purified by RP-HPLC to provide the title compound as its mono-TFA salt. ¹H NMR (300 MHz, DMSO-d6) δ ppm 1.74-1.97 (m, 2H), 1.97-2.13 (m, 2H), 2.37-2.46 (m, 1H), 2.88-2.97 (m, 1H), 3.07-3.12 (m, 2H), 3.35-3.45 (m, 2H), 3.48-3.60 (m, 2H), 4.01-4.12 (m, 1H), 4.96 (d, 1H, J=8.1 Hz), 6.86 (s, 1H), 7.33 (m, 2H), 7.45 (dt, 1H, J=2.7 and 8.8 Hz), 7.61 (dd, 1H, J=6.5 and 8.8 Hz), 8.12 (dd, 1H, J=6.5 and 8.8 Hz), 9.04 d(1H, J=7.5 Hz), 9.44 (br s, 1H), and 11.83 (s, 1H); MS (ESI) m/z 464 (M+H)⁺.

### Example 128

### 7-fluoro-N-{1-[(1-methyl-1H-indol-4-yl)methyl]piperidin-4-yl}-4-oxo-4H-chromene-2-carboxamide

Example 128 was prepared according to the procedure outlined in Example 60, substituting 1-methyl-1H-indole-4-carbaldehyde for piperonal. ¹H NMR (500 MHz, DMSO-d₆) δ ppm 1.82-1.89(m, 2H), 2.04-2.07(m, 2H), 3.22-3.26(m, 2H), 3.46-3.48(m, 2H), 3.83(s, 3H), 3.97(s, 2H), 4.54-4.55(m, 1H), 6.78(d, 1H, J=3.12), 7.02(s, 1H), 7.12(s, 1H), 7.22-7.27(m, 2H), 7.41-7.48(m, 2H), 7.57-7.61(m, 1H), 8.12(dd, 1H, J=6.24, 2.50), 9.06 (d, 1H, J=7.49); MS (ESI) m/e 432.1 (M−H)⁺.

### Example 129

### 7-fluoro-4-oxo-N-[1-(4-{2-oxo-2-[(2-pyrrolidin-1-ylethyl)amino]ethyl}benzyl)piperidin-4-yl]-4H-chromene-2-carboxamide

### Example 129A

### {4-[(7-Fluoro-4-oxo-4H-chromene-2-carbonyl)-amino]-piperidin-1-yl}-acetic acid

Example 109 (0.100 g, 0.214 mmol) was dissolved in 4 mL of 80% THF/H₂O and LiOH monohydrate (18.0 mg, 0.428 mmol) was added. The mixture was stirred at room temperature for about 2 hours, then neutralized with 10% HCl, and extracted with EtOAc. Organic layers was washed with brine, dried over Na₂SO₄, and concentrated to provide a colorless oil (80 mg) in 87% yield. MS (DCI) m/z 349 [M+H]⁺.

### Example 129

### 7-fluoro-4-oxo-N-[1-(4-{2-oxo-2-[(2-pyrrolidin-1-ylethyl)amino]ethyl}benzyl)piperidin-4-yl]-4H-chromene-2-carboxamide

A 10 mL culture tube with screw cap was charged with example 129A (40 mg, 0.091 mmol), 2-pyrrolidin-1-yl-ethylamine (20 mg, 0.18 mmol), EDCI (28 mg, 0.15 mmol), HOBT (20 mg, 0.15 mmol), NMM (32 mg, 0.30 mmol) and 2 mL of DMF, and was placed on a shaker for 6 hours. After this time, the DMF was removed in vacuo, and the residue was dissolved in 1.5 mL of a 1:1 mixture of DMSO/MeOH and purified by preparative reverse-phase HPLC to provide the title product. ¹H NMR (300 MHz, DMSO-d₆) ppm 1.75-2.15 (m, 8H), 2.95-3.25 (m, 8H), 3.35-3.65 (m, 6H), 4.01 (m, 1H), 4.30 (m, 2H), 6.85 (s, 1H), 7.35-7.48 (m, 5H), 7.60 (dd, 1H, J₁=9.49, J₂=2.71), 7.96 (dd, 1H, J₁=8.81, J₂=6.43), 8.37 (m, 1H),9.08 (d, 1H, J=7.46); MS (ESI) m/z 535 [M+H]⁺.

### Example 130

### 7-methoxy-4-oxo-N-[1-(quinolin-7-ylmethyl)piperidin-4-yl]-4H-chromene-2-carboxamide

Example 2C and example 3A were processed as described in example 2 to provide Example 130. ¹H NMR (300 MHz, DMSO-d6) δ ppm 1.78-1.95 (m, 2H), 2.04-2.14 (m, 2H), 3.14-3.29 (m, 2H), 3.48-3.58 (m, 2H), 3.92 (s, 3H), 4.57 (d, 2H, J=4.49 Hz), 6.78 (s, 1H), 7.12 (dd, 1H, J=8.86, 2.42 Hz), 7.19 (d, 1H, J=2.37 Hz), 7.65 (dd, 1H, J=8.27, 4.20 Hz), 7.73 (dd, 1H, J=8.39, 1.36 Hz), 7.96 (d, 1H, J=8.90 Hz), 8.13 (d, 1H, J=8.31 Hz), 8.25 (s, 1H), 8.45-8.50 (m, 1H), 9.01 (dd, 1H, J=4.24, 1.61 Hz), 9.04 (d, 1H, J=7.54 Hz), 9.66 (s, 1H);); MS (ESI) m/z 444 [M+H]⁺.

### Example 131

### 7-fluoro-4-oxo-N-{1-[(2E)-3-phenylprop-2-enyl]piperidin-4-yl}-4H-chromene-2-carboxamide

A vial was charged with 7-Fluoro-4-oxo-4H-chromene-2-carboxylic acid piperidin-4-ylamide (48 mg, 0.12 mmol), cinnamyl chloride (0.026 mL, 0.18 mmol), triethylamine (0.078 mL, 0.56 mmol), and THF (0.4 mL). Solution was shaken at room temperature for 16 hours then purified on a RP-HPLC to provide 7-fluoro-4-oxo-N-{1-[(2E)-3-phenylprop-2-enyl]piperidin-4-yl}-4H-chromene-2-carboxamide. ¹H NMR (300 MHz, DMSO-d6) δ ppm 1.55-1.73 (m, 2H), 1.75-1.91 (m, 2H), 1.94-2.17 (m, 2H), 2.83-3.01 (m, 2H), 3.09-3.16 (m, 2H), 3.71-3.86 (m, 1H), 6.32 (dt, 1H, J=15.86, 6.45 Hz), 6.54 (d, 1H, J=15.96 Hz), 6.83 (s, 1H), 7.25 (t, 1H, J=7.22 Hz), 7.33 (t, 2H, J=7.40 Hz), 7.42 (dd, 1H, J=8.75, 2.39 Hz), 7.42-7.48 (m, 2H), 7.63 (dd, 1H, J=9.47, 2.45 Hz), 8.12 (dd, 1H, J=8.90, 6.36 Hz), 8.79-8.93 (m, 1H) MS (ESI) m/z 407 [M+H]⁺.

### Example 132

### N-[1-(1,3-benzodioxol-5-ylmethyl)piperidin-4-yl]-6-fluoro-7-(methylamino)-4-oxo-4H-chromene-2-carboxamide

Example 132 was prepared using a similar procedure as described for Example 124 substituting methyl amine for 3,4-dimethoxy phenylamine in the step described in Example 124. ¹H NMR (500 MHz, DMSO-d6) δ ppm 1.64 (qd, 2H, J=11.97, 3.76 Hz), 1.74-1.78 (m, 2H), 1.98 (t, 2H, J=11.51 Hz), 2.79-2.84 (m, 2H), 2.83 (d, 3H, J=4.73 Hz), 3.34 (s, 2H), 3.69-3.82 (m, 1H), 5.97 (s, 2H), 6.64 (s, 1H), 6.73-6.75 (m, 2H), 6.83 (d, 1H, J=7.83 Hz), 6.85 (d, 1H, J=1.59 Hz), 6.97-7.01 (m, 1H), 7.46-7.49 (m, 1H), 8.75 (d, 1H, J=8.05 Hz); MS (ESI) m/z 454 [M+H]⁺.

### Example 133

### N-[1-(4-{[(2E)-2-(ethoxyimino)ethyl]oxy}-3-fluorobenzyl)piperidin-4-yl]-7-fluoro-4-oxo-4H-chromene-2-carboxamide

### Example 133A

### 4-(2,2-dimethoxy-ethoxy)-3-fluoro-benzaldehyde

Example 133A was prepared according to the procedure described in Example 79A, substituting 2,2-dimethoxyethanol for 3-piperidin-1-yl-propan-1-ol. MS (ESI) m/z 229 (M+H)⁺.

### Example 133B

### 7-fluoro-4-oxo-4H-chromene-2-carboxylic acid {1-[4-(2,2-dimethoxy-ethoxy)-3-fluoro-benzyl]-piperidin-4-yl]-amide

Example 133B was prepared according to the procedure described in Example 79, substituting 4-(2,2-dimethoxy-ethoxy)-3-fluoro-benzaldehyde for 3-fluoro-4-(3-piperidin-1-yl-propoxy)-benzaldehyde. The crude product was used without further purification. MS (ESI) m/z 503 (M+H)⁺.

### Example 133C

### 7-fluoro-4-oxo-4H-chromene-2-carboxylic acid {1-[3-fluoro-4-(2-oxo-ethoxy)-benzyl]-piperidin-4-yl}-amide

To a solution of 7-fluoro-4-oxo-4H-chromene-2-carboxylic acid {1-[4-(2,2-dimethoxy-ethoxy)-3-fluoro-benzyl]-piperidin-4-yl}-amide (753 mg, 1.50 mmol) in acetone (8 mL) was added 2N HCl (4 mL). The solution was heated to 50° C. overnight and then cooled to room temperature. The mixture was diluted with EtOAc (10 mL) and H₂O (5 mL). The layers were separated, and the aqueous was extracted with additional EtOAc (3×10 mL). The combined organic layers were dried with anhydrous Na₂SO₄, filtered, and concentrated under reduced pressure to provide the title compound as a solid, which was used directly in the subsequent step without further purification. MS (ESI) m/z 457 (M+H)⁺.

### Example 133

### N-[1-(4-{[(2E)-2-(ethoxyimino)ethyl]oxy}-3-fluorobenzyl)piperidin-4-yl]-7-fluoro-4-oxo-4H-chromene-2-carboxamide

To a solution of 7-fluoro-4-oxo-4H-chromene-2-carboxylic acid {1-[3-fluoro-4-(2-oxo-ethoxy)-benzyl]-piperidin-4-yl}-amide (15 mg, 0.033 mmol) and Et₃N (0.018 mL, 0.132 mmol) in EtOH (1 mL) was added O-ethylhydroxylamine hydrochloride salt (6.4 mg, 0.066 mmol). The solution was stirred at room temperature for 16 hours. The mixture was then concentrated under reduced pressure, and the residue was purified by RP-HPLC to provide the title compound as its mono-TFA salt. ¹H NMR (300 MHz, DMSO-d6) δ ppm 1.15-1.26 (m, 3H), 1.74-1.90 (m, 2H), 2.07 (br d, 2H, J=13.2 Hz), 3.01-3.16 (m, 2H), 3.27-3.35 (m, 2H), 3.93-4.12 (m, 4H), 4.22-4.29 (m, 2H), 4.78 (d, 1H, J=5.8 Hz), 4.96 (d, 1H, J=3.7 Hz), 6.85 (s, 1H), 7.22-7.83 (m, 2H), 7.44 (dt, 1H, J=2.4 and 8.8 Hz), 7.60 (dd, 1H, J=2.4 and 9.5 Hz), 7.65 (t, 1H, J=5.4 Hz), 8.12 (dd, 1H, J=6.5 and 9.2 Hz), 9.04 (d, 1H, J=7.5 Hz), and 9.40 (br s, 1H); MS (ESI) m/z 500 (M+H)⁺.

### Example 134

### N-[1-(1,3-benzodioxol-5-ylmethyl)piperidin-4-yl]-6-fluoro-4-oxo-7-{[4-(rifluoromethyl)benzyl]amino}4H-chromene-2-carboxamide

Example 134 was prepared using a similar procedure as described for Example 124 substituting 4-trifluoromethyl benzylamine for 3,4-dimethoxy phenylamine in the step described in Example 124. ¹H NMR (400 MHz, DMSO-d6) δ ppm 1.73-1.85 (m, 2H), 1.93-2.05 (m, 2H), 3.00-3.10 (m, 2H), 3.37-3.43 (m, 2H), 4.18-4.21 (m, 2H), 4.59-4.64 (m, 1H), 6.08 (s, 2H), 6.62-6.68 (m, 2H), 6.90-7.09 (m, 3H), 7.53-7.60 (m, 2H), 7.67-7.74 (m, 2H), 7.88 (d, 1H, J=8 Hz), 8.15 (d, 1H, J=8 Hz), 8.88 (m, 1H), 9.29 (br s, 1H); MS (ESI) m/z 598 [M+H]⁺.

### Example 135

### N-{1-[(5-chloro-2-methyl-1,3-benzoxazol-6-yl)methyl]piperidin-4-yl}-7-fluoro-4-oxo-4H-chromene-2-carboxamide

### Example 135A

Sodium hydroxide (6.50 g, 163 mmol) was added carefully to a solution of 5′-chloro-2′-hydroxy-4′-methylacetophenone (6.00 g, 32.5 mmol), hydroxylamine hydrochloride (3.39 g, 48.8 mmol), ethanol (20 mL), and water (4 mL), and the temperature was raised to 80° C. After 30 minutes the solution was cooled to room temperature and diluted with a mixture of water (200 mL) and concentrated HCl (30 mL). The resulting solid was collected by filtration and air-dried overnight to provide the title compound (5.62 g, 87%) as a white solid. Reference: A. Lachman. Org. Synth. 1943, Coll. Vol. 2, 70-71. ¹H NMR (500 MHz, DMSO-d6): δ ppm 2.23 (s, 3H), 2.27 (s, 3H), 6.87 (s, 1H), 7.44 (s, 1H), 11.53 (s, 1H), 11.61 (s, 1H); MS (APCI) m/z 200 [M+H]⁺, 198 [M−H]⁺.

### Example 135B

### 5-chloro-2,6-dimethyl-1,3-benzoxazole

Example 135A (500 mg, 2.50 mmol), zinc chloride (682 mg, 5.00 mmol), and o-xylene (0.5 mL) were heated thrice in the microwave at 150° C. for twenty minutes. Upon final cooling the drab solid was diluted with a 92:8 mixture of CH₂Cl₂:CH₃OH (100 mL), washed with 1N aqueous NaOH (3×100 mL), washed with brine (1×100 mL), dried (MgSO₄), filtered, and concentrated to a grainy residue that was purified by the Flashmaster (40 g Analogix column, 30 mL/min gradient of 0 to 50% EtOAc in hexane over 30 min) to provide the title compound (336 mg, 74%) as a white solid. Reference: A. Loupey and S. Regnier. Tett. Lett. 40 (1999), 6221-4. ¹H NMR (300 MHz, DMSO-d6): δ ppm 2.42 (s, 3H), 2.60 (s, 3H), 7.70 (s, 1H), 7.74 (s, 1H); MS (APCI) m/z 182 [M+H]⁺, 180 [M−H]⁺.

### Example 135C

### 6-(bromomethyl)-5-chloro-2-methyl-1,3-benzoxazole

N-Bromosuccinimide (318 mg, 1.78 mmol) and AIBN (29 mg, 0.178 mmol) were added sequentially to Example 135B (324 mg, 1.78 mmol) and carbon tetrachloride (6 mL). The orange, heterogeneous mixture was heated at 80° C. for 1 hours. The mixture was cooled, diluted with CH₂Cl₂ (40 mL), washed with 10% aqueous Na₂S₂O₃ (3×40 mL), washed with saturated aqueous NaHCO₃ (3×40 mL), dried (Na₂SO₄), filtered, and concentrated to a residue that was purified by Flashmaster (40 g Analogix column, 30 mL/min gradient of 0 to 50% EtOAc in hexane over 30 min) to provide the title compound (275 mg, 59%) as a white solid. Reference: K. Seio, K. Kumura, J. C. Bologna, M. Sekine. J. O. C. 68 (10), 2003, 3849-59. ¹H NMR (300 MHz, DMSO-d6): δ ppm 2.63 (s, 3H), 4.86 (s, 2H), 7.85 (s, 1H), 8.01 (s, 1H); MS (APCI) m/z 260 [⁷⁹Br+H]⁺, 262 [⁸¹Br+H]⁺.

### Example 135

### N-{1-[(5-chloro-2-methyl-1,3-benzoxazol-6-yl)methyl]piperidin-4-yl}-7-fluoro-4-oxo-4H-chromene-2-carboxamide

Example 60D (40 mg, 0.138 mmol), Example 135C (36 mg, 0.138 mmol), cesium carbonate (67 mg, 0.207 mmol), and DMF (0.5 mL) were combined in a dram vial and agitated on a shaker overnight. After 16 h the mixture was diluted with CH₂Cl₂ (20 mL), washed with saturated aqueous NaHCO₃ (1×20 mL), washed with brine (1×20 mL), dried (Na₂SO₄), filtered, and concentrated. The crude material was loaded onto a sep pack (1 g, Alltech) and eluted with 1:1 hexane:EtOAc to provide the title product (24 mg, 37%) as a white solid. ¹H NMR (500 MHz, DMSO-d6): δ ppm 1.70 (td, J=11.93, 3.59 Hz, 2H), 1.83 (dd, J=12.01, 2.65 Hz, 2H), 2.17-2.24 (m, 2H), 2.62 (s, 3H), 2.90 (d, J=11.23 Hz, 2H), 3.67 (s, 2H), 3.83 (dt, J=7.80, 3.90 Hz, 1H), 6.83 (s, 1H), 7.42 (td, J=8.73, 2.50 Hz, 1H), 7.61 (dd, J=9.36, 2.50 Hz, 1H), 7.74 (s, 1H), 7.76 (s, 1H), 8.11 (dd, J=8.73, 6.24 Hz, 1H), 8.82 (d, J=8.11 Hz, 1H); MS (ESI, MeOH/NH4OH) m/z 470 [M+H]⁺, 468 [M−H]⁺.

### Example 136

### N-[1-(1-benzothien-3-ylmethyl)piperidin-4-yl]-7-fluoro-4-oxo-4H-chromene-2-carboxamide

Example 60C (19 mg, 0.065 mmol) and 1-benzothiophene-3-carbaldehyde were processed in a manner analogous to Example 40 to provide Example 136 (17 mg, 46%) as the title product. ¹H NMR (500 MHz, Pyridine-d5/Deuterium Oxide): δ ppm 2.17-2.35 (m, 4H), 2.52-2.63 (m, 1H), 2.73-2.87 (m, 1H), 3.29 (d, 2H, J=11.59 Hz), 4.11 (s, 2H), 4.27-4.40 (m, 2H), 7.31-7.35 (m, 1H), 7.34 (s, 1H), 7.45-7.53 (m, 0H), 7.53-7.59 (m, 1H), 7.63 (dd, 1H, J=9.18, 2.42 Hz), 7.83 (s, 1H), 7.99 (d, 1H, J=7.98 Hz), 8.11 (d, 1H, J=7.99 Hz), 8.31 (dd, 1H, J=8.88, 6.19 Hz); MS (ESI, MeOH/NH4OH) m/z 437 [M+H]⁺, 435[M−H]⁺.

### Example 137

### N-[1-(2-chloro-4-{[(2-piperidin-1-ylethyl)amino]carbonyl}benzyl)piperidin-4-yl]-7-fluoro-4-oxo-4H-chromene-2-carboxamide

Example 137 was prepared according to the procedure outlined in Example 80, substituting 2-piperidin 1-yl-ethylamine for 2-pyrrolidin 1-yl-ethylamine. ¹H NMR (500 MHz, DMSO-d₆) δ ppm 1.35-1.39(m, 2H), 1.46-1.51 (m, 4H), 1.64-1.70(m, 2H), 1.82(d, 2H, J=9.76), 2.19(t, 2H), 2.37-2.43(m, 6H), 2.87(d, 2H, J=11.90), 3.33-3.38(m, 2H), 3.62(s, 2H), 3.80-3.84(m, 1H), 6.83(s, 1H), 7.40-7.45(m, 1H), 7.57-7.62(m, 2H), 7.80(d, 1H, J=9.52), 7.89(s, 1H), 8.10-8.13 (m, 1H), 8.48(t, 1H), 8.85(d, 1H, J=7.93); MS (ESI) m/e 567.3 (M−H)⁺.

### Example 138

### N-[1-(1,3-benzodioxol-5-ylmethyl)piperidin-4-yl]-6,7,8-trifluoro-4-oxo-4H-chromene-2-carboxamide

### Example 138A

### 6,7,8-Trifluoro-4-oxo-4H-chromene-2-carboxylic acid

Example 138A was prepared using a similar procedure as described for Example 50B substituting 2,3,4-trifluoro phenol for 3,4-dichlorophenol in the step described in Example 50A. ¹H NMR (500 MHz, DMSO-d6) δ ppm 6.98 (s, 1H), 7.86-7.90 (m, 1H).

### Example 138

### N-[1-(1,3-benzodioxol-5-ylmethyl)piperidin-4-yl]-6,78-trifluoro-4-oxo-4H-chromene-2-carboxamide

Example 138 was prepared using a similar procedure as described for Example 49 substituting 6,7,8-trifluoro-4-oxo-4H-chromene-2-carboxylic acid for 7-chloro-6-cyano-4-oxo-4H-chromene-2-carboxylic acid in the step described in Example 49. ¹H NMR (500 MHz, DMSO-d6) δ ppm 1.65 (qd, 2H, J=11.88, 3.79 Hz), 1.74-1.80 (m, 2H), 1.95-2.06 (m, 2H), 2.81 (d, 2H, J=11.22 Hz), 3.38 (s, 2H), 3.70-3.83 (m, 1H), 5.99 (s, 2H), 6.73-6.76 (m, 1H), 6.83-6.86 (m, 2H), 6.93 (s, 1H), 7.85-7.90 (m, 1H), 8.85 (d, 1H, J=7.78 Hz); MS (ESI) m/z 461 [M+H]⁺.

### Example 139

### N-(1-{4-[acetyl(3-pyrrolidin-1-ylpropyl)amino]-3-fluorobenzyl}piperidin-4-yl)-7-fluoro-4-oxo-4H-chromene-2-carboxamide

A flask was charged with 7-fluoro-N-(1-{3-fluoro-4-[(3-pyrrolidin-1-ylpropyl)amino]benzyl}piperidin-4-yl)-4-oxo-4H-chromene-2-carboxamide as prepared in Example 81 (0.5 g, 1.0 mmol), TEA (0.7 mL, 5 mmol), and THF (10 mL). Solution was cooled to −78 C and acetyl chloride (0.132 mL, 1.9 mmol). Solution was stirred and allowed to slowly warm to room temperature. After 3 hours, solution was concentrated and purified on a RP-HPLC to provide N-(1-{4-[acetyl(3-pyrrolidin-1-ylpropyl)amino]-3-fluorobenzyl}piperidin-4-yl)-7-fluoro-4-oxo-4H-chromene-2-carboxamide. 1H NMR (300 MHz, DMSO-D6) δ ppm 1.75-1.91 (m, 8H) 1.95-2.08 (m, 5H) 2.98 (s, 3H) 3.10 (s, 1H) 3.18 (q, J=5.42 Hz, 4H) 3.52 (s, 4H) 3.96 (s, 1H) 4.31-4.41 (m, 1H) 6.85 (d, J=2.03 Hz, 1H) 7.44 (td, J=8.48, 2.03 Hz, 2H) 7.53-7.67 (m, 3H) 8.12 (dd, J=8.82, 6.44 Hz, 1H) 9.06-9.15 (m, 1H) 9.61 (s, 1H) MS (ESI) m/z 567 [M+H]⁺.

### Example 140

### N-{1-[4-({3-[(cyclohexylmethyl)amino]propanoyl}amino)-3-fluorobenzyl]piperidin-4-yl}-7-fluoro-4-oxo-4H-chromene-2-carboxamide

### Example 140A

### 7-Fluoro-4-oxo-4H-chromene-2-carboxylic acid {1-[4-(3-amino-propionylamino)-3-fluoro-benzyl]-piperidin-4-yl}-amide

A flask was charged with N-[1-(4-amino-3-fluorobenzyl)piperidin-4-yl]-7-fluoro-4-oxo-4H-chromene-2-carboxamide prepared as described in Example 90 (1.25 g, 2.0 mmol), 3-tert-Butoxycarbonylamino-propionic acid (0.4 g, 2.1 mmol), EDCI (0.4 g, 2.1 mmol), HOBT (0.3 g, 2.2 mmol), NMM (1 mL, 9.1 mmol), and DMF (6 mL). Solution was stirred at 55° C. for 16 hours. Solution was diluted with EA, washed with 1× saturated sodium bicarbonate, 1× brine, dried over Na₂SO₄, and purified on a FlashMaster II silica column using gradient conditions from 100% CH₂Cl₂ to 5% methanol/CH₂Cl₂. Product fractions were concentrated to dryness, taken up in CH₂Cl₂ (20 mL) and treated with TFA (4 mL) for 16 hours. Solution was concentrated to dryness and triturated with diethyl ether to give 7-Fluoro-4-oxo-4H-chromene-2-carboxylic acid {1-[4-(3-amino-propionylamino)-3-fluoro-benzyl]-piperidin-4-yl}-amide as a di-TFA salt. 1H NMR (300 MHz, DMSO-D6) δ ppm 1.87 (s, 2H) 2.06 (s, 2H) 2.79 (t, J=6.78 Hz, 2H) 3.09 (dd, J=12.38, 6.27 Hz, 4H) 3.43 (s, 3H) 4.28 (s, 2H) 7.31 (d, J=8.48 Hz, 1H) 7.44 (dt, J=7.04, 5.13 Hz, 2H) 7.61 (dd, J=9.49, 2.37 Hz, 1H) 7.75 (s, 2H) 8.01-8.17 (m, 2H) 9.09 (d, J=7.46 Hz, 1H) 9.75 (s, 1H) 10.14 (s, 1H) MS (ESIAPCI) m/z 485 [M+H]⁺.

### Example 140

### N-{1-[4-({3-[(cyclohexylmethyl)amino]propanoyl}amino)-3-fluorobenzyl]piperidin-4-yl}-7-fluoro-4-oxo-4H-chromene-2-carboxamide

A vial was charged with 7-Fluoro-4-oxo-4H-chromene-2-carboxylic acid {1-[4-(3-amino-propionylamino)-3-fluoro-benzyl]-piperidin-4-yl}-amide (30 mg, 0.04 mmol), cyclohexanecarboxaldehyde (0.004 mL, 0.04 mmol), PS-cyanoborohydride (150 mg, 0.45 mmol), and THF (2 mL). The mixture was shaken at room temperature for 16 hours, then filtered and purified on a RP-HPLC to give N-{1-[4-({3-[(cyclohexylmethyl)amino]propanoyl}amino)-3-fluorobenzyl]piperidin-4-yl}-7-fluoro-4-oxo-4H-chromene-2-carboxamide. 1H NMR (300 MHz, DMSO-D6) δ ppm 0.91-1.02 (m, 1H) 1.13-1.27 (m, 2H) 1.64-1.79 (m, 5H) 1.81-1.87 (m, 1H) 2.00-2.14 (m, 4H) 2.84 (dt, J=12.97, 6.91 Hz, 3H) 3.09-3.24 (m, 4H) 3.40 (s, 2H) 3.47 (s, 2H) 4.25-4.35 (m, 1H) 6.84-6.86 (m, 1H) 6.95-7.03 (m, 1H) 7.15 (dd, J=12.37, 1.86 Hz, 1H) 7.31 (d, J=8.14 Hz, 1H) 7.40-7.49 (m, 3H) 7.57-7.68 (m, 2H) 7.99-8.07 (m, 1H) 8.12 (dd, J=8.48, 6.78 Hz, 2H) 8.30 (s, 2H) 9.05 (t, J=7.63 Hz, 1H) 10.15 (s, 1H) MS (ESI) m/z 581 [M+H]⁺.

### Example 141

### 7-fluoro-4-oxo-N-{1-[1-(2-oxo-3-propyl-2,3-dihydro-1,3-benzothiazol-6-yl)ethyl]piperidin-4-yl}-4H-chromene-2-carboxamide

### Example 141A

### 6-Acetyl-3-propyl-3H-benzothiazol-2-one

A vial was charged with 6-Acetyl-3H-benzothiazol-2-one (50 mg, 0.26 mmol), 1-iodopropane (0.025 mL, 0.26 mmol), potassium carbonate (53 mg, 0.38 mmol), and DMF (0.4 mL). Mixture was shaken at 125° C. for 3 hours. Solution was filtered and purified on a FlashMaster II silica column using gradient conditions from 100% CH₂Cl₂ to 5% methanol/CH₂Cl₂ to give 6-Acetyl-3-propyl-3H-benzothiazol-2-one. MS (APCI) m/z 236 [M+H]⁺.

### Example 141

### 7-fluoro-4-oxo-N-{1-[1-(2-oxo-3-propyl-2,3-dihydro-1,3-benzothiazol-6-yl)ethyl]piperidin-4-yl}-4H-chromene-2-carboxamide

A vial was charged with 7-Fluoro-4-oxo-4H-chromene-2-carboxylic acid piperidin-4-ylamide (50 mg, 0.17 mmol), 6-Acetyl-3-propyl-3H-benzothiazol-2-one (41 mg, 0.17 mmol), titanium isopropoxide (0.25 mL, 0.85 mmol), and THF (0.5 mL). Solution was shaken at 50 C for 3 hr then cooled to room temperature. 1M Sodium Cyanoborohydride in THF (1 mL, 10 mmol) was added and solution was shaken at room temperature for 16 hr. Solution was treated with water (1 mL), filtered through celite, and purified on a RP-HPLC to give 7-fluoro-4-oxo-N-{1-[1-(2-oxo-3-propyl-2,3-dihydro-1,3-benzothiazol-6-yl)ethyl]piperidin-4-yl}-4H-chromene-2-carboxamide. 1HNMR (300 MHz, DMSO-D6) δ ppm 0.91 (t, J=7.46 Hz, 3H) 1.62-1.73 (m, 5H) 1.73-1.80 (m, 1H) 1.82-1.96 (m, 2H) 2.07 (s, 2H) 2.91-3.01 (m, 2H) 3.17 (s, 2H) 3.91-4.01 (m, 3H) 4.48-4.70 (m, 1H) 6.84-6.88 (m, 1H) 7.41-7.55 (m, 3H) 7.59-7.64 (m, 1H) 7.84 (s, 1H) 8.12 (dd, J=9.16, 6.44 Hz, 1H) 9.02 (d, J=7.80 Hz, 1H) MS (ESI) m/z 510 [M+H]⁺.

### Example 142

### 7-fluoro-4-oxo-N-[1-(4-{2-oxo-2-[(2-piperidin-1-ylethyl)amino]ethyl}benzyl)piperidin-4-yl]-4H-chromene-2-carboxamide

The title compound was prepared according to the procedure for example 129 substituting 2-piperidin-1-yl-ethylamine for 2-pyrrolidin-1-yl-ethylamine. ¹H NMR (300 MHz, DMSO-d₆) ppm 1.53-1.88 (m, 8H), 2.03-2.12 (m, 2H), 2.80-3.15 (m, 6H), 3.37-3.52 (m, 8H), 4.01 (m, 1H), 4.28 (m, 2H), 6.85 (s, 1H), 7.33-7.47 (m, 5H), 7.60 (dd, 1H, J₁=9.49, J₂=2.37), 8.12 (dd, 1H, J₁=8.81, J₂=6.44), 8.40 (m, 1H), 9.09 (d, 1H, J=7.46); MS (ESI) m/z 549 [M+H]⁺.

### Example 143

### N-[1-(2,1,3-benzothiadiazol-5-ylmethyl)piperidin-4-yl]-7-fluoro-4-oxo-4H-chromene-2-carboxamide

Example 60C (19 mg, 0.065 mmol), 2,1,3-benzothiadiazole-5-carbaldehyde were processed in a manner analogous to Example 40 to provide Example 143 (17 mg, 47%) as the title product. ¹H NMR (500 MHz, Pyridine-d5/Deuterium Oxide) δ ppm 2.21-2.34 (m, 4H), 2.37-2.44 (m, 2H), 3.15 (d, 2H, J=11.33 Hz), 3.87 (s, 2H), 4.28-4.41 (m, 1H), 7.31-7.35 (m, 1H), 7.36 (s, 1H), 7.67 (dd, 1H, J=9.18, 2.44 Hz), 7.86-7.89 (m, 1H), 8.06-8.08 (m, 2H), 8.32 (dd, 1H, J=8.88, 6.17 Hz); MS (ESI, MeOH/NH4OH) m/z 439 [M+H]⁺.

### Example 144

### 7-fluoro-4-oxo-N-{1-[(5-{[(2-piperidin-1-ylethyl)amino]carbonyl}thien-2-yl)methyl]piperidin-4-yl}-4H-chromene-2-carboxamide

### Example 144A

### 5-[(4-{[(7-fluoro-4-oxo-4H-chromen-2-yl carbonyl]amino}piperidin-1-yl)methyl]thiophene-2-carboxylic acid

A scintillation vial was charged with 7-fluoro-4-oxo-4H-chromene-2-carboxylic acid piperidin-4-ylamide hydrochloride (375 mg, 1.15 mmol), 5-formyl-thiophene-2-carboxylic acid (0.220 g, 1.40 mmol), 5.5 mL of a solution of DCE/MeOH (1% AcOH), and MP-CNBH₃ (0.767 g, 2 equiv, ˜3.0 mmol/g loading). The mixture was shaken vigorously for 18 hours, was filtered, and directly purified by RP-HPLC to provide the title compound as its bis-TFA salt. MS (ESI) m/z 431 [M+H]⁺.

### Example 144

### 7-fluoro-4-oxo-N-{1-[(5-{[(2-piperidin-1-ylethyl)amino]carbonyl}thien-2-yl)methyl]piperidin-4-yl}-4H-chromene-2-carboxamide

A portion of 144A (0.030 g, 0.071 mmol) was then dissolved in 2 mL of DMF in a scintillation flask and 2-piperidin-1-yl-ethylamine (12.3 mg, 0.085 mmol), HOBt (14.4 mg, 0.107 mmol), and EDCI (17.9 mg, 0.071 mmol) were added. The flask was placed on a heated shaker for 6 hours, and then cooled to room temperature. Evaporation of the solvents and purification via RP-HPLC provided the desired product. ¹H NMR (500 MHz, Pyridine-d5) δ ppm 1.28 (s, 2H), 1.45 (p, 4H, J=5.63 Hz), 1.79-1.93 (m, 2H), 2.02-2.08 (m, 4H), 2.33 (s, 4H), 2.58 (t, 2H, J=6.61 Hz), 2.90 (d, 2H, J=10.97 Hz), 3.61 (s, 2H), 3.74 (q, 2H, J=6.23 Hz), 4.19-4.29 (m, 1H), 6.65 (dd, 1H, J=9.31, 1.70 Hz), 6.90 (d, 1H, J=3.66 Hz), 7.16 (td, 1H, J=8.52, 2.41 Hz), 7.45 (s, 1H), 7.84 (d, 1H, J=3.66 Hz), 8.29 (dd, 1H, J=8.85, 6.41 Hz), 8.84 (t, 1H, J=5.53 Hz), 9.58 (d, 1H, J=7.78 Hz); MS (ESI) m/z 541 [M+H]⁺.

### Example 145

### 7-fluoro-N-[1-(3-fluoro-4-{[(2-pyrrolidin-1 ylethyl amino]carbonyl}benzyl)piperidin-4-yl]-4-oxo-4H-chromene-2-carboxamide

### Example 145A

### 2-fluoro-4-formyl-benzoic acid

To a mixture of 4-cyano-2-fluoro-benzoic acid (470 mg, 2.85 mmol) and NaH₂PO₂ monohydrate (2.00 g, 22.8 mmol) in pyridine (18 mL), AcOH (9 mL), and H₂O (9 mL) was added W-2 Raney-Nickel (650 mg) as a suspension in H₂O (0.5 mL). The mixture was stirred at room temperature until the starting material was consumed by HPLC analysis. The mixture was filtered through celite, eluting with MeOH. The eluent was concentrated under reduced pressure, and the residue was then partitioned between EtOAc (10 mL) and 1N HCl (5 mL). The layers were separated, and the organic was dried with anhydrous Na₂SO₄, filtered, and concentrated to provide the title compound as a solid that was used without further purification. MS (ESI) m/z 169 (N+H)⁺.

### Example 145B

### 2-fluoro-4-{4-[(7-fluoro-4-oxo-4H-chromene-2-carbonyl)-amino]-piperidin-1ylmethyl}-benzoic acid

Example 145B was prepared according to the procedure described in Example 79, substituting 4-[(7-fluoro-4-oxo-4H-chromene-2-carbonyl)-amino]-piperidine for 4-[(6-fluoro-7-chloro-4-oxo-4H-chromene-2-carbonyl)-amino]-piperidine, and 2-fluoro-4-formyl-benzoic acid for 3-fluoro-4-(3-piperidin-1-yl-propoxy)-benzaldehyde. The crude product obtained after filtration of the mixture and concentration of the eluent was used without further purification. MS (ESI) m/z 443 (M+H)⁺.

### Example 145

### 7-fluoro-N-[1-(3-fluoro-4-{[(2-pyrrolidin-1 ylethyl)amino]carbonyl}benzyl piperidin-4-yl]-4-oxo-4H-chromene-2-carboxamide

To a solution of 2-fluoro-4-{4-[(7-fluoro-4-oxo-4H-chromene-2-carbonyl)-amino]-piperidin-lylmethyl}-benzoic acid (240 mg, 0.543 mmol), 2-pyrrolidin-1-ylethylamine (0.068 mL, 0.543 mmol), NMM (0.135 mL, 1.09 mmol), and HOBt (92.0 mg, 0.679 mmol) in DMF (4 mL) was added EDCI (130 mg, 0.679 mmol), and the mixture was heated to 55° C. for 6 hours. The mixture was quenched by the addition of EtOAc (60 mL) and H₂O (15 mL). The layers were separated, and the organic was washed with H₂O (1×10 mL) and brine (3×10 mL). The organic layer was dried with anhydrous Na₂SO₄, filtered, and concentrated under reduced pressure. The residue was purified by MPLC on SiO₂ gel (20% Et₃N in EtOAc) to provide the title compound as a white solid. ¹H NMR (300 MHz, DMSO-d6) δ ppm 1.65-1.88 (m, 8H), 2.09 (br t, 2H, J=11.5 Hz), 2.53-2.73 (m, 4H), 2.83 (br d, 2H, J=11.5 Hz), 3.32-3.45 (m, 4H), 3.54 (s, 2H), 3.71-3.86 (m, 1H), 6.82 (s, 1H), 7.20 (d, 1H, J=6.4 Hz), 7.24 (s, 1H), 7.43 (dt, 1H, J=2.4 and 8.5 Hz), 7.59-7.65 (m, 2H), 8.11 (dd, 1H, J=6.5 and 9.2 Hz), 8.20 (br s, 1H), and 8.84 (d, 1H, J=7.8 Hz); MS (ESI) m/z 539 (M+H)⁺.

### Example 147

### N-{1-[4-({3-[(cyclopentylmethyl)amino]propanoyl}amino)-3-fluorobenzyl]piperidin-4-yl}-7-fluoro-4-oxo-4H-chromene-2-carboxamide

A vial was charged with 7-Fluoro-4-oxo-4H-chromene-2-carboxylic acid {1-[4-(3-amino-propionylamino)-3-fluoro-benzyl]-piperidin-4-yl}-amide (30 mg, 0.04 mmol), cyclohexanecarboxaldehyde (3.4 mg, 0.04 mmol), PS-cyanoborohydride (150 mg, 0.45 mmol), and THF (2 mL). The mixture was shaken at room temperature for 16 hours, then filtered and purified on a RP-HPLC to provide N-{1-[4-({3-[(cyclopentylmethyl)amino]propanoyl}amino)-3-fluorobenzyl]piperidin-4-yl}-7-fluoro-4-oxo-4H-chromene-2-carboxamide. 1H NMR (300 MHz, DMSO-D6) δ ppm 1.15-1.28 (m, 2H) 1.50-1.65 (m, 3H) 1.75-1.88 (m, 3H) 2.10 (m, 4H) 2.83-2.99 (m, 4H) 3.17-3.27 (m, 4H) 6.85 (s, 1H) 6.98 (m, 1H) 7.15 (m, 1H) 7.31 (d, J=8.82 Hz, 1H) 7.40-7.50 (m, 3H) 7.58-7.68 (m, 2H) 8.04 (t, J=8.31 Hz, 1H) 8.13 (dd, J=8.82, 6.44 Hz, 2H) 8.32 (s, 2H) 9.06 (t, J=7.46 Hz, 1H) 10.16 (s, 1H) MS (ESI) m/z 567 [M+H]⁺.

### Example 148

### N-[1-(1,3-benzodioxol-4-ylmethyl)piperidin-4-yl]-6-fluoro-7-[(4-methoxyphenyl)amino]-4-oxo-4H-chromene-2-carboxamide

Example 148 was prepared using a similar procedure as described for Example 124 substituting 4-methoxy phenylamine for 3,4-dimethoxy phenylamine in the step described in Example 124. ¹H NMR (500 MHz, DMSO-d6) δ ppm 1.75-1.85 (m, 2H), 1.98-2.05 (m, 2H), 3.00-3.10 (m, 2H), 3.37-3.43 (m, 2H), 3.79 (s, 3H), 3.90-3.99 (m, 1H), 4.19 (d, 2H, J=4.52 Hz), 6.08 (s, 2H), 6.69 (s, 1H), 6.94-6.99 (m, 2H), 7.02-7.05 (m, 2H), 7.06 (d, 1H, J=1.40 Hz), 7.26-7.29 (m, 2H), 7.62 (d, 1H, J=11.39 Hz), 8.71-8.72 (m, 1H), 8.93 (d, 1H, J=7.49 Hz), 9.31-9.37 (br s, 1H); MS (ESI) m/z 546 [M+H]⁺.

### Example 149

### 7-fluoro-4-oxo-N-{1-[1-(2-oxo-2,3-dihydro-1,3-benzoxazol-6-yl)ethyl]piperidin-4-yl}-4H-chromene-2-carboxamide

A mixture of 7-fluoro-4-oxo-4H-chromene-2-carboxylic acid piperidin-4-yl-amide (30.0 mg, 0.103 mmol), 6-acetyl-3H-benzoxazole-2-one (18.0 mg, 0.103 mmol), and Ti(OⁱPr)₄ (0.121 mL, 0.412 mmol) in THF (1 mL) and acetonitrile (1 mL) was heated to 70° C. for 5 hours. EtOH (0.5 mL) and NaBH(OAc)₃ (44.0 mg, 0.206 mmol) were added to the mixture, and the mixture was heated to 50° C. for an additional 2 hours. The mixture was quenched by the addition of saturated aqueous NaHCO₃ (5 mL) and EtOAc (5 mL). The layers were separated, and the aqueous was extracted with EtOAc (3×5 mL). The combined organic layers were dried with anhydrous Na₂SO₄, filtered, and concentrated under reduced pressure. The residue was purified by RP-HPLC to yield the title compound. ¹H NMR (300 MHz, DMSO-d6) δ ppm 1.68 (d, 3H, J=7.1 Hz), 1.76-1.92 (m, 2H), 1.99-2.13 (m, 2H), 2.85-3.00 (m, 2H), 3.30-3.37 (m, 2H), 3.64-3.73 (m, 1H), 4.50-4.62 (m, 1H), 6.85 (s, 1H), 7.19 (d, 1H, J=7.8 Hz), 7.29 (t, 1H, J=6.8 Hz), 7.14 (dt, 1H, J=2.4 and 8.8 Hz), 7.52 (s, 1H), 7.61 (dd, 1H, J=2.4 and 9.1 Hz), 8.13 (dd, 1H, J=6.4 and 8.8 Hz), 9.02 (d, 1H, J=7.5 Hz), and 11.87 (s, 1H); MS (ESI) m/z 452 (M+H)⁺.

### Example 150

### N-{1-[1-(3-ethyl-2-oxo-2,3-dihydro-1,3-benzoxazol-6-yl)ethyl]piperidin-4-yl}-7-fluoro-4-oxo-4H-chromene-2-carboxamide

### Example 150A

### 6-acetyl-3-ethyl-3H-benzoxazole-2-one

A suspension of 6-acetyl-3H-benzoxazole-2-one (100 mg, 0.565 mmol), K₂CO₃ (94 mg, 0.678 mmol), and EtI (0.052 mL, 0.678 mmol) in DMF (1 mL) was heated to 130° C. for 2 hours. After cooling to room temperature, the mixture was diluted with MeOH (2 mL) and filtered, eluting with additional MeOH (3 mL). The solution was concentrated under reduced pressure to provide the title compound as a beige solid that was used without further purification. MS (ESI) m/z 206 (M+H)⁺.

### Example 150

### N-{1-[1-(3-ethyl-2-oxo-2,3-dihydro-1,3-benzoxazol-6-yl ethyl]piperidin-4-yl}-7-fluoro-4-oxo-4H-chromene-2-carboxamide

A mixture of 7-fluoro-4-oxo-4H-chromene-2-carboxylic acid piperidin-4-ylamide (0.142 g, 0.488 mmol), 6-acetyl-3-ethyl-3H-benzoxazole-2-one (0.100 g, 0.488 mmol), and Ti(OⁱPr)₄ (0.571 mL, 1.95 mmol) in THF (5 mL) was heated to 70° C. for 5 hours. EtOH (1.0 mL) and NaBH(OAc)₃ (0.206 g, 0.980 mmol) were added to the mixture, and the mixture was heated to 50° C. for an additional 2 hours. The mixture was quenched by the addition of saturated aqueous NaHCO₃ (10 mL) and EtOAc (10 mL). The layers were separated, and the aqueous was extracted with EtOAc (3×10 mL). The combined organic layers were dried with anhydrous Na₂SO₄, filtered, and concentrated under reduced pressure. The residue was purified by MPLC (5% MeOH in EtOAc) to provide the title compound as a white solid. ¹H NMR (300 MHz, DMSO-d6) δ ppm 1.2 (t, 3H, J=7.1 Hz), 1.69 (d, 3H, J=7.1 Hz), 1.77-1.95 (m, 2H), 1.98-2.15 (m, 2H), 2.87-2.99 (m, 2H), 3.29-3.37 (m, 2H), 3.89 (q, 2H, J=7.1 Hz), 3.95-4.00 (m, 1H), 4.52-4.63 (m, 1H), 6.84 (s, 1H), 7.37-7.48 (m, 3H), 7.58 (m, 1H), 7.61 (dd, 1H, J=2.4 and 9.5 Hz), 8.12 (dd, 1H, J=6.4 and 8.8 Hz), and 9.03 (d, 1H, J=7.1 Hz); MS (ESI) m/z 480 (M+H)⁺.

### Example 151

### 7-fluoro-N-(1-{4-[({2-[(3R)-3-fluoropyrrolidin-1-yl]ethyl}amino)carbonyl]benzyl}piperidin-4-yl)-4-oxo-4H-chromene-2-carboxamide

The title product was prepared according to example 103 by substituting 2-(3-(R)-Fluoro-pyrrolidin-1-yl)-ethylamine for N1-Phenyl-ethane-1,2-diamine. MS (ESI) m/z 527 [M+H]⁺.

### Example 152

### N-[1-(1,3-benzodioxol-5-ylmethyl)piperidin-4-yl]-6-chloro-7-fluoro-4-oxo-4H-chromene-2-carboxamide

### Example 152A

### 4-chloro-3-fluorophenyl acetate

4-Chloro-3-fluorophenol (5.00 g, 34.1 mmol) was processed in a manner analogous to Example 55A to provide Example 152A (6.48 g, 100%) as a grainy oil. ¹H NMR (300 MHz, DMSO-D6): δ ppm 2.27 (s, 3H), 7.07 (ddd, J=8.81, 2.71, 1.36 Hz, 1H), 7.37 (dd, J=10.17, 2.37 Hz, 1H), 7.65 (t, J=8.65 Hz, 1H); MS (APCI) m/z 190 [³⁷C1+H]⁺.

### Example 152B

### 1-(5-chloro-4-fluoro-2-hydroxyphenyl)ethanone

Example 152A (6.48 g, 34.4 mmol) was processed in a manner analogous to Example 55B to provide Example 152B (4.95 g, 77% over two steps) as a brown solid. ¹H NMR (300 MHz, DMSO-D6): δ ppm 2.63 (s, 3H), 7.06 (d, J=10.85 Hz, 1H), 8.07 (d, J=8.48 Hz, 1H), 12.15 (s, 1H).

### Example 152C

### 6-chloro-7-fluoro-4-oxo-4H-chromene-2-carboxylic acid

Example 152A (4.95 g, 26.2 mmol) was processed in a manner analogous to Example 55C to provide Example 152C (1.47 g, 23%) as alight brown powder. ¹HNMR (300 MHz, DMSO-D6): δ ppm 6.95 (s, 1H), 8.05 (d, J=9.49 Hz, 1H), 8.18 (d, J=8.14 Hz, 1H); MS (DCI) m/z 243 [M+H]⁺, 260 [M+NH₄]⁺.

### Example 152D

### tert-butyl 4-{[(6-chloro-7-fluoro-4-oxo-4H-chromen-2-yl)carbonyl]amino}piperidine-1-carboxylate

Example 152C (1.47 g, 6.06 mmol) was processed in a manner analogous to Example 55D to give Example 152D (326 mg, 13%) as a yellow solid. ¹H NMR (300 MHz, DMSO-D6): δ ppm 1.38-1.52 (m, 11H), 1.81 (dd, J=12.89, 3.05 Hz, 2H), 2.77-2.96 (m, 2H), 3.91-4.05 (m, 3H), 6.87 (s, 1H), 7.85 (d, J=9.49 Hz, 1H), 8.17 (d, J=8.14 Hz, 1H), 8.86 (d, J=8.14 Hz, 1H); MS (ESI, MeOH/NH4OH) m/z 425 [M+H]⁺, 423 [M−H]⁺, 459 [M+Cl]⁺.

### Example 152E

### 6-chloro-7-fluoro-4-oxo-N-piperidin-4-yl-4H-chromene-2-carboxamide

Example 152D (320 mg, 0.753 mmol) was processed in a manner analogous to Example 55E to give Example 152E (228 mg, 93%) as a yellow solid. ¹H NMR (300 MHz, DMSO-D6): δ ppm 1.47 (td, J=11.87, 4.07 Hz, 2H), 1.75 (dd, J=11.70, 2.54 Hz, 2H), 2.08-2.25 (br. s, 1H), 2.46-2.55 (m, 2H), 2.93-3.01 (m, 2H), 3.82 (dd, J=7.63, 3.90 Hz, 1H), 6.86 (s, 1H), 7.87 (d, J=9.49 Hz, 1H), 8.17 (d, J=8.14 Hz, 1H), 8.83 (d, J=8.14 Hz, 1H); MS (ESI, MeOH/NH4OH) m/z 325 [M+H]⁺, 323 [M−H]⁺, 359 [M+Cl]⁺.

### Example 152

### N-[1-(1,3-benzodioxol-5-ylmethyl)piperidin-4-yl]-6-chloro-7-fluoro-4-oxo-4H-chromene-2-carboxamide

Example 152E (14 mg, 43.1 μmol) and piperonal (7 mg, 43.1 μmol) were processed in a manner analogous to Example 1 to give Example 152 (5 mg, 25%) as an off-white solid. ¹H NMR (500 MHz, DMSO-D6): δ ppm 1.62 (dd, J=12.63, 3.59 Hz, 2H), 1.77-1.82 (m, 2H), 1.98-2.04 (m, 2H), 2.80-2.85 (m, 2H), 3.39-3.45 (m, 3H), 5.98 (s, 2H), 6.75 (dd, J=7.80, 1.56 Hz, 1H), 6.83 (s, 1H), 6.85-6.86 (m, 2H), 7.86 (d, J=9.67 Hz, 1H), 8.17 (d, J=8.11 Hz, 1H), 8.83 (d, J=8.11 Hz, 1H); MS (ESI, MeOH/NH4OH) m/z 459 [M+H]⁺, 457 [M−H]⁺.

### Example 153

### 3-[(4-{[(7-fluoro-4-oxo-4H-chromen-2-yl)carbonyl]amino}piperidin-1-yl)methyl]phenyl methyl carbonate

### Example 153A

### 7-fluoro-N-[1-(3-hydroxybenzyl)piperidin-4-yl]-4-oxo-4H-chromene-2-carboxamide

A solution of example 60D (580 mg, 2 mmol) and 3-hydroxybenzaldehyde (244 mg, 2 mmol) in THF/2% acetic acid (8 mL) was charged with NaBH(OAc)₃ (844 mg, 4 mmol). The heterogeneous mixture was shaken for three days, quenched by slow addition of saturated NaHCO₃, diluted with CH₂Cl₂, washed with saturated aqueous NaHCO₃, distilled water, dried (Na₂SO₄) and concentrated. Purification by flash silica gel chromatography (0 to 10% MeOH/CH₂Cl₂) provided the title compound as a yellow foam (200 mg). MS (ESI, MeOH/NH₄OH) m/z 397 [M+H], 395 [M−H].

### Example 153

### 3-[(4-{[(7-fluoro-4-oxo-4H-chromen-2-yl)carbonyl]amino}piperidin-1-yl)methyl]phenyl methyl carbonate

A solution of example 153A (40 mg, 0.1 mmol) in dimethylformamide (0.4 mL) was charged with K₂CO₃ (21 mg, 0.15 mmol) followed by methyl chloroformate (8.5 uL, 0.11 mmol). After 20 hours, another aliquot of methyl chloroformate (9 uL, 0.11 mmol) was added. The mixture was stirred an additional 4 hours, quenched by the addition of distilled water (0.5 mL), CH₂Cl₂ (2 mL) was added, Na₂SO₄ added, filtered through a plug of silica (2 g SepPak), rinsed through with 50/50 heaxane/ethyl acetate (10 mL), collection tube changed, rinsed with 95/5 CH₂Cl₂/MeOH and concentrated. Purification by flash silica gel chromatography (0 to 100% ethyl acetate/hexane) provided the title compound as a yellow residue (6 mg). MS (ESI, MeOH/NH₄OH) m/z 455 [M+H], 453 [M−H]. ¹H NMR (300 MHz, DMSO-d6) δ ppm 1.58-1.74 (m, 2H), 1.78-1.84 (m, 2H), 2.00-2.13 (m, 2H), 2.89 (s, 2H), 3.52 (s, 2H), 3.73-3.85 (m, 1H), 3.83 (s, 3H), 6.82 (s, 1H), 7.07-7.14 (m, 1H), 7.17-7.19 (m, 1H), 7.23 (d, 1H, J=7.56 Hz), 7.38 (t, 1H, J=7.84 Hz), 7.43 (td, 1H, J=8.73, 2.46 Hz), 7.61 (dd, 1H, J=9.46, 2.45 Hz), 8.11 (dd, 1H, J=8.90, 6.37 Hz), 8.83 (d, 1H, J=7.90 Hz).

### Example 154

### N-[1-(3-bromobenzyl)piperidin-4-yl]-7-fluoro-4-oxo-4H-chromene-2-carboxamide

Example 60C (29 mg, 0.100 mmol), 3-bromobenzaldehyde (19 mg, 0.100 mmol), sodium triacetoxyborohydride (42 mg, 0.200 mmol), Na₂SO₄ (28 mg, 0.200 mmol), acetic acid (20 μL), and THF (1 mL) were processed as described in Example 1 to provide the title compound (27 mg, 59%) as an amber solid. ¹H NMR (300 MHz, DMSO-d6): δ ppm 1.58-1.73 (m, 2H), 1.75-1.87 (m, 2H), 2.01-2.12 (m, 2H), 2.83 (d, 2H, J=11.26 Hz), 3.50 (s, 2H), 3.70-3.87 (m, 1H), 6.82 (s, 1H), 7.21-7.38 (m, 2H), 7.38-7.56 (m, 3H), 7.61 (dd, 1H, J=9.47, 2.46 Hz), 8.11 (dd, 1H, J=8.90, 6.36 Hz), 8.83 (d, 1H, J=7.99 Hz); MS (ESI, MeOH/NH4OH) m/z 459 [⁷⁹Br+H]⁺, 461 [⁸¹Br+H]⁺, 457 [⁷⁹Br-H]⁺, 459 [⁸¹Br-H]⁺.

### Example 155

### N-[1-(1,3-benzodioxol-5-ylmethyl)piperidin-4-yl]-7-fluoro-N-methyl-4-oxo-4H-chromene-2-carboxamide

### Example 155A

### 7-fluoro-N-methyl-4-oxo-N-piperidin-4-yl-4H-chromene-2-carboxamide

A solution of example 60C (125 mg, 0.32 mmol) in dimethylformamide (1.6 mL) at 0° C. was charged with sodium hydride (60% dispersion in mineral oil, 16 mg, 0.4 mmol) (gas evolution). After 45 minutes, iodomethane (21 uL, 0.34 mmol) was added. The mixture was allowed to warm to room temperature and stirred overnight, quenched by slow addition of distilled water, diluted with ethyl acetate, washed with distilled water (2×), brine, dried (MgSO₄), filtered and concentrated to give a yellow residue (135 mg) which was carried forward without further purification. A solution of this yellow residue in CH₂Cl₂ (1 mL) at 0° C. was treated with trifluoroacetic acid (0.5 mL). The resulting yellow, homogeneous solution was allowed to warm to room temperature, stirred one hour and then concentrated to a brown syrup, dissolved in CH₂Cl₂, washed with aqueous K₂CO₃ (1M), dried (Na₂SO₄), filtered and concentrated to provide the title compound as a yellow solid (103 mg). MS (ESI, MeOH/NH4OH) m/z 305 [M+H].

### Example 155

### N-[1-(1,3-benzodioxol-5-ylmethyl)piperidin-4-yl]-7-fluoro-N-methyl-4-oxo-4H-chromene-2-carboxamide

A solution of Example 155A (31 mg) and piperonal (15 mg, 0.1 mmol) in CH₂Cl₂/2% AcOH (0.5 mL) was charged with NaBH(OAc)₃ (42 mg, 0.2 mmol). The resultant heterogeneous mixture turned homogeneous overnight and was quenched by slow addition of 3M H₂SO₄ (100 uL), shaken 5 minutes, basified to pH 10 with K₂CO₃ (1M), CH₂Cl₂ (2 mL) added and then added to a plug of silica (2 g Sep Pak), eluted with 50/50 hexane/ethyl acetate (10 mL), collection tube changed, eluted with 95/5 CH₂Cl₂/MeOH and concentrated. Purification by flash silica gel chromatography (0 to 100% ethyl acetate/hexane, then 0 to 5% MeOH/CH₂Cl₂) provided the title compound as a light yellow solid. MS (ESI, MeOH/NH₄OH) m/z 439 [M+H]. 1H NMR (300 MHz, DMSO-D6) δ ppm 1.6-2.1 (m, 7H), 2.8-3.0 (m, 4H), 3.4 (m, 2H), 4.12-4.26 (m, 1H) 5.75 (s, 1H) 5.98 (d, J=6.44 Hz, 2H) 6.58 (m, 1H), 6.65-6.9 (m, 2H), 7.4 (m, 1H), 7.7 (m, 1H), 8.12 (m, 1H).

### Example 157

### N-[(3S)-1-(1,3-benzodioxol-5-ylmethyl)pyrrolidin-3-yl]-7-methoxy-4-oxo-4H-chromene-2-carboxamide

Example 157 was prepared according to the procedure outlined in Example 2, substituting (4-Aminomethyl-cyclohexyl)-carbamic acid teroom temperature-butyl ester for 4-amino-1-BOC-piperidine in Example 2C. ¹H NMR (300 MHz, DMSO-d₆) δ ppm 2.12-2.48(m, 2H), 3.91-3.94(m, 4H), 4.24-4.66(m, 6H), 6.80(d, 1H, J=4.75), 6.99-7.05(m, 2H), 7.12-7.18(m, 3H), 7.95-7.99(m, 1H), 9.18-9.30(m, 1H); MS (ESI) m/e 423.1 (M+H)⁺.

1. A compound of formula (I), 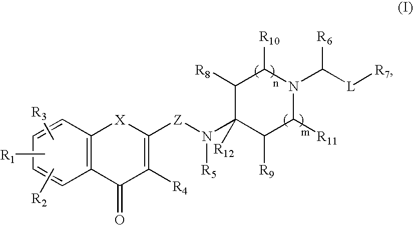
or a therapeutically acceptable salt or prodrug thereof, wherein
L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—;
X is selected from the group consisting of —O— and —N(R₁₃)—;
Z is selected from the group consisting of —CH₂—, —C(N—R_{c})—, —C(O)— and —C(S)—;
m is 1 or 2;
n is 0, 1, or 2
R₁, R₂ and R₃ are each independently selected from the group consisting of hydrogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylthio, alkynyl, cyano, halogen, haloalkyl, haloalkoxy, haloalkylthio, nitro, RₐR_{b}N—, RₐR_{b}NC(O)— or R₁ and R₂ taken together with any intervening atoms form a ring selected from the group consisting of heteroaryl and heterocycle;
R₄ is selected from the group consisting of hydrogen, alkyl, alkylcarbonyl-NH—, alkylsulfonyl-NH—, aryl and halogen;
R₅ is selected from the group consisting of hydrogen and alkyl;
R₆ is selected from the group consisting of hydrogen, alkyl, aryl, cycloalkyl, heteroaryl and heterocycle;
R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle;
R₈ is selected from the group consisting of hydrogen, alkyl and alkoxy;
R₉ is selected from the group consisting of hydrogen and alkyl;
R₁₀ and R₁₁ are independently selected from the group consisting of hydrogen, alkyl, alkoxylalkyl, or R₁₀ and R₁₁ taken together with any intervening atoms form a 5, 6, or 7-membered ring;
R₁₂ is selected from the group consisting of hydrogen and alkyl;
R₁₃ is selected from the group consisting of hydrogen, alkyl, aryl, cycloalkyl, heteroaryl and heterocycle;
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle; and
R_{c} is selected from the group consisting of hydrogen and alkyl;
provided that at least one of R₁, R₂ or R₃ are not hydrogen.

2. A compound of formula (II), 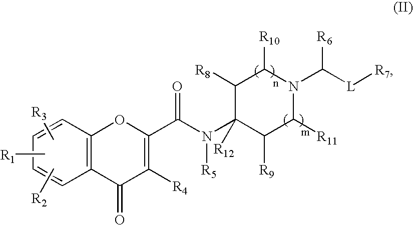
or a therapeutically acceptable salt or prodrug thereof, wherein
L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—;
m is 1 or 2;
n is 0, 1, or 2;
R₁, R₂ and R₃ are each independently selected from the group consisting of hydrogen, halogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N—, RₐR_{b}NC(O)— or R₁ and R₂ taken together with any intervening atoms form a heterocycle;
R₄ is selected from the group consisting of hydrogen, alkyl, alkylcarbonyl-NH—, alkylsulfonyl-NH—, aryl and halogen;
R₅ is selected from the group consisting of hydrogen and alkyl;
R₆ is selected from the group consisting of hydrogen, alkyl, aryl, cycloalkyl, heteroaryl and heterocycle;
R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle;
R₈ is selected from the group consisting of hydrogen, alkyl and alkoxy;
R₉ is selected from the group consisting of hydrogen and alkyl;
R₁₀ and R₁₁ are each independently selected from the group consisting of hydrogen, alkyl, alkoxylalkyl, or R₁₀ and R₁₁ taken together with any intervening atoms form a 5, 6, or 7-membered ring;
R₁₂ is selected from the group consisting of hydrogen and alkyl; and
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attacked form a ring selected from the group consisting of heteroaryl and heterocycle;
provided that at least one of R₁, R₂ or R₃ are not hydrogen.

3. A compound of formula (II), 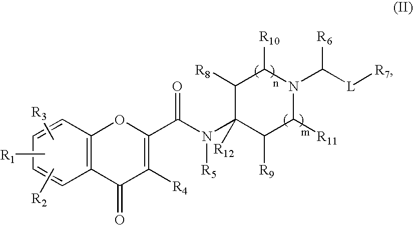
or a therapeutically acceptable salt or prodrug thereof, wherein
L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—;
m is 1;
n is 1;
R₁, R₂ and R₃ are each independently selected from the group consisting of hydrogen, halogen, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N—, RₐR_{b}NC(O)— or R₁ and R₂ taken together with any intervening atoms form a heterocycle;
R₄ is selected from the group consisting of hydrogen, alkyl, alkylcarbonyl-NH—, alkylsulfonyl-NH—, aryl and halogen;
R₅ is selected from the group consisting of hydrogen and alkyl;
R₆ is selected from the group consisting of hydrogen and alkyl;
R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle;
R₈ is selected from the group consisting of hydrogen, alkyl and alkoxy;
R₉ is selected from the group consisting of hydrogen and alkyl;
R₁₀ and R₁₁ are each independently selected from the group consisting of hydrogen, alkyl and alkoxylalkyl, or R₁₀ and R₁₁ taken together with any intervening atoms form a 5, 6, or 7-membered ring;
R₁₂ is hydrogen; and
Rₐ and R_{b} are each individually selected from the group consisting of hydrogen, alkoxycarbonyl, alky, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

4. A compound of formula (II), 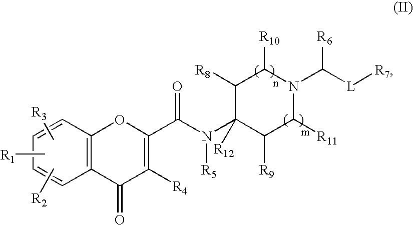
or a therapeutically acceptable salt or prodrug thereof, wherein
L is a bond;
m is 1;
n is 1;
R₁, R₂ and R₃ are each independently selected from the group consisting of hydrogen, halogen, alkoxy, alkyl, cyano, haloalkyl, haloalkoxy nitro, RₐR_{b}N— and RₐR_{b}NC(O)—;
R₄ is selected from the group consisting of hydrogen, alkyl, alkylcarbonyl-NH—, alkylsulfonyl-NH—, aryl and halogen;
R₅ is hydrogen;
R₆ is selected from the group consisting of hydrogen and alkyl;
R₇ is selected from the group consisting of aryl, heteroaryl and heterocycle, or R₆ and R₇ together with the atoms to which they are attached form cycloalkyl;
R₈ is hydrogen;
R₉ is selected from the group consisting of hydrogen and alkyl;
R₁₀ and R₁₁ are hydrogen;
R₁₂ is hydrogen; and
Rₐ and R_{b} are each individually selected from the group consisting of hydrogen, alkoxycarbonyl, alkyl, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

5. A compound of formula (II), 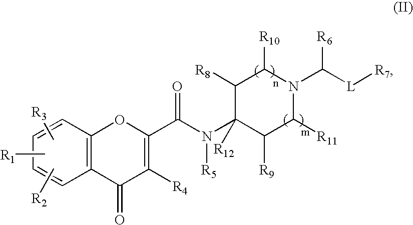
or a therapeutically acceptable salt or prodrug thereof, wherein
L is a bond;
m is 1;
n is 1;
R₁, R₂ and R₃ are each independently selected from the group consisting of hydrogen, halogen, alkoxy, alkyl, cyano, haloalkyl, haloalkoxy nitro, RₐR_{b}N— and RₐR_{b}NC(O)—;
R₄ is selected from the group consisting of hydrogen, alkyl, alkylcarbonyl-NH—, alkylsulfonyl-NH—, aryl and halogen;
R₅ is hydrogen;
R₆ is selected from the group consisting of hydrogen and alkyl;
R₇ is selected from the group consisting of aryl, heteroaryl and heterocycle;
R₈ is hydrogen;
R₉ is selected from the group consisting of hydrogen and alkyl;
R₁₀ and R₁₁ are hydrogen;
R₁₂ is hydrogen; and
Rₐ and R_{b} are each individually selected from the group consisting of hydrogen, alkoxycarbonyl, alkyl alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

6. A compound of formula (II), 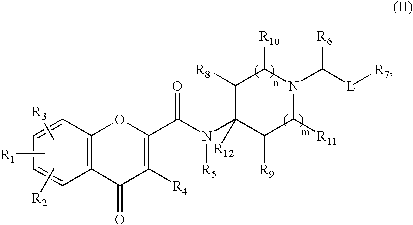
or a therapeutically acceptable salt or prodrug thereof, wherein
L is a bond;
m is 1;
n is 1;
R₁, R₂ and R₃ are each independently selected from the group consisting of hydrogen, halogen, alkoxy, alkyl, cyano, haloalkyl, haloalkoxy nitro, RₐR_{b}N— and RₐR_{b}NC(O)—;
R₄ is selected from the group consisting of hydrogen, alkyl alkylcarbonyl-NH—, alkylsulfonyl-NH—, aryl and halogen;
R₅ is hydrogen;
R₆ and R₇ together with the atoms to which they are attached form cycloalkyl;
R₈ is hydrogen;
R₉ is selected from the group consisting of hydrogen and alkyl;
R₁₀ and R₁₁ are hydrogen;
R₁₂ is hydrogen; and
Rₐ and R_{b} are each individually selected from the group consisting of hydrogen, alkoxycarbonyl, alkyl, alkylcarbonyl, alkyl-NH-carbonyl and alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

7. A compound of formula (III), 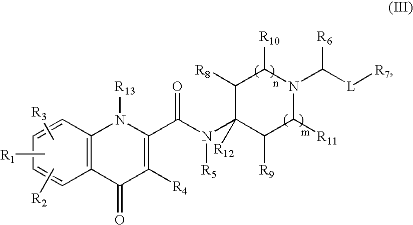
or a therapeutically acceptable salt or prodrug thereof, wherein
L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—;
m is 1 or 2;
n is 0, 1, or 2
R₁, R₂ and R₃ are each independently selected from the group consisting of hydrogen, halogen, alkenyl, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N— and RₐR_{b}NC(O)— or R₁ and R₂ taken together with any intervening atoms form a ring selected from the group consisting of heteroaryl and heterocycle;
R₄ is selected from the group consisting of hydrogen, alkyl, alkylcarbonyl-NH—, alkylsulfonyl-NH—, aryl and halogen;
R₅ is selected from the group consisting of hydrogen and alkyl;
R₆ is selected from the group consisting of hydrogen, alkyl, aryl, cycloalkyl, heteroaryl and heterocycle;
R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle;
R₈ is selected from the group consisting of hydrogen, alkyl and alkoxy;
R₉ is selected from the group consisting of hydrogen and alkyl;
R₁₀ and R₁₁ are each independently selected from the group consisting of hydrogen, alkyl and alkoxylalkyl, or R₁₀ and R₁₁ taken together with any intervening atoms form a 5, 6, or 7-membered ring;
R₁₂ is selected from the group consisting of hydrogen and alkyl;
R₁₃ is selected from the group consisting of hydrogen, alkyl, aryl, cycloalkyl, heteroaryl and heterocycle; and
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

8. A compound of formula (III), 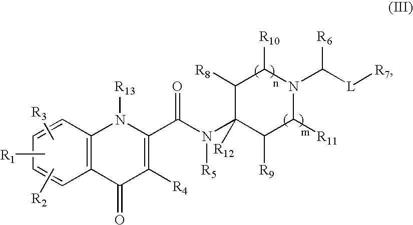
or a therapeutically acceptable salt or prodrug thereof, wherein
L is a bond or is selected from the group consisting of alkylene, alkenylene, alkynylene, —CH₂—O—, —S(O)₂—NH—, —C(O)—NH—, —NH—C(O)—, —NH—S(O)₂—, —C(O)—, —S(O)— and —S(O)₂—;
m is 1;
n is 1;
R₁, R₂ and R₃ are each independently selected from the group consisting of hydrogen, halogen, alkoxy, alkyl, alkylcarbonyl, alkylcarbonyl-NH—, alkyl-NH-carbonyl, alkylsulfonyl-NH—, alkyl-NH-sulfonyl, alkylsulfonyl, alkylsulfinyl, alkylthio, alkynyl, cyano, haloalkyl, haloalkoxy, haloalkylthio, nitro, RₐR_{b}N— and RₐR_{b}NC(O)— or R₁ and R₂ taken together with any intervening atoms form a ring selected from the group consisting of heteroaryl and heterocycle;
R₄ is selected from the group consisting of hydrogen, alkyl, alkylcarbonyl-NH—, alkylsulfonyl-NH—, aryl and halogen;
R₅ is selected from the group consisting of hydrogen and alkyl;
R₆ is selected from the group consisting of hydrogen and alkyl;
R₇ is selected from the group consisting of aryl, arylalkyl, heteroaryl, heteroarylalkyl, heterocycle and heterocyclealkyl, or R₆ and R₇ together with the atoms to which they are attached form a ring selected from the group consisting of cycloalkyl and heterocycle;
R₈ is selected from the group consisting of hydrogen, alkyl and alkoxy;
R₉ is selected from the group consisting of hydrogen and alkyl;
R₁₀ and R₁₁ are each independently selected from the group consisting of hydrogen, alkyl and alkoxylalkyl, or R₁₀ and R₁₁ taken together with any intervening atoms form a 5, 6, or 7-membered ring;
R₁₂ is hydrogen; and
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

9. A compound of formula (III), 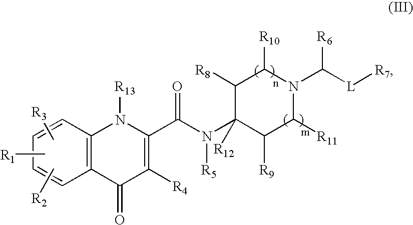
or a therapeutically acceptable salt or prodrug thereof, wherein
L is a bond;
m is 1;
n is 1;
R₁, R₂ and R₃ are each independently selected from the group consisting of hydrogen, halogen, alkoxy, alkyl, cyano, haloalkyl, haloalkoxy, nitro, RₐR_{b}N— and RₐR_{b}NC(O)—;
R₄ is selected from the group consisting of hydrogen, alkyl, alkylcarbonyl-NH—, alkylsulfonyl-NH—, aryl and halogen;
R₅ is hydrogen;
R₆ is selected from the group consisting of hydrogen and alkyl;
R₇ is selected from the group consisting of aryl, heteroaryl and heterocycle, or R₆ and R₇ together with the atoms to which they are attached form cycloalkyl;
R₈ is hydrogen;
R₉ is selected from the group consisting of hydrogen and alkyl;
R₁₀ and R₁₁ are hydrogen;
R₁₂ is hydrogen; and
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

10. A compound of formula (III), 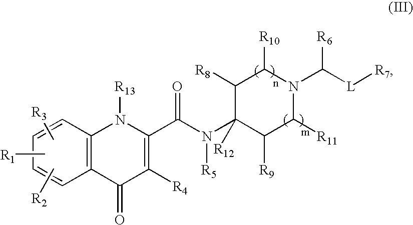
or a therapeutically acceptable salt or prodrug thereof, wherein
L is a bond;
m is 1;
n is 1;
R₁, R₂ and R₃ are each independently selected from the group consisting of hydrogen, halogen, alkoxy, alkyl, cyano, haloalkyl, haloalkoxy, nitro, RₐR_{b}N— and RₐR_{b}NC(O)—;
R₄ is selected from the group consisting of hydrogen, alkyl, alkylcarbonyl-NH—, alkylsulfonyl-NH—, aryl and halogen;
R₅ is hydrogen;
R₆ is selected from the group consisting of hydrogen and alkyl;
R₇ is selected from the group consisting of aryl, heteroaryl and heterocycle;
R₈ is hydrogen;
R₉ is selected from the group consisting of hydrogen and alkyl;
R₁₀ and R₁₁ are hydrogen;
R₁₂ is hydrogen; and
Rₐ and R_{b} are each individually selected from the group consisting of hydrogen, alkoxycarbonyl, alkyl alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

11. A compound of formula (III), 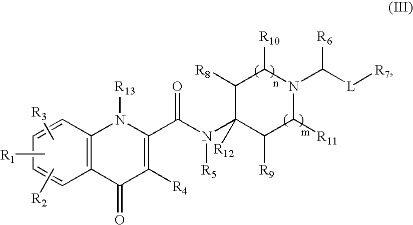
or a therapeutically acceptable salt or prodrug thereof, wherein
L is a bond;
m is 1;
n is 1;
R₁, R₂ and R₃ are each independently selected from the group consisting of hydrogen, halogen, alkoxy, alkyl, cyano, haloalkyl, haloalkoxy, nitro, RₐR_{b}N— and RₐR_{b}NC(O)—;
R₄ is selected from the group consisting of hydrogen, alkyl, alkylcarbonyl-NH—, alkylsulfonyl-NH—, aryl and halogen;
R₅ is hydrogen;
R₆ and R₇ together with the atoms to which they are attached form cycloalkyl;
R₈ is hydrogen;
R₉ is selected from the group consisting of hydrogen and alkyl;
R₁₀ and R₁₁ are hydrogen;
R₁₂ is hydrogen; and
Rₐ and R_{b} are each individually selected from the group consisting of hydrogen, alkoxycarbonyl, alkyl, alkylcarbonyl, alkyl-NH-carbonyl, alkylsulfonyl, aryl and arylalkyl or Rₐ and R_{b} taken together with the nitrogen to which they are attached form a ring selected from the group consisting of heteroaryl and heterocycle;
provided that at least one of R₁, R₂ or R₃ are not hydrogen.

12. The compound according to claim 1 that is selected from the group consisting of
N-[1-(1,3-benzodioxol-5-ylmethyl)piperidin-4-yl]-7-chloro-4-oxo-4H-chromene-2-carboxamide;
N-[1-(1,3-benzodioxol-5-ylmethyl)piperidin-4-yl]-7-methoxy-4-oxo-4H-chromene-2-carboxamide;
7-methoxy-4-oxo-N-[1-(1-quinolin-6-ylethyl)piperidin-4-yl]-4H-chromene-2-carboxamide;
7-chloro-N-[1-(2,3-dihydro-1,4-benzodioxin-6-ylmethyl)piperidin-4-yl]-4-oxo-4H-chromene-2-carboxamide;
7-methoxy-N-[1-(2-naphthylmethyl)piperidin-4-yl]-4-oxo-4H-chromene-2-carboxamide;
N-[1-(1,3-benzodioxol-5-ylmethyl)piperidin-4-yl]-6-fluoro-4-oxo-4H-chromene-2-carboxamide;
7-chloro-N-{1-[(1-methyl-1H-indol-5-yl)methyl]piperidin-4-yl}-4-oxo-4H-chromene-2-carboxamide;
N-[1-(1,3-benzodioxol-5-ylmethyl)piperidin-4-yl]-6-chloro-7-methyl-4-oxo-4H-chromene-2-carboxamide;
N-[1-(1,3-benzodioxol-5-ylmethyl)piperidin-4-yl]-7-(difluoromethoxy)-4-oxo-4H-chromene-2-carboxamide;
N-[1-(1,3-benzodioxol-5-ylmethyl)piperidin-4-yl]-7-chloro-3-methyl-4-oxo-4H-chromene-2-carboxamide;
7-chloro-4-oxo-N-[1-(1-quinolin-6-ylethyl)piperidin-4-yl]-4H-chromene-2-carboxamide;
N-[1-(1H-indol-5-ylmethyl)piperidin-4-yl]-7-methoxy-4-oxo-4H-chromene-2-carboxamide;
N-[1-(2,3-dihydro-1,4-benzodioxin-6-ylmethyl)piperidin-4-yl]-7-methoxy-4-oxo-4H-chromene-2-carboxamide;
N-{1-[(2,2-difluoro-1,3-benzodioxol-5-yl)methyl]piperidin-4-yl}-7-methoxy-4-oxo-4H-chromene-2-carboxamide;
7-methoxy-N-{1-[(1-methyl-1H-indol-5-yl)methyl]piperidin-4-yl}-4-oxo-4H-chromene-2-carboxamide;
N-[1-(1,3-benzodioxol-5-ylmethyl)piperidin-4-yl]-8-chloro-7-methoxy-4-oxo-4H-chromene-2-carboxamide;
N-[1-(3-fluoro-4-methoxybenzyl)piperidin-4-yl]-7-methoxy-4-oxo-4H-chromene-2-carboxamide;
N-[1-(4-chlorobenzyl)piperidin-4-yl]-7-methoxy-4-oxo-4H-chromene-2-carboxamide;
N-[1-(3-bromobenzyl)piperidin-4-yl]-7-methoxy-4-oxo-4H-chromene-2-carboxamide;
6-fluoro-N-{1-[(1-methyl-1H-indol-5-yl)methyl]piperidin-4-yl}-4-oxo-4H-chromene-2-carboxamide;
7-chloro-N-[1-(3-fluoro-4-methoxybenzyl)piperidin-4-yl]-4-oxo-4H-chromene-2-carboxamide;
N-{1-[(2,2-difluoro-1,3-benzodioxol-5-yl)methyl]piperidin-4-yl}-7-(difluoromethoxy)-4-oxo-4H-chromene-2-carboxamide;
N-[1-(2-fluoro-4-methoxybenzyl)piperidin-4-yl]-7-methoxy-4-oxo-4H-chromene-2-carboxamide;
7-chloro-4-oxo-N-[1-(quinolin-6-ylmethyl)piperidin-4-yl]-4H-chromene-2-carboxamide;
N-[1-(4-acetylbenzyl)piperidin-4-yl]-7-methoxy-4-oxo-4H-chromene-2-carboxamide;
7-methoxy-4-oxo-N-[1-(quinolin-6-ylmethyl)piperidin-4-yl]-4H-chromene-2-carboxamide;
N-[1-(1,3-benzodioxol-5-ylmethyl)piperidin-4-yl]-6-chloro-4-oxo-4H-chromene-2-carboxamide;
7-(difluoromethoxy)-4-oxo-N-[1-(1-quinolin-6-ylethyl)piperidin-4-yl]-4H-chromene-2-carboxamide;
N-[1-(4-bromobenzyl)piperidin-4-yl]-7-methoxy-4-oxo-4H-chromene-2-carboxamide;
N-[1-(1,3-benzodioxol-5-ylmethyl)piperidin-4-yl]-7-chloro-6-fluoro-4-oxo-1,4-dihydroquinoline-2-carboxamide;
7-chloro-6-fluoro-N-[1-(2-fluoro-4-methoxybenzyl)piperidin-4-yl]-4-oxo-1,4-dihydroquinoline-2-carboxamide;
N-[1-(1,3-benzodioxol-5-ylmethyl)piperidin-4-yl]-7-methoxy-4-oxo-1,4-dihydroquinoline-2-carboxamide;
N-[1-(1,3-benzodioxol-5-ylmethyl)piperidin-4-yl]-7-chloro-4-oxo-1,4-dihydroquinoline-2-carboxamide;
N-[1-(2-fluoro-4-methoxybenzyl)piperidin-4-yl]-4-oxo-7-(trifluoromethoxy)-1,4-dihydroquinoline-2-carboxamide;
N-[1-(1,3-benzodioxol-5-ylmethyl)piperidin-4-yl]-4-oxo-7-(trifluoromethoxy)-1,4-dihydroquinoline-2-carboxamide;
N-[1-(2-fluoro-4-methoxybenzyl)piperidin-4-yl]-7-methoxy-4-oxo-1,4-dihydroquinoline-2-carboxamide;
7-methoxy-N-[1-(4-methoxybenzyl)piperidin-4-yl]-4-oxo-4H-chromene-2-carboxamide;
7-methoxy-N-{1-[(5-methylthien-2-yl)methyl]piperidin-4-yl}-4-oxo-4H-chromene-2-carboxamide;
N-[1-(2-chlorobenzyl)piperidin-4-yl]-7-methoxy-4-oxo-4H-chromene-2-carboxamide;
N-[1-(2-bromobenzyl)piperidin-4-yl]-7-methoxy-4-oxo-4H-chromene-2-carboxamide;
N-{1-[(4-bromothien-2-yl)methyl]piperidin-4-yl}-7-methoxy-4-oxo-4H-chromene-2-carboxamide;
N-(1-benzylpiperidin-4-yl)-7-methoxy-4-oxo-4H-chromene-2-carboxamide;
7-methoxy-N-[1-(3-methoxybenzyl)piperidin-4-yl]-4-oxo-4H-chromene-2-carboxamide;
N-{1-[(4,5-dimethyl-2-furyl)methyl]piperidin-4-yl}-7-methoxy-4-oxo-4H-chromene-2-carboxamide;
N-[1-(3-chlorobenzyl)piperidin-4-yl]-7-methoxy-4-oxo-4H-chromene-2-carboxamide;
7-methoxy-N-[1-(1-naphthylmethyl)piperidin-4-yl]4-oxo-4H-chromene-2-carboxamide;
N-[1-(1,3-benzodioxol-5-ylmethyl)piperidin-4-yl]-7-chloro-6-cyano-4-oxo-4H-chromene-2-carboxamide;
N-[1-(1,3-benzothiazol-6-ylmethyl)piperidin-4-yl]-6,7-dichloro-4-oxo-4H-chromene-2-carboxamide;
N-[1-(1,3-benzoxazol-5-ylmethyl)piperidin-4-yl]-6,7-dichloro-4-oxo-4H-chromene-2-carboxamide;
N-[1-(1,3-benzodioxol-5-ylmethyl)piperidin-4-yl]-6-chloro-7-methoxy-4-oxo-4H-chromene-2-carboxamide;
6-chloro-N-{1-[(2,2-difluoro-1,3-benzodioxol-5-yl)methyl]piperidin-4-yl}-7-methoxy-4-oxo-4H-chromene-2-carboxamide;
N-[1-(1,3-benzodioxol-5-ylmethyl)piperidin-4-yl]-7-chloro-6-fluoro-4-oxo-4H-chromene-2-carboxamide;
N-[1-(1,3-benzodioxol-5-ylmethyl)piperidin-4-yl]-6,7-difluoro-4-oxo-4H-chromene-2-carboxamide;
N-[1-(1,3-benzodioxol-5-ylmethyl)piperidin-4-yl]-6-fluoro-7-methoxy-4-oxo-4H-chromene-2-carboxamide;
N-{1-[(1-ethyl-2-oxo-1,2-dihydroquinolin-7-yl)methyl]piperidin-4-yl}-7-fluoro-4-oxo-4H-chromene-2-carboxamide;
N-{1-[(5R)-6,7-dihydro-5H-indeno[5,6-d][1,3]dioxol-5-yl]piperidin-4-yl}-7-fluoro-4-oxo-4H-chromene-2-carboxamide;
N-[(3S,4S)-1-(1,3-benzodioxol-5-ylmethyl)-3-methylpiperidin-4-yl]-7-methoxy-4-oxo-4H-chromene-2-carboxamide;
N-[1-(1,3-benzodioxol-5-ylmethyl)piperidin-4-yl]-7-fluoro-4-oxo-4H-chromene-2-carboxamide;
N-{1-[(1R)-1-(2,2-difluoro-1,3-benzodioxol-5-yl)ethyl]piperidin-4-yl}-7-fluoro-4-oxo-4H-chromene-2-carboxamide;
N-[1-(1-benzofuran-5-ylmethyl)piperidin-4-yl]-7-fluoro-4-oxo-4H-chromene-2-carboxamide;
N-{1-[(6-chloro-1,3-benzodioxol-5-yl)methyl]piperidin-4-yl}-7-fluoro-4-oxo-4H-chromene-2-carboxamide;
N-{1-[(1R)-6-(acetylamino)-2,3-dihydro-1H-inden-1-yl]piperidin-4-yl}-7-fluoro-4-oxo-4H-chromene-2-carboxamide;
N-[1-(1,3-benzodioxol-5-ylmethyl)piperidin-4-yl]-7-bromo-6-fluoro-4-oxo-4H-chromene-2-carboxamide;
7-fluoro-4-oxo-N-{1-[(1S)-1-(2-oxo-2H-chromen-7-yl)ethyl]piperidin-4-yl}-4H-chromene-2-carboxamide;
N-{1-[6-(acetylamino)-2,3-dihydro-1H-inden-1-yl]piperidin-4-yl}-7-fluoro-4-oxo-4H-chromene-2-carboxamide;
N-[1-(1,3-benzodioxol-5-ylmethyl)piperidin-4-yl]-6-fluoro-7-methyl-4-oxo-4H-chromene-2-carboxamide;
7-fluoro-N-{1-[(1-methyl-2-oxo-1,2-dihydroquinolin-7-yl)methyl]piperidin-4-yl}-4-oxo-4H-chromene-2-carboxamide;
6,7-difluoro-N-{1-[(1-methyl-1H-indol-5-yl)methyl]piperidin-4-yl}4-oxo-4H-chromene-2-carboxamide;
7-fluoro-4-oxo-N-{1-[(2-oxo-2H-chromen-7-yl)methyl]piperidin-4-yl}-4H-chromene-2-carboxamide;
7-fluoro-N-[1-(4-methylbenzyl)piperidin-4-yl]-4-oxo-4H-chromene-2-carboxamide;
N-[1-(1-benzothien-5-ylmethyl)piperidin-4-yl]-7-fluoro-4-oxo-4H-chromene-2-carboxamide;
methyl 5-[(4-{[(7-fluoro-4-oxo-4H-chromen-2-yl)carbonyl]amino}piperidin-1-yl)methyl]-1H-indole-1-carboxylate;
7-fluoro-4-oxo-N-{1-[1-(2-oxo-2H-chromen-7-yl)ethyl]piperidin-4-yl}-4H-chromene-2-carboxamide;
N-[1-(1,3-benzodioxol-5-ylmethyl)piperidin-4-yl]-7-bromo-4-oxo-4H-chromene-2-carboxamide;
7-fluoro-4-oxo-N-[1-(1-quinolin-6-ylethyl)piperidin-4-yl]-4H-chromene-2-carboxamide;
2-({2-fluoro-4-[(4-{[(7-fluoro-4-oxo-4H-chromen-2-yl)carbonyl]amino}piperidin-1-yl)methyl]phenyl}amino)-2-oxoethyl acetate;
7-chloro-6-fluoro-N-{1-[3-fluoro-4-(3-piperidin-1-ylpropoxy)benzyl]piperidin-4-yl}-4-oxo-4H-chromene-2-carboxamide;
N-[1-(2-chloro-4-{[(2-pyrrolidin-1-ylethyl)amino]carbonyl}benzyl)piperidin-4-yl]-7-fluoro-4-oxo-4H-chromene-2-carboxamide;
7-fluoro-N-(1-{3-fluoro-4-[(3-pyrrolidin-1-ylpropyl)amino]benzyl}piperidin-4-yl)-4-oxo-4H-chromene-2-carboxamide;
7-fluoro-N-[1-(1H-indol-6-ylmethyl)piperidin-4-yl]-4-oxo-4H-chromene-2-carboxamide;
N-[1-(1,3-benzoxazol-6-ylmethyl)piperidin-4-yl]-7-fluoro-4-oxo-4H-chromene-2-carboxamide;
7-fluoro-N-{1-[3-fluoro-4-(3-piperidin-1-ylpropoxy)benzyl]piperidin-4-yl}-4-oxo-4H-chromene-2-carboxamide;
7-fluoro-4-oxo-N-[1-(3-oxo-2,3,4,6,7,8-hexahydroindeno[5,6-b][1,4]oxazin-6-yl)piperidin-4-yl]-4H-chromene-2-carboxamide;
N-[1-(2,3-dihydro-1-benzofuran-5-ylmethyl)piperidin-4-yl]-7-fluoro-4-oxo-4H-chromene-2-carboxamide;
7-fluoro-4-oxo-N-{1-[(2-oxo-1,2-dihydroquinolin-7-yl)methyl]piperidin-4-yl}-4H-chromene-2-carboxamide;
N-{1-[6-(acetylamino)-4-methyl-2,3-dihydro-1H-inden-1-yl]piperidin-4-yl}-7-fluoro-4-oxo-4H-chromene-2-carboxamide;
N-[1-(1,3-benzodioxol-5-ylmethyl)piperidin-4-yl]-7-ethyl-6-fluoro-4-oxo-4H-chromene-2-carboxamide;
N-[1-(4-amino-3-fluorobenzyl)piperidin-4-yl]-7-fluoro-4-oxo-4H-chromene-2-carboxamide;
N-{1-[1-(2,2-difluoro-1,3-benzodioxol-5-yl)ethyl]piperidin-4-yl}-7-fluoro-4-oxo-4H-chromene-2-carboxamide;
N-{1-[1-(3-ethyl-2-oxo-2,3-dihydro-1,3-benzothiazol-6-yl)ethyl]piperidin-4-yl}-7-fluoro-4-oxo-4H-chromene-2-carboxamide;
7-fluoro-4-oxo-N-{1-[6-(propionylamino)-2,3-dihydro-1H-inden-1-yl]piperidin-4-yl}-4H-chromene-2-carboxamide;
7-fluoro-N-{1-[1-(3-methyl-2-oxo-2,3-dihydro-1,3-benzoxazol-6-yl)ethyl]piperidin-4-yl}-4-oxo-4H-chromene-2-carboxamide;
7-fluoro-N-(1-{3-fluoro-4-[(3-piperidin-1-ylpropanoyl)amino]benzyl}piperidin-4-yl)₄-oxo-4H-chromene-2-carboxamide;
N-{1-[5-(acetylamino)-2-chlorobenzyl]piperidin-4-yl}-7-fluoro-4-oxo-4H-chromene-2-carboxamide;
ethyl 3-(4-{[(7-fluoro-4-oxo-4H-chromen-2-yl)carbonyl]amino}piperidin-1-yl)-2,3-dihydro-1H-inden-5-ylcarbamate;
7-fluoro-N-[1-(1H-indol-5-ylmethyl)piperidin-4-yl]-4-oxo-4H-chromene-2-carboxamide;
7-fluoro-4-oxo-N-[1-(5-{3-[(trifluoroacetyl)amino]propoxy}-2,3-dihydro-1H-inden-1-yl)piperidin-4-yl]-4H-chromene-2-carboxamide;
N-[1-(2,3-dihydro-1,4-benzodioxin-6-ylmethyl)piperidin-4-yl]-6-fluoro-7-methoxy-4-oxo-4H-chromene-2-carboxamide;
N-{1-[1-(6-chloro-1,3-benzodioxol-5-yl)ethyl]piperidin-4-yl}-7-fluoro-4-oxo-4H-chromene-2-carboxamide;
N-[1-(1,3-benzodioxol-5-ylmethyl)piperidin-4-yl]-6-fluoro-7-(isobutylamino)-4-oxo-4H-chromene-2-carboxamide;
N-[1-(4-{[(2-anilinoethyl)amino]carbonyl}benzyl)piperidin-4-yl]-7-fluoro-4-oxo-4H-chromene-2-carboxamide;
7-fluoro-4-oxo-N-{1-[(1S)-5-(3-piperidin-1-ylpropoxy)-2,3-dihydro-1H-inden-1-yl]piperidin-4-yl}-4H-chromene-2-carboxamide;
7-fluoro-N-(1-{3-fluoro-4-[(methoxyacetyl)amino]benzyl}piperidin-4-yl)-4-oxo-4H-chromene-2-carboxamide;
7-methoxy-4-oxo-N-{1-[(1S)-1-quinolin-6-ylethyl]piperidin-4-yl}-4H-chromene-2-carboxamide;
7-fluoro-N-[1-(3-fluoro-4-methoxybenzyl)piperidin-4-yl]-4-oxo-4H-chromene-2-carboxamide;
N-[1-(1,3-benzodioxol-5-ylmethyl)piperidin-4-yl]-6-fluoro-7-(isopropylamino)-4-oxo-4H-chromene-2-carboxamide;
ethyl {4-[(4-{[(7-fluoro-4-oxo-4H-chromen-2-yl)carbonyl]amino}piperidin-1-yl)methyl]phenyl}acetate;
N-(1-{4-[3-(dimethylamino)propoxy]benzyl}piperidin-4-yl)-7-fluoro-4-oxo-4H-chromene-2-carboxamide;
7-fluoro-4-oxo-N-[1-(4-{[(2-pyrrolidin-1-ylethyl)amino]carbonyl}benzyl)piperidin-4-yl]-4H-chromene-2-carboxamide;
N-[(3R,4S)-1-(1,3-benzodioxol-5-ylmethyl)-3-methylpiperidin-4-yl]-7-methoxy-4-oxo-4H-chromene-2-carboxamide;
7-fluoro-N-{1-[4-({[2-(3-fluorophenyl)ethyl]amino}carbonyl)benzyl]piperidin-4-yl}-4-oxo-4H-chromene-2-carboxamide;
7-fluoro-4-oxo-N-[1-(4-{[(2-piperidin-1-ylethyl)amino]carbonyl}benzyl)piperidin-4-yl]-4H-chromene-2-carboxamide;
N-[1-(3-amino-4-methylbenzyl)piperidin-4-yl]-7-fluoro-4-oxo-4H-chromene-2-carboxamide;
7-chloro-6-fluoro-4-oxo-N-{1-[(2E)-3-phenylprop-2-enyl]piperidin-4-yl}-4H-chromene-2-carboxamide;
N-[1-(1,3-benzodioxol-5-ylmethyl)piperidin-4-yl]-7-nitro-4-oxo-4H-chromene-2-carboxamide;
7-chloro-N-[1-(5,6-difluoro-2,3-dihydro-1H-inden-1-yl)piperidin-4-yl]-6-fluoro-4-oxo-4H-chromene-2-carboxamide;
N-[1-(2,1,3-benzothiadiazol-5-ylmethyl)piperidin-4-yl]-6,7-difluoro-4-oxo-4H-chromene-2-carboxamide;
7-fluoro-4-oxo-N-{1-[(3-oxo-3,4-dihydro-2H-1,4-benzoxazin-6-yl)methyl]piperidin-4-yl}-4H-chromene-2-carboxamide;
7-fluoro-4-oxo-N-{1-[4-({[2-(pyridin-2-ylamino)ethyl]amino}carbonyl)benzyl]piperidin-4-yl}-4H-chromene-2-carboxamide;
N-{1-[(1-acetyl-1H-indol-6-yl)methyl]piperidin-4-yl}-7-fluoro-4-oxo-4H-chromene-2-carboxamide;
7-fluoro-N-[1-(1-naphthylmethyl)piperidin-4-yl]-4-oxo-4H-chromene-2-carboxamide;
N-[1-(1,3-benzodioxol-4-ylmethyl)piperidin-4-yl]-7-[(3,4-dimethoxyphenyl)amino]-6-fluoro-4-oxo-4H-chromene-2-carboxamide;
7-chloro-N-{1-[(4-ethoxy-2-oxo-2H-chromen-7-yl)methyl]piperidin-4-yl}-6-fluoro-4-oxo-4H-chromene-2-carboxamide;
N-(1-{4-[amino(imino)methyl]benzyl}piperidin-4-yl)-7-fluoro-4-oxo-4H-chromene-2-carboxamide;
7-fluoro-4-oxo-N-[1-(2-oxo-3,5,6,7-tetrahydro-2H-indeno[5,6-d][1,3]oxazol-5-yl)piperidin-4-yl]-4H-chromene-2-carboxamide;
7-fluoro-N-{1-[(1-methyl-1H-indol-4-yl)methyl]piperidin-4-yl}-4-oxo-4H-chromene-2-carboxamide;
7-fluoro-4-oxo-N-[1-(4-{2-oxo-2-[(2-pyrrolidin-1-ylethyl)amino]ethyl}benzyl)piperidin-4-yl]-4H-chromene-2-carboxamide;
7-methoxy-4-oxo-N-[1-(quinolin-7-ylmethyl)piperidin-4-yl]-4H-chromene-2-carboxamide;
7-fluoro-4-oxo-N-{1-[(2E)-3-phenylprop-2-enyl]piperidin-4-yl}-4H-chromene-2-carboxamide;
N-[1-(1,3-benzodioxol-5-ylmethyl)piperidin-4-yl]-6-fluoro-7-(methylamino)-4-oxo-4H-chromene-2-carboxamide;
N-[1-(4-{[(2E)-2-(ethoxyimino)ethyl]oxy}-3-fluorobenzyl)piperidin-4-yl]-7-fluoro-4-oxo-4H-chromene-2-carboxamide;
N-[1-(1,3-benzodioxol-5-ylmethyl)piperidin-4-yl]-6-fluoro-4-oxo-7-{[4-(trifluoromethyl)benzyl]amino}4H-chromene-2-carboxamide;
N-{1-[(5-chloro-2-methyl-1,3-benzoxazol-6-yl)methyl]piperidin-4-yl}-7-fluoro-4-oxo-4H-chromene-2-carboxamide;
N-[1-(1-benzothien-3-ylmethyl)piperidin-4-yl]-7-fluoro-4-oxo-4H-chromene-2-carboxamide;
N-[1-(2-chloro-4-{[(2-piperidin-1-ylethyl)amino]carbonyl}benzyl)piperidin-4-yl]-7-fluoro-4-oxo-4H-chromene-2-carboxamide;
N-[1-(1,3-benzodioxol-5-ylmethyl)piperidin-4-yl]-6,7,8-trifluoro-4-oxo-4H-chromene-2-carboxamide;
N-(1-{4-[acetyl(3-pyrrolidin-1-ylpropyl)amino]-3-fluorobenzyl}piperidin-4-yl)-7-fluoro-4-oxo-4H-chromene-2-carboxamide;
N-{1-[4-({3-[(cyclohexylmethyl)amino]propanoyl}amino)-3-fluorobenzyl]piperidin-4-yl}-7-fluoro-4-oxo-4H-chromene-2-carboxamide;
7-fluoro-4-oxo-N-{1-[1-(2-oxo-3-propyl-2,3-dihydro-1,3-benzothiazol-6-yl)ethyl]piperidin-4-yl}-4H-chromene-2-carboxamide;
7-fluoro-4-oxo-N-[1-(4-{2-oxo-2-[(2-piperidin-1-ylethyl)amino]ethyl}benzyl)piperidin-4-yl]-4H-chromene-2-carboxamide;
N-[1-(2,1,3-benzothiadiazol-5-ylmethyl)piperidin-4-yl]-7-fluoro-4-oxo-4H-chromene-2-carboxamide;
7-fluoro-4-oxo-N-{1-[(5-{[(2-piperidin-1-ylethyl)amino]carbonyl}thien-2-yl)methyl]piperidin-4-yl}-4H-chromene-2-carboxamide;
7-fluoro-N-[1-(3-fluoro-4-{[(2-pyrrolidin-1-ylethyl)amino]carbonyl}benzyl)piperidin-4-yl]-4-oxo-4H-chromene-2-carboxamide;
N-{1-[4-({3-[(cyclopentylmethyl)amino]propanoyl}amino)-3-fluorobenzyl]piperidin-4-yl}-7-fluoro-4-oxo-4H-chromene-2-carboxamide;
N-[1-(1,3-benzodioxol-4-ylmethyl)piperidin-4-yl]-6-fluoro-7-[(4-methoxyphenyl)amino]-4-oxo-4H-chromene-2-carboxamide;
7-fluoro-4-oxo-N-{1-[1-(2-oxo-2,3-dihydro-1,3-benzoxazol-6-yl)ethyl]piperidin-4-yl}-4H-chromene-2-carboxamide;
N-{1-[1-(3-ethyl-2-oxo-2,3-dihydro-1,3-benzoxazol-6-yl)ethyl]piperidin-4-yl}-7-fluoro-4-oxo-4H-chromene-2-carboxamide;
7-fluoro-N-(1-{4-[({2-[(3R)-3-fluoropyrrolidin-1-yl]ethyl}amino)carbonyl]benzyl}piperidin-4-yl)-4-oxo-4H-chromene-2-carboxamide;
N-[1-(1,3-benzodioxol-5-ylmethyl)piperidin-4-yl]-6-chloro-7-fluoro-4-oxo-4H-chromene-2-carboxamide;
3-[(4-{[(7-fluoro-4-oxo-4H-chromen-2-yl)carbonyl]amino}piperidin-1-yl)methyl]phenyl methyl carbonate;
N-[1-(3-bromobenzyl)piperidin-4-yl]-7-fluoro-4-oxo-4H-chromene-2-carboxamide;
N-[1-(1,3-benzodioxol-5-ylmethyl)piperidin-4-yl]-7-fluoro-N-methyl-4-oxo-4H-chromene-2-carboxamide; and
N-[(3S)-1-(1,3-benzodioxol-5-ylmethyl)pyrrolidin-3-yl]-7-methoxy-4-oxo-4H-chromene-2-carboxamide.

13. A method of treating disorders by inhibiting the effects of melanin concentrating hormone (MCH) through the melanin concentrating hormone receptor, comprising administrering a therapeutically effective amount of a compound of formula (I).

14. A method of treating obesity by inhibiting the effects of melanin concentrating hormone (MCH) through the melanin concentrating hormone receptor, comprising administering a therapeutically effective amount of a compound of formula (I).

15. A method of treating abnormalities in reproduction and sexual behavior, thyroid hormone secretion, diuresis and water/electrolyte homeostasis, sensory processing, memory, sleeping and arousal, anxiety and depression, seizure and in treatment of neurodegeneration or psychiatric disorders by inhibiting the effects of melanin concentrating hormone (MCH) through the melanin concentrating hormone receptor, comprising administrering a therapeutically effective amount of a compound of formula (I).

16. A pharmaceutical composition comprising a therapeutically effective amount of a compound of formula (I) in combination with a pharmaceutically suitable carrier.
